(19) United States
(12) Patent Application Publication
(10) Pub. No.: US 2024/0327356 A1
(43) Pub. Date: Oct. 3, 2024

(54) PYRIMIDINE BASED RAS MODULATORS AND USES THEREOF

(71) Applicant: QUANTA THERAPEUTICS, INC., South San Francisco, CA (US)

(72) Inventors: Hong LIN, Exton, PA (US); Juan LUENGO, Phoenixville, PA (US)

(21) Appl. No.: 18/482,674

(22) Filed: Oct. 6, 2023

Related U.S. Application Data
(63) Continuation of application No. PCT/US22/24111, filed on Apr. 8, 2022.
(60) Provisional application No. 63/173,293, filed on Apr. 9, 2021.

(51) Int. Cl. C07D 239/26; A61P 35/00; C07D 403/04; C07D 403/14

(52) U.S. Cl. CPC C07D 239/26; C07D 403/04; C07D 403/14; A61P 35/00

(57) ABSTRACT

The disclosure provides pyrimidine based derivatives of Formula (I), (I-a), (I-b), (I-c), (I-d), (II), or (III), or salts thereof, for the modulation of Ras/Raf signaling. In another aspect, the present disclosure provides methods for the modulation of Ras/Raf signaling using pyrimidine based derivatives of Formula (I), (I-a), (I-b), (I-c), (I-d), (II), or (III), or salts thereof.

### CROSS-REFERENCE

This application is a continuation of International Patent Application PCT/US22/24111, filed on Apr. 8, 2022, which claims the benefit of U.S. Provisional Patent Application No. 63/173,293 filed on Apr. 9, 2021, each of which is incorporated herein by reference in its entirety.

### BACKGROUND OF THE INVENTION

Many different types of cancer cells manipulate the Ras/Raf and related signaling pathways for growth advantages partly due to the crosstalk with other fundamental pathways. Despite the major role played by mutant forms of Ras and Raf in human cancer, Ras is widely considered an undruggable target due to the picomolar affinity of GTP for Ras, GTP's role in several significant cellular processes unrelated to Ras as well as the critical role of protein-protein interactions between Ras and its accessory proteins (e.g., Raf) in a signaling cascade. There exists a need for RAS modulators capable of specifically moderating the behavior of Ras/Raf complexes in cancer cells to decrease the aberrant signaling of downstream Ras effector molecules.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In certain aspects, the present disclosure provides a compound represented by the structure of Formula (I):

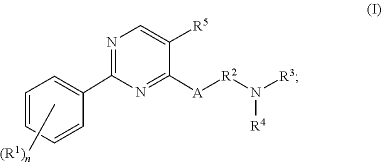

or a pharmaceutically acceptable salt thereof, wherein:
R¹ is independently selected at each occurrence from:
   halogen, —OR⁶, —SR⁶, —N(R⁶)₂, —C(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, and —CN;
   C₁₋₆ alkyl, C₂₋₆ alkenyl, and C₂₋₆ alkynyl, each of which is optionally substituted with one or more substituents independently selected from:
      halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN; and
      C₃₋₆ carbocycle and 3- to 6-membered heterocycle each of which is optionally substituted with one or more substituents independently selected from halogen, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and CN;
   C₃₋₆ carbocycle optionally substituted with one or more substituents independently selected from halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN;
   3 to 6-membered heterocycle optionally substituted with one or more substituents independently selected from halogen, C₁₋₆ alkyl, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and —CN; and
   two R¹ on adjacent carbon atoms can combine to form a C₄₋₆ carbocycle or 4- to 6-membered heterocycle together with the carbon atoms to which they are bound;
A is selected from a bond, —O—, —N(R¹¹)—, —S—, —S(═O)— and —S(═O)₂—, and a 4- to 9-membered heterocyclene optionally substituted with one or more R²;
R² is selected from C₁₋₄ alkylene, C₂₋₄ alkenylene, C₂₋₄ alkynylene, and C₃-C₇ carbocyclene and 4- to 9-membered heterocyclene, any of which are optionally substituted with one or more substituents independently selected from halogen, —OR⁷, —SR⁷, —N(R⁷)₂, —C(O)R⁷, C(O)OR⁷, —OC(O)R⁷, —OC(O)N(R⁷)₂, —C(O)N(R⁷)₂, —N(R⁷)C(O)R⁷, —N(R⁷)C(O)OR⁷, —N(R⁷)C(O)N(R⁷)₂, —N(R⁷)₂S(O)₂(R⁷), —S(O)R⁷, —S(O)₂R⁷, —S(O)₂N(R⁷)₂, —NO₂, ═O, and CN;
R³ and R⁴ are independently selected from hydrogen, C₁₋₃ alkyl, 3- to 7-membered heterocycle, and C₃-C₇ carbocycle, wherein C₁₋₃ alkyl, 3- to 7-membered heterocycle, and C₃-C₇ carbocycle, any of which are optionally substituted with one or more substituents independently selected from halogen, R⁸, —OR⁸, —SR⁸, —N(R)₂, —C(O)R⁸, —C(O)OR⁸, —OC(O)R⁸, —OC(O)N(R⁸)₂, —C(O)N(R⁸)₂, —N(R⁸)C(O)R⁸, —N(R⁸)C(O)OR⁸, —N(R⁸)C(O)N(R⁸)₂, —N(R⁸)₂S(O)₂(R⁸), —S(O)R⁸, —S(O)₂R, —S(O)₂N(R⁸)₂, —NO₂, and —CN; or
R³ and R⁴ can come together to form a 4- to 6-membered heterocycle optionally substituted with one or more substituents independently selected from:
   halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, and —CN; and
   C₁₋₆ alkyl optionally substituted with one or more substituents independently selected from halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, ═O, and —CN; or
R² and R⁴ can come together to form a 4- to 6-membered heterocycle optionally substituted with one or more substituents independently selected from:
   halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN; and
   C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN;
R⁵ is selected from:
   —N(R¹⁰)₂, —NO₂, —C(O)R¹⁰, —C(O)OR¹⁰, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), and —S(O)₂N(R¹⁰)₂;
   C₁ alkyl substituted with one or more substituents selected from halogen, R^{C}, R^{D}, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN;
   C₂₋₆ alkyl, C₂₋₆ alkenyl, and C₂₋₆ alkynyl, each of which is optionally substituted with one or more substituents independently selected from halogen, R^{C}, R^{D}, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and
   3- to 6-membered saturated heterocycle, 3- to 6-membered unsaturated heterocycle, C₃-C₆ saturated carbocycle, C₃-C₆ unsaturated carbocycle, —O—(C₁-C₆ alkyl), —O-(3- to 6-membered heterocycle), and —O—(C₃-C₆ carbocycle), each of which is optionally substituted with one or more substituents independently selected from:
      halogen, —OR¹⁰, —SR¹⁰, —N(R^{A})(R^{B}), —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN; and
      C₁₋₆ alkyl, C₂₋₅ alkenyl, C₂₋₅ alkynyl, each of which is optionally substituted with one or more substituents independently selected from halogen, —OR¹⁰, —SR¹⁰, —N(R¹⁰)₂, —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN;
R^{A} and R^{B} are each independently selected from:
   hydrogen, —OR¹⁸, —C(O)R¹⁸, —C(O)OR¹⁸, —C(O)N(R¹⁸)₂, —S(O)R¹⁸, —S(O)₂R¹⁸, and —S(O)₂N(R¹⁸)₂; and
   C₁₋₆ alkyl, C₂₋₆ alkenyl, C₂₋₆ alkynyl, C₃₋₆ carbocycle, and 3- to 6-membered heterocycle, any of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, and —CN; and
      C₃₋₆ carbocycle and 3- to 6-membered heterocycle each of which is optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, and CN; or
R^{A} and R^{B} can come together to form a 4- to 6-membered heterocycle optionally substituted with one or more substituents independently selected from:
   halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN; and
   C₁₋₆ alkyl optionally substituted with one or more substituents independently selected halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN;
R^{C} and R^{D} are each independently selected from C₁₋₆ alkyl, C₃₋₆ carbocycle, and 3- to 6-membered heterocycle, wherein C₁₋₆ alkyl, C₃₋₆ carbocycle, and 3- to 6-membered heterocycle are each optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)₂S(O)₂(R¹⁶), —S(O)R¹⁶, —S(O)₂R¹⁶, —S(O)₂N(R¹⁶)₂, —NO₂, ═O, and —CN; or
R^{C} and R^{D} can come together to form a 4- to 6-membered heterocycle optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN; and
   C₁₋₆ alkyl optionally substituted with one or more substituents independently selected halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN;
R¹⁸ is independently selected from:
   hydrogen;
   C₁₋₆ alkyl, C₂₋₆ alkenyl, C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
   C₃₋₆ carbocycle and 3- to 6-membered heterocycle each of which are optionally substituted with one or more substituents independently selected from:
      halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
      C₁₋₆ alkyl, C₂₋₆ alkenyl, and C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN;
each of R⁶, R⁷, R⁸, R⁹, R¹⁰, R¹¹, R¹², R¹⁵, R¹⁶, R¹⁷, R¹⁹, and R²² are independently selected at each occurrence from hydrogen, halogen, —OH, —NO₂, —CN, C₁₋₆ alkyl, C₁₋₆ haloalkyl, and C₃₋₆ carbocycle, 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ alkoxy, and C₁₋₆ haloalkyl; and
n is selected from 1, 2, and 3.

In certain aspects, Formula (I) is represented by Formula (I-a):

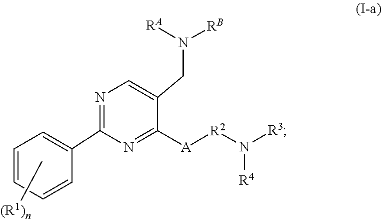

wherein, R^{A}, R^{B}, R¹, R², R³, R⁴, A and n are as described in Formula (I).

In certain aspects, the disclosure provides a pharmaceutical composition comprising a compound or salt of Formula (I) or Formula (I-a) and a pharmaceutically acceptable excipient.

In certain aspects, the disclosure provides a method of treating cancer comprising administering to a subject in need thereof an effective amount of a compound or salt of Formula (I) or Formula (I-a) or a pharmaceutically acceptable excipient thereof.

In certain aspects, the disclosure provides a method of modulating Ras/Raf signaling comprising administering to a subject in need thereof an effective amount of a compound or salt of Formula (I) or Formula (I-a) or a pharmaceutically acceptable excipient thereof.

In certain aspects, the disclosure provides a method of allosterically modulating Ras/Raf signaling comprising administering to a subject in need thereof an effective amount of a compound or salt of Formula (I) or Formula (I-a) or a pharmaceutically acceptable excipient thereof.

In certain aspects, the disclosure provides a method of inhibiting Ras/Raf signaling comprising administering to a subject in need thereof an effective amount of a compound or salt of Formula (I) or Formula (I-a) or a pharmaceutically acceptable excipient thereof.

In certain aspects, the methods disclosed herein further comprises a second cancer therapy selected from a chemotherapeutic, a targeted agent, and an immunotherapeutic.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to saturated hydrocarbon groups, including straight-chain alkyl and branched-chain alkyl groups that contain from 1 to 6 carbons. For example —C₁₋₆ alkyl- may be selected from methyl, ethyl, propyl, butyl, pentyl, and hexyl, any one of which is optionally substituted. The term —C_{x-y}alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

“Alkyl” as used herein refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkenyl” as used herein refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” as used herein refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” as used herein refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Halo” or “halogen” as used herein refers to halogen substituents such as bromo, chloro, fluoro and iodo substituents.

“Haloalkyl” as used herein refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, and any other suitable combinations of alkanes (or substituted alkanes) and halogens. When an alkyl group is substituted with more than one halogen radicals, each halogen may be independently selected, for example 1-chloro,2-bromoethane.

The term “carbocycle” as used herein refers to a saturated, unsaturated or aromatic ring in which each atom of the ring is carbon atom. Carbocycle includes 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene. The term “saturated cycloalkyl” as used herein refers to a saturated carbocycle. Exemplary saturated cycloalkyl rings include cyclopropyl, cyclohexyl, and norbornane. Carbocycles may be optionally substituted by one or more substituents such as those substituents described herein.

The term “C_{x-y} carbocycle” is meant to include groups that contain from x to y carbons in the cycle. For example, the term “C₃₋₆ carbocycle” refers to a saturated, unsaturated, or aromatic ring comprising from 3 to 6 carbons. For example —C₃₋₆ carbocycle- may be selected from cyclopropyl, cyclobutyl, cyclopentyl, cyclohexyl, and phenyl, any one of which is optionally substituted.

The term “carbocyclene” refers to a divalent ring, linking the rest of the molecule to a radical group, consisting solely of carbon and hydrogen atoms. The carbocyclene is attached to the rest of the molecule through a single bond and to the radical group through a single bond. The points of attachment of the carbocyclene are to the rest of the molecule and to the radical group are through any two carbons respectively. Carbocyclene includes arylene and cycloalkylene. The term therefore distinguishes carbocyclene from heterocyclene in which the divalent ring comprises at least one atom that is different from a carbon atom. The heterocyclene is attached to the rest of the molecule through a single bond and to the radical group through a single bond. The points of attachment of the heterocyclene are to the rest of the molecule and to the radical group through any two atoms respectively, valency permitting. Heterocyclene includes heteroarylene and heterocycloalkylene. Carbocyclene and heterocyclene may each be optionally substituted by one or more substituents such as those substituents described herein.

“Aryl” as used herein refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “heterocycle” as used herein refers to a saturated, unsaturated or aromatic ring comprising one or more heteroatoms. Exemplary heteroatoms include N, O, Si, P, B, and S atoms. The heterocycle may be attached to the rest of the molecule through any atom of the heterocycle, valence permitting, such as a carbon or nitrogen atom of the heterocycle Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. Heterocycles may be optionally substituted by one or more substituents such as those substituents described herein.

“Heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

“Substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)R^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

The term “effective amount” or “therapeutically effective amount” refers to that amount of a compound or salt described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, “sum frequency generation” (SFG) is a nonlinear, optical technique whereby light at one frequency (Ω1) is mixed with light at another frequency (Ω2) to yield a response at the sum frequency (Ω1+Ω2) (Shen, 1984, 1989). For example, SFG is particularly useful for the detection of molecules at surfaces through their characteristic vibrational transitions and, in this case, is essentially a surface-selective infrared spectroscopy with Ω1 and Ω2 at visible and infrared frequencies. When the terms “SHG” or “second harmonic generation” are used herein, it is understood that SFG and “sum frequency generation” can substitute and be used in place of SHG with methods well known to one skilled in the art.

“Second harmonic-active moiety” or “second harmonic-active moiety,” as used herein, refers to a nonlinear-active moiety, particle or molecule which can be attached (covalently or non-covalently) to a molecule (e.g., a protein, such as an enzyme), particle or phase (e.g., lipid bilayer) in order to render it more nonlinear optical active.

A “nonlinear active moiety,” as used herein, is a substance which possesses a hyperpolarizability.

“Hyperpolarizability” or “Nonlinear Susceptibility” as used herein refer to the properties of a molecule, particle, interface, or phase which allow for generation of nonlinear light. The terms “hyperpolarizability,” “second-order nonlinear polarizability,” and “nonlinear susceptibility” are some-times used interchangeably.

“Allosteric”, “allosteric modulator”, or “allosteric candidate” as used herein, refers to a molecule, moiety or substance which binds predominantly to a site other than the active site and causes conformational change, which can be determined by SHG or SFG, and thus exert their effect via an allosteric mechanism of action.

The term “inhibit”, “selective inhibition” or “selectively inhibit” as referred to a biologically active agent refers to the agent's ability to preferentially reduce the target signaling activity as compared to off-target signaling activity, via direct or interact interaction with the target.

The term “targeted agent” or “targeted therapy” as described herein referred to a therapy that uses specific drugs to target specific genes, protein, or active sites involved in the development, survival, and proliferation of cancer cells.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The section headings used herein are for organizational purposes only and are not to be construed as limiting the subject matter described.

### Compounds

In certain aspects, the present disclosure provides a compound represented by the structure of Formula (I):

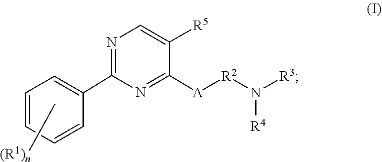

or a pharmaceutically acceptable salt thereof, wherein:
R¹ is independently selected at each occurrence from:
   halogen, —OR⁶, —SR⁶, —N(R⁶)₂, —C(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, and —CN;
   C₁₋₆ alkyl, C₂₋₆ alkenyl, and C₂₋₆ alkynyl, each of which is optionally substituted with one or more substituents independently selected from:
      halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN; and
      C₃₋₆ carbocycle and 3- to 6-membered heterocycle each of which is optionally substituted with one or more substituents independently selected from halogen, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and CN;
   C₃₋₆ carbocycle optionally substituted with one or more substituents independently selected from halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN;
   3 to 6-membered heterocycle optionally substituted with one or more substituents independently selected from halogen, C₁₋₆ alkyl, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and —CN; and
   two R¹ on adjacent carbon atoms can combine to form a C₄₋₆ carbocycle or 4- to 6-membered heterocycle together with the carbon atoms to which they are bound;
A is selected from a bond, —O—, —N(R¹¹)—, —S—, —S(═O)— and —S(═O)₂—, and a 4- to 9-membered heterocyclene optionally substituted with one or more R²;
R² is selected from C₁₋₄ alkylene, C₂₋₄ alkenylene, C₂₋₄ alkynylene, and C₃-C₇ carbocyclene and 4- to 9-membered heterocyclene, any of which are optionally substituted with one or more substituents independently selected from halogen, —OR⁷, —SR⁷, —N(R⁷)₂, —C(O)R⁷, C(O)OR⁷, —OC(O)R⁷, —OC(O)N(R⁷)₂, —C(O)N(R⁷)₂, —N(R⁷)C(O)R⁷, —N(R⁷)C(O)OR⁷, —N(R⁷)C(O)N(R⁷)₂, —N(R⁷)₂S(O)₂(R⁷), —S(O)R⁷, —S(O)₂R⁷, —S(O)₂N(R⁷)₂, —NO₂, ═O, and CN;
R³ and R⁴ are independently selected from hydrogen, C₁₋₃ alkyl, 3- to 7-membered heterocycle, and C₃-C₇ carbocycle, wherein C₁₋₃ alkyl, 3- to 7-membered heterocycle, and C₃-C₇ carbocycle, any of which are optionally substituted with one or more substituents independently selected from halogen, R⁸, —OR⁸, —SR⁸, —N(R)₂, —C(O)R⁸, —C(O)OR⁸, —OC(O)R⁸, —OC(O)N(R⁸)₂, —C(O)N(R⁸)₂, —N(R)C(O)R⁸, —N(R⁸)C(O)OR⁸, —N(R⁸)C(O)N(R⁸)₂, —N(R⁸)₂S(O)₂(R⁸), —S(O)R⁸, —S(O)₂R⁸, —S(O)₂N(R⁸)₂, —NO₂, and —CN; or
R³ and R⁴ can come together to form a 4- to 6-membered heterocycle optionally substituted with one or more substituents independently selected from:
   halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, and —CN; and
   C₁₋₆ alkyl optionally substituted with one or more substituents independently selected from halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, ═O, and —CN; or
R² and R⁴ can come together to form a 4- to 6-membered heterocycle optionally substituted with one or more substituents independently selected from:
   halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN; and
   C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN;
R⁵ is selected from:
   —N(R¹⁰)₂, —NO₂, —C(O)R¹⁰, —C(O)OR¹⁰, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), and —S(O)₂N(R¹⁰)₂;
   C₁ alkyl substituted with one or more substituents selected from halogen, R^{C}, R^{D}, R¹⁰, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN;
   C₂₋₆ alkyl, C₂₋₆ alkenyl, and C₂₋₆ alkynyl, each of which is optionally substituted with one or more substituents independently selected from halogen, R^{C}, R^{D}, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and
   3- to 6-membered saturated heterocycle, 3- to 6-membered unsaturated heterocycle, C₃-C₆ saturated carbocycle, C₃-C₆ unsaturated carbocycle, —O—(C₁-C₆ alkyl), —O-(3- to 6-membered heterocycle), and —O—(C₃-C₆ carbocycle), each of which is optionally substituted with one or more substituents independently selected from:
      halogen, —OR¹⁰, —SR¹⁰, —N(R^{A})(R^{B}), —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN; and
      C₁₋₆ alkyl, C₂₋₅ alkenyl, C₂₋₅ alkynyl, each of which is optionally substituted with one or more substituents independently selected from halogen, —OR¹⁰, —SR¹⁰, —N(R¹⁰)₂, —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN;
R^{A} and R^{B} are each independently selected from:
   hydrogen, —OR¹⁸, —C(O)R¹⁸, —C(O)OR¹⁸, —C(O)N(R¹⁸)₂, —S(O)R¹⁸, —S(O)₂R¹⁸, and —S(O)₂N(R¹⁸)₂; and
   C₁₋₆ alkyl, C₂₋₆ alkenyl, C₂₋₆ alkynyl, C₃₋₆ carbocycle, and 3- to 6-membered heterocycle, any of which is optionally substituted with one or more substituents independently selected from:
      halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, and —CN; and
      C₃₋₆ carbocycle and 3- to 6-membered heterocycle each of which is optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, and CN; or
R^{A} and R^{B} can come together to form a 4- to 6-membered heterocycle optionally substituted with one or more substituents independently selected from:
   halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN; and
   C₁₋₆ alkyl optionally substituted with one or more substituents independently selected halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN;
R^{C} and R^{D} are each independently selected from C₁₋₆ alkyl, C₃₋₆ carbocycle, and 3- to 6-membered heterocycle, wherein C₁₋₆ alkyl, C₃₋₆ carbocycle, and 3- to 6-membered heterocycle are each optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)₂S(O)₂(R¹⁶), —S(O)R¹⁶, —S(O)₂R¹⁶, —S(O)₂N(R¹⁶)₂, —NO₂, ═O, and —CN; or
R^{C} and R^{D} can come together to form a 4- to 6-membered heterocycle optionally substituted with one or more substituents independently selected from:
   halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN; and
   C₁₋₆ alkyl optionally substituted with one or more substituents independently selected halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN;
R¹⁸ is independently selected from:
   hydrogen;
   C₁₋₆ alkyl, C₂₋₆ alkenyl, C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
   C₃₋₆ carbocycle and 3- to 6-membered heterocycle each of which are optionally substituted with one or more substituents independently selected from:
      halogen, R²², —OR²², —SR²², —N(R²²)₂, —C(O)R²², —C(O)OR²², —OC(O)R²², OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
      C₁₋₆ alkyl, C₂₋₆ alkenyl, and C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN;
each of R⁶, R⁷, R⁸, R⁹, R¹⁰, R¹¹, R¹², R¹⁵, R¹⁶, R¹⁷, R¹⁹, and R²² are independently selected at each occurrence from hydrogen, halogen, —OH, —NO₂, —CN, C₁₋₆ alkyl, C₁₋₆ haloalkyl, and C₃₋₆ carbocycle, 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ alkoxy, and C₁₋₆ haloalkyl; and
n is selected from 1, 2, and 3.

In some embodiments, for the compound or salt of Formula (I), n is selected from 1 and 2. In some embodiments, n is selected from 2 and 3. In some embodiments, n is 1. In some embodiments, n is 2. In some embodiments, n is 3. In some embodiments, n is selected from 2 and 3; and R¹ is not 2,6-substituted around the phenyl ring.

In some embodiments, for the compound or salt of Formula (I), R¹ is independently selected from halogen, —CN, —N(R⁶)C(O)R⁶; and C₁₋₆ alkyl optionally substituted with one or more substituents independently selected from halogen, —OR⁶, —SR⁶, —N(R⁶)₂, —C(O)R⁶, —C(O)OR⁶, —OC(O)R⁶, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and —CN. In some embodiments, R¹ is independently selected from halogen, —C(O)N(R⁶)₂, —CN, C₁₋₆ alkyl, and C₁₋₆ haloalkyl. In some embodiments, R¹ is selected from chloro, fluoro, methyl, CHF₂, CF₃, —CN,

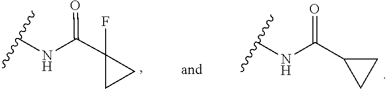 [SMILES: *NC(=O)C1(F)CC1 ; *NC(=O)C1CC1]

In some embodiments, R¹ is selected from chloro, fluoro, methyl, CHF₂, CF₃, and —CN. In some embodiments, R¹ is selected from

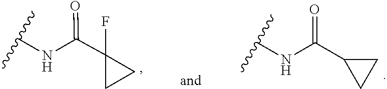 [SMILES: *NC(=O)C1(F)CC1 ; *NC(=O)C1CC1]

In some embodiments, for the compound or salt of Formula (I), R¹ is independently selected from:
halogen, —OR⁶, —C(O)N(R⁶)₂, —CN, and —NO₂;
C₁₋₆ alkyl, C₂₋₆ alkenyl, and C₂₋₆ alkynyl, each of which is optionally substituted with one or more substituents independently selected from halogen, —OR⁶, —C(O)N(R⁶)₂, —CN,
   —NO₂, and C₃₋₆ carbocycle; and
C₃₋₆ carbocycle optionally substituted with one or more substituents independently selected from halogen, —OR⁶, —C(O)N(R⁶)₂, —CN, and —NO₂.

In some embodiments, R¹ is selected from chloro, fluoro, methyl, ethyne, CHF₂, CF₃, —O—CF₃, —CN, —NO₂

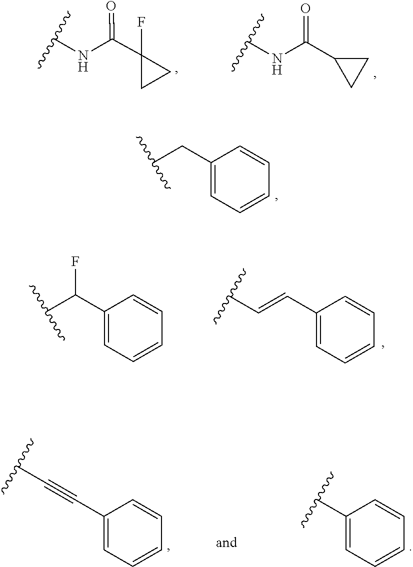

In some embodiments, for the compound or salt of Formula (I), A is selected from a bond, —O—, and 4- to 9-membered heterocyclene optionally substituted with one or more R¹². In some embodiments, A is selected from —N(R¹¹)—, —S—, —S(═O)— and —S(═O)₂—, In some embodiments, A is a bond. In some embodiments, A is —O—. In some embodiments, A is 4- to 9-membered heterocyclene optionally substituted with one or more R¹². In some embodiments, A is 4- to 9-membered saturated heterocyclene optionally substituted with one or more R¹². In some embodiments, A is 4- to 9-membered unsaturated heterocyclene optionally substituted with one or more R¹². In some embodiments, A is selected from a bond, —O—, and 5-membered saturated heterocyclene optionally substituted with one or more R¹².

In some embodiments, for the compound or salt of Formula (I), R² is selected from C₁₋₃ alkylene and 5- to 6-membered saturated heterocyclene each of which is optionally substituted with one or more substituents selected from —OH and NH₂.

In some embodiments, for the compound or salt of Formula (I), R³ and R⁴ are each independently selected from hydrogen, C₁₋₃ alkyl, and C₁₋₃ haloalkyl. In some embodiments, R³ and R⁴ are each independently hydrogen. In some embodiments, R³ and R⁴ are each independently from C₁₋₃ alkyl and C₁₋₃ haloalkyl.

In some embodiments, for the compound or salt of Formula (I),

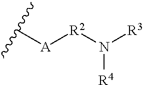

is selected from:

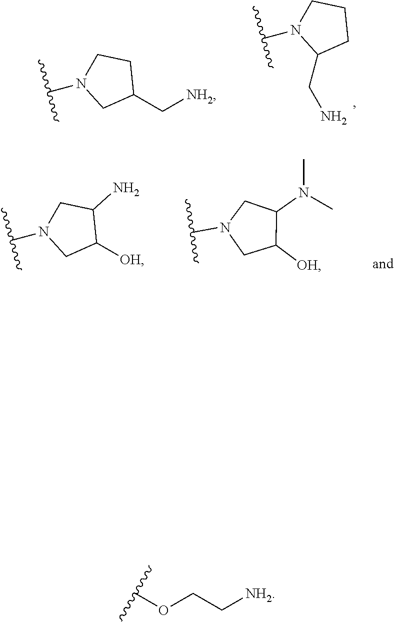

In some embodiments,

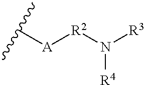

is selected from:

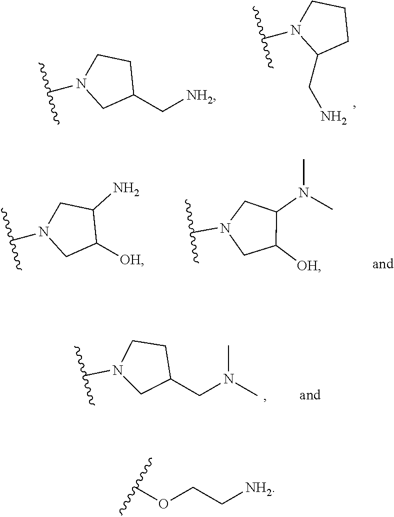

In some embodiments, for the compound or salt of Formula (I), R⁵ is selected from 3- to 6-membered saturated heterocycle, 3- to 6-membered unsaturated heterocycle, C₃-C₆ saturated carbocycle, any one of which is optionally substituted with one or more substituents independently selected from:
halogen, —OR¹⁰, —N(R¹⁰)₂, —C(O)R¹⁰, —C(O)N(R¹⁰)₂, —N(R¹⁰)C(O)R¹⁰, —N(R¹⁰)₂S(O)₂(R¹⁰), —S(O)₂N(R¹⁰)₂, —NO₂, ═O, and —CN; and
C₁₋₆ alkyl optionally substituted with one or more substituents independently selected from halogen, —OR¹⁰, —SR¹⁰, —N(R¹⁰)₂, —C(O)R¹⁰, —C(O)OR¹⁰, —OC(O)R¹⁰, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN.

In some embodiments, for the compound or salt of Formula (I), wherein R⁵ is selected from 5-membered saturated heterocycle and 5-membered unsaturated heterocycle each of which is optionally substituted. In some embodiments, R⁵ is selected from:

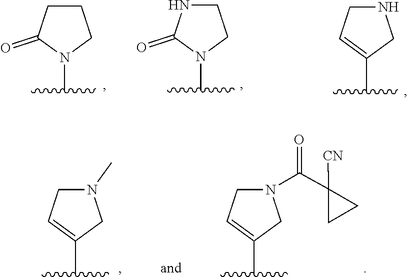

In some embodiments, for the compound or salt of Formula (I), R⁵ is selected from C₃-C₆ saturated carbocycle. In some embodiments, R⁵ is

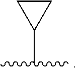 [SMILES: *C1CC1]

In some embodiments, for the compound or salt of Formula (I), R⁵ is selected from —O—(C₁-C₆ alkyl), —O-(3- to 6-membered heterocycle), and —O—(C₃-C₆ carbocycle), any one of which is optionally substituted with one or more substituents independently selected from:
—OR¹⁰, —N(R¹⁰)₂, —C(O)R¹⁰, —C(O)OR¹⁰, —OC(O)R¹⁰, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)₂N(R¹⁰)₂, —NO₂, ═O, and —CN; and
C₁₋₆ alkyl optionally substituted with one or more substituents independently selected from —OR¹⁰, —N(R¹⁰)₂, —C(O)R¹⁰, —C(O)OR¹⁰, —OC(O)R¹⁰, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)₂N(R¹⁰)₂, —NO₂, ═O, and —CN.

In some embodiments, for the compound or salt of Formula (I), R⁵ is —O—(C₁-C₆ alkyl) optionally substituted with one or more substituents independently selected from —N(R¹⁰)₂ and —C(O)N(R¹⁰)₂. In some embodiments, R⁵ is selected from:

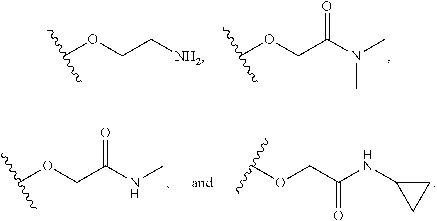

In some embodiments, R⁵ is selected from:

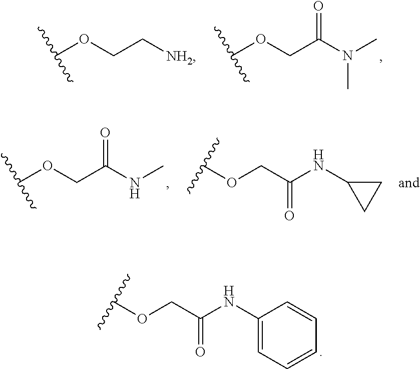

In some embodiments, for the compound or salt of Formula (I), R⁵ is selected from optionally substituted —O-(3- to 6-membered heterocycle). In some embodiments, R⁵ is selected from:

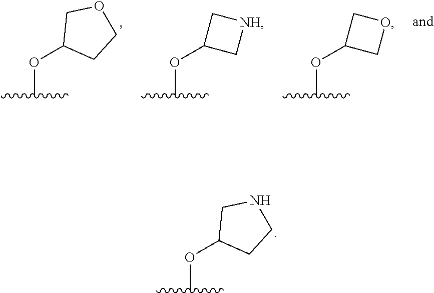 [SMILES: *OC1CCOC1 ; *OC1CNC1 ; *OC1COC1 ; *OC1CCNC1]

In some embodiments, for the compound or salt of Formula (I), R⁵ is selected from —NO₂, —N(R¹⁰)C(O)R¹⁰, —N(R¹⁰)₂S(O)₂(R¹⁰), and —S(O)₂N(R¹⁰)₂. In some embodiments, R⁵ is selected from:

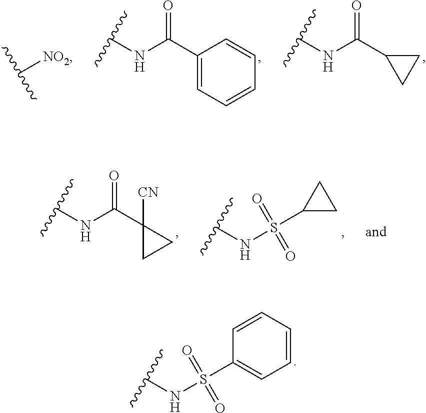

In some embodiments, R⁵ is selected from:

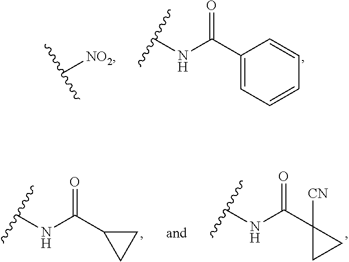

In some embodiments, R⁵ is selected from

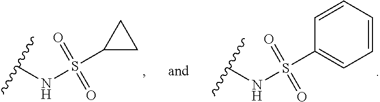 [SMILES: *NS(=O)(=O)C1CC1 ; *NS(=O)(=O)c1ccccc1]

In some embodiments, for the compound or salt of Formula (I), wherein R⁵ is selected from:
C₁ alkyl substituted with one or more substituents selected from halogen, R^{C}, R^{D}, R¹⁰, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and
C₂₋₆ alkyl, C₂₋₆ alkenyl, and C₂₋₆ alkynyl, each of which is optionally substituted with one or more substituents independently selected from halogen, R^{C}, R^{D}, R¹⁰, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN.

In some embodiments, for the compound or salt of Formula (I), R⁵ is selected from C₁ alkyl substituted with —N(R^{A})(R^{B}).

In certain aspects, Formula (I) is represented by Formula (I-a):

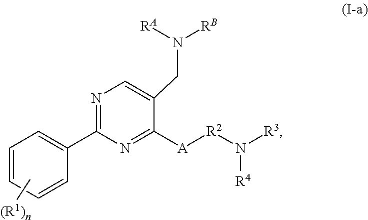

wherein R^{A}, R^{B}, R¹, R², R³, R⁴, A and n are as defined in Formula (I).

In some embodiments, for the compound or salt of Formula (I) and (I-a), n is selected from 1 and 2. In some embodiments, n is selected from 2 and 3. In some embodiments, n is 1. In some embodiments, n is 2. In some embodiments, n is 3. In some embodiments, n is selected from 2 and 3; and R¹ is not 2,6-substituted around the phenyl ring.

In some embodiments, for the compound or salt of Formula (I) and (I-a), R¹ is independently selected from halogen, —CN, N(R⁶)C(O)R⁶; and C₁₋₆ alkyl optionally substituted with one or more substituents independently selected from halogen, —OR⁶, —SR⁶, —N(R⁶)₂, —C(O)R⁶, —C(O)OR⁶, —OC(O)R⁶, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and —CN.

In some embodiments, for the compound or salt of Formula (I) and (I-a), R¹ is independently selected from halogen, —C(O)N(R⁶)₂, —CN, C₁₋₆ alkyl, and C₁₋₆ haloalkyl. In some embodiments, R¹ is selected from chloro, fluoro, methyl, CHF₂, CF₃, —CN, and

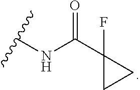 [SMILES: *NC(=O)C1(F)CC1]

In some embodiments, R¹ is selected from chloro, fluoro, methyl, CHF₂, CF₃. In some embodiments, R¹ is selected from chloro, fluoro, and CF₃.

In some embodiments, for the compound or salt of Formula (I) and (I-a), two R¹ on adjacent carbon atoms combine to form a 4- to 6-membered carbocycle together with the carbon atoms on the phenyl ring to which they are bound, thereby forming a bicyclic ring system. In some embodiments, the bicyclic ring system, is a 6-4, 6-5, and 6-6 ring bicyclic system. In some embodiments, the bicyclic ring system is represented by:

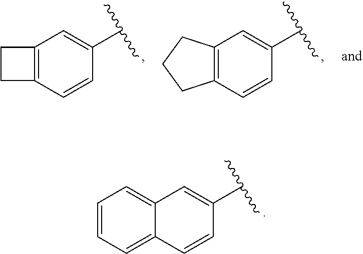 [SMILES: *c1ccc2c(c1)CC2 ; *c1ccc2c(c1)CCC2 ; *c1ccc2ccccc2c1]

In some embodiments, for the compound or salt of Formula (I) and (I-a), A is selected from —O—; and 4- to 6-membered saturated heterocyclene optionally substituted with one or more R¹².

In some embodiments, for the compound or salt of Formula (I) and (I-a), R² is selected from C₁₋₃ alkylene.

In some embodiments, for the compound or salt of Formula (I) and (I-a),

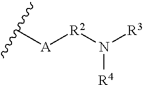

is selected from:

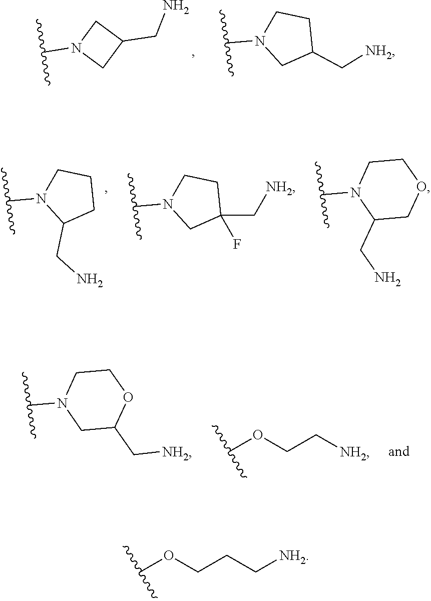

In some embodiments,

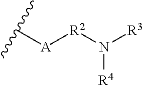

is selected from:

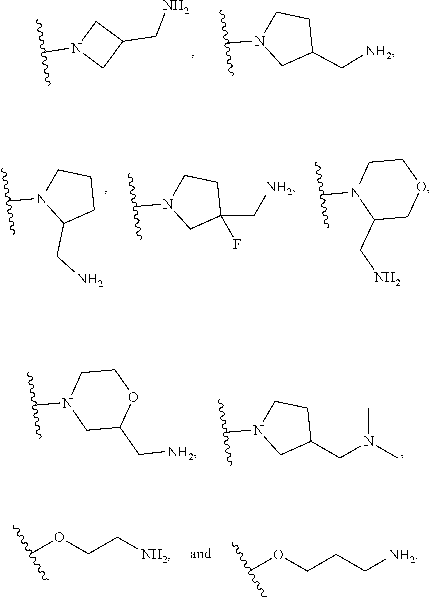

In some embodiments, for the compound or salt of Formula (I) and (I-a), A is a bond.

In some embodiments, for the compound or salt of Formula (I) and (I-a), R² and R⁴ come together to form the 5- to 7-membered heterocycle optionally substituted with C₁₋₆ alkyl optionally substituted with one or more substituents independently selected from halogen, —OR¹⁵, —N(R¹⁵)₂, —NO₂, and —CN. In some embodiments,

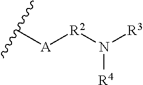

is selected from:

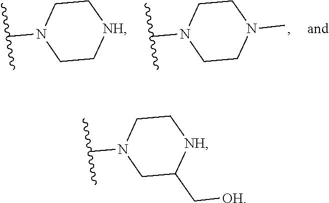

In some embodiments, for the compound or salt of Formula (I) and (I-a), A is a bond and R² is selected from 5- to 9-membered saturated heterocyclene and C₄-C₆ carbocyclene any of which is optionally substituted with one or more substituents selected from halogen and —OH. In some embodiments,

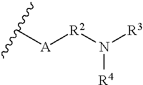

is selected from:

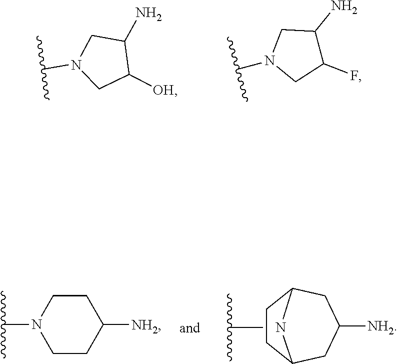

In some embodiments, A is a bond and R² is selected from 5- to 9-membered unsaturated heterocyclene. In some embodiments, R² is

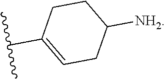 [SMILES: *C1=CCC(N)CC1]

In some embodiments, for the compound or salt of Formula (I) and (I-a), R² and R⁴ come together to form the 4- to 5-membered heterocycle optionally substituted with C₁₋₆ alkyl optionally substituted with one or more substituents independently selected from halogen, —OR¹⁵, —N(R¹⁵)₂, —NO₂, and —CN. In some embodiments, R² is C₄ carbocyclene.

In some embodiments, for the compound or salt of Formula (I) and (I-a),

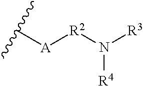

is selected from:

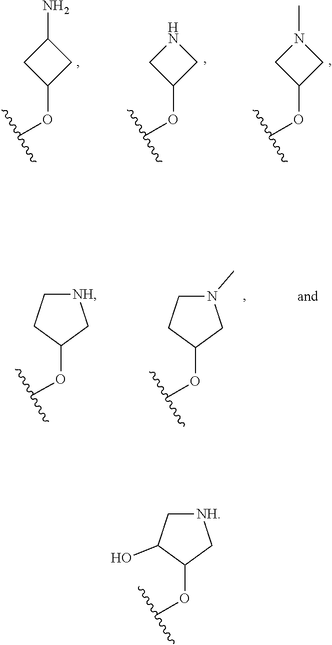

In some embodiments, for the compound or salt of Formula (I) and (I-a), R^{C} and R^{D} are each independently selected from hydrogen; and C₁₋₆ alkyl and C₃₋₆ carbocycle, each of which is optionally substituted with one or more substituents selected from: halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)₂S(O)₂(R¹⁶), —S(O)R¹⁶, —S(O)₂R¹⁶, —S(O)₂N(R¹⁶)₂, —NO₂, ═O, and —CN. In some embodiments, R^{C} and R^{D} are both selected from hydrogen. In some embodiments, R^{A} and R^{B} are both selected from hydrogen.

In some embodiments, for the compound or salt of Formula (I) and (I-a), R^{A} is selected from:
hydrogen and C₁₋₆ alkyl optionally substituted with one or more substituents independently selected halogen, —OR¹⁸, —N(R¹⁸)₂, —C(O)R¹⁸, —NO₂, ═O, and —CN; and
R^{B} is selected from: —C(O)R¹⁸, —C(O)OR¹⁸, —C(O)N(R¹⁸)₂, —S(O)₂R¹⁸; and C₁₋₆ alkyl optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, and —CN.

In some embodiments, for the compound or salt of Formula (I) and (I-a), R^{B} is —C(O)R¹⁸ and R¹⁸ is selected from C₂₋₄ alkenyl, C₃₋₆ carbocycle and 3- to 6-membered heterocycle, each of which is optionally substituted with one or more substituents independently selected from: halogen, —OR²², —N(R²²)₂, —C(O)R²², —C(O)N(R²²)₂, —CN; and C₁₋₆ alkyl each of which are optionally substituted with one or more substituents independently selected from halogen, —OR²² and —N(R²²)₂.

In some embodiments, for the compound or salt of Formula (I) and (I-a),

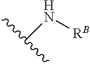

is selected from:

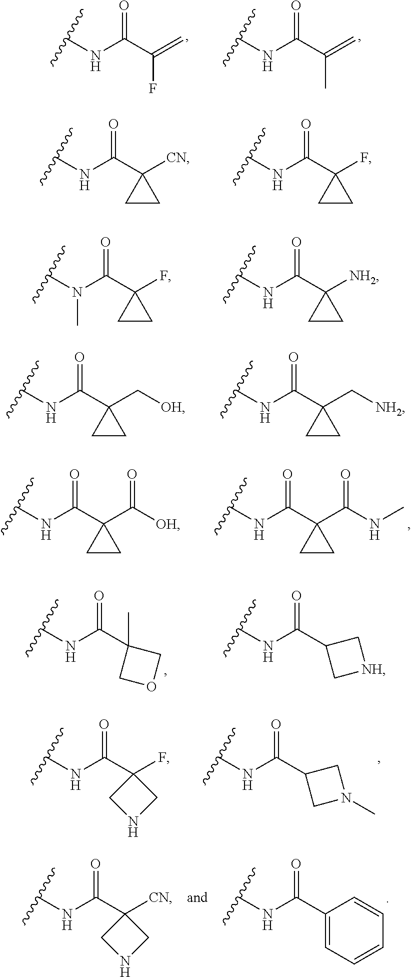

In some embodiments

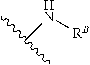

is selected from:

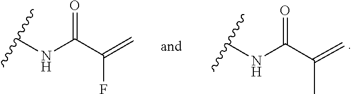

In some embodiments,

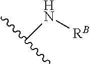

is selected from:

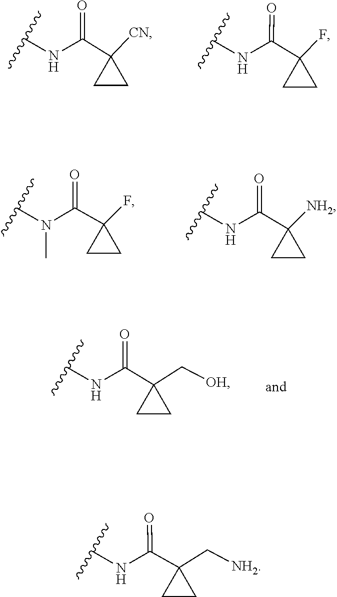

In some embodiments,

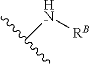

is selected from:

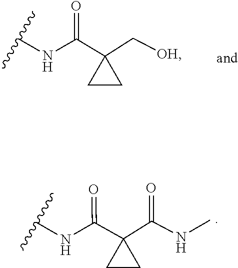 [SMILES: *NC(=O)C1(CO)CC1 ; *NC(=O)C1(C(=O)NC)CC1]

In some embodiments,

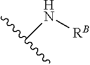

is selected from:

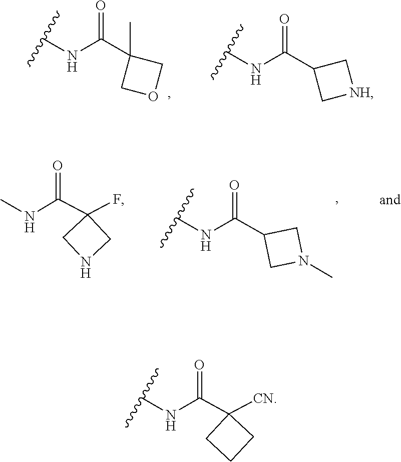

In some embodiments

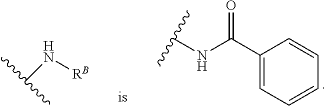

In some embodiments, for the compound or salt of Formula (I) and (I-a), R^{B} is —C(O)R¹⁸ and R¹⁸ is selected from C₂₋₄ alkenyl, C₃₋₆ carbocycle and 3- to 6-membered heterocycle, each of which is optionally substituted with one or more substituents independently selected from: halogen, —OR²², —N(R²²)₂, —C(O)R²², —C(O)N(R²²)₂, —CN; and C₁₋₆ alkyl, C₂₋₆ alkenyl, and C₂₋₆ alkynyl, wherein C₁₋₆ alkyl, C₂₋₆ alkenyl, and C₂₋₆ alkynyl are each optionally substituted with one or more substituents independently selected from halogen, —OR²² and —N(R²²)₂. In some embodiments,

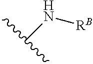

is selected from:

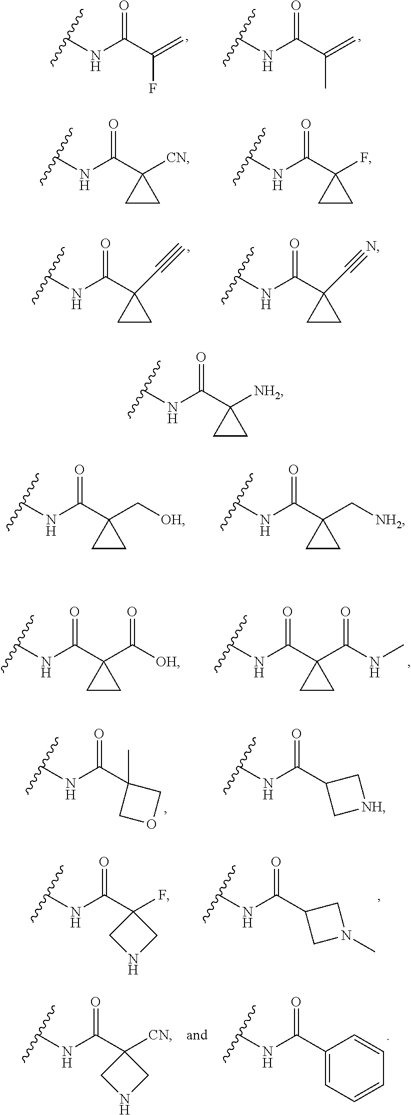

In some embodiments,

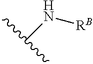

is selected from:

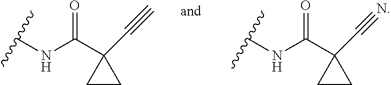

In some embodiments,

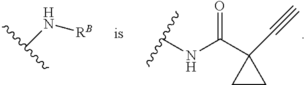

In some embodiments,

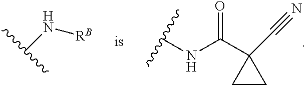

In some embodiments, for the compound or salt of Formula (I) and (I-a), R^{B} is selected from —C(O)OR¹⁸, —C(O)N(R¹⁸)₂, —S(O)₂R¹⁸; and C₁₋₆ alkyl and C₃₋₆ carbocycle any of which is optionally substituted with one or more substituents independently selected from halogen, —OR¹⁸, —N(R⁸)₂, and —CN. In some embodiments,

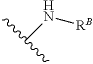

is selected from:

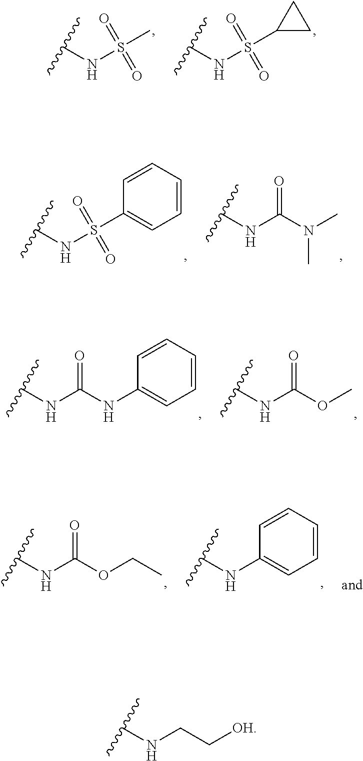

In some embodiments,

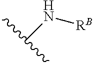

is selected from:

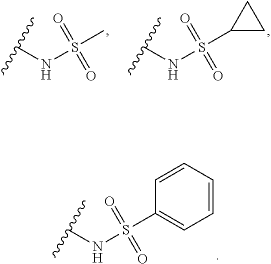

In some embodiments,

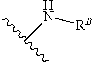

is selected from:

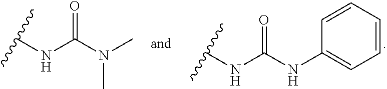

In some embodiments,

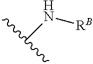

is selected from:

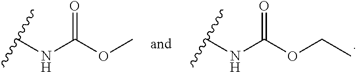

In some embodiments,

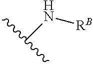

is selected from:

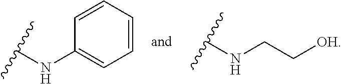 [SMILES: *Nc1ccccc1 ; *NCCO]

In some embodiments, for the compound or salt of Formula (I) and (I-a), R^{B} is —C(O)R¹⁸ and R¹⁸ is selected from C₁₋₆ alkyl, C₂₋₆ alkenyl, and C₂₋₆ alkynyl, each of which is optionally substituted with one or more substituents independently selected from halogen, R²², —OR²², —SR²², —N(R²²)₂, —NO₂, ═O, and —CN. In some embodiments,

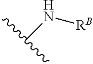

is selected from:

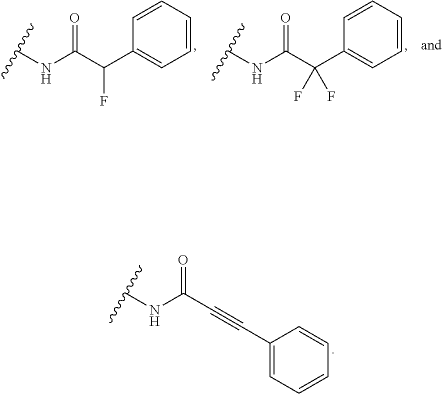

In some embodiments,

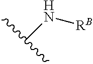

is

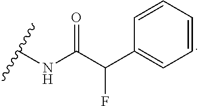 [SMILES: *NC(=O)C(F)c1ccccc1]

In some embodiments,

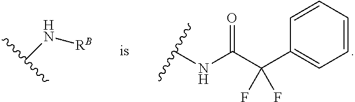

In some embodiments,

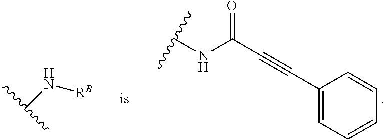

In some embodiments, for the compound or salt of Formula (I) and (I-a), R^{A} is selected from hydrogen and C₁₋₆ alkyl optionally substituted with one or more substituents independently selected halogen, —OR¹⁸, —N(R¹⁸)₂, —C(O)R¹⁸, —NO₂, ═O, and —CN; and
R^{B} is C₁₋₆ alkyl optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, —CN, and C₃₋₆ carbocycle. In some embodiments, R^{B} is C₁₋₆ alkyl optionally substituted with halogen, —OR¹⁸, —N(R¹⁸)₂, —C(O)R¹⁸, —NO₂, ═O, —CN, and C₃₋₆ carbocycle. In some embodiments,

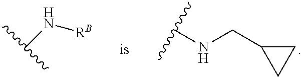

In some embodiments, for the compound or salt of Formula (I) and (I-a), R^{A} and R^{B} come together to form a 4- to 6-membered heterocycle optionally substituted with one or more substituents independently selected from: halogen, —OR¹⁹, —N(R¹⁹)₂, —C(O)R¹⁹, —NO₂, ═O, and —CN; and C₁₋₆ alkyl optionally substituted with one or more substituents independently selected halogen, —OR¹⁹, —N(R¹⁹)₂, —C(O)R¹⁹, —NO₂, ═O, and —CN. In some embodiments,

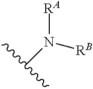

is selected from:

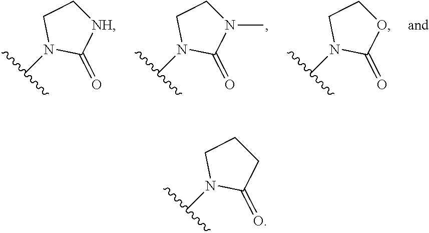

In some embodiments, the compound of Formula (I) is represented by the structure of Formula (I-b):

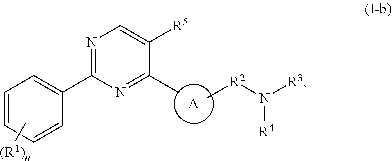

or a salt thereof, wherein R¹, R², R³, R⁴, R⁵, and n are as defined in Formula (I) and Ring A is 4- to 9-membered heterocyclene optionally substituted with one or more R¹². In some embodiments, Ring A is a saturated 4- to 9-membered heterocyclene comprising at least one nitrogen, oxygen, or sulfur heteroatom. In some embodiments, Ring A is a saturated 4- to 9-membered heterocyclene comprising at least one nitrogen or oxygen heteroatom. In some embodiments, Ring A is a saturated 4- to 9-membered heterocyclene comprising at least one nitrogen heteroatom. In some embodiments, Ring A is a saturated 4- to 9-membered heterocyclene selected from azetidinylene, diazetidinylene, pyrrolidinylene, pyrazolidinylene, imdiazolidinylene, piperidinylene, and piperazinylene, any of which is optionally substituted with one or more R¹². In some embodiments, Ring A is selected from pyrrolidinylene, pyrazolidinylene, and imdiazolidinylene, any of which is optionally substituted with one or more R¹². In some embodiments, Ring A is selected from piperidinylene, and piperazinylene, each of which is optionally substituted with one or more R¹².

In some embodiments, the compound of Formula (I) is represented by the structure of Formula (I-c):

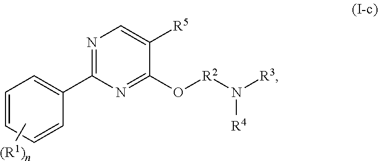

or a salt thereof, wherein R¹, R², R³, R⁴, R⁵, and n are as defined in Formula (I).

In some embodiments, the compound or salt of Formula I is represented by the structure of Formula (I-d):

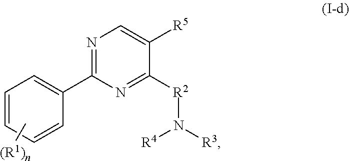

or a salt thereof, wherein R¹, R², R³, R⁴, R⁵, and n are as defined in Formula (I).

In another aspect, the present disclosure provides a compound represented by the structure of Formula (II):

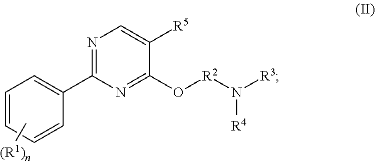

or a pharmaceutically acceptable salt thereof, wherein R¹, R², R³, R⁴, R⁵, A, and n are as defined in Formula (I) and
R¹⁸ is independently selected from:
   hydrogen;
   C₁₋₆ alkyl, C₂₋₆ alkenyl, C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
   C₃₋₁₀ carbocycle and 3- to 10-membered heterocycle each of which are optionally substituted with one or more substituents independently selected from:
      halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
      C₁₋₆ alkyl, C₂₋₆ alkenyl, and C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN.

In some embodiments, for the compound or salt of Formula (A), R⁵ is C₁ alkyl substituted with —N(R^{A})(R^{B}). In some embodiments, R^{A} or R^{B} of —N(R^{A})(R^{B}) is selected from: —OR¹⁸,

—C(O)R¹⁸, —C(O)OR¹⁸, —C(O)N(R¹⁸)₂, —S(O)R¹⁸, —S(O)₂R⁸, and —S(O)₂N(R¹⁸)₂. In some embodiments one of R^{A} or R^{B} of —N(R^{A})(R^{B}) is —C(O)R¹⁸, wherein R¹⁸ is selected from a 3- to 10-membered heterocycle. In some embodiments, R¹⁸ is

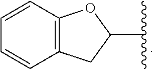 [SMILES: *C1Cc2ccccc2O1]

In some embodiments, R⁵ is

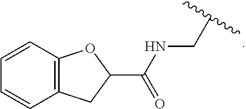 [SMILES: *CNC(=O)C1Cc2ccccc2O1]

In another aspect, the present disclosure provides a compound represented by the structure of Formula (III):

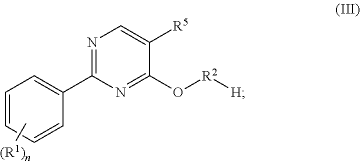

or a pharmaceutically acceptable salt thereof, wherein R¹, R², R⁵, A, and n are as defined in Formula (I). In certain embodiments, for the compound or salt of Formula (III), A is absent or —O—. In certain embodiments, for the compound or salt of Formula (III), R² is an optionally substituted 4 to 9-membered heterocyclene, such as an optionally substituted 4 to 9-membered saturated heterocyclene. In some embodiments, for the compound or salt of Formula (III), R² may be selected from bivalent piperidine, pyrrolidine, and piperazine, any of which is optionally substituted.

In some embodiments, for the compound or salt of Formula (III), R² is 4 to 9-membered heterocyclene optionally substituted with one or more substituents independently from:
halogen, —OR⁷, —SR⁷, —N(R⁷)₂, —C(O)R⁷, C(O)OR⁷, —OC(O)R⁷, —OC(O)N(R⁷)₂, —C(O)N(R⁷)₂, —N(R⁷)C(O)R⁷, —N(R⁷)C(O)OR⁷, —N(R⁷)C(O)N(R⁷)₂, —N(R⁷)₂S(O)₂(R⁷), —S(O)R⁷, —S(O)₂R⁷, —S(O)₂N(R⁷)₂, —NO₂, ═O, and CN; and
C₁₋₆ alkyl optionally substituted with one or more substituents independently selected from halogen, —OR⁷, —SR⁷, —N(R⁷)₂, —C(O)R⁷, C(O)OR⁷, —OC(O)R⁷, —OC(O)N(R⁷)₂, —C(O)N(R⁷)₂, —N(R⁷)C(O)R⁷, —N(R⁷)C(O)OR⁷, —N(R⁷)C(O)N(R⁷)₂, —N(R⁷)₂S(O)₂(R⁷), —S(O)R⁷, —S(O)₂R⁷, —S(O)₂N(R⁷)₂, —NO₂, ═O, and CN.

In some embodiments, the compound or salt of Formula (I), (I-a), (I-b), (I-c), (I-d), (II), or (III) is selected from:

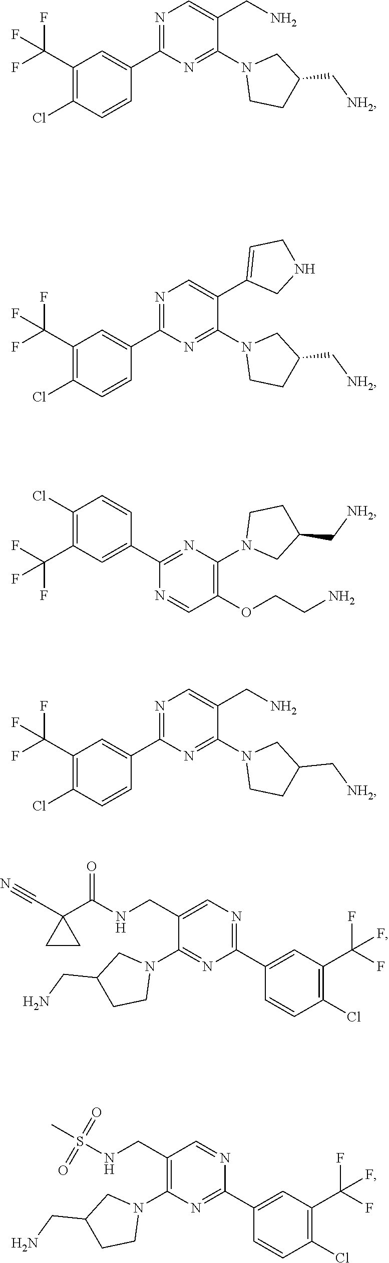 [SMILES: NCc1cnc(-c2ccc(Cl)c(C(F)(F)F)c2)nc1N1CC[C@@H](CN)C1 ; NC[C@@H]1CCN(c2nc(-c3ccc(Cl)c(C(F)(F)F)c3)ncc2C2=CCNC2)C1 ; NCCOc1cnc(-c2ccc(Cl)c(C(F)(F)F)c2)nc1N1CC[C@@H](CN)C1 ; NCc1cnc(-c2ccc(Cl)c(C(F)(F)F)c2)nc1N1CCC(CN)C1 ; N#CC1(C(=O)NCc2cnc(-c3ccc(Cl)c(C(F)(F)F)c3)nc2N2CCC(CN)C2)CC1 ; CS(=O)(=O)NCc1cnc(-c2ccc(Cl)c(C(F)(F)F)c2)nc1N1CCC(CN)C1]

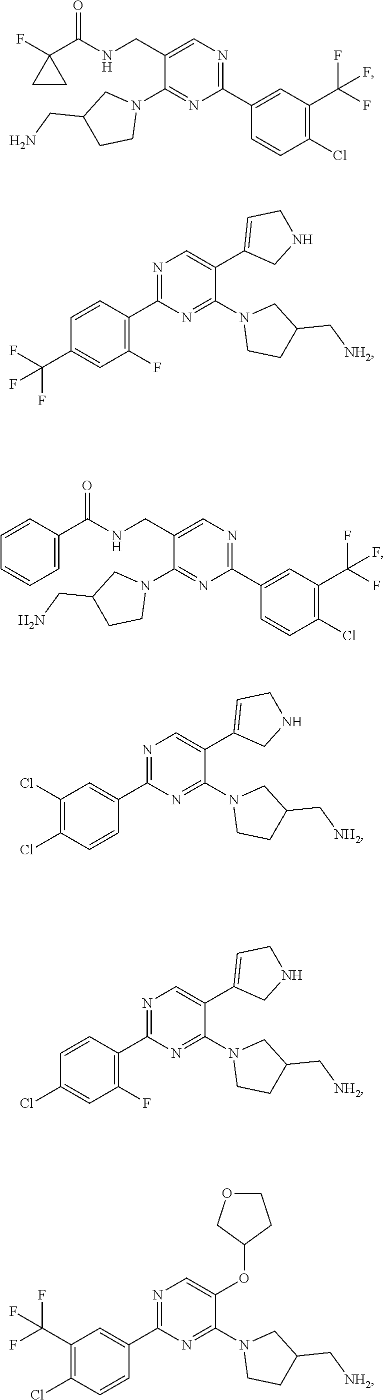 [SMILES: NCC1CCN(c2nc(-c3ccc(Cl)c(C(F)(F)F)c3)ncc2CNC(=O)C2(F)CC2)C1 ; NCC1CCN(c2nc(-c3ccc(C(F)(F)F)cc3F)ncc2C2=CCNC2)C1 ; NCC1CCN(c2nc(-c3ccc(Cl)c(C(F)(F)F)c3)ncc2CNC(=O)c2ccccc2)C1 ; NCC1CCN(c2nc(-c3ccc(Cl)c(Cl)c3)ncc2C2=CCNC2)C1 ; NCC1CCN(c2nc(-c3ccc(Cl)cc3F)ncc2C2=CCNC2)C1 ; NCC1CCN(c2nc(-c3ccc(Cl)c(C(F)(F)F)c3)ncc2OC2CCOC2)C1]

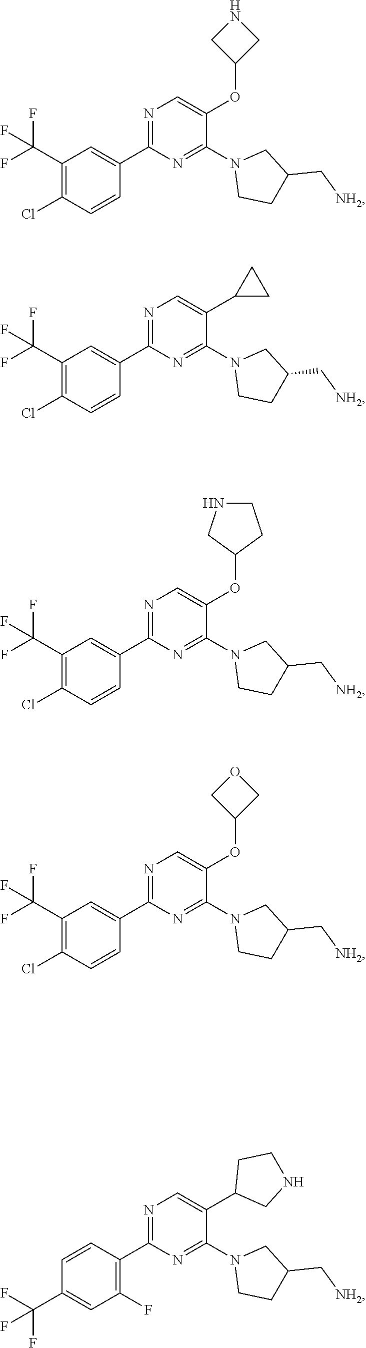 [SMILES: NCC1CCN(c2nc(-c3ccc(Cl)c(C(F)(F)F)c3)ncc2OC2CNC2)C1 ; NC[C@@H]1CCN(c2nc(-c3ccc(Cl)c(C(F)(F)F)c3)ncc2C2CC2)C1 ; NCC1CCN(c2nc(-c3ccc(Cl)c(C(F)(F)F)c3)ncc2OC2CCNC2)C1 ; NCC1CCN(c2nc(-c3ccc(Cl)c(C(F)(F)F)c3)ncc2OC2COC2)C1 ; NCC1CCN(c2nc(-c3ccc(C(F)(F)F)cc3F)ncc2C2CCNC2)C1]

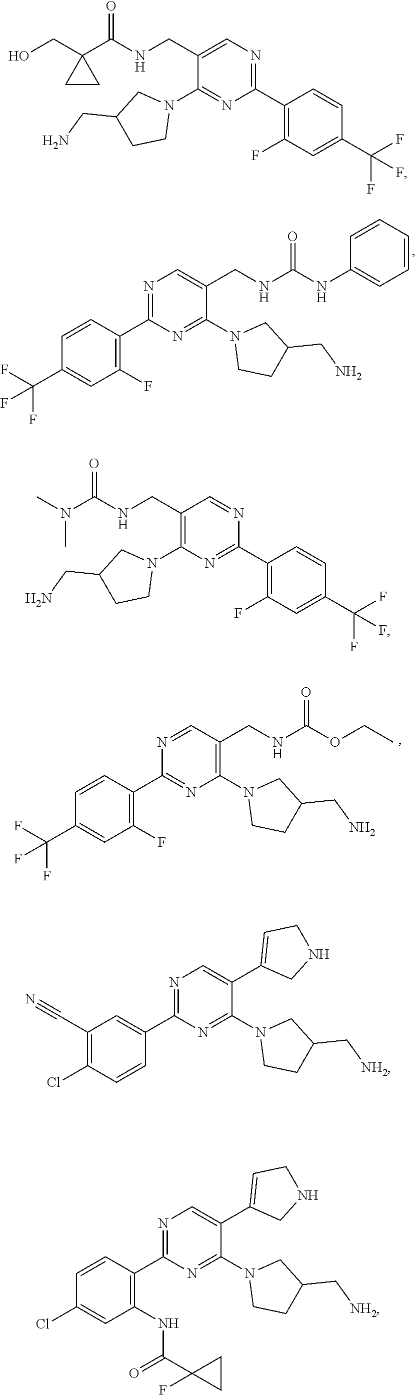

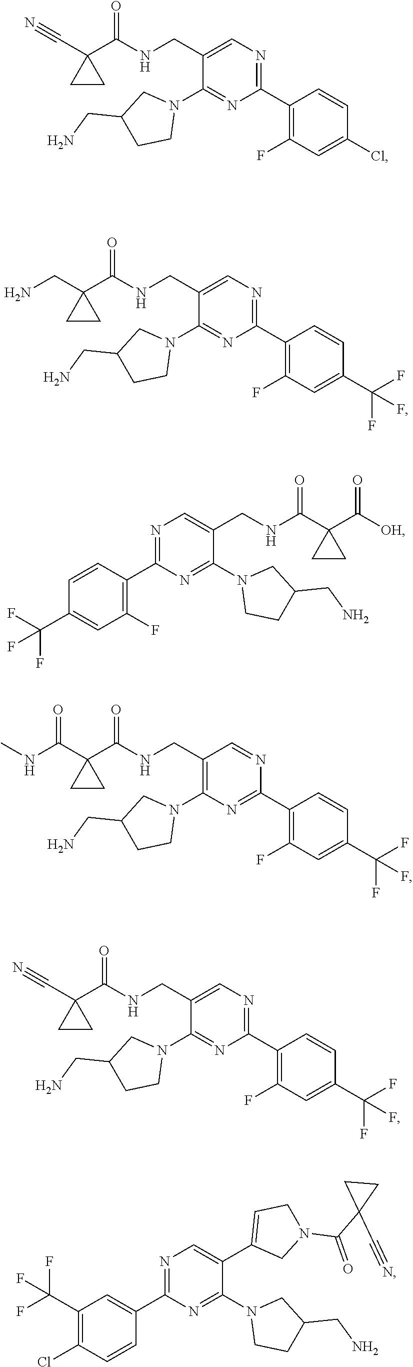 [SMILES: N#CC1(C(=O)NCc2cnc(-c3ccc(Cl)cc3F)nc2N2CCC(CN)C2)CC1 ; NCC1CCN(c2nc(-c3ccc(C(F)(F)F)cc3F)ncc2CNC(=O)C2(CN)CC2)C1 ; NCC1CCN(c2nc(-c3ccc(C(F)(F)F)cc3F)ncc2CNC(=O)C2(C(=O)O)CC2)C1 ; CNC(=O)C1(C(=O)NCc2cnc(-c3ccc(C(F)(F)F)cc3F)nc2N2CCC(CN)C2)CC1 ; N#CC1(C(=O)NCc2cnc(-c3ccc(C(F)(F)F)cc3F)nc2N2CCC(CN)C2)CC1 ; N#CC1(C(=O)N2CC=C(c3cnc(-c4ccc(Cl)c(C(F)(F)F)c4)nc3N3CCC(CN)C3)C2)CC1]

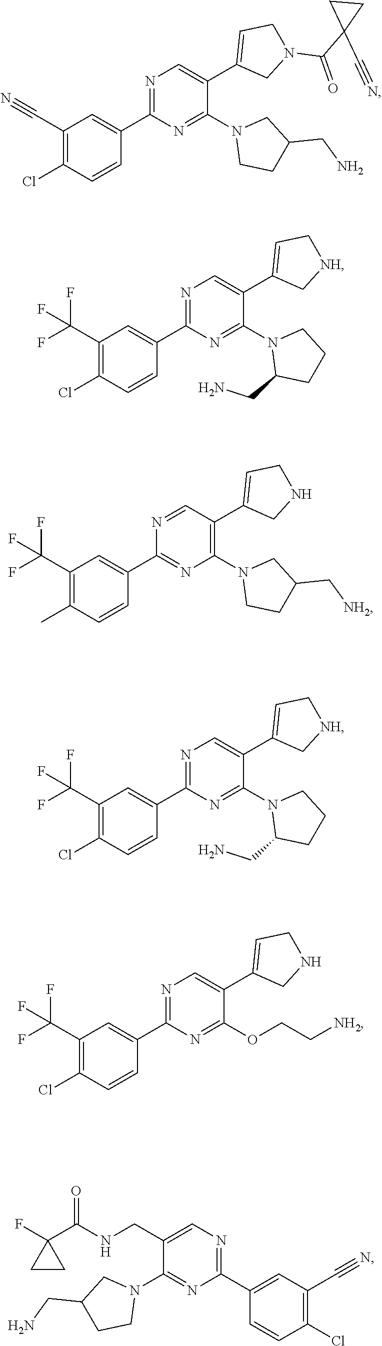 [SMILES: N#Cc1cc(-c2ncc(C3=CCN(C(=O)C4(C#N)CC4)C3)c(N3CCC(CN)C3)n2)ccc1Cl ; NC[C@@H]1CCCN1c1nc(-c2ccc(Cl)c(C(F)(F)F)c2)ncc1C1=CCNC1 ; Cc1ccc(-c2ncc(C3=CCNC3)c(N3CCC(CN)C3)n2)cc1C(F)(F)F ; NC[C@H]1CCCN1c1nc(-c2ccc(Cl)c(C(F)(F)F)c2)ncc1C1=CCNC1 ; NCCOc1nc(-c2ccc(Cl)c(C(F)(F)F)c2)ncc1C1=CCNC1 ; N#Cc1cc(-c2ncc(CNC(=O)C3(F)CC3)c(N3CCC(CN)C3)n2)ccc1Cl]

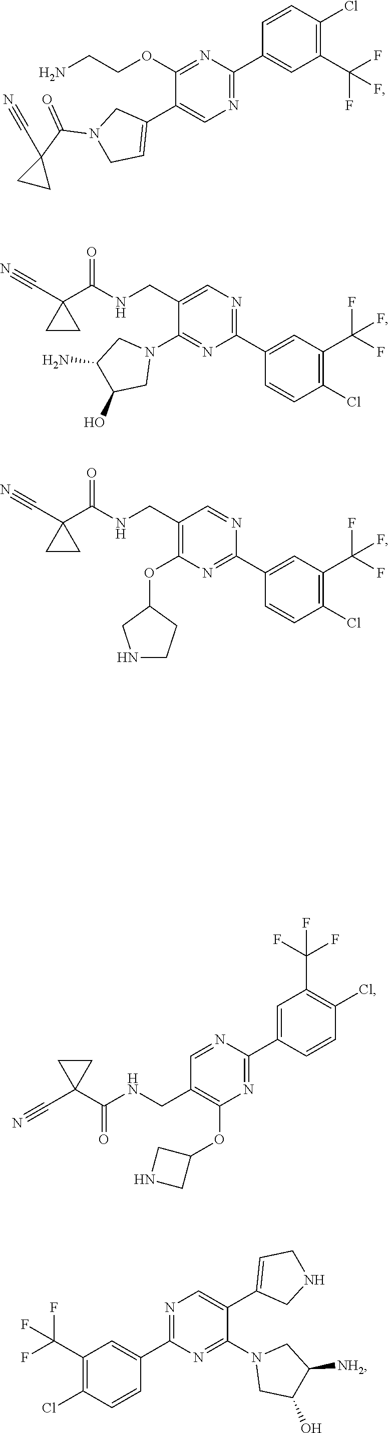 [SMILES: N#CC1(C(=O)N2CC=C(c3cnc(-c4ccc(Cl)c(C(F)(F)F)c4)nc3OCCN)C2)CC1 ; N#CC1(C(=O)NCc2cnc(-c3ccc(Cl)c(C(F)(F)F)c3)nc2N2C[C@@H](N)[C@H](O)C2)CC1 ; N#CC1(C(=O)NCc2cnc(-c3ccc(Cl)c(C(F)(F)F)c3)nc2OC2CCNC2)CC1 ; N#CC1(C(=O)NCc2cnc(-c3ccc(Cl)c(C(F)(F)F)c3)nc2OC2CNC2)CC1 ; N[C@@H]1CN(c2nc(-c3ccc(Cl)c(C(F)(F)F)c3)ncc2C2=CCNC2)C[C@H]1O]

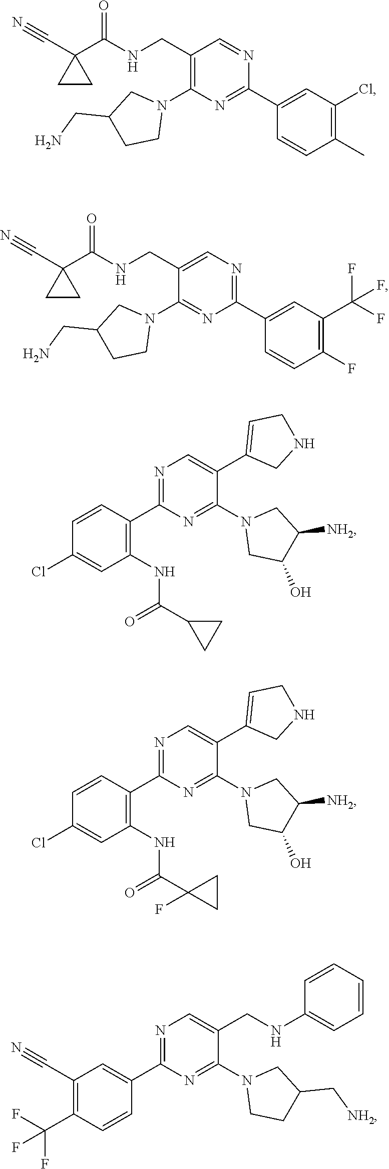 [SMILES: Cc1ccc(-c2ncc(CNC(=O)C3(C#N)CC3)c(N3CCC(CN)C3)n2)cc1Cl ; N#CC1(C(=O)NCc2cnc(-c3ccc(F)c(C(F)(F)F)c3)nc2N2CCC(CN)C2)CC1 ; N[C@@H]1CN(c2nc(-c3ccc(Cl)cc3NC(=O)C3CC3)ncc2C2=CCNC2)C[C@H]1O ; N[C@@H]1CN(c2nc(-c3ccc(Cl)cc3NC(=O)C3(F)CC3)ncc2C2=CCNC2)C[C@H]1O ; N#Cc1cc(-c2ncc(CNc3ccccc3)c(N3CCC(CN)C3)n2)ccc1C(F)(F)F]

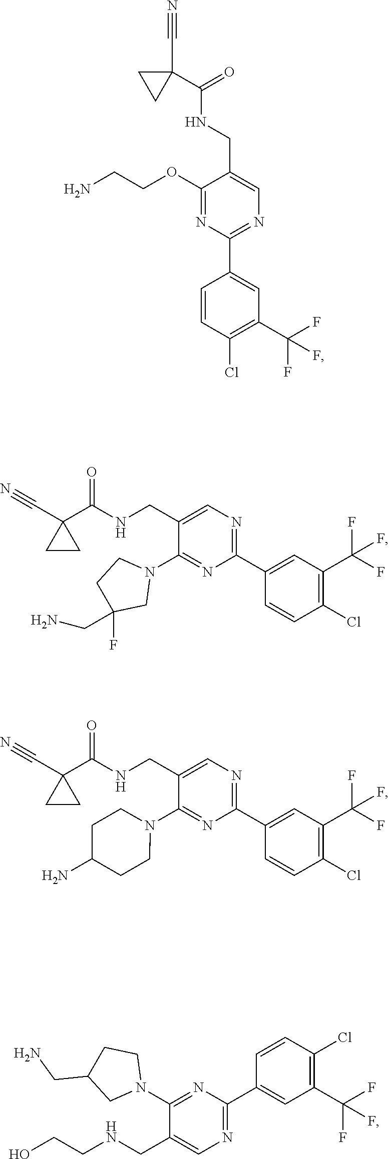 [SMILES: N#CC1(C(=O)NCc2cnc(-c3ccc(Cl)c(C(F)(F)F)c3)nc2OCCN)CC1 ; N#CC1(C(=O)NCc2cnc(-c3ccc(Cl)c(C(F)(F)F)c3)nc2N2CCC(F)(CN)C2)CC1 ; N#CC1(C(=O)NCc2cnc(-c3ccc(Cl)c(C(F)(F)F)c3)nc2N2CCC(N)CC2)CC1 ; NCC1CCN(c2nc(-c3ccc(Cl)c(C(F)(F)F)c3)ncc2CNCCO)C1]

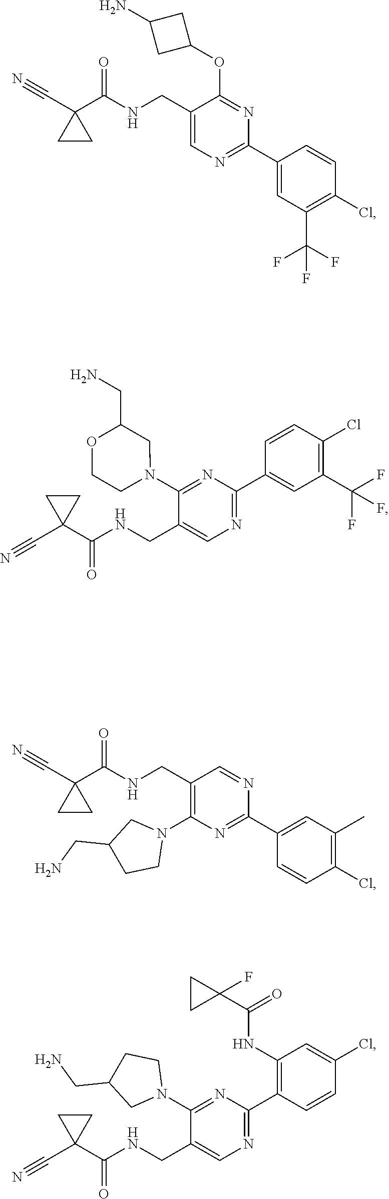 [SMILES: N#CC1(C(=O)NCc2cnc(-c3ccc(Cl)c(C(F)(F)F)c3)nc2OC2CC(N)C2)CC1 ; N#CC1(C(=O)NCc2cnc(-c3ccc(Cl)c(C(F)(F)F)c3)nc2N2CCOC(CN)C2)CC1 ; Cc1cc(-c2ncc(CNC(=O)C3(C#N)CC3)c(N3CCC(CN)C3)n2)ccc1Cl ; N#CC1(C(=O)NCc2cnc(-c3ccc(Cl)cc3NC(=O)C3(F)CC3)nc2N2CCC(CN)C2)CC1]

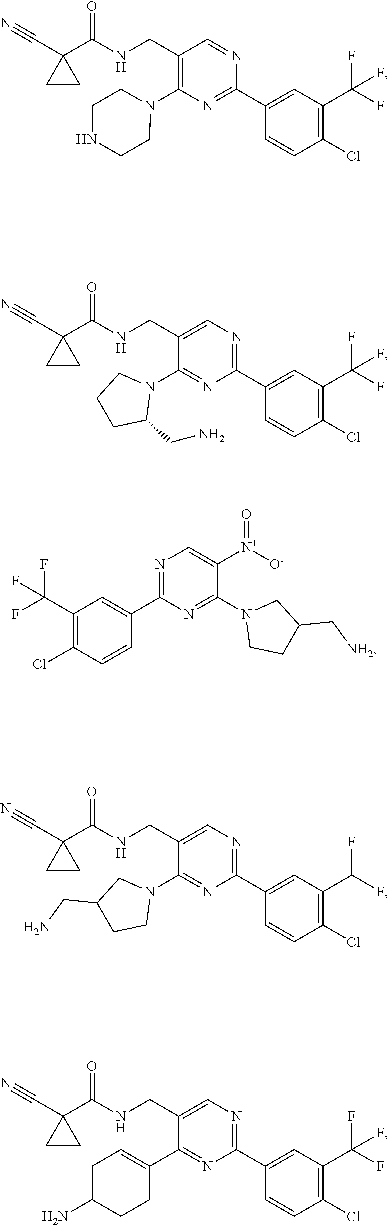 [SMILES: N#CC1(C(=O)NCc2cnc(-c3ccc(Cl)c(C(F)(F)F)c3)nc2N2CCNCC2)CC1 ; N#CC1(C(=O)NCc2cnc(-c3ccc(Cl)c(C(F)(F)F)c3)nc2N2CCC[C@H]2CN)CC1 ; NCC1CCN(c2nc(-c3ccc(Cl)c(C(F)(F)F)c3)ncc2[N+](=O)[O-])C1 ; N#CC1(C(=O)NCc2cnc(-c3ccc(Cl)c(C(F)F)c3)nc2N2CCC(CN)C2)CC1 ; N#CC1(C(=O)NCc2cnc(-c3ccc(Cl)c(C(F)(F)F)c3)nc2C2=CCC(N)CC2)CC1]

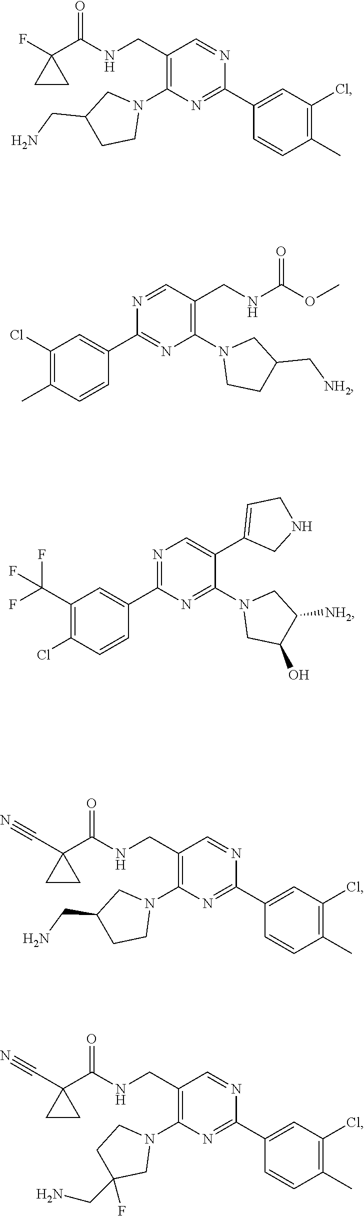 [SMILES: Cc1ccc(-c2ncc(CNC(=O)C3(F)CC3)c(N3CCC(CN)C3)n2)cc1Cl ; COC(=O)NCc1cnc(-c2ccc(C)c(Cl)c2)nc1N1CCC(CN)C1 ; N[C@H]1CN(c2nc(-c3ccc(Cl)c(C(F)(F)F)c3)ncc2C2=CCNC2)C[C@@H]1O ; Cc1ccc(-c2ncc(CNC(=O)C3(C#N)CC3)c(N3CC[C@@H](CN)C3)n2)cc1Cl ; Cc1ccc(-c2ncc(CNC(=O)C3(C#N)CC3)c(N3CCC(F)(CN)C3)n2)cc1Cl]

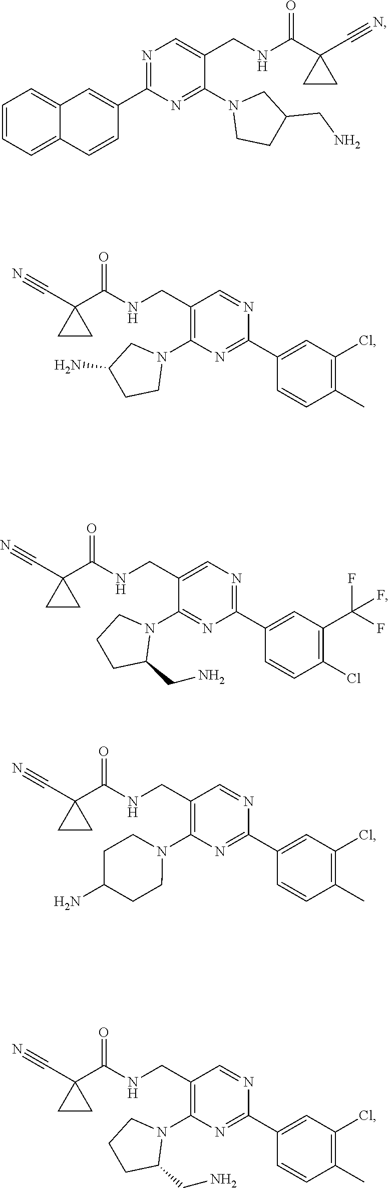 [SMILES: N#CC1(C(=O)NCc2cnc(-c3ccc4ccccc4c3)nc2N2CCC(CN)C2)CC1 ; Cc1ccc(-c2ncc(CNC(=O)C3(C#N)CC3)c(N3CC[C@H](N)C3)n2)cc1Cl ; N#CC1(C(=O)NCc2cnc(-c3ccc(Cl)c(C(F)(F)F)c3)nc2N2CCC[C@@H]2CN)CC1 ; Cc1ccc(-c2ncc(CNC(=O)C3(C#N)CC3)c(N3CCC(N)CC3)n2)cc1Cl ; Cc1ccc(-c2ncc(CNC(=O)C3(C#N)CC3)c(N3CCC[C@H]3CN)n2)cc1Cl]

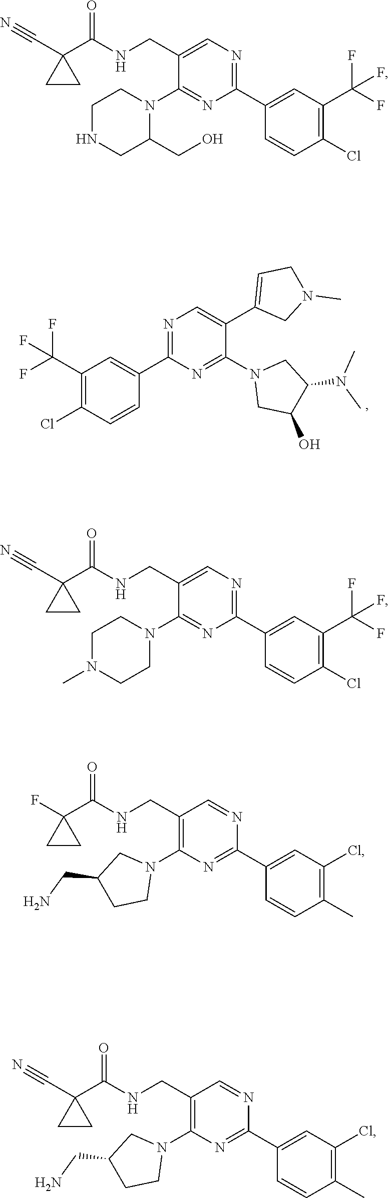 [SMILES: N#CC1(C(=O)NCc2cnc(-c3ccc(Cl)c(C(F)(F)F)c3)nc2N2CCNCC2CO)CC1 ; CN1CC=C(c2cnc(-c3ccc(Cl)c(C(F)(F)F)c3)nc2N2C[C@H](O)[C@@H](N(C)C)C2)C1 ; CN1CCN(c2nc(-c3ccc(Cl)c(C(F)(F)F)c3)ncc2CNC(=O)C2(C#N)CC2)CC1 ; Cc1ccc(-c2ncc(CNC(=O)C3(F)CC3)c(N3CC[C@@H](CN)C3)n2)cc1Cl ; Cc1ccc(-c2ncc(CNC(=O)C3(C#N)CC3)c(N3CC[C@H](CN)C3)n2)cc1Cl]

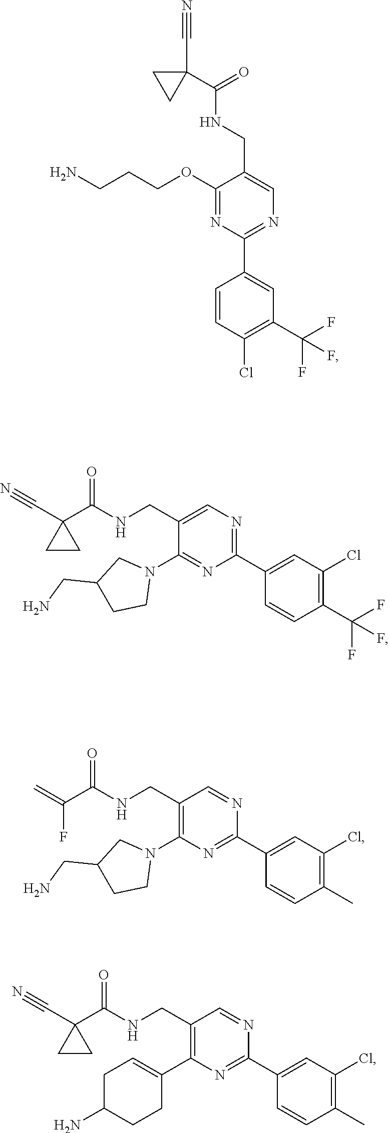 [SMILES: N#CC1(C(=O)NCc2cnc(-c3ccc(Cl)c(C(F)(F)F)c3)nc2OCCCN)CC1 ; N#CC1(C(=O)NCc2cnc(-c3ccc(C(F)(F)F)c(Cl)c3)nc2N2CCC(CN)C2)CC1 ; C=C(F)C(=O)NCc1cnc(-c2ccc(C)c(Cl)c2)nc1N1CCC(CN)C1 ; Cc1ccc(-c2ncc(CNC(=O)C3(C#N)CC3)c(C3=CCC(N)CC3)n2)cc1Cl]

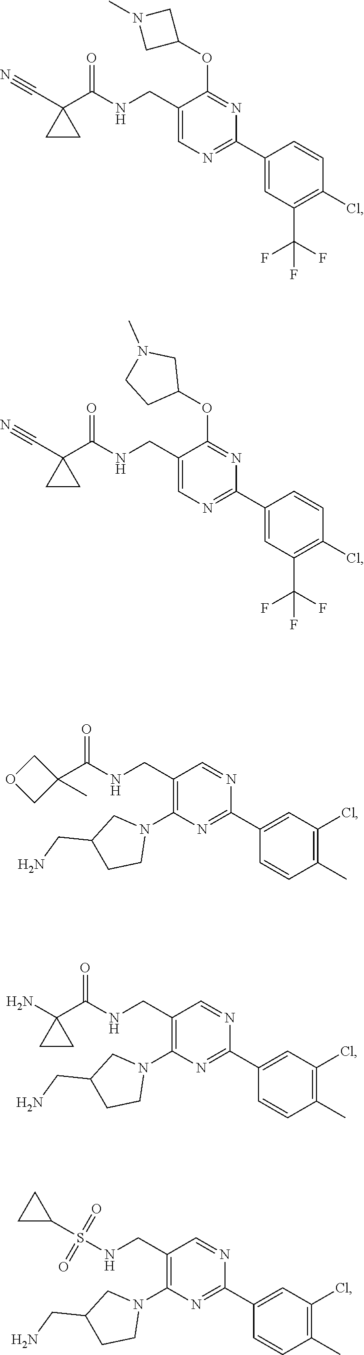 [SMILES: CN1CC(Oc2nc(-c3ccc(Cl)c(C(F)(F)F)c3)ncc2CNC(=O)C2(C#N)CC2)C1 ; CN1CCC(Oc2nc(-c3ccc(Cl)c(C(F)(F)F)c3)ncc2CNC(=O)C2(C#N)CC2)C1 ; Cc1ccc(-c2ncc(CNC(=O)C3(C)COC3)c(N3CCC(CN)C3)n2)cc1Cl ; Cc1ccc(-c2ncc(CNC(=O)C3(N)CC3)c(N3CCC(CN)C3)n2)cc1Cl ; Cc1ccc(-c2ncc(CNS(=O)(=O)C3CC3)c(N3CCC(CN)C3)n2)cc1Cl]

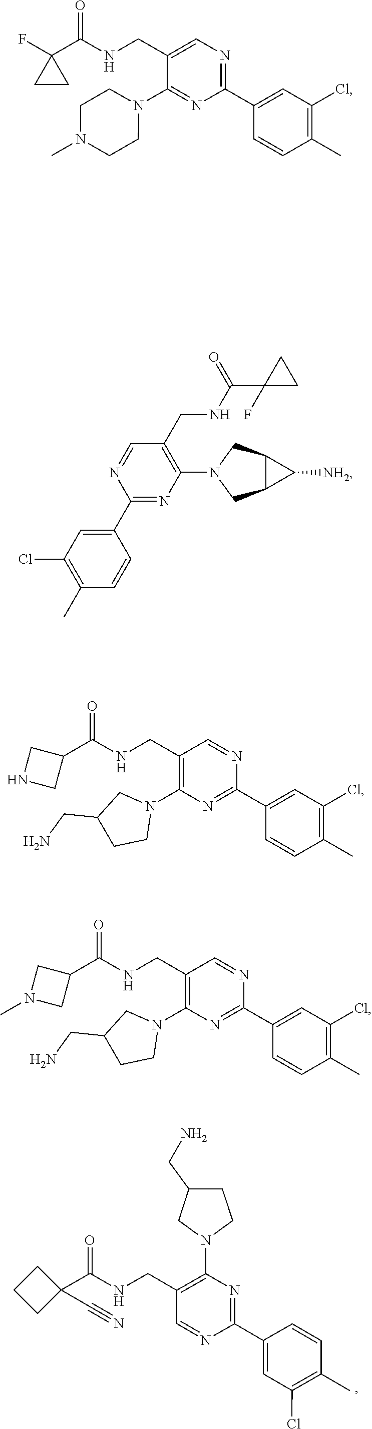

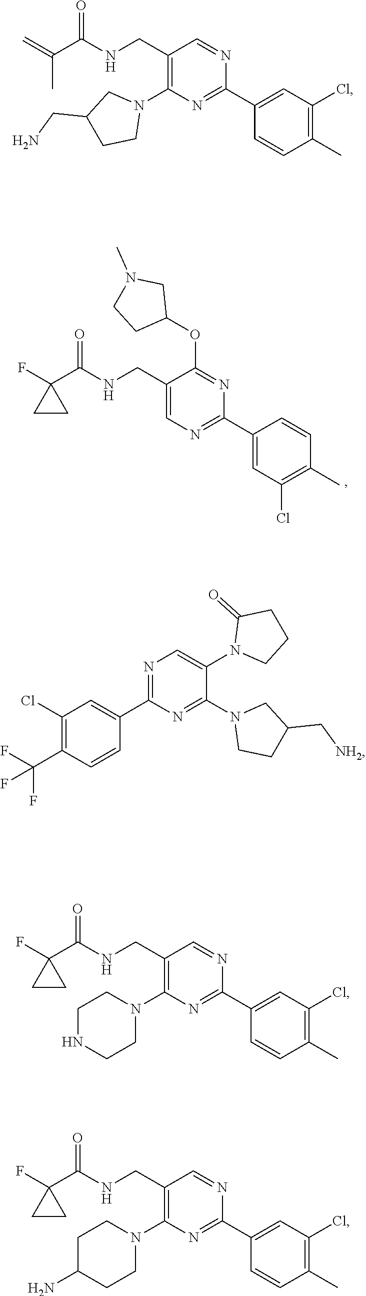 [SMILES: C=C(C)C(=O)NCc1cnc(-c2ccc(C)c(Cl)c2)nc1N1CCC(CN)C1 ; Cc1ccc(-c2ncc(CNC(=O)C3(F)CC3)c(OC3CCN(C)C3)n2)cc1Cl ; NCC1CCN(c2nc(-c3ccc(C(F)(F)F)c(Cl)c3)ncc2N2CCCC2=O)C1 ; Cc1ccc(-c2ncc(CNC(=O)C3(F)CC3)c(N3CCNCC3)n2)cc1Cl ; Cc1ccc(-c2ncc(CNC(=O)C3(F)CC3)c(N3CCC(N)CC3)n2)cc1Cl]

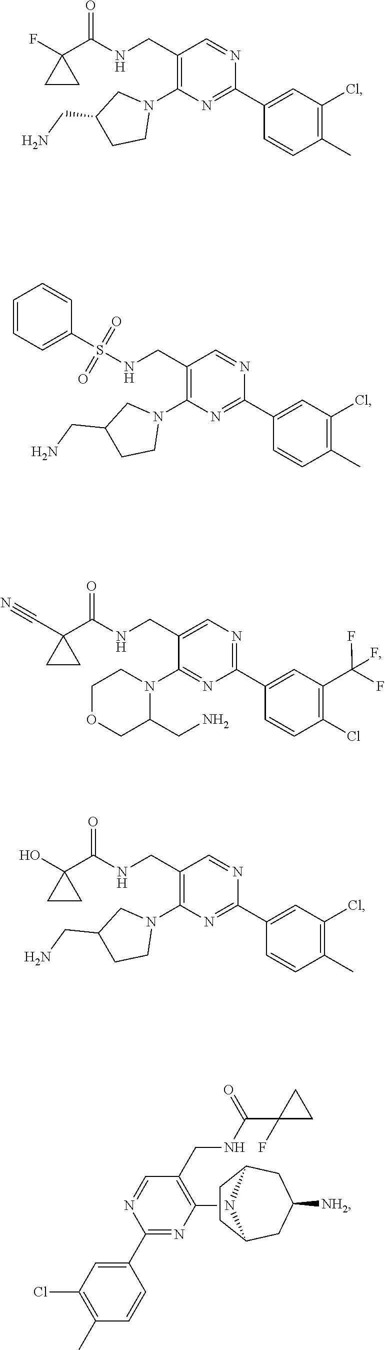 [SMILES: Cc1ccc(-c2ncc(CNC(=O)C3(F)CC3)c(N3CC[C@H](CN)C3)n2)cc1Cl ; Cc1ccc(-c2ncc(CNS(=O)(=O)c3ccccc3)c(N3CCC(CN)C3)n2)cc1Cl ; N#CC1(C(=O)NCc2cnc(-c3ccc(Cl)c(C(F)(F)F)c3)nc2N2CCOCC2CN)CC1 ; Cc1ccc(-c2ncc(CNC(=O)C3(O)CC3)c(N3CCC(CN)C3)n2)cc1Cl ; Cc1ccc(-c2ncc(CNC(=O)C3(F)CC3)c(N3[C@@H]4CC[C@H]3C[C@@H](N)C4)n2)cc1Cl]

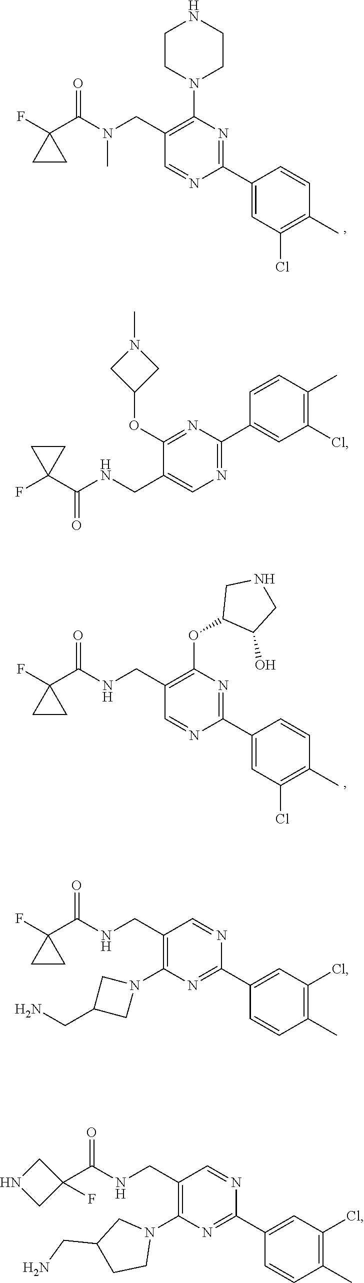

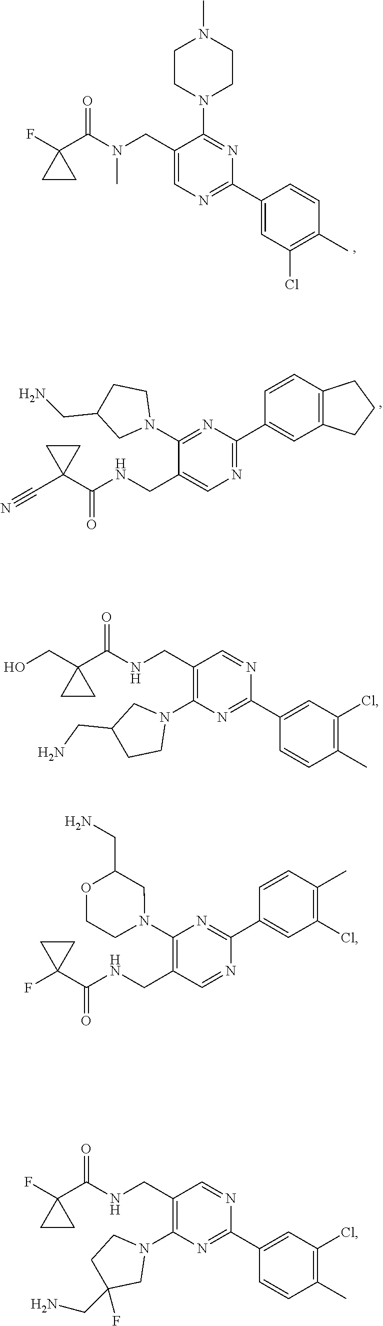 [SMILES: Cc1ccc(-c2ncc(CN(C)C(=O)C3(F)CC3)c(N3CCN(C)CC3)n2)cc1Cl ; N#CC1(C(=O)NCc2cnc(-c3ccc4c(c3)CCC4)nc2N2CCC(CN)C2)CC1 ; Cc1ccc(-c2ncc(CNC(=O)C3(CO)CC3)c(N3CCC(CN)C3)n2)cc1Cl ; Cc1ccc(-c2ncc(CNC(=O)C3(F)CC3)c(N3CCOC(CN)C3)n2)cc1Cl ; Cc1ccc(-c2ncc(CNC(=O)C3(F)CC3)c(N3CCC(F)(CN)C3)n2)cc1Cl]

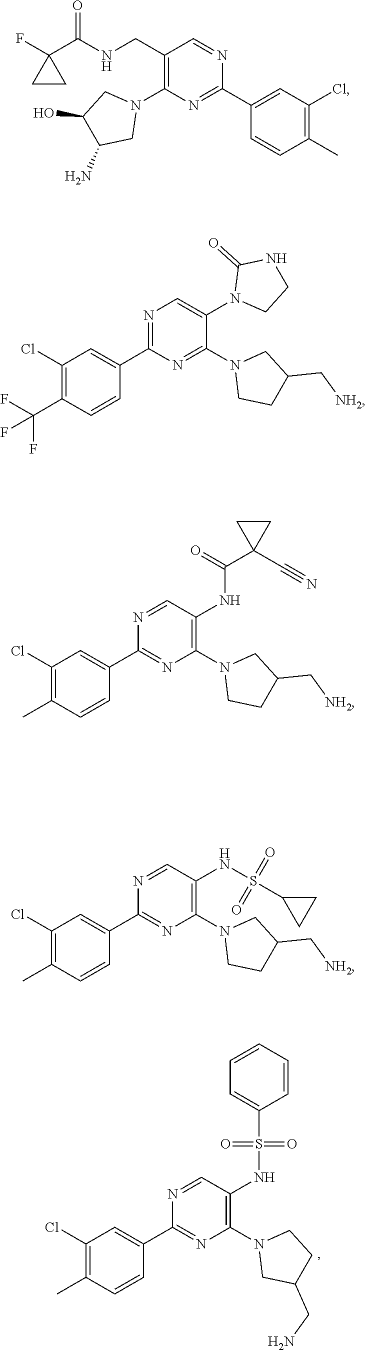 [SMILES: Cc1ccc(-c2ncc(CNC(=O)C3(F)CC3)c(N3C[C@H](N)[C@@H](O)C3)n2)cc1Cl ; NCC1CCN(c2nc(-c3ccc(C(F)(F)F)c(Cl)c3)ncc2N2CCNC2=O)C1 ; Cc1ccc(-c2ncc(NC(=O)C3(C#N)CC3)c(N3CCC(CN)C3)n2)cc1Cl ; Cc1ccc(-c2ncc(NS(=O)(=O)C3CC3)c(N3CCC(CN)C3)n2)cc1Cl ; Cc1ccc(-c2ncc(NS(=O)(=O)c3ccccc3)c(N3CCC(CN)C3)n2)cc1Cl]

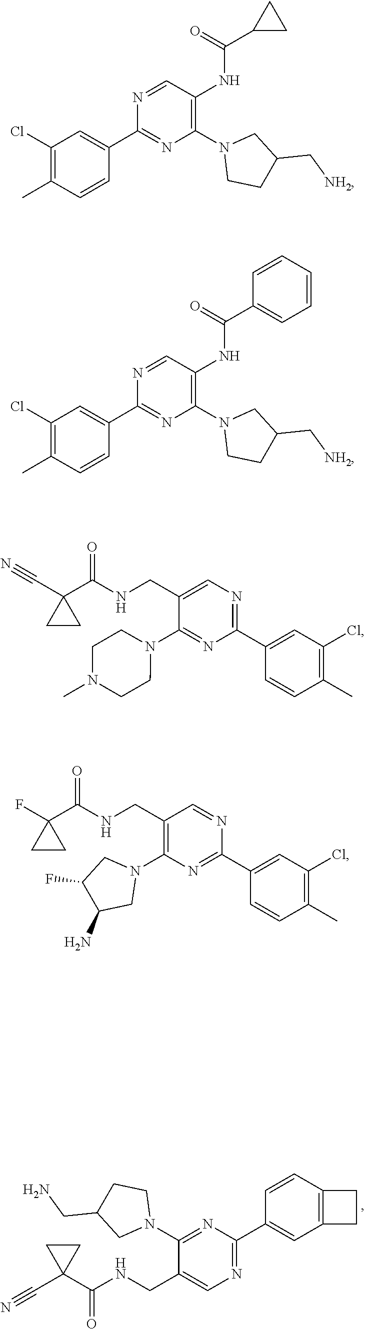 [SMILES: Cc1ccc(-c2ncc(NC(=O)C3CC3)c(N3CCC(CN)C3)n2)cc1Cl ; Cc1ccc(-c2ncc(NC(=O)c3ccccc3)c(N3CCC(CN)C3)n2)cc1Cl ; Cc1ccc(-c2ncc(CNC(=O)C3(C#N)CC3)c(N3CCN(C)CC3)n2)cc1Cl ; Cc1ccc(-c2ncc(CNC(=O)C3(F)CC3)c(N3C[C@@H](N)[C@H](F)C3)n2)cc1Cl ; N#CC1(C(=O)NCc2cnc(-c3ccc4c(c3)CC4)nc2N2CCC(CN)C2)CC1]

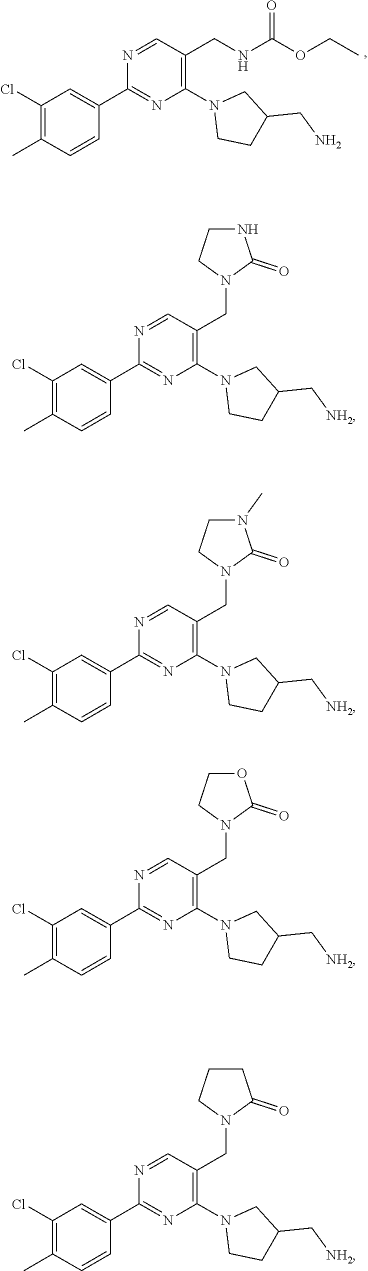 [SMILES: CCOC(=O)NCc1cnc(-c2ccc(C)c(Cl)c2)nc1N1CCC(CN)C1 ; Cc1ccc(-c2ncc(CN3CCNC3=O)c(N3CCC(CN)C3)n2)cc1Cl ; Cc1ccc(-c2ncc(CN3CCN(C)C3=O)c(N3CCC(CN)C3)n2)cc1Cl ; Cc1ccc(-c2ncc(CN3CCOC3=O)c(N3CCC(CN)C3)n2)cc1Cl ; Cc1ccc(-c2ncc(CN3CCCC3=O)c(N3CCC(CN)C3)n2)cc1Cl]

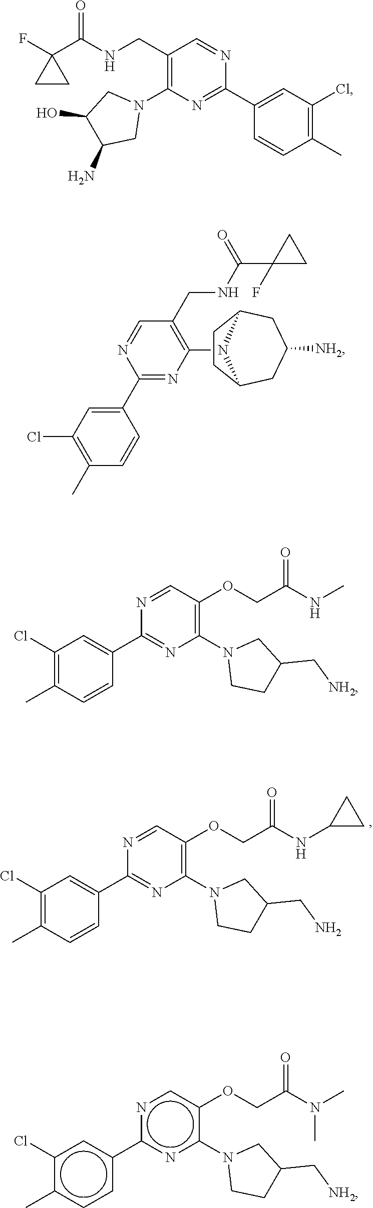

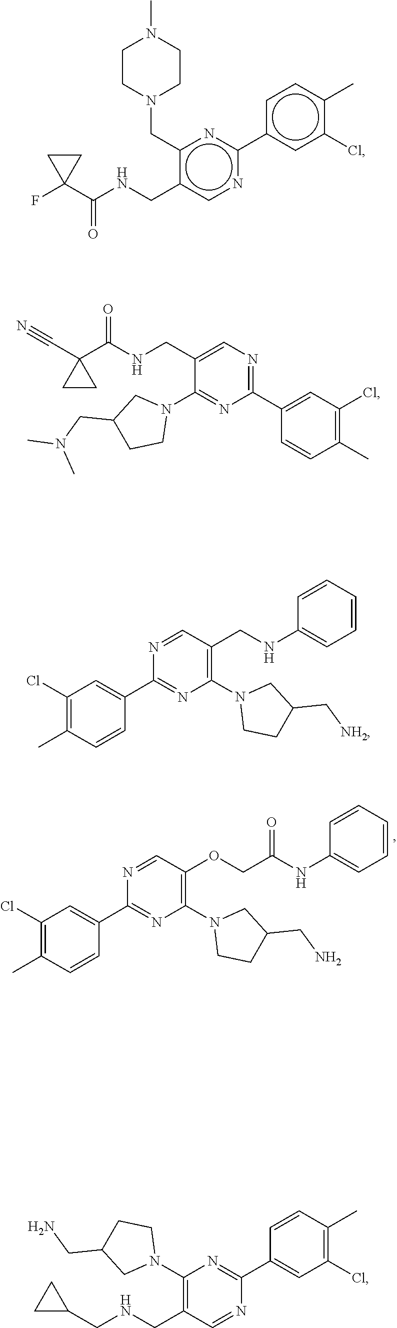

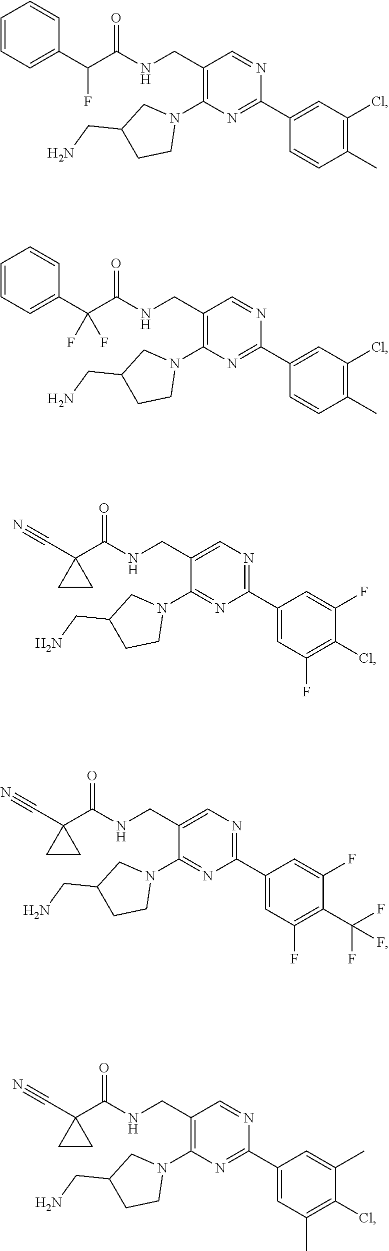 [SMILES: Cc1ccc(-c2ncc(CNC(=O)C(F)c3ccccc3)c(N3CCC(CN)C3)n2)cc1Cl ; Cc1ccc(-c2ncc(CNC(=O)C(F)(F)c3ccccc3)c(N3CCC(CN)C3)n2)cc1Cl ; N#CC1(C(=O)NCc2cnc(-c3cc(F)c(Cl)c(F)c3)nc2N2CCC(CN)C2)CC1 ; N#CC1(C(=O)NCc2cnc(-c3cc(F)c(C(F)(F)F)c(F)c3)nc2N2CCC(CN)C2)CC1 ; Cc1cc(-c2ncc(CNC(=O)C3(C#N)CC3)c(N3CCC(CN)C3)n2)cc(C)c1Cl]

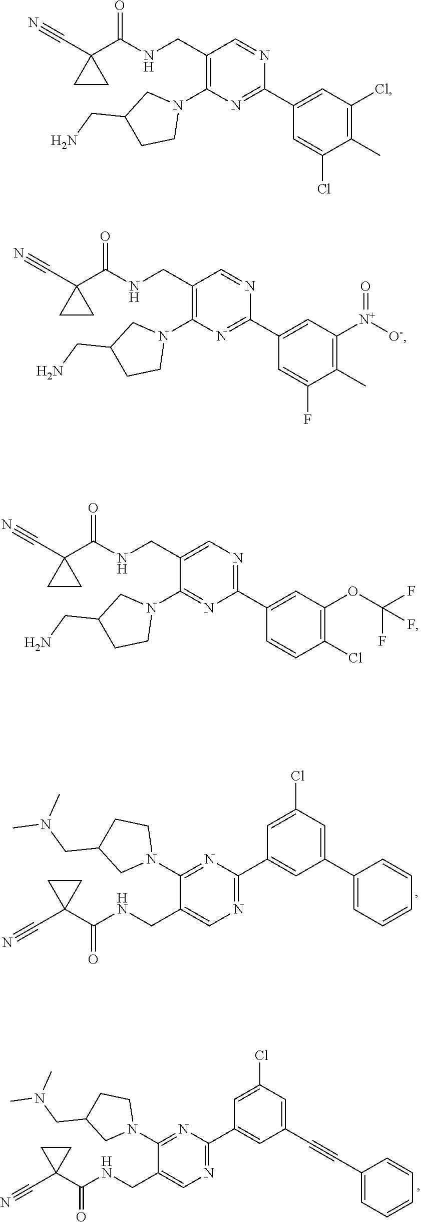

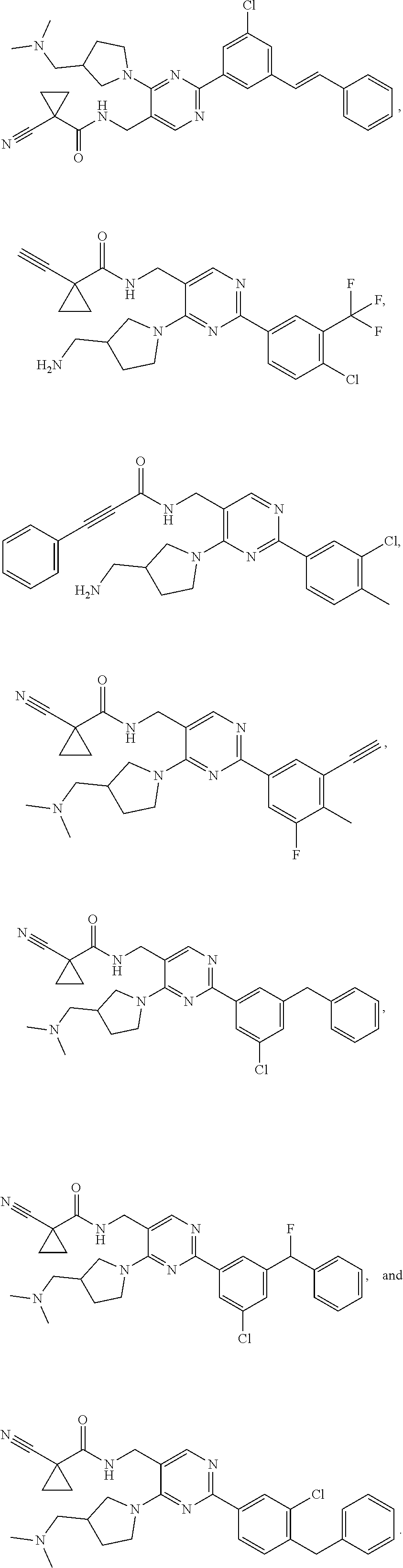

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I), (I-a), (I-b), (I-c), (I-d), (II), or (III), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formulas (I), (I-a), (I-b), (I-c), (I-d), (II), or (III), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formulas (I), (I-a), (I-b), (I-c), (I-d), (II), or (III), may comprise two or more enantiomers or diastereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formulas (I), (I-a), (I-b), (I-c), (I-d), (II), or (III), exist as tautomers. In circumstances where tautomerization is possible, a chemical equilibrium of the tautomers may exist. The exact ratio of the tautomers depends on several factors, including physical state, temperature, solvent, and pH. Some non-limiting examples of tautomeric equilibrium include:

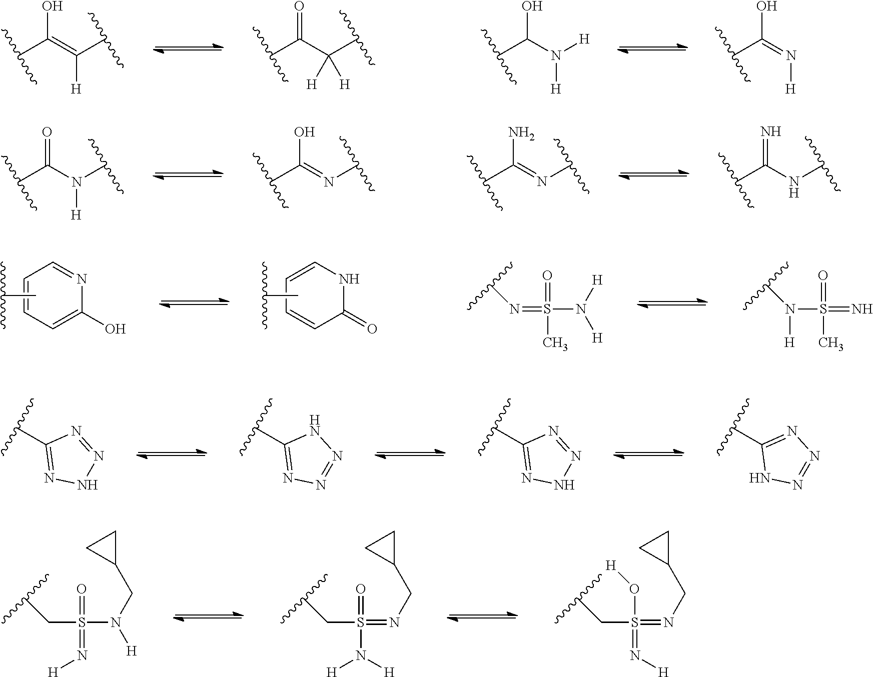

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formulas (I), (I-a), (I-b), (I-c), (I-d), (II), or (III). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formulas (I), (I-a), (I-b), (I-c), (I-d), (II), or (III), include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formulas (I), (I-a), (I-b), (I-c), (I-d), (II), or (III), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formulas (I), (I-a), (I-b), (I-c), (I-d), (II), or (III). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formulas (I), (I-a), (I-b), (I-c), (I-d), (II), or (III), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J Pharmaceutics, 37, 87 (1987); J. Larsen et al., Int. J Pharmaceutics, 47, 103 (1988); Sinkula et al., J Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formulas (I), (I-a), (I-b), (I-c), (I-d), (II), or (III), and at least one pharmaceutically acceptable excipient. In some embodiments, the pharmaceutical compositions comprises a compound or salt of Formula (I), and at least one pharmaceutically acceptable excipient. In some embodiments, the pharmaceutical compositions comprises a compound or salt of Formula (I-a), and at least one pharmaceutically acceptable excipient. In some embodiments, the pharmaceutical compositions comprises a compound or salt of Formula (I-b), and at least one pharmaceutically acceptable excipient. In some embodiments, the pharmaceutical compositions comprises a compound or salt of Formula (I-c), and at least one pharmaceutically acceptable excipient. In some embodiments, the pharmaceutical compositions comprises a compound or salt of Formula (I-d), and at least one pharmaceutically acceptable excipient. In some embodiments, the pharmaceutical compositions comprises a compound or salt of Formula (II), and at least one pharmaceutically acceptable excipient. In some embodiments, the pharmaceutical compositions comprises a compound or salt of Formula (III), and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Methods for formulation of the conjugates can include formulating any of the compounds, salts or conjugates with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates can be lyophilized or in powder form for re-constitution with a suitable vehicle, e.g., sterile pyrogen-free water, before use.

Pharmaceutical compositions can comprise at least one active ingredient (e.g., a compound, salt or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further can comprise more than one active compound (e.g., a compound, salt or conjugate and other agents) as necessary for the particular indication being treated. The active compounds can have complementary activities that do not adversely affect each other. For example, the composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

A compound or salt of any one of Formulas (I), (I-a), (I-b), (I-c), (I-d), (II), or (III), may be formulated in any suitable pharmaceutical formulation. A pharmaceutical formulation of the present disclosure typically contains an active ingredient (e.g., compound or salt of any one of Formulas (I), (I-a), (I-b), (I-c), (I-d), (II), or (III)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

Pharmaceutical compositions may also be prepared from a compound or salt of any one of Formulas (I), (I-a), (I-b), (I-c), (I-d), (II), or (III), and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

Without being bound to any particular mechanism, provided herein by the disclosure are compositions and methods for the modulation of protein structures and protein complexes (such as Ras and Raf) into alternate conformational states. Ras proteins are small GTPases that regulate cell differentiation, growth, and proliferation. Mutant KRas is one of the most common driver oncogenes. KRas mutations often occur earlier in tumor genesis and a variety of KRas mutations are implicated in many cancers. KRas mutated cancer cell lines can also be also Ras1 dependent, making Raf1 a viable target for KRas driven cancers. Provided herein are compositions and methods that can alter Raf/Ras activity. Such compositions and methods can alter Raf/Ras binding, induce conformational changes in an oncogenic KRas-Raf1 complex, and/or alter downstream signaling.

Any Ras protein is contemplated for use in any of the methods disclosed herein. In some cases, the Ras protein can include H-Ras (NC_000011.10), K-Ras (including K-Ras 4A and K-Ras 4B) (NC_000012.12) and N-Ras (NC_000001.11), members of the Ras subfamily that are clinically notable as being implicated in many types of cancer. The three human Ras genes generally encode homologous proteins made up of chains of 188 to 189 amino acids, designated H-Ras, N-Ras and K-RAS4A and K-Ras 4B (the two K-Ras proteins arise from alternative splicing). In some aspects, the Ras protein can include other GTPase members from the Ras super family, for example, GTPases from the Rho, Rab, Ran and Arf small GTPase sub families. In some cases, the Ras protein can include many other members of the Ras subfamily, (see also, Wennerberg et al., 2005, “The Ras superfamily at a glance,” J. Cell. Sci. 118 (Pt 5): 843-6, the disclosure of which is incorporated by reference in its entirety).

Without being bound to any particular mechanism, the present disclosure provides compounds or salts of Formula (I), (I-a), (I-b), (I-c), (I-d), (II), or (III), or a pharmaceutical composition comprising a compound or salt of any one of Formulas (I), (I-a), (I-b), (I-c), (I-d), (II), or (III), can cause a conformational change to Ras and alter Raf/Ras signaling. In some embodiments, the compounds or salts of Formulas (I), (I-a), (I-b), (I-c), (I-d), (II), or (III), or pharmaceutical compositions thereof, can cause a conformational change to a Ras/Raf protein complex and alter Raf/Ras signaling. In some embodiments, alteration of Raf/Ras signaling occurs in the presence of a second cancer therapy a chemotherapeutic, a targeted agent, and an immunotherapeutic. In some embodiments, the conformational change to Ras or the Ras/Raf complex occurs in the presence of a second cancer therapy a chemotherapeutic, a targeted agent, and an immunotherapeutic. In some embodiments, the compounds or salts of Formula (I), (I-a), (I-b), (I-c), (I-d), (II), or (III), or pharmaceutical compositions thereof, cause a conformational change to a Ras/Raf protein complex and can be effective for the treatment of cancer to a subject in need thereof.

In some aspects, the present disclosure provides a method for treatment, comprising administering to a subject in need thereof an effective amount of a compound or salt of Formula (I), (I-a), (I-b), (I-c), (I-d), (II), or (III). In embodiments, the method of treatment further comprises a second cancer therapy selected from a chemotherapeutic, a targeted agent, and an immunotherapeutic.

In some aspects, the present disclosure provides a method for the treatment of cancer, comprising administering to a subject in need thereof an effective amount of a compound or salt of Formula (I), (I-a), (I-b), (I-c), (I-d), (II), or (III), or a pharmaceutical composition comprising a compound or salt of any one of Formulas (I), (I-a), (I-b), (I-c), (I-d), (II), or (III). In embodiments, the method of treating cancer further comprises a second cancer therapy selected from a chemotherapeutic, a targeted agent, and an immunotherapeutic.

In some aspects, the present disclosure provides a method of modulating Ras/Raf signaling, comprising administering to a subject in need thereof an effective amount of a compound or salt of Formula (I), (I-a), (I-b), (I-c), (I-d), (II), or (III), or a pharmaceutical composition comprising a compound or salt of any one of Formulas (I), (I-a), (I-b), (I-c), (I-d), (II), or (III). In embodiments, the method of modulating Ras/Raf signaling further comprises a second cancer therapy selected from a chemotherapeutic, a targeted agent, and an immunotherapeutic.

In some aspects, the present disclosure provides a method of allosterically modulating Ras/Raf signaling, comprising administering to a subject in need thereof an effective amount of a compound or salt of Formula (I), (I-a), (I-b), (I-c), (I-d), (II), or (III), or a pharmaceutical composition comprising a compound or salt of any one of Formulas (I), (I-a), (I-b), (I-c), (I-d), (II), or (III)). In embodiments, the method of allosterically modulating Ras/Raf signaling further comprises a second cancer therapy selected from a chemotherapeutic, a targeted agent, and an immunotherapeutic.

In some aspects, the present disclosure provides a method of inhibiting Ras/Raf signaling comprising administering to a subject in need thereof an effective amount of a compound or salt of Formula (I), (I-a), (I-b), (I-c), (I-d), (II), or (III), or a pharmaceutical composition comprising a compound or salt of any one of Formulas (I), (I-a), (I-b), (I-c), (I-d), (II), or (III). In embodiments, the method of inhibiting Ras/Raf signaling further comprises a second cancer therapy selected from a chemotherapeutic, a targeted agent, and an immunotherapeutic.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-16 show general and exemplary procedures for the preparation of the claimed RAS Modulators. Examples 17-19 show compound screening, SHG bioassay and pERK inhibition bioassay data, respectively.

### Example 1: Exemplary Synthesis of Compound 2

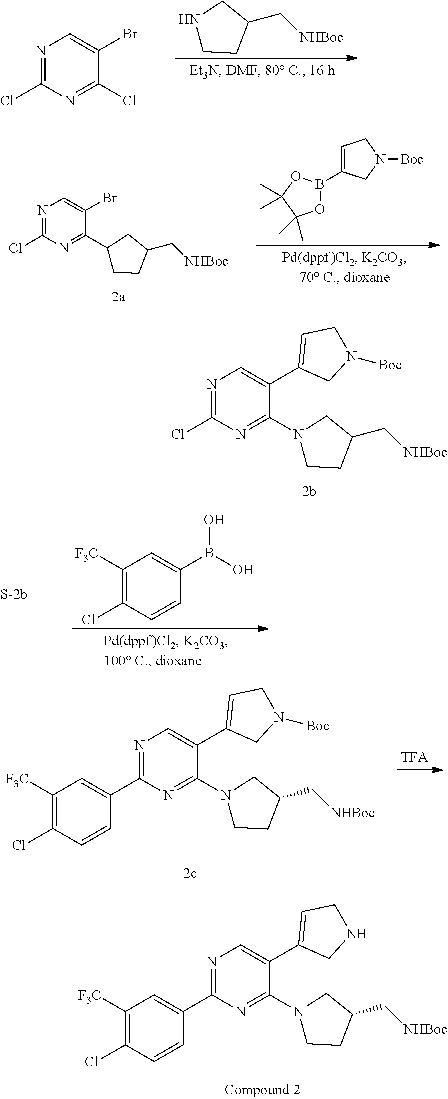

Step 1. Synthesis of tert-butyl N-[[1-(5-bromo-2-chloro-pyrimidin-4-yl)pyrrolidin-3-yl]methyl] carbamate (2a): To a solution of 5-bromo-2,4-dichloro-pyrimidine (3.4 g, 14.92 mmol) and tert-butyl N-(pyrrolidin-3-ylmethyl) carbamate (3.0 g, 14.92 mmol) in DMF (20 mL) was added Et₃N (4.52 g, 44.76 mmol). The solution was stirred at 25° C. for 2 hrs. The mixture was diluted with EtOAc (150 mL), washed with water (50 mL) and brine (50 mL). The organic phase was concentrated, and the residue was purified by silica gel colunm (PE:EtOAc=4:1) to give tert-butyl N-[[1-(5-bromo-2-chloro-pyrimidin-4-yl)pyrrolidin-3-yl]methyl]carbamate (2a) (3.0 g, 7.66 mmol, 51.3% yield) as a pink oil. LCMS (ESI): m/z calcd for C14H20BrClN4O2+H⁺: 393.1, found: 393.1.

Step 2. Synthesis of tert-butyl 3-[4-[3-[(tert-butoxycarbonylamino) methyl] pyrrolidin-1-yl]-2-chloro-pyrimidin-5-yl]-2,5-dihydropyrrole-1-carboxylate (2b): To a solution of tert-butyl N-[[1-(5-bromo-2-chloro-pyrimidin-4-yl)pyrrolidin-3-yl] methyl]carbamate (2a) (750.0 mg, 1.91 mmol) and tert-butyl 3-(4,4,5,5-tetramethyl-1,3,2-dioxaborolan-2-yl)-2,5-dihydropyrrole-1-carboxylate (509 mg, 1.72 mmol) in water (3.5 mL) and 1,4-dioxane (14 mL) were added Pd(dppf)Cl₂ (140 mg, 0.19 mmol) and K₂CO₃ (793 mg, 5.74 mmol) at 25° C. The mixture was stirred at 70° C. for 2 h under argon. The mixture was diluted with EtOAC (100*2 mL), washed with water (50 mL) and brine (50 mL), dried over Na₂SO₄, concentrated. The crude product was purified by flash chromatography (H₂O:CH₃CN=90:10 to 5:95) to afford tert-butyl 3-[4-[3-[(tert-butoxycarbonylamino)methyl]pyrrolidin-1-yl]-2-chloro-pyrimidin-5-yl]-2,5-dihydropyrrole-1-carboxylate (2b) (580 mg, 1.21 mmol, 63% yield) as brown solid. LCMS (ESI): m/z calcd for C23H34ClN5O4+H⁺: 480.23, found 480.3.

Step 3. Synthesis of tert-butyl 3-[2-[4-chloro-3-(trifluoromethyl)phenyl]-4-[(3S)-3-[(tert-butoxy-carbonylamino)methyl]pyrrolidin-1-yl]pyrimidin-5-yl]-2,5-dihydropyrrole-1-carboxylate (2c): To a solution of [4-chloro-3-(trifluoromethyl)phenyl]boronic acid (46.74 mg, 0.21 mmol), tert-butyl 3-[2-chloro-4-[(3S)-3-[(tert-butoxycarbonylamino)methyl]pyrrolidin-1-yl]pyrimidin-5-yl]-2,5-dihydropyrrole-1-carboxylate (S-2b) (100.0 mg, 0.21 mmol; synthesized according to steps 1 and 2 using the R-enantiomer of the amine in step 1) and K₂CO₃ (86.25 mg, 0.6300 mmol) in 1,4-dioxane (5 mL) and water (1 mL) was added Pd(dppf)Cl₂ (15.23 mg, 0.020 mmol). The mixture was stirred at 90° C. overnight under Ar. The mixture was extracted with EtOAc (3×10 mL), and washed with H2O (3×10 mL). All the organic phases were collected, washed with brine (20 mL), dried over Na₂SO₄, and filtered. The organic solution was concentrated to provide the crude, which was purified by slica gel chromatography (PE:EtOAc=5:1) to provide tert-butyl 3-[2-[4-chloro-3-(trifluoromethyl)phenyl]-4-[(3S)-3-[(tert-butoxycarbonylamino)methyl]pyrrolidin-1-yl]pyrimidin-5-yl]-2,5-dihydropyrrole-1-carboxylate (70.0 mg, 0.11 mmol, 54% yield) as a brown solid. LCMS (ESI): m/z calcd for C30H37ClF3N5O4+H: 624.1, found: 624.3.

Step 4. Synthesis of [(3S)-1-[2-[4-chloro-3-(trifluoromethyl)phenyl]-5-(2,5-dihydro-1H-pyrrol-3-yl) pyrimidin-4-yl]pyrrolidin-3-yl]methanamine (2): To a solution of tert-butyl 3-[2-[4-chloro-3-(trifluoromethyl)phenyl]-4-[(3S)-3-[(tert-butoxy-carbonylamino)methyl]pyrrolidin-1-yl]pyrimidin-5-yl]-2,5-dihydropyrrole-1-carboxylate (2c) (50.0 mg, 0.080 mmol) in 1,4-dioxane (2 mL) was added HCl (5.0 mL, 1 mol/L in dioxane, 0.5 mmol). The reaction mixture was stirred at 25° C. overnight. The solvent was removed to get the crude, which was then purified by prep-HPLC (eluting with 0-90% acetonitrile in water (0.1% TFA)) to give [(3S)-1-[2-[4-chloro-3-(trifluoromethyl)phenyl]-5-(2,5-dihydro-1H-pyrrol-3-yl)pyrimidin-4-yl]pyrrolidin-3-yl]methanamine; 2,2,2-trifluoroacetic acid (2) (32.37 mg, 0.050 mmol, 61% yield) as white solid. LCMS (ESI): m/z calcd for C20H21ClF3N5+H: 424.1, found: 424.1. ¹H NMR (400 MHz, MeOD) δ 8.70 (s, 1H), 8.57-8.50 (m, 1H), 8.28 (s, 2H), 7.84-7.74 (m, 1H), 6.03-5.94 (m, 1H), 4.53-4.26 (m, 4H), 3.96-3.72 (m, 3H), 3.55-3.43 (m, 1H), 3.17-3.07 (m, 2H), 2.68-2.58 (m, 1H), 2.34-2.25 (m, 1H), 1.88-1.78 (m, 1H).

### Example 2: Exemplary Synthesis of Compound 3

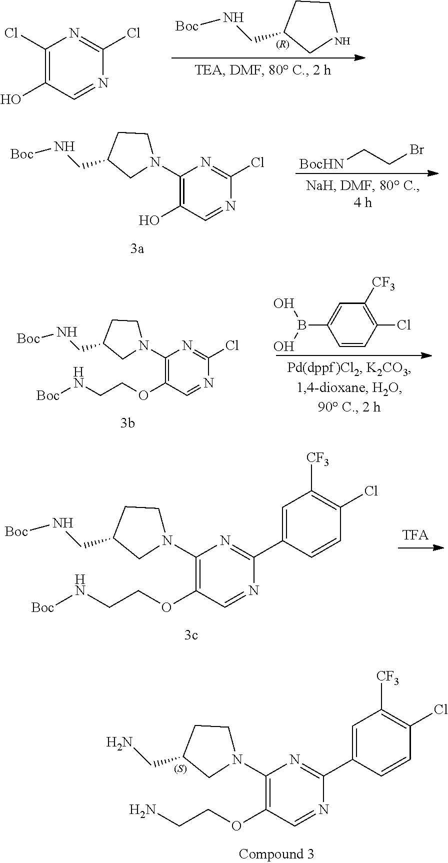

Step 1. Synthesis of tert-butyl (S)-((1-(2-chloro-5-hydroxypyrimidin-4-yl)pyrrolidin-3-yl)methyl) carbamate (3a): To a solution of 2, 4-dichloropyrimidin-5-ol (120 mg, 0.73 mmol) and tert-butyl (R)-(pyrroli-din-3-ylmethyl)carbamate hydrochloride (189.41 mg, 0.80 mmol) in DMF (15 mL) was added NEt₃ (1.5 mL, 0.73 mmol) at rt. The mixture was stirred at 80° C. for 2 h. The mixture was diluted with water (20 mL) and extracted with EtOAc (50 mL×3). The organic solution was washed with water (50 mL) and brine (50 mL), dried over Na₂SO₄, concentrated under reduced pressure. The crude product was purified by silica chromatography column (100-200 mesh size) eluting with 50% EtOAc in PE to obtain tert-butyl (S)-((1-(2-chloro-5-hydroxypyrimidin-4-yl)pyrrolidin-3-yl)methyl)carbamate (3a) (150 mg, 0.46 mmol, 62.72% yield) as a yellow solid. LCMS (ESI): m/z calcd for C14H21ClN4O3+H⁺: 328.1; found: 329.2.

Step 2. Synthesis of tert-butyl N-[2-[2-chloro-4-[(3S)-3-[(tert-butoxycarbonylamino)methyl]pyrroli-din-1-yl]pyrimidin-5-yl]oxyethyl]carbamate (3b): To a solution of tert-butyl N-[[(3S)-1-(2-chloro-5-hydroxy-pyrimidin-4-yl) pyrrolidin-3-yl] methyl]carbamate (3a) (150.0 mg, 0.46 mmol) in DMF (10 mL) were added NaH (32.9 mg, 1.37 mmol) and tert-butyl N-(2-bromoethyl) carbamate (123 mg, 0.55 mmol) at rt. The mixture was stirred at 80° C. for 4 h. The mixture was diluted with water (20 mL), extracted with EtOAc (50 mL×3), dried over Na₂SO₄, and concentrated. The crude product was purified by silica chromatography column (100-200 mesh size) eluting with 20% EtOAc in PE to obtain tert-butyl N-[2-[2-chloro-4-[(3S)-3-[(tert-butoxycarbonylamino)methyl]pyrrolidin-1-yl]pyrimidin-5-yl]oxyethyl]carbamate (3b) (90 mg, 0.19 mmol, 41.8% yield) as a brown solid. LCMS (ESI) m/z calcd for C21H34ClN5O5+H⁺: 471.22, found: 472.2.

Step 3. Synthesis of tert-butyl (S)-((1-(5-(2-((tert-butoxycarbonyl) amino) ethoxy)-2-(4-chloro-3-(trifluoromethyl) phenyl) pyrimidin-4-yl) pyrrolidin-3-yl) methyl) carbamate (3c): To a solution of tert-butyl N-[2-[2-chloro-4-[(3S)-3-[(tert-butoxycarbonylamino) methyl] pyrrolidin-1-yl] pyrimidin-5-yl] oxyethyl] carbamate (3b) (90.0 mg, 0.19 mmol) and [4-chloro-3-(tri-fluoromethyl) phenyl] boronic acid (64.2 mg, 0.29 mmol) in 1,4-dioxane (2 mL) and water (2 mL) were added Pd(dppf)Cl₂ (2.63 mg, 0.0200 mmol) and K₂CO₃ (52.2 mg, 0.38 mmol) at rt. The mixture was stirred at 100° C. under Ar for 2 h. The mixture was diluted with water (20 mL) and extracted with EtOAc (50 mL×3), dried over Na₂SO₄, and concentrated. The crude product was purified by silica column chromatography (100-200 mesh size) eluting with 50% EtOAc in PE to obtain tert-butyl N-[2-[2-[4-chloro-3-(trifluoromethyl) phenyl]-4-[(3S)-3-[(tert-butoxycarbonylamino) methyl]pyrrolidin-1-yl] pyrimidin-5-yl] oxyethyl] carbamate (3c) (70 mg, 0.11 mmol, 59.6 yield) as a brown solid. LCMS (ESI): m/z calcd for C28H37ClF3N₅O₅+H⁺:616.2; found: 616.3.

Step 4. Synthesis of (S)-2-((4-(3-(aminomethyl)pyrrolidin-1-yl)-2-(4-chloro-3-(trifluoromethyl) phenyl)pyrimidin-5-yl)oxy)ethan-1-amine (3): To a solution of tert-butyl N-[2-[2-[4-chloro-3-(trifluoromethyl)phenyl]-4-[(3S)-3-[(tert-butoxy carbonylamino) methyl]pyrrolidin-1-yl] pyrimidin-5-yl]oxyethyl] carbamate (3c) (70.0 mg, 0.11 mmol) in DCM (4 mL) was added TFA (1.0 mL, 0.11 mmol) at rt. The mixture was stirred at 25° C. for 2 h. The mixture was concentrated and purified by prep-HPLC (eluting with H₂O:CH₃CN (0.1% TFA as additive) from 90:10 to 5:95) to give (S)-2-((4-(3-(aminomethyl) pyrrolidin-1-yl)-2-(4-chloro-3-(trifluoromethyl)phenyl)pyrimidin-5-yl)oxy)ethan-1-amine; 2,2,2-trifluoroacetic acid (3) (20.66 mg, 0.0314 mmol, 27% yield) as a white solid. LCMS calcd for C18H21ClF3N5O+H⁺: 416.1, found: 416.2. ¹H NMR (400 MHz, MeOD) δ 8.58 (s, 1H), 8.43 (s, 1H), 7.99 (s, 1H), 7.79 (d, J=8.6 Hz, 1H), 4.36 (d, J=3.8 Hz, 2H), 4.23 (d, J=24.7 Hz, 2H), 3.99 (s, 1H), 3.72 (s, 1H), 3.48 (d, J=3.2 Hz, 2H), 3.14 (tt, J=20.3, 10.4 Hz, 2H), 2.65 (s, 1H), 2.30 (d, J=6.1 Hz, 1H), 1.93-1.72 (m, 1H).

### Example 3A: Exemplary Synthesis of Compound 5

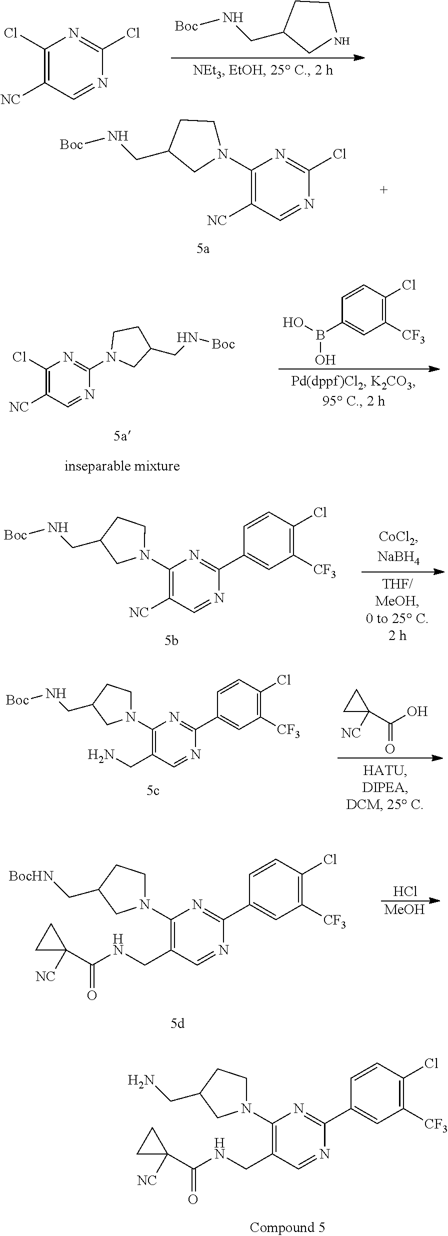

Step 1. Synthesis of tert-butyl ((1-(2-chloro-5-cyanopyrimidin-4-yl)pyrrolidin-3-yl)methyl)carbamate (5a) and tert-butyl ((1-(4-chloro-5-cyanopyrimidin-2-yl)pyrrolidin-3-yl)methyl) carbamate (5a′): A mixture of 2,4-dichloropyrimidine-5-carbonitrile (12.0 g, 68.97 mmol), tert-butyl N-(pyrrolidin-3-ylmethyl)carbamate (17.27 g, 68.97 mmol) and Et₃N (20.9 g, 206.91 mmol) in DMF (120 mL) was stirred at 25° C. for 2 h. The mixture was diluted with EtOAc (500 mL), washed with water (3×500 mL) and brine (200 mL). The organic phase was concentrated to afford a crude mixture of tert-butyl N-[[1-(2-chloro-5-cyano-pyrimidin-4-yl)pyrrolidin-3-yl]methyl]carbamate (5a) and tert-butyl N-[[1-(4-chloro-5-cyano-pyrimidin-2-yl)pyrrolidin-3-yl]methyl]carbamate (5a′) (total of 23.2 g, 64.64 mmol, 99.6% total yield) as an yellow oil which was used without further purification. LCMS (ESI): m/z calcd for C₁₅H₂OClN₅O₂+H⁺: 338.1, found: 338.1.

Step 2. Synthesis of tert-butyl ((1-(2-(4-chloro-3-(trifluoromethyl)phenyl)-5-cyanopyrimidin-4-yl) pyrrolidin-3-yl)methyl)carbamate (5b): A degassed solution of [4-chloro-3-(trifluoromethyl)phenyl] boronic acid (21.9 g, 97.69 mmol), K₂CO₃ (40.50 g, 293.1 mmol), Pd(dppf)Cl₂ (7.15 g, 9.77 mmol) and tert-butyl N-[[1-(2-chloro-5-cyano-pyrimidin-4-yl)pyrrolidin-3-yl] methyl]carbamate (33.0 g crude oil; mixture with regio-isomer of tert-butyl N-[[1-(4-chloro-5-cyano-pyrimidin-2-yl)pyrrolidin-3-yl] methyl]carbamate) in 1,4-dioxane (432 mL) and water (87 mL) was stirred at 95° C. under argon for 2 h. The mixture was concentrated under vacuo, and the residue was purified by silica gel column (petroleum ether/EtOAc=5:1 to 2:1) to get tert-butyl N-[[1-[2-[4-chloro-3-(trifluoromethyl)phenyl]-5-cyano-pyrimidin-4-yl]pyrrolidin-3-yl]methyl]carbamate (5b) (15.5, 30.98 mmol, 63.4% yield) as a white solid. LCMS (ESI): m/z calcd for C₂₂H₂₃ClF₃N₅O₂+H⁺: 482.1, found: 482.1. ¹H NMR (400 MHz, DMSO) δ 8.79 (s, 1H), 8.69 (s, 1H), 8.59 (d, J=8.4 Hz, 1H), 7.91 (d, J=8.4 Hz, 1H), 7.10 (s, 1H), 4.07-3.47 (m, 4H), 3.04 (s, 2H), 2.44-2.29 (m, 1H), 1.99 (s, 1H), 1.76 (s, 1H), 1.38 (s, 9H).

Step 3. Synthesis of tert-butyl ((1-(5-(aminomethyl)-2-(4-chloro-3-(trifluoromethyl)phenyl) pyrimidin-4-yl)pyrrolidin-3-yl)methyl)carbamate (5c): To a solution of tert-butyl N-[[1-[2-[4-chloro-3-(trifluoromethyl)phenyl]-5-cyano-pyrimidin-4-yl]pyrrolidin-3-yl]methyl]carbamate (5b) (8.5 g, 17.64 mmol) and CoCl₂ (2.75 g, 21.17 mmol) in THF (240 mL) and methanol (160 mL) was added NaBH₄ (6.67 g, 176.39 mmol) slowly by three portions at 0° C. The solution was stirred at 0° C. to rt for 1 h. The mixture was quenched by sat. NH₄Cl solution (30 mL) and diluted with EtOAc (500 mL). The mixture was filtered over celite, and the filter cake was rinsed with EtOAc/THF/MeOH/NH₄OH (600 mL, 10:10:1:0.01) until no product was found in the eluent monitored by TLC. Then the filtrate was concentrated under vacuo to afford a residue which was further triturated with EtOAc/THF/MeOH/NH₄OH (10:10:1:0.01). The mixture was filtered, and the solution was concentrated to give crude tert-butyl N-[[1-[5-(aminomethyl)-2-[4-chloro-3-(trifluoromethyl)phenyl]pyrimidin-4-yl]pyrrolidin-3-yl]methyl]carbamate (5c) (8 g) as a light yellow solid which was used without further purification. LCMS (ESI): m/z calcd for C₂₂H₂₇ClF₃N₅O₂+H⁺: 486.1, found: 486.1.

Step 4. Synthesis of tert-butyl ((1-(2-(4-chloro-3-(trifluoromethyl)phenyl)-5-((1-cyanocyclopropane-1-carboxamido)methyl)pyrimidin-4-yl)pyrrolidin-3-yl)methyl)carbamate (5d): To a solution of 1-cyanocyclopropanecarboxylic acid (3.66 g, 32.91 mmol) and tert-butyl N-[[1-[5-(aminomethyl)-2-[4-chloro-3-(trifluoromethyl)phenyl]pyrimidin-4-yl]pyrrolidin-3-yl]methyl]carbamate (5c) (12.3 g, 25.31 mmol) in DCM (240 mL) was added HTAU (19.25 g, 50.62 mmol) and DIPEA (26.0 mL, 152 mmol) at room temperature. The mixture was stirred at 30° C. for 16 h under Ar. The mixture was diluted with DCM (500 mL) and washed with water (4×300 mL) and brine (2×300 mL). The organic layer was separated and dried over anhydrous Na₂SO₄, filtered and concentrated to give a crude product which was purified by silica gel column (petroleum ether/EtOAc=5:1 to 1:2) to obtain tert-butyl N-[[1-[2-[4-chloro-3-(trifluoromethyl)phenyl]-5-[[(1-cyanocyclopropane carbonyl)amino]methyl]pyrimidin-4-yl]pyrrolidin-3-yl]methyl]carbamate (5d) (8.50 g, 14.68 mmol, 58% yield) as a white powder. LCMS (ESI): m/z calcd for C₂₇H₃₀ClF₃N₆O₃+H⁺: 579.1, found: 579.1. 1H NMR (400 MHz, DMSO) δ 8.71 (s, 1H), 8.65 (t, J=4.4 Hz, 1H), 8.58 (d, J=8.3 Hz, 1H), 8.16 (s, 1H), 7.85 (d, J=8.4 Hz, 1H), 7.04 (t, J=5.0 Hz, 1H), 4.51-4.37 (m, 2H), 3.80 (dd, J=10.2, 6.8 Hz, 2H), 3.75-3.64 (m, 1H), 3.46 (dd, J=10.7, 6.7 Hz, 1H), 3.10-2.95 (m, 2H), 2.42-2.32 (m, 1H), 2.08-1.94 (m, 1H), 1.73-1.64 (m, 1H), 1.62-1.50 (m, 4H), 1.39 (s, 9H).

Step 5. Synthesis of N-((4-(3-(aminomethyl)pyrrolidin-1-yl)-2-(4-chloro-3-(trifluoromethyl)phenyl) pyrimidin-5-yl)methyl)-1-cyanocyclopropane-1-carboxamide (5): To a solution of tert-butyl N-[[1-[2-[4-chloro-3-(trifluoromethyl)phenyl]-5-[[(1-cyano cyclopropanecarbonyl)amino]methyl]pyrimidin-4-yl]pyrrolidin-3-yl]methyl]carbamate (5d) (4.0 g, 6.91 mmol) in MeOH (30 mL) was added HCl (80.0 mL, 3 mol/L in MeOH, 240 mmol) at 20° C. The solution was stirred for 2 h at 20° C. The mixture was concentrated. The residue was triturated in MeCN/MeOH (100 mL, 10/1, V/V) at 80° C. for 5 h, and 50° C. for 5 h. The same operation was repeated for three times. The suspension was filtered, and the solid was dried to afford N-[[4-[3-(aminomethyl)pyrrolidin-1-yl]-2-[4-chloro-3-(trifluoromethyl)phenyl]pyrimidin-5-yl]methyl]-1-cyano-cyclopropanecarboxamide; hydrochloride (5) (3.5 g, 8.64 mmol, 98% yield, HCl salt) as a white powder. LCMS (ESI): m/z calcd for C₂₂H₂₂ClF₃N₆O+H⁺: 479.2, found: 479.1. ¹H NMR (400 MHz, MeOD) δ 8.62 (s, 1H), 8.46 (d, J=8.0 Hz, 1H), 8.18 (s, 1H), 7.82 (d, J=8.4 Hz, 1H), 4.67-4.55 (m, 2H), 4.23-4.12 (m, 2H), 4.02-3.95 (m, 1H), 3.75 (t, J=8.4 Hz, 1H), 3.20-3.09 (m, 2H), 2.72-2.65 (m, 1H), 2.38-2.31 (m, 1H), 1.95-1.86 (m, 1H), 1.64-1.57 (m, 4H).

### Example 3B: Exemplary Synthesis of Compound 5

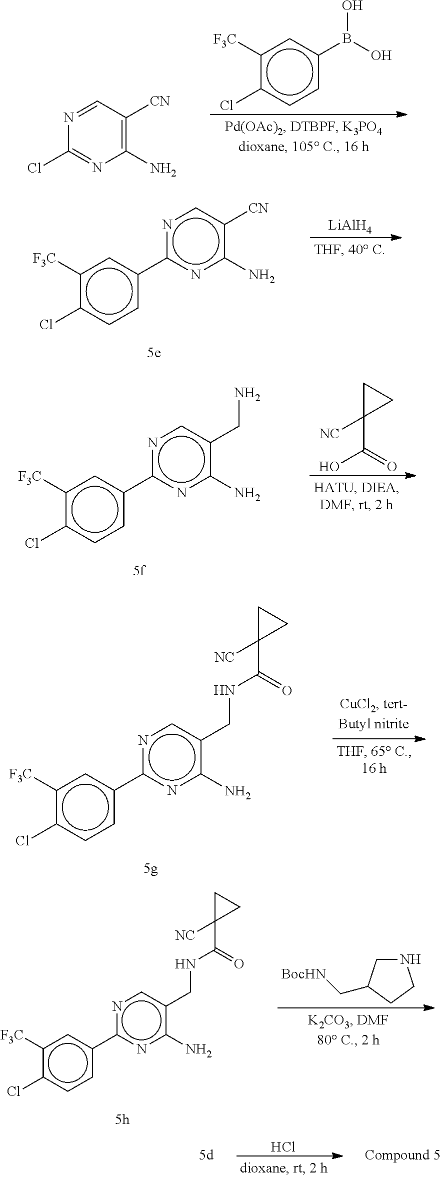

Step 1. Synthesis of 4-amino-2-(4-chloro-3-(trifluoromethyl)phenyl)pyrimidine-5-carbonitrile (5e): To a solution of 4-amino-2-chloro-pyrimidine-5-carbonitrile (10.0 g, 64.7 mmol) and [4-chloro-3-(trifluoromethyl)phenyl]boronic acid (14.52 g, 64.7 mmol) in 1,4-dioxane (300 mL) were added K₃PO4 (17.86 g, 129.4 mmol), Pd(OAc)₂ (0.72 g, 3.23 mmol) and DTBPF (1.53 g, 3.23 mmol) under argon. The mixture was stirred at 105° C. for 16 h. The mixture was diluted with H₂O (100 mL) and extracted with EtOAc (2×100 mL). The organic solution was washed with brine (100 mL×2), concentrated and purified by silica chromatography column eluting with 50% EtOAc in petroleum ether to obtain 4-amino-2-[4-chloro-3-(trifluoromethyl)phenyl]pyrimidine-5-carbonitrile (5e) (12.0 g, 40.18 mmol, 62% yield) as a yellow solid. LCMS (ESI): m/z calcd for C₁₂H₆ClF₃N₄+H⁺: 299.1, found: 299.1.

Step 2. Synthesis of 5-(aminomethyl)-2-(4-chloro-3-(trifluoromethyl)phenyl)pyrimidin-4-amine (5f): To a solution of 4-amino-2-[4-chloro-3-(trifluoromethyl)phenyl]pyrimidine-5-carbonitrile (5e) (14.5 g, 48.55 mmol) in THF (200 mL) was added LiAlH₄ (5.53 g, 146 mmol). The mixture was stirred at 40° C. for 3 h. The reaction mixture was quenched with H₂O (5.5 mL), 15% of aqueous NaOH (5.5 mL) and H₂O (16.5 mL) at 0° C. The mixture was diluted with EtOAc (100 mL), filtered through celite pad and concentrated to obtain 5-(aminomethyl)-2-[4-chloro-3-(trifluoromethyl)phenyl] pyrimidin-4-amine (5f) (6.5 g, 21.48 mmol, 44.2% yield) as a yellow oil. LCMS (ESI): m/z calcd for C₁₂H₁₀ClF₃N₄+H⁺: 303.1; found: 303.1.

Step 3. Synthesis of N-((4-amino-2-(4-chloro-3-(trifluoromethyl)phenyl)pyrimidin-5-yl)methyl)-1-cyanocyclopropane-1-carboxamide (5g): To a solution of 5-(aminomethyl)-2-[4-chloro-3-(trifluoromethyl)phenyl]pyrimidin-4-amine (5f) (6.5 g, 21.47 mmol) in DMF (100 mL) was added DIEA (8.31 g, 64.4 mmol), 1-cyanocyclopropane carboxylic acid (2.86 g, 25.8 mmol) and HATU (16.33 g, 42.95 mmol) at 25° C. The mixture was stirred at 25° C. for 3 h. The mixture was diluted with water (100 mL), extracted with EtOAc (100 mL×3), washed with brine (100 mL), dried over Na₂SO₄, concentrated, and purified by pre-HPLC (eluted with CH₃CN in H₂O from 5.0% to 95%) to obtain N-((4-amino-2-(4-chloro-3-(trifluoromethyl)phenyl)pyrimidin-5-yl)methyl)-1-cyanocyclopropane-1-carboxamide (5g) (3.80 g, 9.60 mmol, 44.7%) as a pale yellow solid. LCMS (ESI): m/z calcd for C₁₇H₁₃ClF₃N₅O+H⁺: 396.2; found: 396.2.

Step 4. Synthesis of N-((4-chloro-2-(4-chloro-3-(trifluoromethyl)phenyl)pyrimidin-5-yl)methyl)-1-cyanocyclopropane-1-carboxamide (5h): To a solution of N-[[4-amino-2-[4-chloro-3-(trifluoromethyl)phenyl]pyrimidin-5-yl]methyl]-1-cyano-cyclopropanecarboxamide (5g) (1.8 g, 4.55 mmol) in THF (30 mL) were added CuCl₂ (0.91 g, 6.82 mmol), and tert-Butyl nitrite (0.94 g, 9.1 mmol) at rt. The mixture was stirred at 65° C. for 16 h. The mixture was diluted with water (50 mL), extracted with EtOAc (50 mL×2), washed with brine (50 mL), dried over Na₂SO₄, concentrated to obtain N-[[4-chloro-2-[4-chloro-3-(trifluoromethyl) phenyl]pyrimidin-5-yl]methyl]-1-cyano-cyclopropanecarboxamide (5h) (720 mg, 1.73 mmol, 38.1% yield) as a crude yellow solid. LCMS (ESI): m/z calcd for C₁₇H₁₁Cl₂F₃N₄O+H⁺: 415.1; found: 415.1.

Step 5. Synthesis of tert-butyl ((1-(2-(4-chloro-3-(trifluoromethyl)phenyl)-5-((1-cyanocyclo propane-1-carboxamido)methyl)pyrimidin-4-yl)pyrrolidin-3-yl)methyl)carbamate (5d): To a solution of N-[[4-chloro-2-[4-chloro-3-(trifluoromethyl)phenyl]pyrimidin-5-yl]methyl]-1-cyano-cyclopropanecarboxamide (5h) (500.0 mg, 1.2 mmol) in DMF (10 mL) was added K₂CO₃ (397 mg, 2.88 mmol) and tert-butyl N-(pyrrolidin-3-ylmethyl)carbamate (241.2 mg, 1.2 mmol) at rt. The mixture was stirred at 80° C. for 2 h. LCMS showed the reaction was completed. The mixture was diluted with H₂O (30 mL) and extracted with EtOAc (50 mL×2). The organic solution was washed with brine (30 mL×2), concentrated, and purified by silica chromatography column (200-300 mesh size, eluting with 60% EtOAc in petroleum ether to obtain tert-butyl N-[[1-[2-[4-chloro-3-(trifluoromethyl)phenyl]-5-[[(1-cyanocyclopropanecarbonyl)amino]methyl]pyrimidin-4-yl]pyrrolidin-3-yl]methyl]carbamate (5d) (350 mg, 0.60 mmol, 50.2% yield) as white solid. LCMS (ESI): m/z calcd for C₂₇H₃₀ClF₃N₆O₃+H⁺: 579.1, found: 579.2.

Step 6. Synthesis of N-((4-(3-(aminomethyl)pyrrolidin-1-yl)-2-(4-chloro-3-(trifluoromethyl)phenyl) pyrimidin-5-yl)methyl)-1-cyanocyclopropane-1-carboxamide (5): To a solution of tert-butyl N-[[1-[2-[4-chloro-3-(trifluoromethyl)phenyl]-5-[[(1-cyanocyclo-propanecarbonyl)amino]methyl]pyrimidin-4-yl]pyrrolidin-3-yl]methyl]carbamate (5d) (350.0 mg, 0.60 mmol) in dioxane (10 mL) was added HCl (10.0 mL, 3.02 mmol, 4 mol/L in MeOH) at 25° C. The mixture was stirred at 25° C. for 3 h. LCMS showed the reaction was completed. The mixture was concentrated. The residue was stirred in MeCN at 80° C. for 5 h and at 20° C. for 2 h. The solid was filtered and dried to give N-[[4-[3-(aminomethyl)pyrrolidin-1-yl]-2-[4-chloro-3-(trifluoromethyl) phenyl]pyrimidin-5-yl]methyl]-1-cyano-cyclopropanecarboxamide hydrochloride (5) (260.0 mg, 0.43 mmol, 70.9% yield) as a yellow solid. LCMS (ESI): m/z calcd for C₂₂H₂₂ClF₃N₆O+H⁺: 479.2; found: 479.1. ¹H NMR (400 MHz, MeOD) δ 8.62 (d, J=1.2 Hz 1H), 8.46 (d, J=8.4 Hz, 1H), 8.18 (s, 1H), 7.82 (d, J=8.4 Hz, 1H), 4.67-4.59 (m, 2H), 4.23-4.12 (m, 2H), 4.03-3.96 (m, 1H), 3.78-3.73 (m, 1H), 3.20-3.09 (m, 2H), 2.74-2.63 (m, 1H), 2.39-2.31 (m, 1H), 1.95-1.86 (m, 1H), 1.64-1.57 (m, 4H).

### Example 4: Exemplary Synthesis of Compound 6

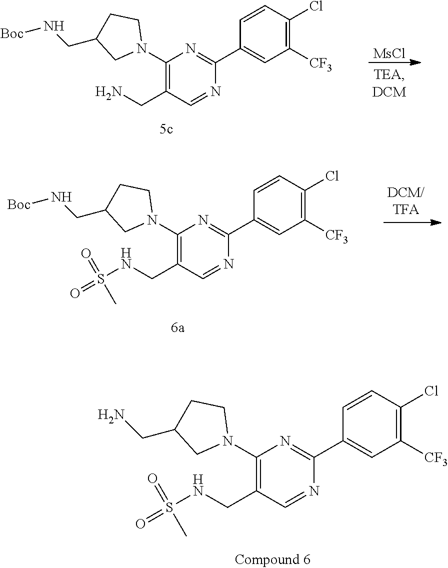 [SMILES: "MsCl" ; CC(C)(C)OC(=O)NCC1CCN(c2nc(-c3ccc(Cl)c(C(F)(F)F)c3)ncc2CN)C1 ; CC(C)(C)OC(=O)NCC1CCN(c2nc(-c3ccc(Cl)c(C(F)(F)F)c3)ncc2CNS(C)(=O)=O)C1 ; CS(=O)(=O)NCc1cnc(-c2ccc(Cl)c(C(F)(F)F)c2)nc1N1CCC(CN)C1]

Step 1. Synthesis of tert-butyl ((1-(2-(4-chloro-3-(trifluoromethyl)phenyl)-5-(methylsulfonamido methyl)pyrimidin-4-yl)pyrrolidin-3-yl)methyl)carbamate (6): To a solution of tert-butyl N-[[1-[5-(aminomethyl)-2-[4-chloro-3-(trifluoromethyl)phenyl] pyrimidin-4-yl]pyrrolidin-3-yl]methyl]carbamate (5c) (50.0 mg, 0.10 mmol) and Et₃N (31.18 mg, 0.31 mmol) in DCM (3 mL) was added methanesulfonyl chloride (14.14 mg, 0.12 mmol). The solution was stirred at 25° C. for 1 hr. The mixture was diluted with EtOAc (20 mL), and washed with water. The organic phase was concentrated and the residue was purified by silica column (DCM:MeOH=20:1) to get tert-butyl N-[[1-[2-[4-chloro-3-(trifluoromethyl)phenyl]-5-(methanesulfonamidomethyl) pyrimidin-4-yl] pyrrolidine-3-yl]methyl]carbamate (6a) (50.0 mg, 0.089 mmol, 86.2% yield) as a white solid. LCMS m/z calcd for C₂₃H₂₉ClF₃N₅O₄S+H⁺: 564.02, found: 564.2.

Step 2. Synthesis of N-((4-(3-(aminomethyl)pyrrolidin-1-yl)-2-(4-chloro-3-(trifluoromethyl)phenyl) pyrimidin-5-yl)methyl)methanesulfonamide (6): To a solution of tert-butyl N-[[1-[2-[4-chloro-3-(trifluoromethyl)phenyl]-5-(methanesulfon-amido-methyl)pyrimidin-4-yl]pyrrolidin-3-yl]methyl]carbamate (6a) (50.0 mg, 0.09 mmol) in DCM (7 mL) was added TFA (1.0 mL, 0.11 mmol). The solution was stirred at 25° C. for 2 hrs. The mixture was concentrated and the residue was purified by prep-HPLC (eluting with H₂O:CH₃CN (0.1% TFA) from 90:10 to 5:95) to get N-[[4-[3-(aminomethyl)pyrrolidin-1-yl]-2-[4-chloro-3-(trifluoromethyl) phenyl]pyrimidin-5-yl]methyl]methanesulfonamide; 2,2,2-trifluoroacetic acid (6) (31.4 mg, 0.054 mmol, 60.5% yield) as a colorless oil. LCMS (ESI): m/z calcd for C₁₈H₂₁ClF₃N₅O₂S+H⁺: 464.2, found: 464.2. ¹H NMR (400 MHz, MeOD) δ 8.64 (d, J=4.0 Hz, 1H), 8.48 (d, J=8.8 Hz, 1H), 8.28 (s, 1H), 7.81 (t, J=9.2 Hz, 1H), 4.45 (s, 2H), 4.32-4.21 (m, 2H), 4.05-4.00 (m, 1H), 3.80-3.72 (m, 1H), 3.19-3.07 (m, 2H), 3.06 (s, 3H), 2.72-2.65 (m, 1H), 2.38-2.30 (m, 1H), 1.95-1.85 (m, 1H).

### Example 5: Exemplary Synthesis of Compound 17

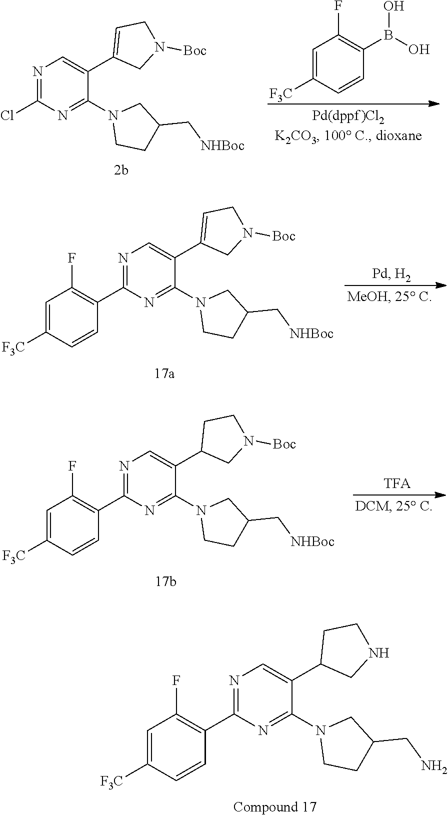 [SMILES: CC(C)(C)OC(=O)NCC1CCN(c2nc(Cl)ncc2C2=CCN(C(=O)OC(C)(C)C)C2)C1 ; OB(O)c1ccc(C(F)(F)F)cc1F ; CC(C)(C)OC(=O)NCC1CCN(c2nc(-c3ccc(C(F)(F)F)cc3F)ncc2C2=CCN(C(=O)OC(C)(C)C)C2)C1 ; CC(C)(C)OC(=O)NCC1CCN(c2nc(-c3ccc(C(F)(F)F)cc3F)ncc2C2CCN(C(=O)OC(C)(C)C)C2)C1 ; NCC1CCN(c2nc(-c3ccc(C(F)(F)F)cc3F)ncc2C2CCNC2)C1]

Step 1. Synthesis of tert-butyl 3-[4-[3-[(tert-butoxycarbonylamino)methyl]pyrrolidin-1-yl]-2-[2-fluoro-4-(trifluoromethyl)phenyl]pyrimidin-5-yl]-2,5-dihydropyrrole-1-carboxylate (17a): To a mixture of tert-butyl 3-[4-[3-[(tert-butoxycarbonylamino)methyl]pyrrolidin-1-yl]-2-chloro-pyrimidin-5-yl]-2,5-dihydropyrrole-1-carboxylate (2b) (400.0 mg, 0.83 mmol) in 1,4-dioxane (10 mL) was added [2-fluoro-4-(trifluoromethyl)phenyl]boronic acid (225.25 mg, 1.08 mmol), Na₂CO₃ (264.98 mg, 2.5 mmol) and Pd(dppf)Cl₂ (11.5 mg, 0.0800 mmol). The mixture was stirred at 80° C. for 16 h. The mixture was diluted with EtOAc (30 mL) and washed with H₂O (20 mL×2) and brine (20 mL). The organic layer was separated and dried over anhydrous sodium sulfate, filtered and concentrated under vacuo to give crude product which was purified by silica gel column (EtOAc:PE=2:1) to give tert-butyl 3-(4-(3-(((tert-butoxycarbonyl)amino)methyl)pyrrolidin-1-yl)-2-(2-fluoro-4-(trifluoromethyl)phenyl)pyrimidin-5-yl)-2,5-dihydro-TH-pyrrole-1-carboxylate (17a) (401 mg, 0.66 mmol, 79.7% yield) as a white solid. LCMS (ESI): m/z calcd for C30H37F4N5O4+H⁺: 608.2, found: 608.3.

Step 2. Synthesis of tert-butyl 3-[4-[3-[(tert-butoxycarbonylamino)methyl]pyrrolidin-1-yl]-2-[2-fluoro-4-(trifluoromethyl)phenyl]pyrimidin-5-yl]pyrrolidine-1-carboxylate (17b): To a mixture of tert-butyl 3-[4-[3-[(tert-butoxycarbonylamino)methyl]pyrrolidin-1-yl]-2-[2-fluoro-4-(trifluoromethyl)phenyl]pyrimidin-5-yl]-2,5-dihydropyrrole-1-carboxylate (17a) (240.0 mg, 0.39 mmol) in Methanol (10 mL) was added Pd/C (45.42 mg, 0.39 mmol). The mixture was purged with H₂ (23.72 mg, 0.39 mmol) three times and stirred under H₂ (1 atm). The mixture was stirred at 25° C. for 3 h. The mixture was diluted with EtOAc (30 mL), filtered over celite and concentrated under vacuo to give tert-butyl 3-[4-[3-[(tert-butoxycarbonylamino)methyl]pyrrolidin-1-yl]-2-[2-fluoro-4-(trifluoromethyl) phenyl]pyrimidin-5-yl]pyrrolidine-1-carboxylate (17b) (80 mg, 0.13 mmol, 33.2% yield) as a white solid. LCMS (ESI): m/z calcd for C30H39F4N5O4+H⁺: 610.3, found: 610.8.

Step 3. Synthesis of [1-[2-[2-fluoro-4-(trifluoromethyl) phenyl]-5-pyrrolidin-3-yl-pyrimidin-4-yl] pyrrolidin-3-yl] methanamine; 2,2,2-trifluoroacetic acid (17): To a mixture of tert-butyl 3-[4-[3-[(tert-butoxycarbonylamino) methyl] pyrrolidin-1-yl]-2-[2-fluoro-4-(trifluoromethyl)phenyl]pyrimidin-5-yl]pyrrolidine-1-carboxylate (17b) (80.27 mg, 0.13 mmol) in DCM (5 mL) was added TFA (1.0 mL, 0.13 mmol). The mixture was stirred at 25° C. for 2 h. The mixture was concentrated and purified by prep-HPLC (eluting with H₂O:CH₃CN (0.1% TFA) from 5% to 95%) to give [1-[2-[2-fluoro-4-(trifluoromethyl)phenyl]-5-pyrrolidin-3-yl-pyrimidin-4-yl] pyrrolidin-3-yl]methanamine; 2,2,2-trifluoroacetic acid (17) (71.2 mg, 0.11 mmol, 83.6% yield) as a white solid. LCMS (ESI): m/z calcd for C20H23F4N5+H⁺: 410.2, found: 410.2. ¹H NMR (400 MHz, MeOD): δ8.40 (s, 1H), 8.22 (t, J=7.6 Hz, 2H), 7.74 (m, 2H), 4.18 (s, 1H), 4.07 (d, 2H), 3.77 (m, 2H), 3.59 (m, 1H), 3.47 (m, 1H), 3.37 (s, 1H), 3.27 (s, 1H), 3.15 (t, J=7.2 Hz, 2H), 2.70 (s, 1H), 2.55 (m, 1H), 2.33 (m, 2H). 1.89 (m, 1H).

### Example 6: Exemplary Synthesis of Compound 19

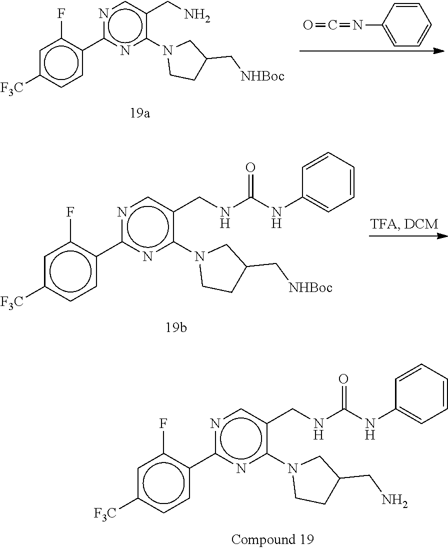

Step 1. Synthesis of tert-butyl N-[[1-[2-[2-fluoro-4-(trifluoromethyl)phenyl]-5-[(phenylcarbamoyl-amino)methyl]pyrimidin-4-yl]pyrrolidin-3-yl]methyl]carbamate (19b): To a solution of isocyanatobenzene (30.45 mg, 0.26 mmol) in DCM (5 mL) was added tert-butyl N-[[1-[5-(aminomethyl)-2-[2-fluoro-4-(trifluoromethyl)phenyl]pyrimidin-4-yl]pyrrolidin-3-yl]methyl]carbamate (19a, prepared with a similar procedure described for 5c) (60.0 mg, 0.130 mmol) at 25° C. The mixture was stirred at 25° C. for 18 h. The mixture was filtered, and concentrated to obtain tert-butyl N-[[1-[2-[2-fluoro-4-(trifluoromethyl)phenyl]-5-[(phenylcarbamoylamino)methyl] pyrimidin-4-yl] pyrrolidin-3-yl]methyl]carbamate (19b) (40 mg, 0.068 mmol, 53% yield) as a yellow solid. LCMS (ESI): m/z calcd for C29H32F4N6O3+H: 589.2, found: 589.3.

Step 2. Synthesis of 1-[[4-[3-(aminomethyl)pyrrolidin-1-yl]-2-[2-fluoro-4-(trifluoromethyl) phenyl]pyrimidin-5-yl]methyl]-3-phenyl-urea; 2,2,2-trifluoroacetic acid (19): To a solution of tert-butyl N-[[1-[2-[2-fluoro-4-(trifluoromethyl)phenyl]-5-[(phenylcarbamoyl-amino)methyl]pyrimidin-4-yl]pyrrolidin-3-yl]methyl]carbamate (19b) (40.0 mg, 0.070 mmol) in DCM (2 mL) was added TFA (93.78 mg, 0.6800 mmol) at rt under N₂. The mixture was stirred at 25° C. for 2 h. The mixture was concentrated and purified by prep-HPLC (H₂O:CH₃CN (0.1% TFA) from 90:10 to 5:95) to obtained 1-[[4-[3-(aminomethyl)pyrrolidin-1-yl]-2-[2-fluoro-4-(trifluoromethyl) phenyl]pyrimidin-5-yl]methyl]-3-phenyl-urea; 2,2,2-trifluoroacetic acid (39.88 mg, 0.064 mmol, 94% yield) as a white solid. LCMS (ESI): m/z calcd for C24H24F4N6O+H: 489.1, found: 489.2. ¹H NMR (400 MHz, MeOD-d4) δ 8.25-8.22 (m, 2H), 7.76-7.72 (m, 2H), 7.39-7.36 (m, 2H), 7.27-7.23 (m, 2H), 7.02-6.98 (m, 1H), 4.64 (s, 2H), 4.30-4.18 (m, 2H), 4.06-4.01 (m, 1H), 3.79-3.74 (m, 1H), 3.16-3.10 (m, 2H), 2.70-2.66 (m, 1H), 2.36-2.32 (m, 1H), 1.92-1.87 (m, 1H).

### Example 7: Exemplary Synthesis of Compound 21

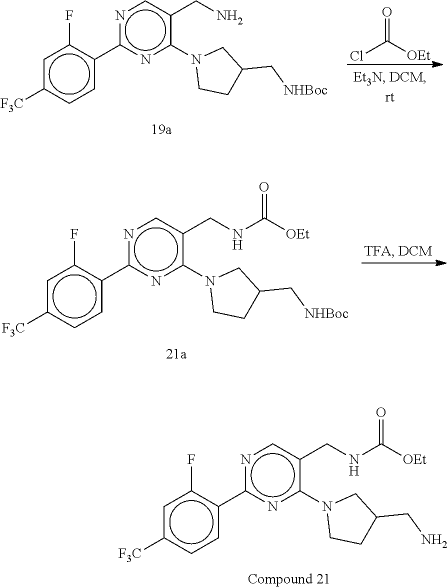

Step 1. Synthesis of ethyl N-[[4-[3-[(tert-butoxycarbonylamino)methyl]pyrrolidin-1-yl]-2-[2-fluoro-4-(trifluoromethyl)phenyl]pyrimidin-5-yl]methyl]carbamate (21a): To a solution of ethyl carbonochloridate (27.74 mg, 0.26 mmol) in DCM (5 mL) was added tert-butyl N-[[1-[5-(aminomethyl)-2-[2-fluoro-4-(trifluoromethyl)phenyl]pyrimidin-4-yl] pyrolidin-3-yl]methyl]carbamate (19a) (60.0 mg, 0.13 mmol) and Et₃N (0.05 mL, 0.38 mmol) at 25° C. The mixture was stirred at 25° C. for 18 h. The mixture was quenched by water (20 mL), extracted by EtOAc (20 mL×3), dried over MgSO₄, concentrated to obtain ethyl N-[[4-[3-[(tert-butoxycarbonylamino) methyl]pyrrolidin-1-yl]-2-[2-fluoro-4-(trifluoromethyl)phenyl]pyrimidin-5-yl]methyl]carbamate (21a) (50 mg, 0.092 mmol, 72% yield) as a yellow oil. LCMS (ESI): m/z calcd for C25H31F4N5O4+H: 542.1, found: 542.2.

Step 2. Synthesis of ethyl N-[[4-[3-(aminomethyl)pyrrolidin-1-yl]-2-[2-fluoro-4-(trifluoromethyl) phenyl]pyrimidin-5-yl]methyl]carbamate (21): To a solution of ethyl N-[[4-[3-[(tert-butoxycarbonylamino)methyl]pyrrolidin-1-yl]-2-[2-fluoro-4-(trifluoromethyl)phenyl]pyrimidin-5-yl]methyl]carbamate (21a) (50.0 mg, 0.09 mmol) in DCM (2 mL) was added TFA (1 mL, 0.92 mmol) at rt. The mixture was stirred at 25° C. for 2 h. The mixture was concentrated and purified by prep-HPLC (H₂O:CH₃CN (0.1% TFA) from 90:10 to 5:95) to obtained ethyl N-[[4-[3-(aminomethyl)pyrrolidin-1-yl]-2-[2-fluoro-4-(trifluoromethyl) phenyl]pyrimidin-5-yl]methyl]carbamate; 2,2,2-trifluoroacetic acid (21) (43.32 mg, 0.076 mmol, 82% yield) as a white solid. LCMS (ESI): m/z calcd for C20H23F4N5O2+H: 442.1, found: 442.2. ¹H NMR (400 MHz, MeOD-d4) δ 8.26-8.24 (m, 1H), 8.22-8.18 (m, 1H), 7.77-7.73 (m, 2H), 4.54 (s, 2H), 4.28-4.11 (m, 4H), 4.09-3.97 (m, 1H), 3.77-3.71 (m, 1H), 3.18-3.08 (m, 2H), 2.70-2.66 (m, 1H), 2.36-2.32 (m, 1H), 1.92-1.87 (m, 1H), 1.27-1.23 (m, 3H).

### Example 8: Exemplary Synthesis of Compound 34

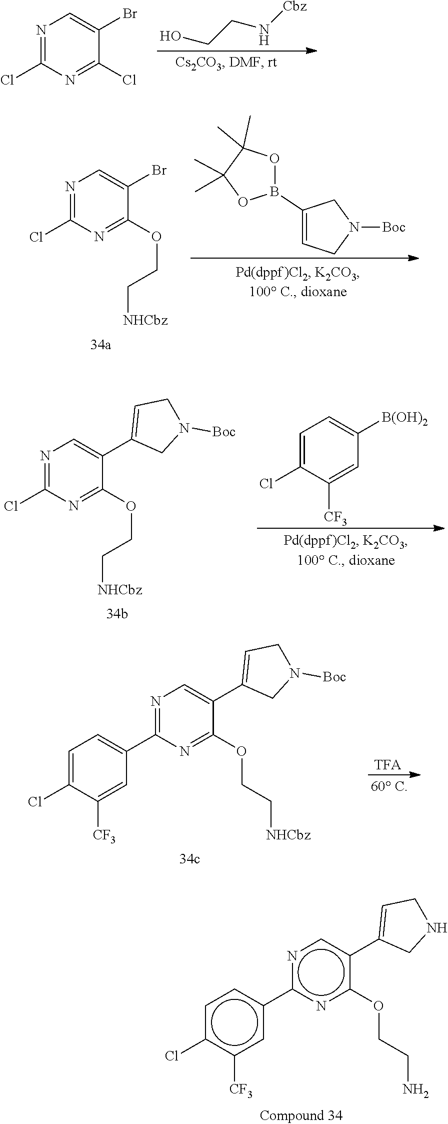

Step 1. Synthesis of benzyl N-[2-(5-bromo-2-chloro-pyrimidin-4-yl)oxyethyl]carbamate (34a): To a solution of benzyl N-(2-hydroxyethyl)carbamate (100.0 mg, 0.51 mmol) in dry DMF (2 mL) were added 5-bromo-2,4-dichloro-pyrimidine (116.72 mg, 0.51 mmol) and Cs₂CO₃ (61.52 mg, 1.02 mmol). The mixture was stirred at 25° C. for 16 h under Ar. The mixture was quenched by water (20 mL), extracted by EtOAc (10 mL×3), dried over MgSO₄, concentrated to obtain benzyl N-[2-(5-bromo-2-chloro-pyrimidin-4-yl)oxyethyl]carbamate (34a) (90 mg, 0.23 mmol, 45% yield) as a crude oil. LCMS (ESI): m/z calcd for C14H13BrClN3O3+H⁺: 388.0, found: 388.1.

Step 2. Synthesis of tert-butyl 3-[4-[2-(benzyloxycarbonylamino)ethoxy]-2-chloro-pyrimidin-5-yl]-2,5-dihydropyrrole-1-carboxylate (34b): To a solution of benzyl N-[2-(5-bromo-2-chloro-pyrimidin-4-yl)oxyethyl]carbamate (34a) (600.0 mg, 1.55 mmol) in 1,4-dioxane (30 mL) and water (6 mL) were added tert-butyl 3-(4,4,5,5-tetramethyl-1,3,2-dioxaborolan-2-yl)-2,5-dihydropyrrole-1-carboxylate (458.08 mg, 1.55 mmol), K₂CO₃ (428.32 mg, 3.1 mmol) and Pd(dppf)Cl₂ (63.37 mg, 0.080 mmol) at rt under N2. The mixture was stirred at 100° C. for 2 h. The mixture was quenched by water (20 mL), extracted by EtOAc (10 mL×3), dried over MgSO₄, concentrated to obtain tert-butyl 3-[4-[2-(benzyloxycarbonylamino) ethoxy]-2-chloro-pyrimidin-5-yl]-2,5-dihydropyrrole-1-carboxylate (34b) (460 mg, 0.97 mmol, 62% yield) as a yellow solid. LCMS (ESI): m/z calcd for C23H27ClN4O5-Boc+H: 375.1, found 375.2.

Step 3. Synthesis of tert-butyl 3-[4-[2-(benzyloxycarbonylamino)ethoxy]-2-[4-chloro-3-(trifluoromethyl)phenyl]pyrimidin-5-yl]-2,5-dihydropyrrole-1-carboxylate (34c): To a solution of tert-butyl 3-[4-[2-(benzyloxycarbonylamino)ethoxy]-2-chloro-pyrimidin-5-yl]-2,5-dihydropyrrole-1-carboxylate (34b) (530.0 mg, 1.12 mmol) in 1,4-dioxane (20 mL) and water (4 mL) were added [4-chloro-3-(trifluoromethyl)phenyl]boronic acid (300.46 mg, 1.34 mmol), K₂CO₃ (308.0 mg, 2.23 mmol) and Pd(dppf)Cl₂ (45.57 mg, 0.06 mmol) at rt under N2. The mixture was stirred at 90° C. for 2 h. The mixture was quenched by water (20 mL), extracted by EtOAc (10 mL×3), dried over MgSO₄, concentrated and purified by silica chromatography column (PE:EtOAc=5:1 to 2:1) to obtain tert-butyl 3-[4-[2-(benzyloxycarbonylamino)ethoxy]-2-[4-chloro-3-(trifluoromethyl) phenyl]pyrimidin-5-yl]-2,5-dihydropyrrole-1-carboxylate (34c) (380 mg, 0.62 mmol, 55% yield) as a yellow solid. LCMS (ESI): m/z calcd for C30H30ClF3N4O5+H: 619.1, found 619.1.

Step 4. Synthesis of 2-[2-[4-chloro-3-(trifluoromethyl)phenyl]-5-(2,5-dihydro-1H-pyrrol-3-yl)pyrimidin-4-yl]oxyethanamine (34): A mixture of tert-butyl 3-[4-[2-(benzyloxycarbonylamino)ethoxy]-2-[4-chloro-3-(trifluoromethyl) phenyl]pyrimidin-5-yl]-2,5-dihydropyrrole-1-carboxylate (34c) (80.0 mg, 0.13 mmol) in TFA (2 mL, 26.93 mmol) under N2 was stirred at 60° C. for 2 h. The mixture was concentrated and purified by prep-HPLC (H₂O:CH₃CN (0.1% TFA) from 90:10 to 5:95) to obtained 2-[2-[4-chloro-3-(trifluoromethyl)phenyl]-5-(2,5-dihydro-1H-pyrrol-3-yl)pyrimidin-4-yl]oxyethanamine; 2,2,2-trifluoroacetic acid (34) (29.53 mg, 0.0476 mmol, 36.857% yield) as a white solid. LCMS (ESI): m/z calcd for C₁₇H16ClF3N40+H: 385.1, found: 385.0. ¹H NMR (400 MHz, MeOD-d4) δ 8.82-8.81 (m, 1H), 8.70 (s, 1H), 8.67-8.64 (m, 1H), 7.79-7.76 (m, 1H), 6.74-6.73 (m, 1H), 4.95-4.93 (m, 2H), 4.59-4.58 (m, 2H), 4.32-4.31 (m, 2H), 3.57-3.55 (m, 2H).

### Example 9: Exemplary Synthesis of Compound 45

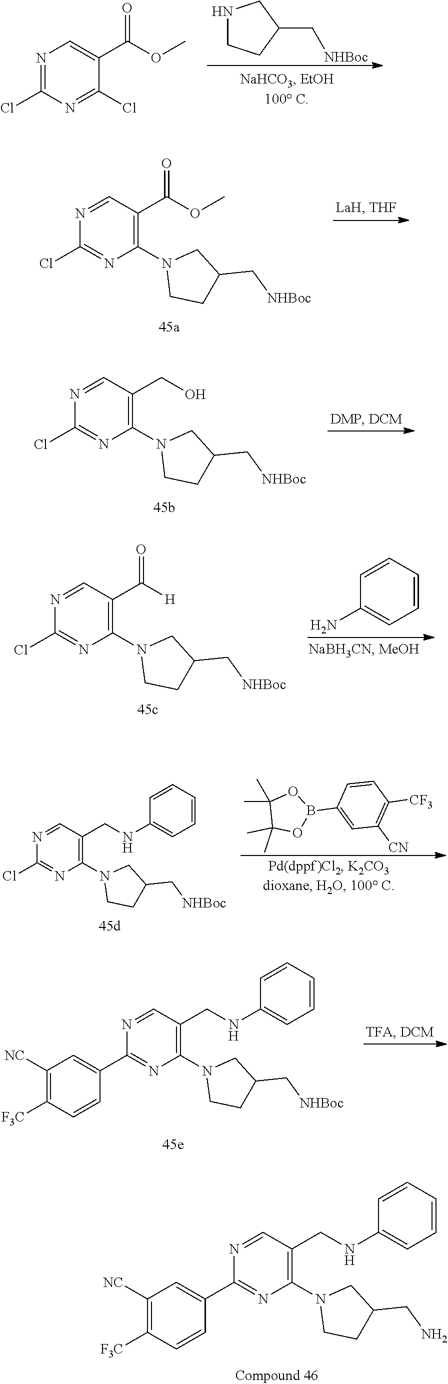

Step 1. Synthesis of methyl 4-(3-(((tert-butoxycarbonyl)amino)methyl)pyrrolidin-1-yl)-2-chloropyrimidine-5-carboxylate (45a): To a solution of tert-butyl N-(pyrrolidin-3-ylmethyl)carbamate (507.93 mg, 2.54 mmol) and methyl 2,4-dichloropyrimidine-5-carboxylate (500.0 mg, 2.42 mmol) in ethanol (20 mL) was added NaHCO₃ (731.85 mg, 7.25 mmol) at 25° C. The mixture was stirred at 100° C. for 2 h. The mixture was diluted with EtOAC (100 mL), washed with water (50 mL) and brine (50 mL), dried over Na₂SO₄, concentrated. The crude product was purified by flash chromatography (H₂O:CH₃CN=90:10 to 50:50). The product methyl 4-[3-[(tert-butoxycarbonylamino)methyl]pyrrolidin-1-yl]-2-chloro-pyrimidine-5-carboxylate (45a) (427 mg, 1.15 mmol, 47.67% yield) was obtained as brown solid. LCMS (ESI): m/z calcd for C16H23ClN4O4+H⁺: 371.1, found 371.2.

Step 2. Synthesis of tert-butyl N-[[1-[2-chloro-5-(hydroxymethyl)pyrimidin-4-yl]pyrrolidin-3-yl]methyl]carbamate (45b): To a solution of methyl 4-[3-[(tert-butoxycarbonylamino)methyl]pyrrolidin-1-yl]-2-chloro-pyrimidine-5-carboxylate (45a) (300.0 mg, 0.810 mmol) in THF (10 mL) was added LiAlH₄ (61.6 mg, 1.62 mmol) at 0° C. The mixture was stirred at 0° C. for 1 h. The mixture was quenched with NaOH (0.12 mL, 4 mol/L in water), diluted with EtOAc (30 mL), filtered and concentrated. The crude product was purified by flash chromatography eluting with 0-90% acetonitrile in water (0.1% TFA) to give tert-butyl N-[[1-[2-chloro-5-(hydroxymethyl)pyrimidin-4-yl]pyrrolidin-3-yl]methyl]carbamate (45b) (50 mg, 0.146 mmol, 18% yield) as a yellow solid. LCMS (ESI): m/z calcd for C15H23ClN4O3+H: 343.1, found: 343.0.

Step 3. Synthesis of tert-butyl N-[[1-(2-chloro-5-formyl-pyrimidin-4-yl)pyrrolidin-3-yl]methyl]carbamate (45c): To a solution of tert-butyl N-[[1-[2-chloro-5-(hydroxymethyl)pyrimidin-4-yl]pyrrolidin-3-yl]methyl]carbamate (45b) (50.0 mg, 0.150 mmol) in DCM (5 mL) was added Dess-Martin periodinane (186 mg, 0.440 mmol) at 0° C. The mixture was stirred at R.T. for overnight. The reaction was quenched by aqueous NaHCO₃ (2 mL) at 0° C. The resulting mixture was filtered, extracted with DCM (3×10 mL), dried over Na₂SO₄, and concentrated. The crude product was purified by slica gel chromatography (PE:EtOAc=3:1) to give tert-butyl N-[[1-(2-chloro-5-formyl-pyrimidin-4-yl) pyrrolidin-3-yl]methyl]carbamate (45c) (30 mg, 0.088 mmol, 60% yield) as a yellow solid. LCMS (ESI): m/z calcd for C₁₅H21C₁N403+H: 341.2, found: 341.3.

Step 4. Synthesis of tert-butyl N-[[1-[5-(anilinomethyl)-2-chloro-pyrimidin-4-yl]pyrrolidin-3-yl] methyl]carbamate (45d): To a solution of tert-butyl N-[[1-(2-chloro-5-formyl-pyrimidin-4-yl)pyrrolidin-3-yl] methyl]carbamate (45c) (30.0 mg, 0.090 mmol) in Methanol (3 mL) were added aniline (9.84 mg, 0.110 mmol) and acetic acid (0.1 mL, 0.090 mmol) at rt. The mixture was stirred at rt for 1 h. Then, to the mixture was added NaBH3CN (11.06 mg, 0.180 mmol) at rt. The mixture was stirred at R.T. for 16 h. The mixture was concentrated, then purified by flash column chromatography eluting with H₂O:CH₃CN=50:50 (0.1% TFA) to afford tert-butyl N-[[1-[5-(anilinomethyl)-2-chloro-pyrimidin-4-yl]pyrrolidin-3-yl]methyl]carbamate (45d) (30 mg, 0.072 mmol, 82% yield) as a brown solid. LCMS (ESI): m/z calcd for C21H28ClN5O2+H: 418.2, found: 418.4.

Step 5. Synthesis of tert-butyl N-[[1-[5-(anilinomethyl)-2-[3-cyano-4-(trifluoromethyl)phenyl] pyrimidin-4-yl]pyrrolidin-3-yl]methyl]carbamate (45e): To a solution of tert-butyl N-[[1-[5-(anilinomethyl)-2-chloro-pyrimidin-4-yl]pyrrolidin-3-yl]methyl]carbamate (45d) (30.0 mg, 0.070 mmol) in 1,4-dioxane (5 mL) and water (1 mL) were added 5-(4,4,5,5-tetramethyl-1,3,2-dioxaborolan-2-yl)-2-(trifluoromethyl)benzonitrile (25.59 mg, 0.090 mmol), Na₂CO₃ (22.82 mg, 0.220 mmol) and Pd(PPh₃)₄ (8.29 mg, 0.010 mmol). The mixture was stirred at 80° C. for overnight under Ar. The mixture was extracted with EtOAc (3×20 mL) and H₂O (3×10 mL). All the organic phases were collected, washed with brine (10 mL), dried over Na₂SO₄, and filtered. The filtration was concentrated to provide the crude, which was purified by slica gel chromatography (PE:EtOAc=3:1) to provide tert-butyl N-[[1-[5-(anilinomethyl)-2-[3-cyano-4-(trifluoromethyl)phenyl]pyrimidin-4-yl]pyrrolidin-3-yl]methyl]carbamate (45e) (20 mg, 0.036 mmol, 50% yield) as a yellow solid. LCMS (ESI): m/z calcd for C29H31F3N6O2+H: 553.2, found: 553.5.

Step 6. Synthesis of 5-[4-[3-(aminomethyl)pyrrolidin-1-yl]-5-(anilinomethyl)pyrimidin-2-yl]-2-(trifluoromethyl)benzonitrile; 2,2,2-trifluoroacetic acid (45): To a solution of tert-butyl N-[[1-[5-(anilinomethyl)-2-[3-cyano-4-(trifluoromethyl)phenyl] pyrimidin-4-yl]pyrrolidin-3-yl]methyl]carbamate (45e) (20.0 mg, 0.040 mmol) in 1,4-dioxane (2 mL) was added HCl (5.0 mL, 4 mol/L in dioxane, 20 mmol). The reaction mixture was stirred at 25° C. for overnight. The solvent was removed to get the crude, which was then purified by prep-HPLC eluting with 0-90% acetonitrile in water (0.1% TFA) to give 5-[4-[3-(aminomethyl) pyrrolidin-1-yl]-5-(anilinomethyl)pyrimidin-2-yl]-2-(trifluoromethyl)benzonitrile; 2,2,2-trifluoro-acetic acid (10.17 mg, 0.0177 mmol, 49% yield) as white solid. LCMS (ESI): m/z calcd for C26H24F6N6O2+H: 453.2, found: 453.4. ¹H NMR (400 MHz, MeOD) δ 8.81 (s, 1H), 8.69 (d, J=9.2 Hz, 1H), 8.25 (s, 1H), 8.10 (d, J=8.0 Hz, 1H), 7.20-7.11 (m, 2H), 6.75-6.65 (m, 3H), 4.53-4.36 (m, 2H), 4.34-4.18 (m, 2H), 4.11-4.00 (m, 1H), 3.81-3.71 (m, 1H), 3.20-3.02 (m, 2H), 2.72-2.59 (m, 1H), 2.37-2.24 (m, 1H), 1.95-1.80 (m, 1H).

### Example 10: Exemplary Synthesis of Compound 49

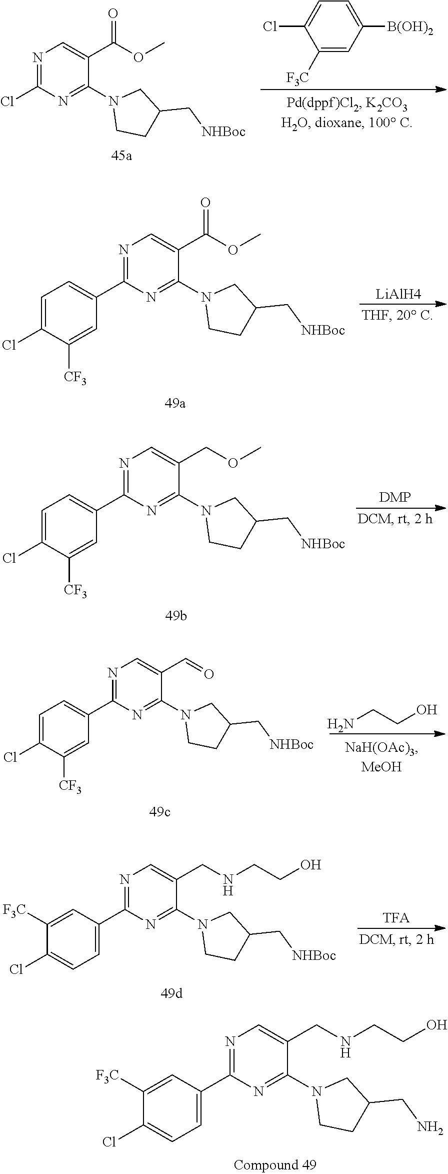

Step 1. Synthesis of methyl 4-(3-(((tert-butoxycarbonyl)amino)methyl)pyrrolidin-1-yl)-2-(4-chloro-3-(trifluoromethyl)phenyl)pyrimidine-5-carboxylate (49a): To a solution of methyl 4-[3-[(tert-butoxycarbonylamino)methyl]pyrrolidin-1-yl]-2-chloro-pyrimidine-5-carboxylate (45a) (420 mg, 1.13 mmol) and [4-chloro-3-(trifluoromethyl)phenyl] boronic acid (280 mg, 1.25 mmol) in 1,4-dioxane (12 mL) and water (3 mL) were added K₂CO₃ (720.13 mg, 3.4 mmol) and Pd(dppf)Cl₂ (92.49 mg, 0.11 mmol) at 25° C. The mixture was stirred at 100° C. for 2 h. The mixture was diluted with EtOAc (100 mL), washed with water (50 mL) and brine (50 mL), dried over Na₂SO₄, concentrated. The crude product was purified by flash chromatography (H₂O:CH₃CN=90:10 to 50:50). The product methyl 4-[3-[(tert-butoxy-carbonylamino)methyl]pyrrolidin-1-yl]-2-[4-chloro-3-(trifluoromethyl)phenyl]pyrimidine-5-carboxylate (49a) (340 mg, 0.6603 mmol, 58% yield) was obtained as a brown solid. LCMS (ESI): m/z calcd for C23H26ClF3N4O4+H⁺: 515.2, found 515.2.

Step 2. Synthesis of tert-butyl ((1-(2-(4-chloro-3-(trifluoromethyl)phenyl)-5-(hydroxymethyl) pyrimidin-4-yl)pyrrolidin-3-yl)methyl)carbamate (49b): To a solution of methyl 4-[3-[(tert-butoxycarbonylamino)methyl]pyrrolidin-1-yl]-2-[4-chloro-3-(trifluoromethyl)phenyl]pyrimidine-5-carboxylate (49a) (200.0 mg, 0.39 mmol) in THF (1 mL) was added LiAlH₄ (30 mg, 0.78 mmol) at 20° C. The mixture was stirred at 20° C. for 30 min. The mixture was quenched with H₂O (0.1 mL) and 15% NaOH (0.1 mL), extracted with EtOA_{C}(5 mL), dried over MgSO₄, concentrated. The crude product was purified by flash chromatography (H₂O: CH₃CN=90:10 to 50:50). The product tert-butyl N-[[1-[2-[4-chloro-3-(trifluoromethyl)phenyl]-5-(hydroxymethyl)pyrimidin-4-yl]pyrrolidin-3-yl]methyl]carbamate (49b) (180 mg, 0.37 mmol, 95.2% yield) was obtained as white solid. LCMS (ESI): m/z calcd for C22H26ClF3N4O3+H⁺: 487.16, found: 487.2.

Step 3. Synthesis of tert-butyl N-[[1-[2-[4-chloro-3-(trifluoromethyl)phenyl]-5-formyl-pyrimidin-4-yl]pyrrolidin-3-yl]methyl]carbamate (49c): To a solution of tert-butyl N-[[1-[2-[4-chloro-3-(trifluoromethyl)phenyl]-5-(hydroxymethyl) pyrimidin-4-yl]pyrrolidin-3-yl]methyl]carbamate (49b) (218.0 mg, 0.45 mmol) in DCM (8 mL) was added DMP (379.79 mg, 0.90 mmol) at r.t. The reaction mixture was then stirred at 25° C. for 2 hours. The mixture was quenched by water (30 mL) and extracted by EtOAc (15 mL×3), dried over Na₂SO₄, concentrated. The crude product was purified by silica gel chromatography (PE:EtOAc=20:1 to 1:1) to get tert-butyl N-[[1-[2-[4-chloro-3-(trifluoromethyl)phenyl]-5-formyl-pyrimidin-4-yl]pyrrolidin-3-yl]methyl]carbamate (49c) (176 mg, 0.363 mmol, 81.1% yield) as yellow solid. LCMS (ESI): m/z calcd for C22H24ClF3N4O3+H⁺: 485.2; found: 485.2.

Step 4. Synthesis of tert-butyl N-[[1-[2-[4-chloro-3-(trifluoromethyl)phenyl]-5-[(2-hydroxyethylamino) methyl]pyrimidin-4-yl]pyrrolidin-3-yl]methyl]carbamate (49d): To a solution of tert-butyl N-[[1-[2-[4-chloro-3-(trifluoromethyl)phenyl]-5-formyl-pyrimidin-4-yl]pyrrolidin-3-yl]methyl]carbamate (49c) (176.0 mg, 0.36 mmol) in methanol (8 mL) was added 2-aminoethanol (33.25 mg, 0.54 mmol) and NaBH(OAc)₃ (230.78 mg, 1.09 mmol) at r.t. The reaction mixture was then stirred at 25° C. for 2 hours. The mixture was quenched by water (30 mL) and extracted by EtOAc (15 mL×3), dried over Na₂SO₄, concentrated. The mixture was filtered, and the solution was concentrated to give crude tert-butyl N-[[1-[2-[4-chloro-3-(trifluoromethyl)phenyl]-5-[(2-hydroxyethylamino)methyl]pyramid-in-4-yl]pyrrolidin-3-yl]methyl]carbamate (49d) (100 mg, 0.19 mmol, 52% yield) as a yellow solid. LCMS (ESI): m/z calcd for C24H31ClF3N5O3+H⁺: 530.1, found: 530.2.

Step 5. Synthesis of 2-[[4-[3-(aminomethyl)pyrrolidin-1-yl]-2-[4-chloro-3-(trifluoromethyl) phenyl]pyrimidin-5-yl]methylamino]ethanol (49): To a solution of tert-butyl N-[[1-[2-[4-chloro-3-(trifluoromethyl)phenyl]-5-[(2-hydroxyethyl amino)methyl]pyrimidin-4-yl]pyrrolidin-3-yl]methyl]carbamate (49d) (100.0 mg, 0.19 mmol) in DCM (10 mL) was added TFA (2 mL, 0.60 mmol) at r.t. The reaction mixture was then stirred at 25° C. for 2 hours. The mixture was filtered, and the solution was concentrated. The crude product was purified by Prep-HPLC (H₂O:CH₃CN=90:10 to 50:50, 0.1% TFA as additive) to get 2-[[4-[3-(amino-methyl)pyro-lidin-1-yl]-2-[4-chloro-3-(trifluoromethyl)phenyl]pyrimidin-5-yl]methylamino]ethanol; 2,2,2-trifluoroacetic acid (49) (12.96 mg, 0.020 mmol, 10.4% yield) as white solid. LCMS (ESI): m/z calcd for C₂₂H₂₇ClF₃N₅O₂+H⁺: 430.2, found: 430.2. ¹H NMR (400 MHz, DMSO-d6) 8.74 (d, J=2.0 Hz, 1H), 8.59-8.57 (m, 1H), 8.43 (s, 1H), 7.74 (d, J=8.4 Hz, 1H), 4.50 (t, J=5.2 Hz, 2H), 4.12-4.08 (m, 1H), 3.99-3.86 (m, 4H), 3.66-3.62 (m, 1H), 3.27 (t, J=5.2 Hz, 2H), 3.21-3.09 (m, 2H), 2.69 (d, J=7.6 Hz, 1H), 2.35 (t, J=2.0 Hz, 1H), 1.93-1.88 (m, 1H).

### Example 11: Exemplary Synthesis of Compound 58

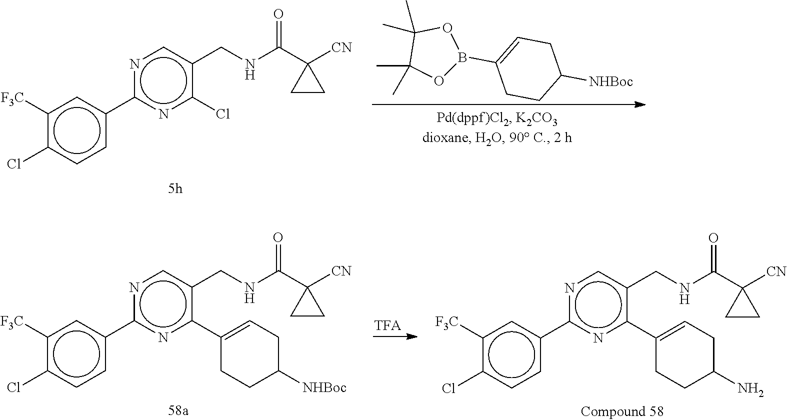

Step 1. Synthesis of tert-butyl N-[4-[2-[4-chloro-3-(trifluoromethyl)phenyl]-5-[[(1-cyanocyclo-propanecarbonyl)amino]methyl]pyrimidin-4-yl]cyclohex-3-en-1-yl]carbamate (58a): To a solution of tert-butyl N-[4-(4,4,5,5-tetramethyl-1,3,2-dioxaborolan-2-yl)cyclohex-3-en-1-yl]carbamate (64.23 mg, 0.20 mmol) and N-[[4-chloro-2-[4-chloro-3-(trifluoromethyl)phenyl] pyrimidin-5-yl]methyl]-1-cyano-cyclopropanecarboxamide (5h) (75.0 mg, 0.180 mmol) in 1,4-dioxane (5 mL) and water (1 mL) were added Pd(dppf)Cl₂ (7.38 mg, 0.010 mmol) and K₂CO₃ (74.78 mg, 0.540 mmol) under argon atmosphere at r.t. The reaction mixture was then stirred at 95° C. for 2 hours. The mixture was extracted with EtOAc (15×3 mL), dried over Na₂SO₄, concentrated. The crude product was purified by silica gel chromatography (PE:EA=4:1) to afford tert-butyl N-[4-[2-[4-chloro-3-(trifluoromethyl)phenyl]-5-[[(1-cyanocyclopropanecarbonyl)amino]methyl]pyrimidin-4-yl]cyclohex-3-en-1-yl]carbamate (58a) (50 mg, 0.087 mmol, 48% yield) as yellow solid. LCMS (ESI): m/z calcd for C28H29ClF3N5O3+H: 576.1, found: 576.4.

Step 2. Synthesis of N-[[4-(4-aminocyclohexen-1-yl)-2-[4-chloro-3-(trifluoromethyl)phenyl] pyrimidin-5-yl]methyl]-1-cyano-cyclopropanecarboxamide (58): To a solution of tert-butyl N-[4-[2-[4-chloro-3-(trifluoromethyl)phenyl]-5-[[(1-cyanocyclo-propanecarbonyl)amino]methyl]pyrimidin-4-yl]cyclohex-3-en-1-yl]carbamate (58a) (50.0 mg, 0.090 mmol) in DCM (3 mL) was added TFA (1 mL, 0.090 mmol) at r.t. The reaction mixture was then stirred at 25° C. for 2 hours. The mixture was concentrated to afford a crude product. The crude product was purified by prep-HPLC (H₂O:CH₃CN=90:10 to 50:50, 0.1% TFA as additive) to afford N-[[4-(4-aminocyclohexen-1-yl)-2-[4-chloro-3-(trifluoromethyl)phenyl]pyrimidin-5-yl]methyl]-1-cyano-cyclopropanecarboxamide; 2,2,2-trifluoroacetic acid (58) (15.99 mg, 0.027 mmol, 31% yield) as light yellow solid. LCMS (ESI): m/z calcd for C23H21ClF3N5O+H: 476.1, found: 476.3. ¹H NMR (400 MHz, MeOD-d4) δ 8.85-8.76 (m, 2H), 8.67-8.59 (m, 1H), 7.75 (d, 1H), 5.97 (d, 1H), 4.56 (s, 2H), 3.65-3.53 (m, 1H), 2.80-2.68 (m, 3H), 2.43-2.22 (m, 2H), 2.06-1.88 (m, 1H), 1.62-1.54 (m, 4H).

### Example 12: Exemplary Synthesis of Compound 90

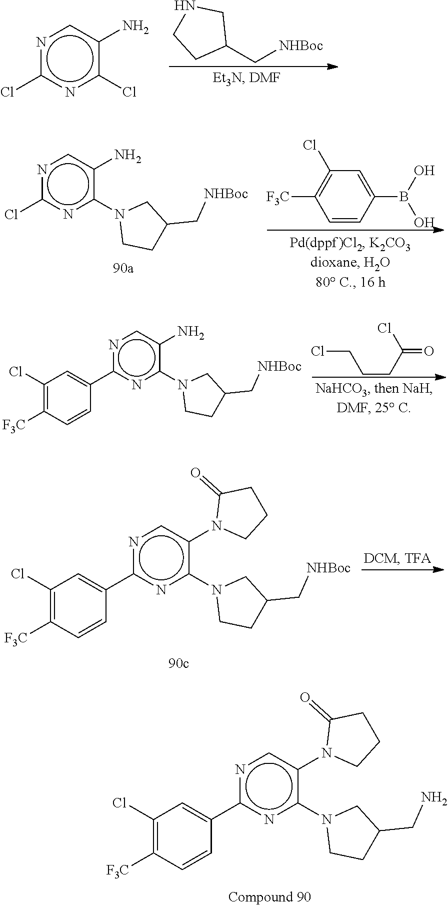

Step 1. Synthesis of tert-butyl N-[[1-(5-amino-2-chloro-pyrimidin-4-yl)pyrrolidin-3-yl]methyl] carbamate (90a): To a solution of tert-butyl N-(pyrrolidin-3-ylmethyl) carbamate (366.39 mg, 1.83 mmol) in THF (5 mL) were added 2,4-dichloropyrimidin-5-amine (300.0 mg, 1.83 mmol) and Et₃N (0.77 mL, 5.49 mmol). The mixture was stirred at 25° C. for 2 h under Ar. The mixture was quenched by water (20 mL), extracted by EtOAc (10 mL×3), dried over MgSO₄, concentrated to obtain tert-butyl N-[[1-(5-amino-2-chloro-pyrimidin-4-yl)pyrrolidin-3-yl]methyl]carbamate (90a) (500 mg, 1.52 mmol, 83.4% yield) as yellow oil which was used without further purification. LCMS (ESI): m/z calcd for C₁₄H₂₂ClN₅O₂+H⁺: 328.1, found: 328.2.

Step 2. Synthesis of tert-butyl N-[[1-[5-amino-2-[3-chloro-4-(trifluoromethyl)phenyl]pyrimidin-4-yl]pyrrolidin-3-yl]methyl]carbamate (90b): To a solution of tert-butyl N-[[1-(5-amino-2-chloro-pyrimidin-4-yl)pyrrolidin-3-yl] methyl]carbamate (90a) (500.0 mg, 1.53 mmol) in 1,4-dioxane (5 mL) and water (1 mL) were added [3-chloro-4-(trifluoromethyl)phenyl] boronic acid (342.23 mg, 1.53 mmol), K₂CO₃ (420.98 mg, 3.05 mmol) and Pd(dppf)Cl₂ (62.28 mg, 0.08 mmol) at rt under N2. The mixture was stirred at 90° C. for 2 h. The mixture was quenched by water (20 mL), extracted by EtOAc (10 mL×3), dried over MgSO₄, concentrated and purified by silica chromatography column (PE:EtOAc=5:1 to 1:1) to obtain tert-butyl N-[[1-[5-amino-2-[3-chloro-4-(trifluoromethyl)phenyl]pyrimidin-4-yl]pyrrolidin-3-yl]methyl]carba-mate (90b) (700 mg, 1.48 mmol, 97.252% yield) as a solid. LCMS (ESI): m/z calcd for C₂₁H₂₅ClF₃N₅O₂+H⁺: 472.1, found: 472.2.

Step 3. Synthesis of tert-butyl N-[[1-[2-[3-chloro-4-(trifluoromethyl)phenyl]-5-(2-oxopyrrolidin-1-yl) pyrimidin-4-yl]pyrrolidin-3-yl]methyl]carbamate (90c): To a solution of tert-butyl N-[[1-[5-amino-2-[3-chloro-4-(trifluoromethyl)phenyl]pyrimidin-4-yl] pyrrolidin-3-yl]methyl]carbamate (90b) (30.0 mg, 0.060 mmol) in THF (2 mL) were added K₂CO₃ (26.32 mg, 0.19 mmol) and 4-chlorobutanoyl chloride (10.76 mg, 0.080 mmol) at rt under N₂. The mixture was stirred at 25° C. for 1 h. Then NaH (3.81 mg, 0.16 mmol) was added and the mixture was stirred at 25° C. for 3 h. The mixture was quenched by sat. NH₄Cl solution (5 mL) and extracted by EtOAc (10 mL×2), dried over MgSO₄, concentrated to give crude tert-butyl N-[[1-[2-[3-chloro-4-(trifluoro methyl)phenyl]-5-(2-oxopyrrolidin-1-yl)pyrimidin-4-yl]pyrrolidin-3-yl]methyl]carbamate (90c) (25 mg, crude) as a light yellow solid which was used without further purification. LCMS (ESI): m/z calcd for C₂₅H₂₉ClF₃N₅O₃+H⁺: 540.2, found: 540.2.

Step 4. Synthesis of 1-[4-[3-(aminomethyl)pyrrolidin-1-yl]-2-[3-chloro-4-(trifluoromethyl)phenyl] pyrimidin-5-yl]pyrrolidin-2-one (90): To a solution of tert-butyl N-[[1-[2-[3-chloro-4-(trifluoromethyl)phenyl]-5-(2-oxopyrro-lidin-1-yl)pyrimidin-4-yl]pyrrolidin-3-yl]methyl]carbamate (90c) (25.0 mg, 0.05 mmol) in DCM (3 mL) was added TFA (1.0 mL, 0.11 mmol) at 25° C. The mixture was stirred at 25° C. for 1 h. The reaction was concentrated and purified by prep-HPLC (eluting with H₂O:CH₃CN (0.1% TFA) form 90:10 to 10:90) to obtain 1-[4-[3-(aminomethyl)pyrrolidin-1-yl]-2-[3-chloro-4-(trifluoromethyl)phenyl] pyrimidin-5-yl]pyrrolidin-2-one; 2,2,2-trifluoroacetic acid (90) (7.06 mg, 0.0126 mmol, 27% yield) as a white powder. LCMS (ESI): m/z calcd for C₂₀H₂₁ClF₃N₅O+H⁺: 440.2; found: 440.2. ¹H NMR (400 MHz, MeOD) δ 8.51 (s, 1H), 8.40 (d, J=8.4 Hz, 1H), 8.28 (s, 1H), 7.89 (d, J=8.0 Hz, 1H), 4.02-3.46 (m, 6H), 3.16-3.05 (m, 2H), 2.66-2.60 (m, 3H), 2.32-2.27 (m, 3H), 1.90-1.85 (m, 1H).

### Example 13: Exemplary Synthesis of Compound 109

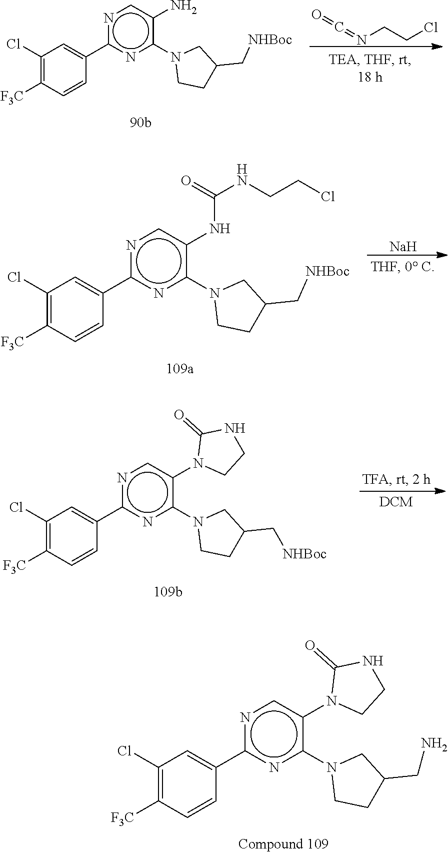

Step 1. Synthesis of tert-butyl N-[[1-[5-(2-chloroethylcarbamoylamino)-2-[3-chloro-4-(trifluoromethyl)phenyl]pyrimidin-4-yl]pyrrolidin-3-yl]methyl]carbamate (109a): The solution of tert-butyl N-[[1-[5-amino-2-[3-chloro-4-(trifluoromethyl)phenyl]pyrimidin-4-yl] pyrrolidin-3-yl]methyl]carbamate (90b) (150.0 mg, 0.32 mmol), 1-chloro-2-isocyanato-ethane (40.25 mg, 0.380 mmol) and Triethylamine (19.07 mg, 0.79 mmol) in THF (5 mL) was stirred at 25° C. for 48 h. The solvent was removed, and the residue was purified by flash chromatography (DCM:MeOH=20:1) to obtain tert-butyl N-[[1-[5-(2-chloroethyl-carbamoylamino)-2-[3-chloro-4-(trifluoromethyl)phenyl]pyrimidin-4-yl]pyrrolidin-3-yl]methyl]carbamate (109a) (150 mg, 0.26 mmol, 82% yield) as yellow oil. LCMS (ESI): m/z calcd for C24H29Cl2F3N6O3+H: 577.2, found 577.2.

Step 2. Synthesis of tert-butyl N-[[1-[2-[3-chloro-4-(trifluoromethyl)phenyl]-5-(2-oxoimidazolidin-1-yl)pyrimidin-4-yl]pyrrolidin-3-yl]methyl]carbamate (109b): A mixture of NaH (15.6 mg, 0.65 mmol) in THF (5 mL) was added to tert-butyl N-[[1-[5-(2-chloroethylcarbamoylamino)-2-[3-chloro-4-(trifluoromethyl)phenyl]pyrimidin-4-yl]pyrrolidin-3-yl]methyl]carbamate (109a) (150.0 mg, 0.260 mmol) in THF (1 mL) at 0° C. The mixture was stirred for 1 h. The mixture was diluted with EA (15 mL×3), washed with water (10 mL), dried over Na₂SO₄, concentrated to get tert-butyl N-[[1-[2-[3-chloro-4-(trifluoromethyl)phenyl]-5-(2-oxoimi-dazolidin-1-yl)pyrimidin-4-yl]pyrrolidin-3-yl]methyl]carbamate (109b) (80 mg, 0.15 mmol, 56% yield) as brown solid. LCMS (ESI): m/z calcd for C24H28ClF3N6O3+H: 541.2, found: 541.2.

Step 3. Synthesis of methy 1-[4-[3-(aminomethyl) pyrrolidin-1-yl]-2-[3-chloro-4-(trifluoromethyl) phenyl]pyrimidin-5-yl]imidazolidin-2-one hydrochloride (109): The solution of tert-butyl N-[[1-[2-[3-chloro-4-(trifluoromethyl)phenyl]-5-(2-oxoimidazolidin-1-yl)pyrimidin-4-yl]pyrrolidin-3-yl]methyl]carbamate (109b) (80.0 mg, 0.1500 mmol) and 2,2,2-trifluoro-acetic acid (0.03 mL, 0.37 mmol) in DCM (5 mL) was stirred at 25° C. for 2 h. The mixture was then concentrated under a reduced pressure. The resulting residue was purified by prep-HPLC (eluting with H₂O:CH₃CN (0.1% HCl) from 5% to 95%) to give methy 1-[4-[3-(aminomethyl) pyrrolidin-1-yl]-2-[3-chloro-4-(trifluoromethyl)phenyl]pyrimidin-5-yl]imidazolidin-2-one hydro-chloride (109) (24.1 mg, 0.050 mmol, 34% yield) as a white solid. LCMS (ESI): m/z calcd for C19H20ClF3N6O+H: 441.14, found: 441.1. ¹H NMR (400 MHz, DMSO) δ 8.56 (s, 1H), 8.48 (d, J=8.5 Hz, 1H), 8.36 (s, 1H), 8.29 (s, 3H), 8.03 (d, J=8.4 Hz, 1H), 6.93 (s, 1H), 4.00 (dd, J=11.6, 7.1 Hz, 1H), 3.91-3.67 (m, 4H), 3.52 (dt, J=15.8, 7.8 Hz, 3H), 2.95 (td, J=12.7, 6.0 Hz, 2H), 2.59 (dt, J=14.4, 7.2 Hz, 1H), 2.17 (td, J=12.0, 6.5 Hz, 1H), 1.93-1.74 (m, 1H).

### Example 14: Exemplary Synthesis of Compound 121

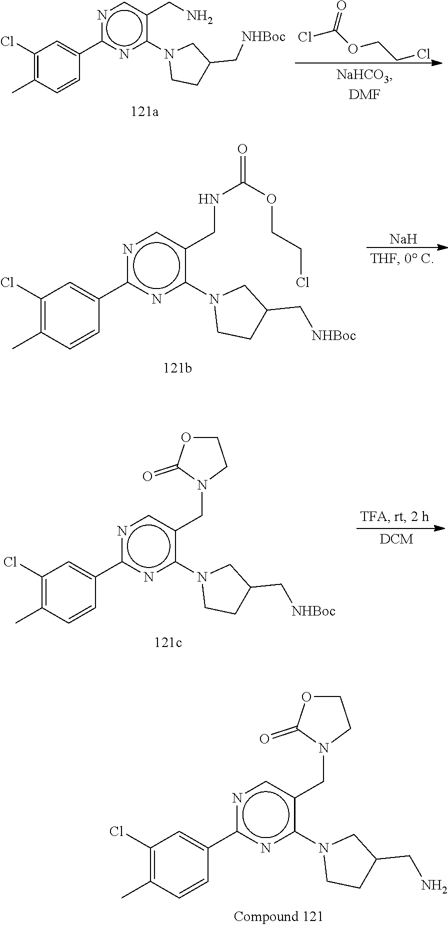

Step 1. Synthesis of 2-chloroethyl N-[[4-[3-[(tert-butoxycarbonylamino)methyl]pyrrolidin-1-yl]-2-(3-chloro-4-methyl-phenyl)pyrimidin-5-yl]methyl]carbamate (121b): To a solution of tert-butyl N-[[1-[5-(aminomethyl)-2-(3-chloro-4-methyl-phenyl)pyrimidin-4-yl]pyrrolidin-3-yl]methyl]carbamate (121a, prepared similarly with the procedures described for 5c) (60.0 mg, 0.14 mmol) in DMF (8 mL) was added 2-chloroethyl carbonochloridate (21.84 mg, 0.15 mmol), NaHCO₃ (35.3 mg, 0.42 mmol) at r.t. The reaction mixture was then stirred at 25° C. for 2 hours. The solvent was removed, and the residue was purified by flash chromatography (DCM:MeOH=20:1) to obtain 2-chloroethyl N-[[4-[3-[(tert-butoxycarbonylamino)methyl]pyrrolidin-1-yl]-2-(3-chloro-4-methyl-phenyl)pyrimidin-5-yl]methyl]carbamate (121b) (60 mg, 0.11 mmol, 80% yield) as yellow oil. LCMS (ESI): m/z calcd for C25H33Cl2N5O4+H: 538.2, found 538.2.

Step 2. Synthesis of tert-butyl N-[[1-[2-(3-chloro-4-methyl-phenyl)-5-[(2-oxooxazolidin-3-yl) methyl]pyrimidin-4-yl]pyrrolidin-3-yl]methyl]carbamate (121c): To a solution of 2-chloroethyl N-[[4-[3-[(tert-butoxycarbonylamino)methyl]pyrrolidin-1-yl]-2-(3-chloro-4-methyl-phenyl)pyrimidin-5-yl]methyl]carbamate (121b) (74.79 mg, 0.14 mmol) in THF (8 mL) was added NaH (10.0 mg, 0.4200 mmol) at r.t. The reaction mixture was then stirred at 25° C. for 2 hours. The mixture was diluted with EtAOc (15 mL×3), washed with water (10 mL), dried over Na₂SO₄, concentrated to get tert-butylN-[[1-[2-(3-chloro-4-methyl-phenyl)-5-[(2-oxooxazolidin-3-yl) methyl]pyrimidin-4-yl]pyrrolidin-3-yl]methyl]carbamate (121c) (60 mg, 0.072 mmol, 52% yield) as a crude yellow solid. LCMS (ESI): m/z calcd for C25H32ClN5O4+H: 502.2, found: 502.2.

Step 3. Synthesis of 3-[[4-[3-(aminomethyl)pyrrolidin-1-yl]-2-(3-chloro-4-methyl-phenyl)pyrimidin-5-yl]methyl]oxazolidin-2-one; 2,2,2-trifluoroacetic acid (121): To a solution of tert-butyl N-[[1-[2-(3-chloro-4-methyl-phenyl)-5-[(2-oxooxazolidin-3-yl)methyl] pyrimidin-4-yl]pyrrolidin-3-yl]methyl]carbamate (121c) (60.0 mg, 0.12 mmol) in DCM (5 mL) was added TFA (1.0 mL, 0.38 mmol) at r.t. The reaction mixture was then stirred at 25° C. for 2 hours. The mixture was then concentrated under a reduced pressure. The resulting residue was purified by prep-HPLC (eluting with H₂O:CH₃CN (0.1% TFA) from 5% to 95%) to give 3-[[4-[3-(aminomethyl)pyrrolidin-1-yl]-2-(3-chloro-4-methyl-phenyl)pyrimidin-5-yl]methyl]oxazolidin-2-one; 2,2,2-trifluoroacetic acid (121) (6.83 mg, 0.013 mmol, 11% yield) was obtained as a white solid. LCMS (ESI): m/z calcd for C20H24ClN5O2+H: 402.2, found: 402.3. ¹H NMR (400 MHz, MeOD-d4) 8.24 (d, J=2.0 Hz, 1H), 8.16 (s, 1H), 8.07-8.05 (m, 1H), 7.52 (d, J=8.0 Hz, 1H), 4.72-4.61 (m, 2H), 4.41 (d, J=6.4 Hz, 2H), 4.27-4.15 (m, 2H), 4.04-3.96 (m, 1H), 3.76-3.73 (m, 1H), 3.59-3.55 (m, 2H), 3.19-3.08 (m, 2H), 2.68 (d, J=8.0 Hz, 1H), 2.48 (s, 1H), 2.36-2.32 (m, 1H), 1.92-1.87 (m, 1H).

### Example 15: Exemplary Synthesis of Compound 125

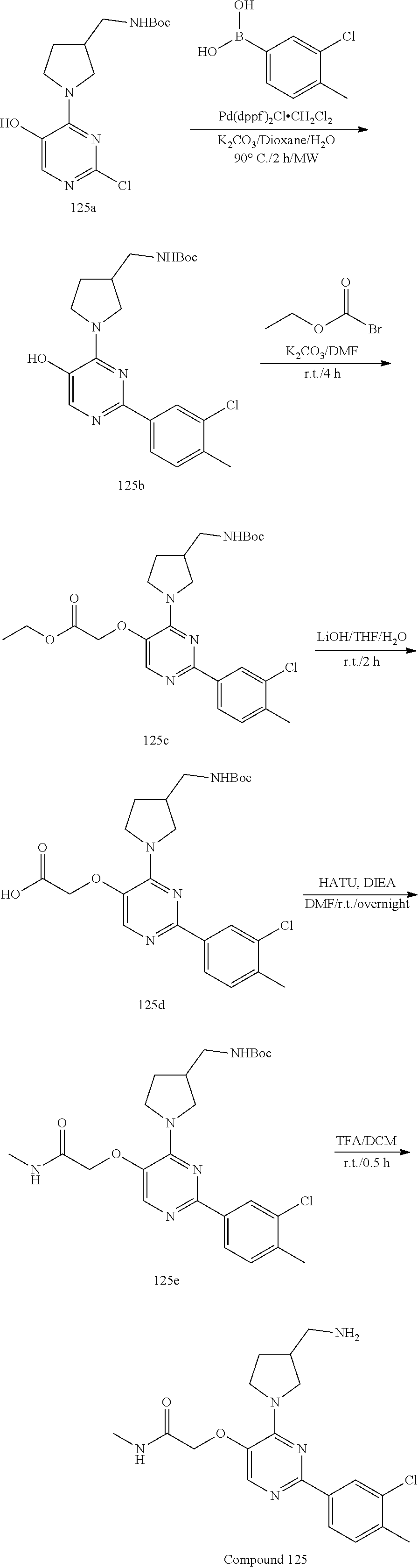

Step 1. Synthesis of tert-butyl ((1-(2-(3-chloro-4-methylphenyl)-5-hydroxypyrimidin-4-yl)pyrrolidin-3-yl)methyl)carbamate (125b): To a mixture of tert-butyl N-[[1-(2-chloro-5-hydroxy-pyrimidin-4-yl)pyrrolidin-3-yl]methyl]carbamate (125a, prepared similarly as described for 3a) (500 mg, 1.52 mmol, 1.0 eq) in 1,4-dioxane/water (10 mL/1 mL) were added (3-chloro-4-methyl-phenyl)boronic acid (259 mg, 1.52 mmol, 1.0 eq), [1,1-Bis(diphenylphosphino)ferrocene]palladium(II) chloride dichloromethane complex (124 mg, 0.150 mmol, 0.1 eq) and potassium carbonate (420 mg, 3.04 mmol, 2.0 eq) under N₂. The sealed vial was heated for 2 h in a Biotage Initiator Eight Microwave Reactor at a constant temperature of 90° C. The resulting mixture was filtered, and then the filtrate was concentrated and diluted with EA (20 mL). The solution was washed with water (3×10 mL) and brine (10 mL), dried over Na₂SO₄, concentrated and purified by flash column chromatography (silica gel, eluting with 50% EA/PE) to afford tert-butyl N-[[1-[2-(3-chloro-4-methyl-phenyl)-5-hydroxy-pyrimidin-4-yl]pyrrolidin-3-yl]methyl]carbamate (125b) (400 mg, 0.955 mmol, 62.8% yield) as a yellow solid. LCMS calculated for C₂₁H₂₈ClN₄O₃(M+H)⁺ m/z=419.2; found: 419.2/421.2.

Step 2. Synthesis of ethyl 2-((4-(3-(((tert-butoxycarbonyl)amino)methyl)pyrrolidin-1-yl)-2-(3-chloro-4-methylphenyl)pyrimidin-5-yl)oxy)acetate (125c): To a solution of tert-butyl N-[[1-[2-(3-chloro-4-methyl-phenyl)-5-hydroxy-pyrimidin-4-yl]pyrrolidin-3-yl]methyl]carbamate (125b) (400 mg, 0.950 mmol, 1.0 eq) in DMF (4 mL) were added potassium carbonate (395 mg, 2.86 mmol, 3.0 eq) and ethyl 2-bromoacetate (319 mg, 1.91 mmol, 2.0 eq) at 15° C. The mixture was stirred at 15° C. for 4 h. The reaction mixture was concentrated and diluted with EA (20 mL). The solution was washed with water (2×10 mL) and brine (10 mL), dried over Na₂SO₄, concentrated and purified by flash column chromatography (silica gel, eluting with 50% EA/PE) to afford ethyl 2-[4-[3-[(tert-butoxycarbonylamino)methyl]pyrrolidin-1-yl]-2-(3-chloro-4-methyl-phenyl)pyrimidin-5-yl]oxyacetate (125c) (350 mg, 0.693 mmol, 72.6% yield) as a yellow solid. LCMS calculated for C₂₅H₃₄ClN₄O₅(M+H)⁺ m/z=505.2; found: 505.3/507.3.

Step 3. Synthesis of 2-((4-(3-(((tert-butoxycarbonyl)amino)methyl)pyrrolidin-1-yl)-2-(3-chloro-4-methylphenyl)pyrimidin-5-yl)oxy)acetic acid (125d): To a solution of ethyl 2-[4-[3-[(tert-butoxycarbonylamino)methyl]pyrrolidin-1-yl]-2-(3-chloro-4-methyl-phenyl)pyrimidin-5-yl]oxyacetate (125c) (360 mg, 0.710 mmol, 1.0 eq) in water/THF (1 mL/5 mL) was added lithium hydroxide monohydrate (59.8 mg, 1.43 mmol, 2.0 eq) at 15° C. The mixture was stirred at 15° C. for 2 h. The pH value of the reaction solution was adjusted to 4-5 with 1N HCl aqueous solution. The reaction mixture is filtered, and the filter cake was dried in vacuum to afford 2-[4-[3-[(tert-butoxycarbonylamino)methyl]pyrrolidin-1-yl]-2-(3-chloro-4-methyl-phenyl)pyrimidin-5-yl]oxyacetic acid (125d) (300 mg, 0.629 mmol, 88.2% yield) as a yellow solid. LCMS calculated for C₂₃H₃₀ClN₄O₅(M+H)⁺ m/z=477.2; found: 477.3/479.2.

Step 4. tert-butyl ((1-(2-(3-chloro-4-methylphenyl)-5-(2-(methylamino)-2-oxoethoxy)pyrimidin-4-yl)pyrrolidin-3-yl)methyl)carbamate (125e): To a solution of 2-[4-[3-[(tert-butoxycarbonylamino)methyl]pyrrolidin-1-yl]-2-(3-chloro-4-methyl-phenyl)pyrimidin-5-yl]oxyacetic acid (125d) (50.0 mg, 0.100 mmol, 1.0 eq) in DMF (2 mL) were added DIEA (54.2 mg, 0.420 mmol, 4.2 eq) and HATU (59.8 mg, 0.160 mmol. 1.6 eq) at 15° C. The mixture was stirred at 15° C. for 0.5 h. Methylamine hydrochloride (14.2 mg, 0.210 mmol, 2.1 eq) was added into the mixture. The mixture was stirred at 15° C. overnight. The reaction mixture was concentrated and purified by Prep-HPLC on a C18 column (5 uM, 50×150 mm) with mobile phase: H₂O (0.1% NH₄HCO₃)/MeOH at flow rate: 50 mL/min to afford tert-butyl N-[[1-[2-(3-chloro-4-methyl-phenyl)-5-[2-(methylamino)-2-oxo-ethoxy]pyrimidin-4-yl]pyrrolidin-3-yl]methyl]carbamate (125e) (40.0 mg, 0.0816 mmol, 77.9% yield) as a white solid. LCMS calculated for C₂₄H₃₃ClN₅O₄(M+H)⁺ m/z=490.2; found: 490.3/492.3.

Step 5. 2-((4-(3-(aminomethyl)pyrrolidin-1-yl)-2-(3-chloro-4-methylphenyl)pyrimidin-5-yl)oxy)-N-methylacetamide (125): To a mixture of tert-butyl N-[[1-[2-(3-chloro-4-methyl-phenyl)-5-[2-(methylamino)-2-oxo-ethoxy]pyrimidin-4-yl]pyrrolidin-3-yl]methyl]carbamate (125e) (30.0 mg, 0.0600 mmol, 1.0 eq) in DCM (3 mL) was added TFA (1.0 mL). The mixture was stirred at room temperature for 0.5 h. The reaction mixture was concentrated and purified by Prep-HPLC on a C18 column (5 uM, 50×150 mm) with mobile phase: H₂O (0.1% NH₄HCO₃)/MeOH at flow rate: 50 mL/min to afford 2-[4-[3-(aminomethyl)pyrrolidin-1-yl]-2-(3-chloro-4-methyl-phenyl)pyrimidin-5-yl]oxy-N-methyl-acetamide (125) (20.8 mg, 0.0527 mmol, 86.1% yield) as a white solid. LCMS calculated for C₁₉H₂₅ClN₅O₂(M+H)⁺ m/z=390.2; found: 390.2/392.2. ¹H NMR (400 MHz, CD₃OD) δ 8.18 (d, J=1.6 Hz, 1H), 8.04 (dd, J=7.6, 1.6 Hz, 1H), 7.78 (s, 1H), 7.32 (d, J=8.0 Hz, 1H), 4.54 (s, 2H), 4.05-4.13 (m, 1H), 3.97-4.04 (m, 1H), 3.76-3.87 (m, 1H), 3.51-3.59 (m, 1H), 2.82 (s, 3H), 2.78 (d, J=6.8 Hz, 2H), 2.40 (s, 3H), 2.32-2.38 (m, 1H), 2.10-2.21 (m, 1H), 1.66-1.77 (m, 1H).

### Example 16: Exemplary Synthesis of Compound 128

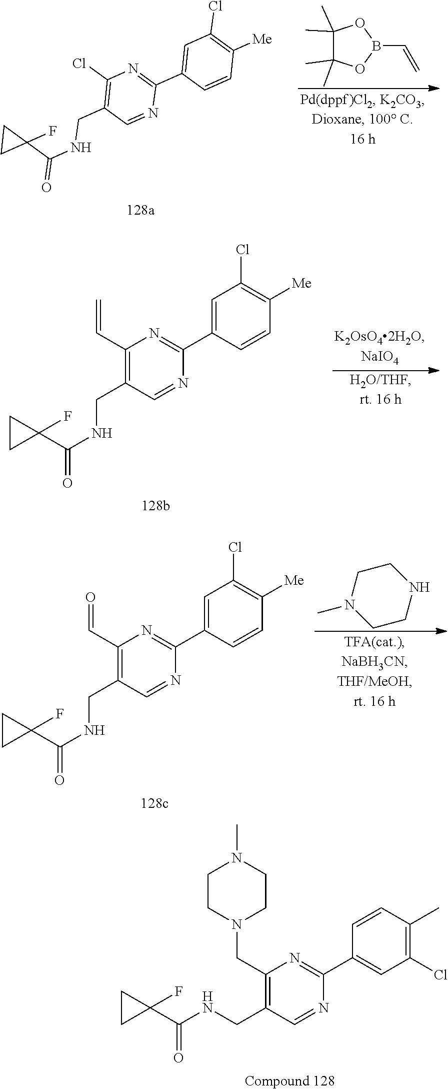

Step 1. Synthesis of N-[[2-(3-chloro-4-methyl-phenyl)-4-vinyl-pyrimidin-5-yl]methyl]-1-fluoro-cyclopropanecarboxamide (128b): To a degassed solution of 4,4,5,5-tetramethyl-2-vinyl-1,3,2-dioxaborolane (141.31 mg, 0.92 mmol) in 1,4-dioxane (10 mL) was added N-[[4-chloro-2-(3-chloro-4-methyl-phenyl)pyrimidin-5-yl]methyl]-1-fluoro-cyclopropanecarboxamide (128a, prepared similarly as described for 5 h) (325.0 mg, 0.92 mmol), Pd(dppf)Cl₂ (33.57 mg, 0.050 mmol) and K₂CO₃ (253.24 mg, 1.84 mmol) at rt. The degassed mixture was stirred at 100° C. for 16 h under Ar. The reaction mixture was concentrated under vacuo and purified by silica gel chromatography column (PE:EtOAc=6:1 to 3:1) to give the product N-[[2-(3-chloro-4-methyl-phenyl)-4-vinyl-pyrimidin-5-yl]methyl]-1-fluoro-cyclopropanecarboxamide (128b) (130 mg, 0.38 mmol, 41% yield) as a white solid. LCMS (ESI): m/z calcd for C₁₈H₁₇ClFN₃O+H: 346.1, found: 346.1.

Step 2. Synthesis of N-[[2-(3-chloro-4-methyl-phenyl)-4-formyl-pyrimidin-5-yl]methyl]-1-fluoro-cyclopropanecarboxamide (128c): To a mixture of N-[[2-(3-chloro-4-methyl-phenyl)-4-vinyl-pyrimidin-5-yl]methyl]-1-fluoro-cyclopropanecarboxamide (128b) (130.0 mg, 0.38 mmol) in THF (6 mL) and water (2 mL) was added K₂OsO₄·2H₂O (13.85 mg, 0.040 mmol) and NaIO₄ (402 mg, 1.88 mmol). The mixture was stirred at 25° C. for 16 h. The mixture was diluted with H₂O (50.0 mL), extracted with EtOAc (30.0 mL×3), dried over Na₂SO₄, filtered and concentrated under vacuum to give crude N-[[2-(3-chloro-4-methyl-phenyl)-4-formyl-pyrimidin-5-yl]methyl]-1-fluoro-cyclopropanecarboxamide (128c) (130 mg, 0.38 mmol, 99% yield) as a white solid which was used for next step without further purification. LCMS (ESI): m/z calcd for Cl₇H₁₅ClFN₃O₂+H: 348.1, found: 348.1.

Step 3. Synthesis of N-[[2-(3-chloro-4-methyl-phenyl)-4-[(4-methylpiperazin-1-yl)methyl]pyrimidin-5-yl]methyl]-1-fluoro-cyclopropanecarboxamide; 2,2,2-trifluoroacetic acid (128): To a degassed solution of N-[[2-(3-chloro-4-methyl-phenyl)-4-formyl-pyrimidin-5-yl]methyl]-1-fluoro-cyclopropanecarboxamide (128c) (113.0 mg, 0.32 mmol) and 1-methylpiperazine (65.09 mg, 0.65 mmol) in methanol (5 mL) was added TFA (1 drop). The resulting mixture was stirred for 3 h, then NaBH₃CN (40.84 mg, 0.65 mmol) was added at rt. The degassed mixture was stirred at 20° C. for 16 h under Ar. The reaction mixture was concentrated under vacuo and purified by prep-HPLC (eluting with H₂O:CH₃CN (0.1% TFA) from 5:95 to 90:10) to get N-[[2-(3-chloro-4-methyl-phenyl)-4-[(4-methylpiperazin-1-yl)methyl]pyrimidin-5-yl]methyl]-1-fluoro-cyclopropanecarboxamide; 2,2,2-trifluoroacetic acid (128) (39 mg, 0.059 mmol, 18% yield) as an off-white solid. LCMS (ESI): m/z calcd for C₂₂H₂₇ClFN₅O+H: 432.2, found: 432.1. ¹H NMR (400 MHz, MeOD) δ 8.75 (s, 1H), 8.40 (d, J=1.7 Hz, 1H), 8.25 (dd, J=8.0, 1.7 Hz, 1H), 7.41 (d, J=8.1 Hz, 1H), 4.70 (s, 2H), 4.00 (d, J=2.6 Hz, 2H), 3.31 (dt, J=3.3, 1.6 Hz, 2H), 3.06-2.75 (m, 6H), 2.44 (s, 3H), 1.52-1.08 (m, 4H).

### Example 17: Exemplary Synthesis of Compound 147

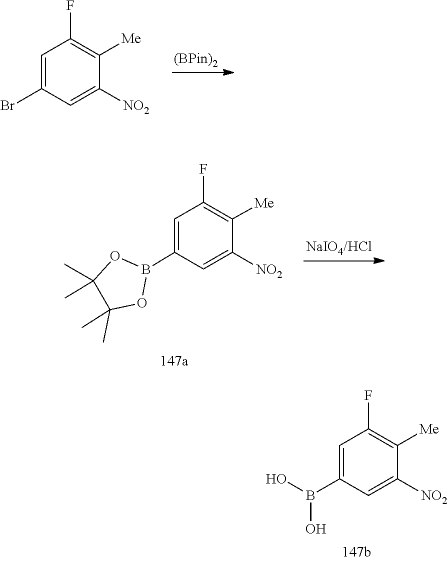 [SMILES: Cc1c(F)cc(Br)cc1[N+](=O)[O-] ; Cc1c(F)cc(B2OC(C)(C)C(C)(C)O2)cc1[N+](=O)[O-] ; Cc1c(F)cc(B(O)O)cc1[N+](=O)[O-]]

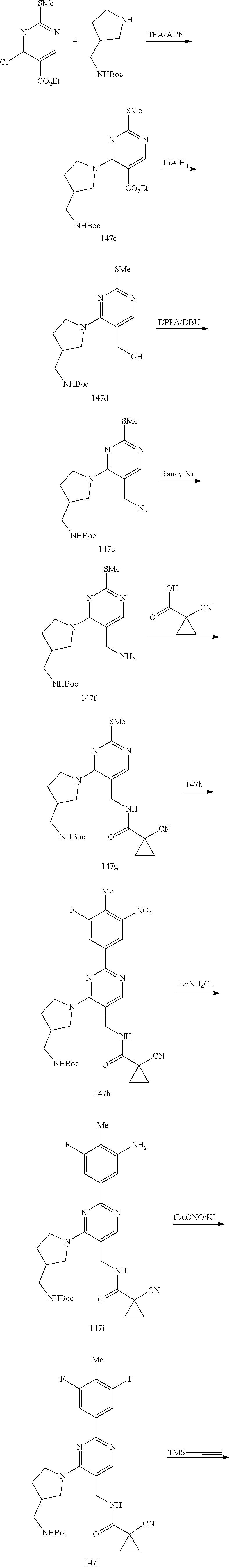

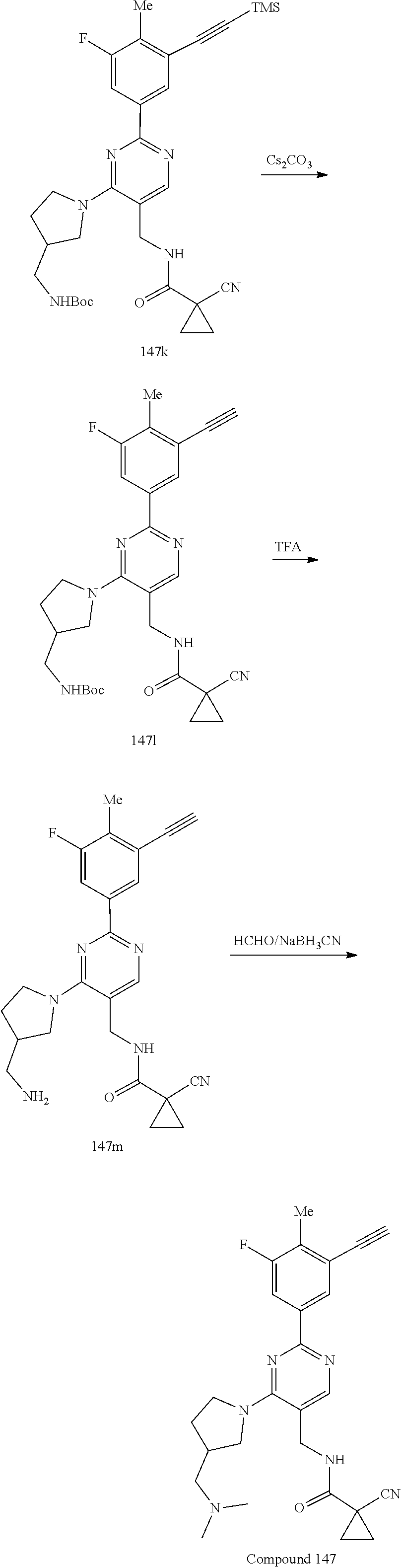

Step 1. Synthesis of 2-(3-fluoro-4-methyl-5-nitrophenyl)-4,4,5,5-tetramethyl-1,3,2-dioxaborolane (147a). To a mixture of 5-bromo-1-fluoro-2-methyl-3-nitro-benzene (13.0 g, 55.55 mmol) in 1,4-dioxane (80 mL) was added 4,4,5,5-tetramethyl-2-(4,4,5,5-tetramethyl-1,3,2-dioxaborolan-2-yl)-1,3,2-dioxaborolane (18.4 g, 72.22 mmol) and Pd(dppf)Cl2 (4.06 g, 5.56 mmol) and KOAc (7.67 g, 55.55 mmol). The mixture was stirred at 100° C. for 16 h under argon. The reaction mixture was diluted with EtOAc (200 mL) and washed with H₂O (200 mL×2) and brine (100 mL). The organic layer was separated and dried over anhydrous sodium sulfate, filtered and concentrated under vacuo to give crude product which was purified by silica gel column (EtOAc:PE=10:1) to give the 3-fluoro-2-methyl-5-(4,4,5,5-tetramethyl-1,3,2-dioxaborolan-2-yl)aniline (147a, 10.0 g, 35.58 mmol, 64% yield) as a white solid. ¹H NMR (400 MHz, CDCl₃) δ 8.11 (s, 1H), 7.67 (d, J=9.2 Hz, 1H), 2.48 (d, J=2.0 Hz, 3H), 1.33 (s, 12H).

Step 2. Synthesis of (3-fluoro-4-methyl-5-nitrophenyl)boronic acid (147b). To a mixture of 2-(3-fluoro-4-methyl-5-nitro-phenyl)-4,4,5,5-tetramethyl-1,3,2-dioxaborolane (147a, 20.0 g, 71.15 mmol) in THF (100 mL) and water (30 mL) was added NaIO₄ (45.65 g, 213.5 mmol) and HCl (23.6 mL, 142.3 mmol, 6 mol/L in water). The mixture was stirred at 25° C. for 2 h. The mixture was quenched with H₂O (100 mL) at 25° C., extracted with EtOAC (100 mL×3), dried over Na₂SO₄, concentrated. The crude product was purified by silica gel chromatography (PE:EA=10:1 to 1:1) to give (3-fluoro-4-methyl-5-nitro-phenyl)-boronic acid (147b, 12 g, 60.32 mmol, 85% yield) as a yellow solid. LCMS (ESI): m/z calcd for C₁₄H₁₂B₂F₂N₂O₇−H ([2M−H]): 379.1; found: 379.0.

Step 3. Synthesis of ethyl 4-[3-[(tert-butoxycarbonylamino)methyl]pyrrolidin-1-yl]-2-methylsulfanyl-pyrimidine-5-carboxylate (147c). To a solution of ethyl 4-chloro-2-methylsulfanyl-pyrimidine-5-carboxylate (10 g, 42.98 mmol) in acetonitrile (100 mL) was added Et₃N (9.13 g, 90.25 mmol) and tert-butyl N-(pyrrolidin-3-ylmethyl)carbamate (9.46 g, 47.27 mmol). The mixture was stirred at 25° C. for 15 min. The mixture was diluted with EtOAc (300 mL), and washed with H₂O (3×100 mL), dried over Na₂SO₄ and filtered. The filtrate was concentrated to afford a crude product ethyl 4-[3-[(tert-butoxycarbonylamino)methyl]pyrrolidin-1-yl]-2-methylsulfanyl-pyrimidine-5-carboxylate (147c, 16.7 g, 41.6 mmol, 96.8% yield) as yellow oil. LCMS (ESI): m/z calcd for C₁₈H₂₈N₄O₄S+H: 397.5, found: 397.4.

Step 4. Synthesis of tert-butyl N-[[1-[5-(hydroxymethyl)-2-methylsulfanyl-pyrimidin-4-yl] pyrrolidin-3-yl]methyl]carbamate (147d). To a solution of ethyl 4-[3-[(tert-butoxycarbonylamino)methyl]pyrrolidin-1-yl]-2-methylsulfanyl-pyrimidine-5-carboxylate (147c, 5.0 g, 12.61 mmol) in THF (400 mL) was added LiAlH₄ (957.12 mg, 25.22 mmol) at r.t under Ar. The reaction mixture was then stirred at 25° C. for 10 min. The mixture was quenched carefully with NaOH (6.4 mL, 15% in water) at 0° C. The mixture was stirred for 15 min and filtered over celite. The filtrate was concentrated to get tert-butyl N-[[1-[5-(hydroxymethyl)-2-methylsulfanyl-pyrimidin-4-yl]pyrrolidin-3-yl]methyl]carbamate (147d, 4.5 g, crude) as a white solid which was used directly for the next step. LCMS (ESI): m/z calcd for C₁₆H₂₆N₄O₃S+H: 355.1, found: 355.3.

Step 5. Synthesis of tert-butyl N-[[1-[5-(azidomethyl)-2-methylsulfanyl-pyrimidin-4-yl]pyrrolidin-3-yl]methyl]carbamate (147e). To a solution of tert-butyl N-[[1-[5-(hydroxymethyl)-2-methylsulfanyl-pyrimidin-4-yl] pyrrolidin-3-yl]methyl]carbamate (147d, 13.82 g, 38.99 mmol, crude) in THF (160 mL) was added DBU (5.92 g, 42.89 mmol) and DPPA (1.78 g, 46.79 mmol) slowly at 25° C. The mixture was stirred at 25° C. for 0.5 h. The mixture was diluted with water (300 mL) and extracted with EtOAc (150 mL×3), dried over Na₂SO₄, and concentrated. The crude product was purified by flash chromatography (H₂O:CH₃CN=90:10 to 50:50) to get tert-butyl N-[[1-[5-(azidomethyl)-2-methylsulfanyl-pyrimidin-4-yl]pyrrolidin-3-yl]methyl]carbamate (147e, 3.35 g, 8.56 mmol, 22.0% yield) as white solid. LCMS (ESI): m/z calcd for C₁₆H₂₅N₇O₂S+H: 380.1, found: 380.4.

Step 6. Synthesis of tert-butyl N-[[1-[5-(aminomethyl)-2-methylsulfanyl-pyrimidin-4-yl]pyrrolidin-3-yl]methyl]carbamate (147f). To a solution of tert-butyl N-[[1-[5-(azidomethyl)-2-methylsulfanyl-pyrimidin-4-yl]pyrrolidin-3-yl] methyl]carbamate (147e, 3.35 g, 8.56 mmol) in methanol (50 mL) and THF (50 mL) was added Raney-Ni (194.48 mg, 0.86 mmol). The mixture was purged with H₂ three times and stirred under H₂ (1 atm) at 20° C. for 18 hours. The mixture was diluted with EtOAc (200 mL) and filtered through celite. The filtrate was concentrated to afford tert-butyl N-[[1-[5-(aminomethyl)-2-methylsulfanyl-pyrimidin-4-yl]pyrrolidin-3-yl]methyl]carbamate (147f, 3.3 g, 9.34 mmol, crude) as yellow oil. LCMS m/z calcd for C₁₆H₂₇N₅O₂S+H: 354.1, found: 354.1.

Step 7. Synthesis of tert-butyl N-[[1-[5-[[(1-cyanocyclopropanecarbonyl)amino]methyl]-2-methylsulfanyl-pyrimidin-4-yl]pyrrolidin-3-yl]methyl]carbamate (147g). To a solution of tert-butyl N-[[1-[5-(aminomethyl)-2-methylsulfanyl-pyrimidin-4-yl] pyrrolidin-3-yl]methyl]carbamate (147f, 6.6 g, 18.67 mmol), 1-cyanocyclopropanecarboxylic acid (2.28 mg, 20.54 mmol) and NEt₃ (2.27 g, 22.41 mmol) in DMF (70 mL) was added HATU (7.81 g, 20.54 mmol). The mixture was stirred at 25° C. for 1 h. The mixture was diluted with water (350 mL), extracted with EtOAc (100 mL×3). The organic solution was washed with water (150 mL×3) and brine (50 mL), dried over Na₂SO₄, concentrated. The product was purified on silica gel column to afford tert-butyl N-[[1-[5-[[(1-cyanocyclopropanecarbonyl)amino]methyl]-2-methylsulfanyl-pyrimidin-4-yl]pyrrolidin-3-yl]methyl]carbamate (147g, 7.52 g, 16.8 mmol, 90% yield) as white solid. LCMS (ESI): m/z calcd for C₂₁H₃₀N₆O₃S+H: 447.1, found: 447.4.

Step 8. Synthesis of tert-butyl N-[[1-[5-[[(1-cyanocyclopropanecarbonyl)amino]methyl]-2-(3-fluoro-4-methyl-5-nitro-phenyl)pyrimidin-4-yl]pyrrolidin-3-yl]methyl]carbamate (147h). A mixture of tert-butyl N-[[1-[5-[[(1-cyanocyclopropanecarbonyl)amino]methyl]-2-methylsulfanyl-pyrimidin-4-yl]pyrrolidin-3-yl]methyl]carbamate (147g, 10.0 g, 22.39 mmol), (3-fluoro-4-methyl-5-nitro-phenyl)boronic acid (5.35 g, 26.87 mmol), Pd(Ph₃P)₄ (1.29 g, 1.12 mmol), and copper(I)thiophene-2-carboxylate (8.58 g, 44.79 mmol) in 1,4-dioxane (100 mL) was stirred at 100° C. 16 h. The mixture was diluted with EtOAc (150 mL), filtered. The filtrate was washed with NaOH (20 mL, 5% in water), NH₄Cl (30 mL) and brine (20 mL), dried, concentrated. The crude material was purified on a column (PE:EA=2:1, then DCM:MeOH=10:1). Tert-butyl N-[[1-[5-[[(1-cyanocyclopropanecarbonyl)amino]methyl]-2-(3-fluoro-4-methyl-5-nitro-phenyl)pyrimidin-4-yl]pyrrolidin-3-yl]methyl]carbamate (147h, 5.8 g, 10.5 mmol, 46.8% yield) were obtained as yellow solid. LCMS (ESI): m/z calcd for C₂₇H₃₂FN₇O₅+H: 554.2, found: 554.2.

Step 9. Synthesis of tert-butyl N-[[1-[2-(3-amino-5-fluoro-4-methyl-phenyl)-5-[[(1-cyanocyclopropanecarbonyl)amino]methyl]pyrimidin-4-yl]pyrrolidin-3-yl]methyl]carbamate (147i). To a mixture of tert-butyl N-[[1-[5-[[(1-cyanocyclopropanecarbonyl)amino]methyl]-2-(3-fluoro-4-methyl-5-nitro-phenyl)pyrimidin-4-yl]pyrrolidin-3-yl]methyl]carbamate (147h, 8.7 g, 15.72 mmol) in ethanol (100 mL), THF (20 mL) and water (30 mL) was added NH₄Cl (4.2 g, 78.58 mmol) and Fe (4.4 g, 78.58 mmol). The mixture was stirred at 80° C. for 2 h under argon. The mixture was filtered, concentrated and purified by silica chromatography column (DCM:MeOH=10:1) to obtain tert-butyl N-[[1-[2-(3-amino-5-fluoro-4-methyl-phenyl)-5-[[(1-cyanocyclopropanecarbonyl)amino]-methyl]pyrimidin-4-yl]pyrrolidin-3-yl]methyl]carbamate (147i, 6.1 g, 11.65 mmol, 74% yield) as a yellow solid. LCMS (ESI): m/z calcd for C₂₇H₃₄FN₇O₃+H: 524.3; found: 524.4.

Step 10. Synthesis of tert-butyl N-[[1-[5-[[(1-cyanocyclopropanecarbonyl)amino]methyl]-2-(3-fluoro-5-iodo-4-methyl-phenyl)pyrimidin-4-yl]pyrrolidin-3-yl]methyl]carbamate (147j). To a solution of tert-butyl N-[[1-[2-(3-amino-5-fluoro-4-methyl-phenyl)-5-[[(1-cyanocyclo-propanecarbonyl)amino]methyl]pyrimidin-4-yl]pyrrolidin-3-yl]methyl]carbamate (147i, 6.1 g, 11.65 mmol) in MeCN (30 mL) was added tert-Butyl nitrite (12.0 g, 116.5 mmol) and CuI (13.28 g, 69.9 mmol). The mixture was stirred at 25° C. for 3 h. The mixture was filtered and concentrated, and purified by silica chromatography column (PE:EtOAc=5:1) to obtain tert-butyl N-[[1-[5-[[(1-cyanocyclopropanecarbonyl)amino]methyl]-2-(3-fluoro-5-iodo-4-methyl-phenyl)pyrimidin-4-yl]pyrrolidin-3-yl]methyl]carbamate (147j, 3.4 g, 5.36 mmol, 46% yield) as a brown solid. LCMS (ESI): m/z calcd for C₂₇H₃₂FIN₆O₃+H: 635.2; found: 635.3.

Step 11. Synthesis of tert-butyl N-[[1-[5-[[(1-cyanocyclopropanecarbonyl)amino]methyl]-2-[3-fluoro-4-methyl-5-(2-trimethylsilylethynyl)phenyl]pyrimidin-4-yl]pyrrolidin-3-yl]methyl]carbamate (147k). To a solution of tert-butyl N-[[1-[5-[[(1-cyanocyclopropanecarbonyl)amino]methyl]-2-(3-fluoro-5-iodo-4-methyl-phenyl)pyrimidin-4-yl]pyrrolidin-3-yl]methyl]carbamate (147j, 3.1 g, 4.89 mmol) in THF (80 mL) were added Pd(PPh₃)₂Cl₂ (0.34 g, 0.49 mmol), CuI (0.26 g, 0.98 mmol), TEA (1.483 g, 14.66 mmol) and ethynyl(trimethyl)silane (959.78 mg, 9.77 mmol) at rt under nitrogen. The mixture was stirred at 25° C. for 5 h. The mixture was diluted with water (50 mL), extracted with EtOAc (50 mL×2), washed with brine (50 mL), dried over Na₂SO₄, concentrated and purified by silica chromatography column (PE:EtOAc=1:1) to obtain tert-butyl N-[[1-[5-[[(1-cyanocyclopropanecarbonyl)amino]methyl]-2-[3-fluoro-4-methyl-5-(2-trimethylsilylethynyl)phenyl]pyrimidin-4-yl]pyrrolidin-3-yl]methyl]carbamate (147k, 1.8 g, 2.98 mmol, 61% yield) as a brown solid. LCMS (ESI): m/z calcd for C₃₂H₄₁FN₆O₃Si+H: 605.3; Found: 605.4.

Step 12. 1.10 Synthesis of tert-butyl N-[[1-[5-[[(1-cyanocyclopropanecarbonyl) amino]methyl]-2-(3-ethynyl-5-fluoro-4-methyl-phenyl)pyrimidin-4-yl]pyrrolidin-3-yl]methyl]carbamate (147l). A mixture of tert-butyl N-[[1-[5-[[(1-cyanocyclopropanecarbonyl)amino]methyl]-2-[3-fluoro-4-methyl-5-(2-trimethylsilylethynyl)phenyl] pyrimidin-4-yl]pyrrolidin-3-yl]methyl]carbamate (147k, 1.8 g, 2.98 mmol) and Cs₂CO₃ (357 mg, 5.95 mmol) in methanol (30 mL) was stirred at 25° C. for 0.5 h under Ar. The mixture was filtered, concentrated and purified by silica chromatography column (PE:EtOAc=1:1) to obtain tert-butyl N-[[1-[5-[[(1-cyanocyclopropanecarbonyl)amino]methyl]-2-(3-ethynyl-5-fluoro-4-methyl-phenyl)pyrimidin-4-yl]pyrrolidin-3-yl]methyl]carbamate (147l, 1.2 g, 2.25 mmol, 76% yield) as a brown solid. LCMS (ESI): m/z calcd for C₂₉H₃₃FN₆O₃+H: 533.3; found: 533.4.

Step 13. Synthesis of N-[[4-[3-(aminomethyl)pyrrolidin-1-yl]-2-(3-ethynyl-5-fluoro-4-methyl-phenyl)pyrimidin-5-yl]methyl]-1-cyano-cyclopropanecarboxamide (147m). To a solution of tert-butyl N-[[1-[5-[[(1-cyanocyclopropanecarbonyl)amino]methyl]-2-(3-ethynyl-5-fluoro-4-methyl-phenyl)pyrimidin-4-yl]pyrrolidin-3-yl]methyl]carbamate (147l, 1.26 g, 2.37 mmol) in DCM (10 mL) was added TFA (2.0 mL, 2.37 mmol) at 25° C. for 2 h. The mixture was concentrated to get N-[[4-[3-(aminomethyl)pyrrolidin-1-yl]-2-(3-ethynyl-5-fluoro-4-methyl-phenyl) pyrimidin-5-yl]methyl]-1-cyano-cyclopropanecarboxamide (147m, 0.97 g, 2.24 mmol, 99% yield) as brown crude oil. LCMS (ESI): m/z calcd for C₂₄H₂₅FN₆O+H: 433.2; found: 433.4.

Step 14. Synthesis of 1-cyano-N-[[4-[3-[(dimethylamino)methyl]pyrrolidin-1-yl]-2-(3-ethynyl-5-fluoro-4-methyl-phenyl)pyrimidin-5-yl]methyl]cyclopropanecarboxamide (147). To a solution of N-[[4-[3-(aminomethyl)pyrrolidin-1-yl]-2-(3-ethynyl-5-fluoro-4-methyl-phenyl)pyrimidin-5-yl]methyl]-1-cyano-cyclopropanecarboxamide (147m, 0.97 g, 2.24 mmol) in methanol (10 mL) was added CH₂O (829.9 mg, 22.43 mmol, 37% in water) and NaCNBH₃ (1409 mg, 22.43 mmol) at 25° C. for 2 h. The mixture was concentrated and purified by prep-HPLC (eluting with H₂O:CH₃CN (0.1% NH₄HCO₃) from 90:10 to 10:90). The purified product was dried through lyophilization to obtained a white solid as free base. The free base was dissolved in MeOH (20 mL) and neutralized with HCl (0.46 mL, 3 mol/L in MeOH). Removal of solvent under vacuum afforded the product 1-cyano-N-[[4-[3-[(dimethylamino)methyl]pyrrolidin-1-yl]-2-(3-ethynyl-5-fluoro-4-methyl-phenyl)pyrimidin-5-yl]methyl]cyclopropanecarboxamide; hydrochloride (147, 700 mg, 1.37 mmol, 61% yield) as white solid. LCMS (ESI): m/z calcd for C₂₆H₂₉FN₆O+H: 461.2; found: 461.2. ¹H NMR (400 MHz, MeOD) δ 8.15 (m, 2H), 7.97 (dd, J=10.4, 1.6 Hz, 1H), 4.62 (s, 2H), 4.26 (m, 1H), 4.12 (m, 1H), 4.04-3.90 (m, 2H), 3.73 (m, 1H), 3.11-3.12 (m, 2H), 2.99 (s, 6H), 2.91-2.75 (m, 1H), 2.44 (d, J=2.1 Hz, 3H), 2.41-2.29 (m, 1H), 1.90 (m, 1H), 1.62 (m, 2H), 1.59 (m, 2H). ¹⁹F NMR (377 MHz, MeOD) δ −117.54 (s).

Table 1 includes spectroscopic data for compounds synthesized as described in Examples 1-17 or analogously synthesized using the exemplary procedures of Examples 1-17.

TABLE 1
Spectroscopic data of Compounds 1-150

| | | Spectroscopic Data |
|---|---|---|
| No. | Structure | (NMR and LCMS) |
| 1 | 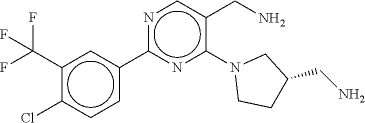 | LCMS (ESI): m/z calcd for C17H19ClF3N5 + H: 386.1, found: 386.1. ¹H NMR (400 MHz, MeOD) δ 8.72 (d, J = 2.0 Hz, 1H), 8.56 (dd, J = 8.4, 2.0 Hz, 1H), 8.38 (s, 1H), 7.75 (d, J = 8.4 Hz, 1H), 4.45-4.32 (m, 2H), 4.13-4.09 (m, 1H), 4.04-3.94 (m, 1H), 3.92-3.85 (m, 1H), 3.67- 3.63 (m, 1H), 3.21-3.10 (m, 2H), 2.72-2.65 (m, 1H), 2.39-2.32 (m, 1H), 1.95-1.86 (m, 1H). |
| 2 | 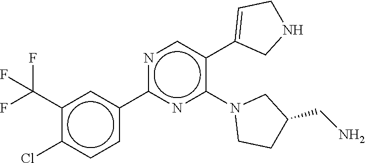 | See Ex. 1 |
| 3 | 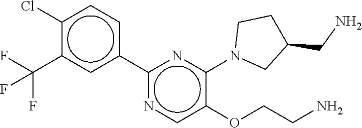 | See Ex. 2 |
| 4 | 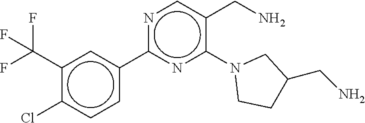 | Same as compound 1 |
| 5 | 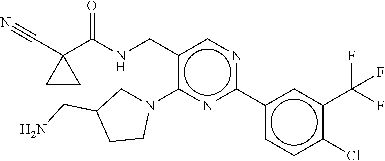 | See Ex. 3A and 3B |
| 6 | 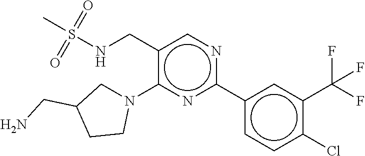 | See Ex. 4 |
| 7 | 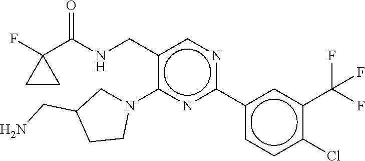 | LCMS (ESI): m/z calcd for C21H22ClF4N5O + H: 472.2, found: 472.2. ¹H NMR (400 MHz, MeOD) δ 8.64 (d, J = 2.0 Hz, 1H), 8.48 (dd, J = 8.4, 2.0 Hz, 1H), 8.20 (s, 1H), 7.81 (d, J = 8.4 Hz, 1H), 4.68 (s, 2H), 4.26-4.07 (m, 2H), 4.05-3.90 (m, 1H), 3.73 (dd, J = 11.6, 8.0 Hz, 1H), 3.13 (qd, J = 13.0, 7.6 Hz, 2H), 2.67 (dt, J = 15.2, 8.0 Hz, 1H), 2.33 (dd, J = 10.8, 5.2 Hz, 1H), 1.98-1.78 (m, 1H), 1.44-1.24 (m, 4H). |
| 8 | 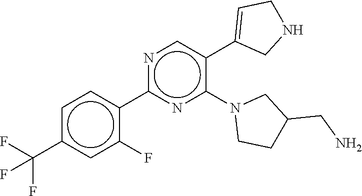 | LCMS (ESI): m/z calcd for C20H21F4N5 + H: 408.2, found: 408.2. ¹H NMR (400 MHz, MeOD): δ8.28 (s, 1H), 8.20 (m, 1H), 7.65 (m, 2H), 5.98 (s, 1H), 4.93 (m, 1H), 4.34 (m, 3H), 3.88 (m, 1H), 3.74 (m, 2H), 3.46 (m, 1H), 3.12 (m, 2H). 2.61 (m, 1H), 2.25 (m, 1H), 1.82 (m, 1H). |
| 9 | 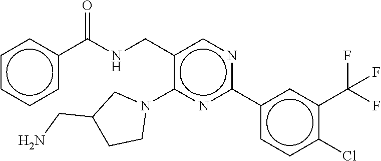 | LCMS (ESI): m/z calcd for C24H23ClF3N5O + H: 490.2, found: 490.2. 1H NMR (400 MHz, MeOD) δ 8.66 (s, 1H), 8.49 (d, J = 8.4 Hz, 1H), 8.25 (s, 1H), 7.87 (d, J = 8.0 Hz, 2H), 7.79 (d, J = 8.4 Hz, 1H), 7.56 (d, J = 7.6 Hz, 1H), 7.48 (t, J = 7.6 Hz, 2H), 4.78 (s, 2H), 4.14 (s, 2H), 4.00 (s, 1H), 3.75 (s, 1H), 3.11 (dd, J = 24.4, 10.4 Hz, 2H), 2.65 (s, 1H), 2.32 (s, 1H), 1.88 (s, 1H). |
| 10 | 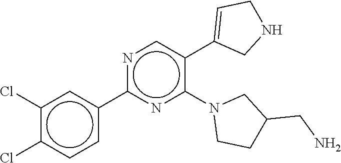 | LCMS (ESI): m/z calcd for C19H21Cl2N5 + H: 390.2, found: 390.1. 1H NMR (400 MHz, MeOD) δ 8.45 (s, 1H), 8.25 (s, 2H), 7.69-7.65 (m, 1H), 5.95 (s, 1H), 4.46 (d, J = 14.8, 1H), 4.33 (s, 3H), 3.93-3.89 (m, 1H), 3.76- 3.73 (m, 2H), 351-3.46 (m, 1H), 3.12 (d, J = 7.2, 2H), 2.63-2.59 (m, 1H), 2.32-2.25 (m, 1H), 1.87-1.77 (m, 1H). |
| 11 | 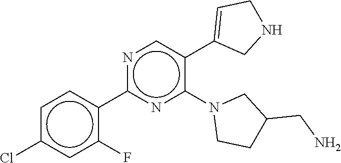 | LCMS (ESI): m/z calcd for C19H21ClFN5 + H: 374.1, found: 374.2. 1H NMR (400 MHz, MeOD) δ 8.24 (s, 1H), 8.05 (t, J = 8.4 Hz, 2H), 7.48-7.41 (m, 1H), 6.03 (s, 1H), 4.48 (d, J = 13.2 Hz, 1H), 4.37-4.30 (m, 3H), 3.96- 3.92 (m, 1H), 3.85-3.74 (m, 2H), 3.52-3.47 (m, 1H), 3.12 (d, J = 7.2 Hz, 2H), 2.67-2.59 (m, 1H), 2.32-2.25 (m, 1H), 1.88-1.78 (m, 1H), 1.20 (s, 1H). |
| 12 | 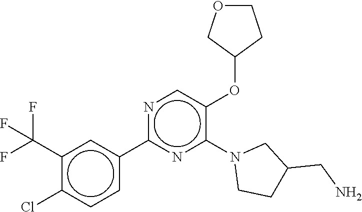 | LCMS (ESI): m/z calcd for C20H22ClF3N4O2 + H: 443.2, found: 443.1. 1H NMR (400 MHz, MeOD-d4) δ 8.56-8.55 (m, 1 H), 8.40-8.38 (m, 1 H), 7.88 (s, 1 H), 7.88-7.79 (m, 1 H), 5.16-5.15 (m, 1 H), 4.29-4.21 (m, 2 H), 4.10-3.89 (m, 5 H), 3.73-3.70 (m, 1 H), 3.16-3.05 (m, 2 H), 2.64-2.61 (m, 1 H), 2.36-2.21 (m, 3H), 1.87- 1.82 (m, 1 H). |
| 13 | 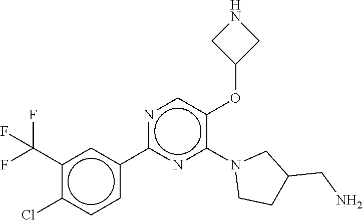 | LCMS (ESI): m/z calcd for C19H21ClF3N5O + H: 428.1, found: 428.2. 1H NMR (400 MHz, MeOD-d4) δ 8.61-8.60 (m, 1 H), 8.45-8.43 (m, 1 H), 7.73-7.70 (m, 1 H), 7.67 (s, 1 H), 5.23-5.20 (m, 1 H), 4.62-4.57 (m, 2 H), 4.34-4.15 (m, 4 H), 3.98-3.96 (m, 1 H), 3.71-3.66 (m, 1 H), 3.18-3.07 (m, 2 H), 2.64-2.61 (m, 1 H), 2.32- 2.28 (m, 1 H), 1.87-1.82 (m, 1 H). |
| 14 | 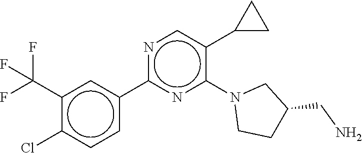 | LCMS (ESI): m/z calcd for C19H20ClF3N4 + H: 397.1, found: 397.1. 1H NMR (400 MHz, MeOD) δ 8.55 (d, J = 2.0 Hz, 1H), 8.45-8.37 (m, 1H), 8.02-7.84 (m, 2H), 4.57-4.38 (m, 2H), 4.27-4.13 (m, 1H), 3.96-3.85 (m, 1H), 3.26-3.07 (m, 2H), 2.77-2.64 (m, 1H), 2.43-2.17 (m, 2H), 2.00-1.84 (m, 1H), 1.20-1.03 (m, 2H), 0.98- 0.83 (m, 2H). |
| 15 | 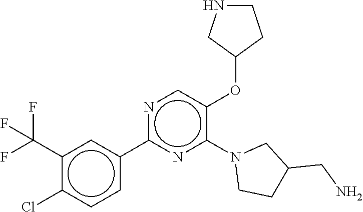 | LCMS (ESI): m/z calcd for C20H23ClF3N5O + H: 442.1, found: 442.1. 1H NMR (400 MHz, MeOD-d₄) δ 8.59-8.58 (m, 1 H), 8.43-8.41 (m, 1H), 7.95 (s, 1H), 7.78-7.75 (m, 1H), 5.28-5.27 (m, 1H), 4.16-4.14 (m, 2H), 3.98-3.95 (m, 1H), 3.77-3.55 (m, 3H), 3.53-3.51 (m, 2H), 3.18-3.06 (m, 2H), 2.64-2.60 (m, 1H), 2.48- 2.27 (m, 3H), 1.87-1.84 (m, 1H). |
| 16 | 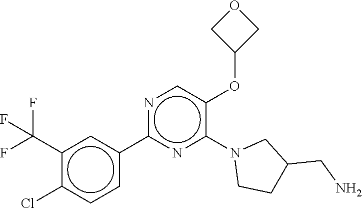 | LCMS (ESI): m/z calcd for C19H20ClF3N4O2 + H: 429.1, found: 429.1. 1H NMR (400 MHz, MeOD-d4) δ 8.60-8.59 (m, 1H), 8.43-8.41 (m, 1H), 7.73-7.70 (m, 1 H), 7.52 (s, 1H), 5.37-5.33 (m, 1H), 5.06-5.03 (m, 2H), 4.78-4.74 (m, 2H), 4.28-4.16 (m, 2H), 4.00-3.96 (m, 1H), 3.72-3.68 (m, 2H), 3.18-3.06 (m, 2H), 2.64-2.60 (m, 1H), 2.31-2.27 (m, 1H), 1.87-1.82 (m, 1H). |
| 17 | 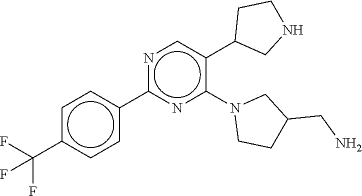 | See Ex. 5 |
| 18 | 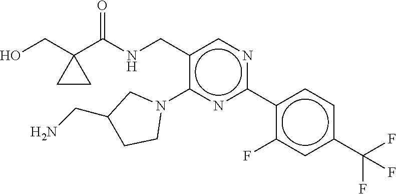 | LCMS [M + H] found: 468.1. 1H NMR (400 MHz, MeOD) δ 8.29-8.12 (m, 2H), 7.80-7.70 (m, 2H), 4.75- 4.60 (m, 2H), 4.32-4.13 (m, 2H), 4.08-3.96 (m, 1H), 3.82-3.63 (m, 3H), 3.22-3.05 (m, 2H), 2.76-2.61 (m, 1H), 2.41-2.27 (m, 1H), 1.98-1.82 (m, 1H), 1.18-1.08 (m, 2H), 0.84-0.74 (m, 2H). |
| 19 | 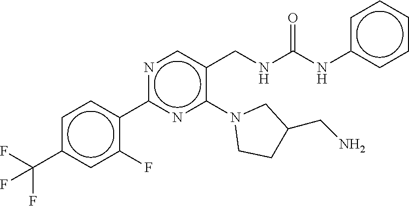 | See Ex. 6 |
| 20 | 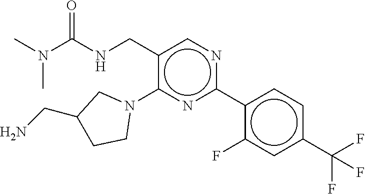 | LCMS (ESI): m/z calcd for C20H24F4N6O + H: 441.2, found: 441.2. 1H NMR (400 MHz, MeOD-d4) δ 8.26- 8.23 (m, 1 H), 8.13 (s, 1 H), 7.78-7.74 (m, 2 H), 4.58 (s, 2 H), 4.31-4.18 (m, 2 H), 4.04-4.01 (m, 1 H), 3.15-3.12 (m, 2 H), 2.95 (s, 6 H), 2.70-2.66 (m, 1 H), 2.34-2.32 (m, 1 H), 1.92-1.87 (m, 1 H). |
| 21 | 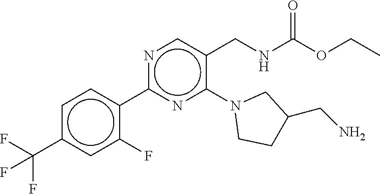 | See Ex. 7 |
| 22 | 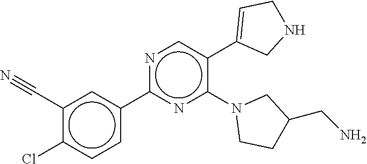 | LCMS (ESI): m/z calcd for C20H21ClN6 + H: 381.2; found: 381.2. 1H NMR (400 MHz, MeOD-d4) δ 8.64 (d, J = 2.0 Hz, 1H), 8.55-8.52 (m, 1H), 8.27 (s, 1H), 7.77 (d, J = 8.8 Hz, 1H), 5.97 (d, J = 1.6 Hz, 1H), 4.50- 4.46 (m, 1H), 4.40-4.30 (m, 3H), 3.94-3.84 (m, 1H), 3.78-3.71 (m, 2H), 3.52-3.47 (m, 1H), 3.14 (d, J = 7.2 Hz, 2H), 2.68-2.60 (m, 1H), 2.33-2.26 (m, 1H), 1.89- 1.79 (m, 1H). |
| 23 | 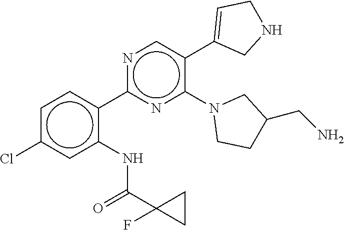 | LCMS (ESI): m/z calcd for C27H28ClF7N6O5 + H: 457.18, found 457.3. 1H NMR (400 MHz, MeOD) δ 8.72 (d, J = 2.1 Hz, 1H), 8.56 (d, J = 8.7 Hz, 1H), 8.22 (s, 1H), 7.20 (dd, J = 8.7, 2.1 Hz, 1H), 5.91 (s, 1H), 4.45 (d, J = 13.4 Hz, 1H), 4.31 (d, J = 11.9 Hz, 3H), 3.85 (dd, J = 11.2, 7.1 Hz, 1H), 3.69 (t, J = 7.4 Hz, 2H), 3.49- 3.39 (m, 1H), 3.11 (d, J = 7.2 Hz, 2H), 2.66-2.51 (m, 1H), 2.27 (dd, J = 11.1, 5.1 Hz, 1H), 1.82 (dd, J = 19.7, 10.6 Hz, 1H), 1.42 (m, 4H). |
| 24 | 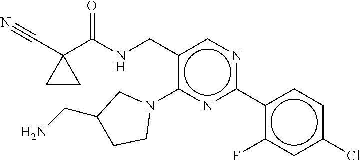 | LCMS (ESI): m/z calcd for C21H22ClFN6O + H: 429.2, found: 429.3. 1H NMR (400 MHz, MeOD): δ 8.72 (d, J = 4.4 Hz, 1H), 8.17 (s, 1H), 8.02 (d, J = 8.4 Hz, 1H), 7.92 (s, 3H), 7.64-7.60 (m, 1H), 7.47-7.44 (m, 1H), 4.97 (s, 2H), 3.94-3.86 (m, 2H), 3.74 (t, J = 8.0 Hz, 1H), 3.52 (d, J = 4.0 Hz, 1H), 2.94 (s, 2H), 2.51-2.50 (m, 1H), 2.48-2.43 (m, 1H), 1.62-1.59 (m, 1H), 1.55-1.52 (m, 4H). |
| 25 | 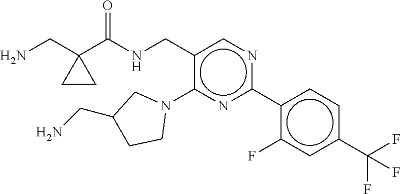 | LCMS (ESI): m/z calcd for C22H26F4N6O + H: 467.2, found: 467.2. 1H NMR (400 MHz, MeOD) δ 8.27-8.18 (m, 1H), 8.13 (s, 1H), 7.79-7.69 (m, 2H), 4.68-4.53 (m, 2H), 4.29-4.09 (m, 2H), 4.05-3.92 (m, 1H), 3.78-3.67 (m, 1H), 3.23-3.05 (m, 4H), 2.75-2.63 (m, 1H), 2.39- 2.28 (m, 1H), 1.96-1.84 (m, 1H), 1.46-1.35 (m, 2H), 1.18-1.08 (m, 2H). |
| 26 | 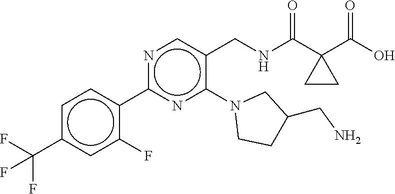 | LCMS (ESI): m/z calcd for C22H23F4N5O3 + H: 482.2, found: 482.1. 1H NMR (400 MHz, MeOD) δ 8.30-8.17 (m, 2H), 7.84-7.71 (m, 2H), 4.79-4.64 (m, 2H), 4.35- 4.16 (m, 2H), 4.10-3.96 (m, 1H), 3.84-3.73 (m, 1H), 3.23-3.06 (m, 2H), 2.77-2.63 (m, 1H), 2.42-2.29 (m, 1H), 1.99-1.83 (m, 1H), 1.67-1.49 (m, 4H). |
| 27 | 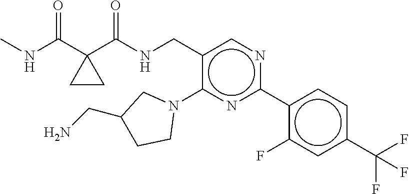 | LCMS (ESI): m/z calcd for C23H26F4N6O2 + H: 495.2, found: 495.1. 1H NMR (400 MHz, MeOD) δ 8.31-8.18 (m, 2H), 7.82-7.72 (m, 2H), 4.72-4.60 (m, 2H), 4.36- 4.16 (m, 2H), 4.10-3.96 (m, 1H), 3.85-3.75 (m, 1H), 3.22-3.08 (m, 2H), 2.77-2.63 (m, 4H), 2.42-2.29 (m, 1H), 2.00-1.84 (m, 1H), 1.48-1.32 (m, 4H). |
| 28 | 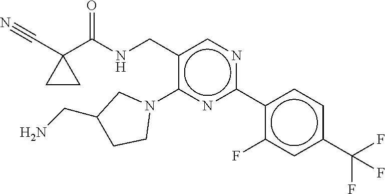 | LCMS (ESI): m/z calcd for C22H22F4N6O + H: 463.2, found: 463.1. 1H NMR (400 MHz, MeOD): δ8.73 (t, J = 4.8 Hz, 1H), 8.19 (t, J = 8.8 Hz, 2H), 7.93 (s, 2H), 7.84 (d, J = 10.4 Hz, 1H), 7.72 (d, J = 8.0 Hz, 1H), 4.49 (s, 2H), 3.95-3.90 (m, 2H), 3.73 (t, J = 8.0 Hz, 1H), 3.54- 3.49 (m, 1H), 2.97-2.93 (m, 2H), 2.53-2.48 (m, 1H), 2.13 (t, J = 6.0 Hz, 1H), 1.75 (t, J = 4.0 Hz, 1H), 1.59- 1.52 (m, 4H). |
| 29 | 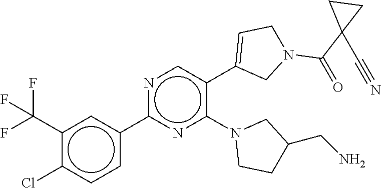 | LCMS (ESI): m/z calcd for C25H24ClF3N6O + H: 517.2, found: 517.2. 1H NMR (400 MHz, MeOD-d4) δ 8.71-8.69 (m, 1 H), 8.55-8.52 (m, 1 H), 8.32-8.27 (m, 1 H), 7.79-7.77 (m, 1 H), 6.01-6.00 (m, 1 H), 5.01-4.90 (m, 2 H), 4.59-4.47 (m, 2 H), 3.97-3.73 (m, 3 H), 3.56- 3.49 (m, 1 H), 3.15-3.08 (m, 2 H), 2.63-2.62 (m, 1 H), 2.31-2.27 (m, 1 H), 1.87-1.84 (m, 1 H), 1.68-1.59 (m, 4 H). |
| 30 | 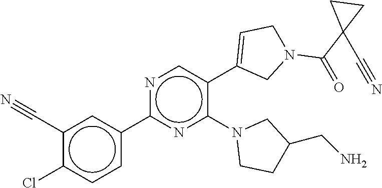 | LCMS (ESI): m/z calcd for C25H24ClN7O + H: 474.2, found: 474.1. 1H NMR (400 MHz, MeOD-d4) δ 8.72- 8.70 (m, 1 H), 8.63-8.59 (m, 1 H), 8.252-8.21 (m, 1 H), 7.74-7.71 (m, 1 H), 5.92-5.90 (m, 1 H), 5.00-4.84 (m, 2 H), 4.58-4.47 (m, 2 H), 3.84-3.63 (m, 3 H), 3.42-3.36 (m, 1 H), 2.85-2.83 (m, 2 H), 2.43-2.42 (m, 1 H), 2.19- 2.18 (m, 1 H), 1.76-1.73 (m, 1 H), 1.68-1.61 (m, 4 H). |
| 31 | 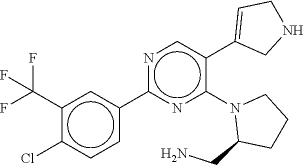 | LCMS (ESI): m/z calcd for C20H21ClF3N5 + H: 424.2, found: 424.2. 1H NMR (400 MHz, MeOD): δ8.71 (s, 1H), 8.57 (d, J = 2.0 Hz, 1H), 8.37 (s, 1H), 7.76 (d, J = 8 Hz, 1H), 6.04 (s, 1H), 4.57 (d, J = 2.4 Hz, 1H), 4.33- 4.24 (m, 3H), 3.64 (m, 1H), 3.50 (d, J = 4.4 Hz, 1H), 3.31-3.29 (m, 2H), 2.29 (m, 1H), 2.10 (m, 1H), 1.92 (m, 2H). |
| 32 | 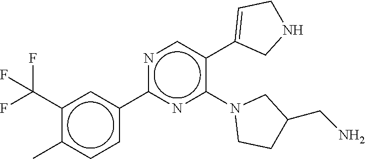 | LCMS (ESI): m/z calcd for C21H24F3N5 + H: 404.44, found: 404.2. 1H NMR (400 MHz, MeOD): δ8.54 (s, 1H), 8.40 (d, J = 8.0 Hz, 1H), 8.25 (s, 1H), 7.58 (d, J = 8.0 Hz, 1H), 6.00 (s, 1H), 4.47 (d, J = 14 Hz, 1H), 4.37- 4.33 (m, 3H), 3.97-3.78 (m, 3H), 3.54-3.49 (m, 1H), 3.13 (d, J = 6.4 Hz, 2H), 2.64 (m, 1H), 2.58 (s, 3H), 2.30-2.28 (m, 1H), 1.87-1.84 (m, 1H). |
| 33 | 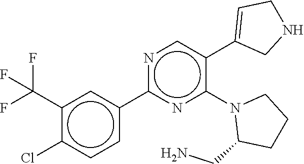 | LCMS (ESI): m/z calcd for C20H21ClF3N5 + H: 424.86, found: 424.2. 1H NMR (400 MHz, MeOD): δ8.71 (s, 1H), 8.57 (d, J = 2.0 Hz, 1H), 8.37 (s, 1H), 7.76 (d, J = 8 Hz, 1H), 6.04 (s, 1H), 4.57 (d, J = 2.4 Hz, 1H), 4.33- 4.24 (m, 3H), 3.64 (m, 1H), 3.50 (d, J = 4.4 Hz, 1H), 3.31-3.29 (m, 2H), 2.29 (m, 1H), 2.10 (m, 1H), 1.92 (m, 2H). |
| 34 | 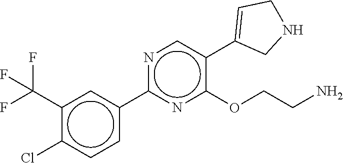 | See Ex. 8 |
| 35 | 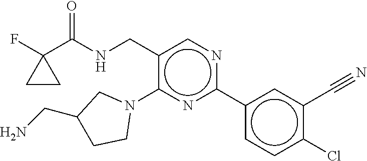 | LCMS (ESI): m/z calcd for C21H22ClFN6O + H: 429.2; found: 429.2. 1H NMR (400 MHz, MeOD-d4) δ 8.64 (s, 1H), 8.49 (d, J = 8.4 Hz, 1H), 8.20 (s, 1H), 7.83 (d, J = 8.0 Hz, 1H), 4.68 (s, 2H), 4.23-3.95 (m, 3H), 3.77-3.70 (m, 1H), 3.19-3.08 (m, 2H), 2.69-2.63 (m, 1H), 2.36- 2.30 (m, 1H), 1.92-1.85 (m, 1H), 1.38-1.26 (m, 4H). |
| 36 | 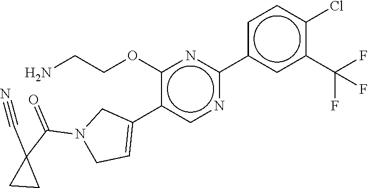 | LCMS (ESI): m/z calcd for C22H19ClF3N5O2 + H: 478, found: 478. 1H NMR (400 MHz, MeOD-d4) δ 8.82- 8.63 (m, 3H), 7.78-7.76 (m, 1H), 6.77-6.68 (m, 1H), 5.28-5.27 (m, 1H), 4.94-4.89 (m, 3H), 4.76-4.75 (m, 1H), 4.48-4.46 (m, 1H), 3.59-3.55 (m, 2H), 1.70-1.62 (m, 4H). |
| 37 | 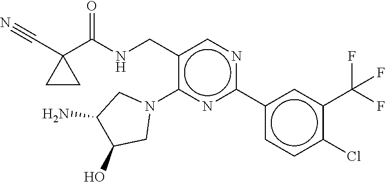 | LCMS (ESI): m/z calcd for C21H20ClF3N6O2 + H: 481.1, found : 481.1. 1H NMR (400 MHz, MeOD) δ 8.70 (d, J = 1.6 Hz, 1H), 8.52 (dd, J = 8.4, 2.0 Hz, 1H), 8.29 (s, 1H), 7.76 (d, J = 8.4 Hz, 1H), 4.61 (q, J = 16.0 Hz, 2H), 4.48-4.46 (m, 1H), 4.34-4.28 (m, 1H), 4.26- 4.22 (m, 1H), 4.03-3.98 (m, 1H), 3.89-3.83 (m, 1H), 3.80-3.77 (m, 1H), 1.64-1.57 (m, 4H). |
| 38 | 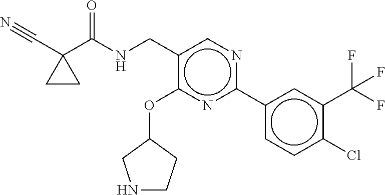 | LCMS (ESI): m/z calcd for C21H19ClF3N5O2 + H: 466.1; found: 466.1. 1H NMR (400 MHz, MeOD-d4) δ 8.76 (s, 1H), 8.62-8.56 (m, 2H), 7.75 (d, J = 8.4 Hz, 1H), 6.04 (s, 1H), 4.44 (s, 2H), 3.73 (d, J = 2.4 Hz, 2H), 3.64-3.43 (m, 2H), 2.52-2.47 (m, 2H), 1.63-1.56 (m, 4H). |
| 39 | 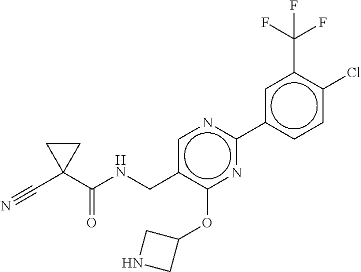 | LCMS (ESI): m/z calcd for C20H17ClF3N5O2 + H: 452.1; found: 452.1. 1H NMR (400 MHz, MeOD-d4) δ 8.70 (d, J = 1.6 Hz, 1H), 8.59 (s, 1H), 8.55-8.52 (m, 1H), 7.72 (d, J = 8.8 Hz, 1H), 5.80-5.76 (m, 1H), 4.68- 4.64 (m, 2H), 4.49 (s, 2H) 4.42-3.37 (m, 2H), 1.65-1.57 (m, 4H). |
| 40 | 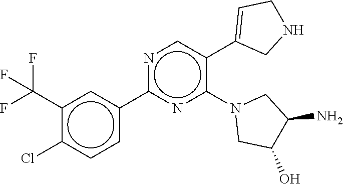 | LCMS (ESI): m/z calcd for C19H19ClF3N5O + H: 426.1, found 426.2. 1H NMR (400 MHz, MeOD) δ 8.76 (d, J = 1.7 Hz, 1H), 8.59 (d, J = 8.4 Hz, 1H), 8.33 (s, 1H), 7.75 (d, J = 8.4 Hz, 1H), 5.99 (s, 1H), 4.49-4.36 (m, 3H), 4.34 (s, 2H), 4.17 (dd, J = 12.7, 6.4 Hz, 1H), 3.95 (dd, J = 12.0, 5.5 Hz, 1H), 3.86 (dd, J = 12.6, 4.1 Hz, 1H), 3.77 (dd, J = 10.1, 4.2 Hz, 1H), 3.55 (dd, J = 12.0, 3.5 Hz, 1H). |
| 41 | 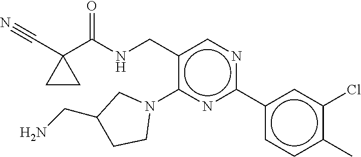 | LCMS (ESI): m/z calcd for C22H25ClN6O + H: 425.2, found: 425.2. 1H NMR (400 MHz, MeOD) δ 8.21 (d, J = 1.8 Hz, 1H), 8.10 (s, 1H), 8.03 (dd, J = 8.0, 1.8 Hz, 1H), 7.53 (d, J = 8.0 Hz, 1H), 4.68-4.56 (m, 2H), 4.28 (m, 1H), 4.22-4.15 (m, 1H), 4.03 (m, 1H), 3.78 (dd, J = 12.0, 8.4 Hz, 1H), 3.21-3.06 (m, 2H), 2.69 (dt, J = 14.6, 7.4 Hz, 1H), 2.48 (s, 3H), 2.40-2.28 (m, 1H), 1.92 (m, 1H), 1.66-1.51 (m, 4H). |
| 42 | 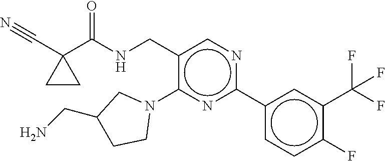 | LCMS (ESI): m/z calcd for C22H25ClF3N5O + H: 468.17, found 468.2. 1H NMR (400 MHz, MeOD) δ 8.57 (d, J = 6.9 Hz, 2H), 8.17 (s, 1H), 7.55 (dd, J = 12.5, 7.2 Hz, 1H), 4.67-4.57 (m, 2H), 4.27-4.05 (m, 2H), 4.05-3.92 (m, 1H), 3.75 (dd, J = 11.6, 8.0 Hz, 1H), 3.14 (tt, J = 13.1, 6.5 Hz, 2H), 2.74-2.64 (m, 1H), 2.35 (dd, J = 10.5, 6.0 Hz, 1H), 1.91 (dq, J = 17.2, 8.5 Hz, 1H), 1.68-1.50 (m, 4H). |
| 43 | 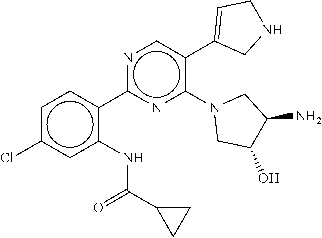 | LCMS [M + H] found: 441.4. 1H NMR (400 MHz, MeOD) δ 8.67-8.60 (m, 1H), 8.57-8.49 (m, 1H), 8.42- 8.35 (m, 1H), 7.19-7.10 (m, 1H), 6.00 (s, 1H), 4.50-4.30 (m, 5H), 4.21-4.10 (m, 1H), 4.00-3.90 (m, 1H), 3.89- 3.70 (m, 2H), 3.60-3.50 (m, 1H), 1.82-1.72 (m, 1H), 1.07-0.87 (m, 4H). |
| 44 | 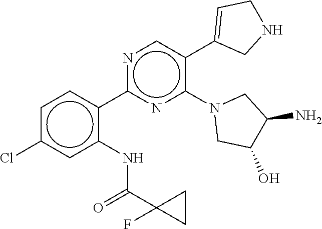 | LCMS [M + H] found: 459.3. ¹H NMR (400 MHz, MeOD) δ 8.41 (s, 1H), 8.11 (d, J = 2.0 Hz, 1H), 7.88 (d, J = 8.4 Hz, 1H), 7.51-7.41 (m, 1H), 6.24 (s, 2H), 4.60- 4.16 (m, 7H), 4.10-4.00 (m, 1H), 3.85-3.73 (m, 2H), 1.52-1.40 (m, 2H), 1.36-1.25 (m, 2H). |
| 45 | 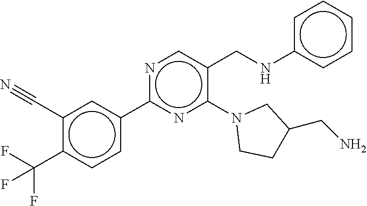 | See Ex. 9 |
| 46 | 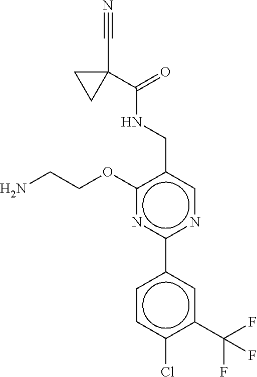 | LCMS (ESI): m/z calcd for C19H17ClF3N5O2 + H: 440.1; found: 440.1 1H NMR (400 MHz, MeOD-d4) δ 8.78-7.75 (m, 1H), 8.63-8.58 (m, 2H), 7.76-7.71 (m, 1H), 4.86-4.81 (m, 2H), 4.50 (s, 2H), 3.49 (t,d, J = 4.8 Hz, 2H), 1.64-1.59 (m, 4H). |
| 47 | 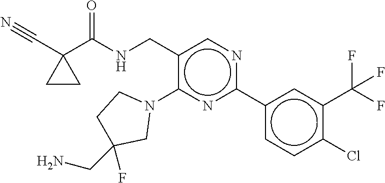 | LCMS (ESI): m/z calcd for C22H21ClF4N6O + H: 497.2; found: 497.2. 1H NMR (400 MHz, MeOD-d4) δ 8.66 (s, 1H), 8.52-8.50 (m, 1H), 8.26 (s, 1H), 7.78 (d, J = 8.4 Hz, 1H), 4.67-4.58 (m, 2H), 4.34-4.10 (m, 4H), 3.64-3.48 (m, 2H), 2.56-2.47 (m, 1H), 2.41-2.22 (m, 1H), 1.66-1.57 (m, 4H). |
| 48 | 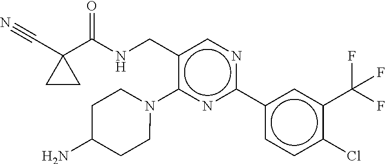 | LCMS (ESI): m/z calcd for C24H23ClF6N6O3 + H: 479.2, found: 479.2. 1H NMR (400 MHz, DMSO-d6): 88.53 (t, J = 5.2 Hz, 1H), 8.43 (s, 1H), 7.97 (s, 4H), 7.91-7.84 (m, 2H), 4.69 (d, J = 13.6 Hz, 1H), 4.20 (d, J = 5.2 Hz, 2H), 3.33 (s, 1H), 2.99 (t, J = 12 Hz, 2H), 1.97 (d, J = 10.4 Hz, 2H), 1.51-1.36 (m, 6H). |
| 49 | 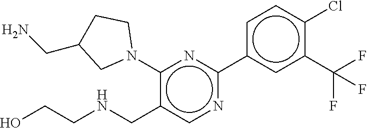 | See. Ex. 10 |
| 50 | 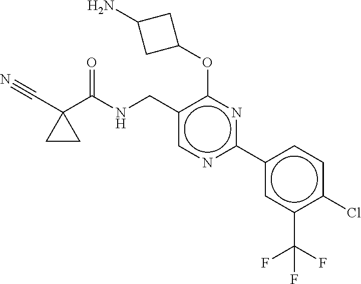 | LCMS (ESI): m/z calcd for C21H19ClF3N5O2 + H: 466.2; found: 466.2 1H NMR (400 MHz, MeOD-d4) δ 8.74 (d, J = 1.2 Hz, 1H), 8.58 (d, J = 8.8 Hz, 1H), 8.50 (s, 1H), 7.73 (d, J = 8.4 Hz, 1H), 5.37-5.30 (m, 1H), 4.45 (s, 2H), 3.71-3.65 (m, 1H), 3.16-3.09 (m, 2H), 2.50-2.43 (m, 2H), 1.65-1.57 (m, 4H). |
| 51 | 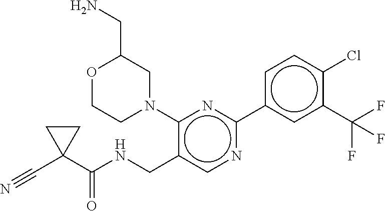 | LCMS (ESI): m/z calcd for C22H22ClF3N6O2 + H: 495.2; found: 495.2. 1H NMR (400 MHz, MeOD-d4) δ 8.72 (d, J = 1.6 Hz, 1H), 8.58-8.55 (m, 1H), 8.44 (s, 1H), 7.75 (d, J = 8.4 Hz, 1H), 4.50 (s, 2H), 4.13-4.07 (m, 2H), 3.99-3.82 (m, 3H), 3.30-3.21 (m, 2H), 3.09- 3.02 (m, 2H), 1.62-1.58 (m, 4H). |
| 52 | 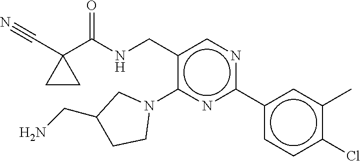 | LCMS (ESI): m/z calcd for C22H25ClN6O + H: 425.2, found: 425.3. 1H NMR (400 MHz, MeOD) δ 8.11 (s, 1H), 8.04-7.98 (m, 1H), 7.65-7.58 (m, 1H), 4.65 (s, 2H), 4.37-4.18 (m, 2H), 4.12-3.99 (m, 1H), 3.86-3.75 (m, 1H), 3.24-3.08 (m, 2H), 2.78-2.64 (m, 1H), 2.50 (s, 3H), 2.44-2.30 (m, 1H), 2.00-1.85 (m, 1H), 1.68-1.56 (m, 4H). |
| 53 | 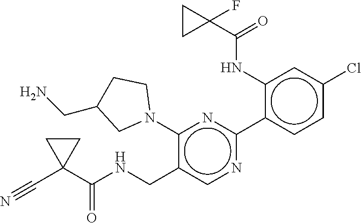 | LCMS (ESI): m/z calcd for C25H27ClFN7O2 + H: 512.2, found: 512.4. 1H NMR (400 MHz, MeOD) δ 8.51 (d, J = 2.0 Hz, 1H), 8.32 (d, J = 8.8 Hz, 1H), 8.18 (s, 1H), 7.31-7.22 (m, 1H), 2.60 (s, 2H), 4.18-3.98 (m, 2H), 3.95-3.84 (m, 1H), 3.70-3.61 (m, 1H), 3.20-3.04 (m, 2H), 7.71-2.57 (m, 1H), 2.38-2.75 (m, 1H), 1.95- 1.80 (m, 1H), 1.67-1.53 (m, 4H), 1.51-1.32 (m, 4H). |
| 54 | 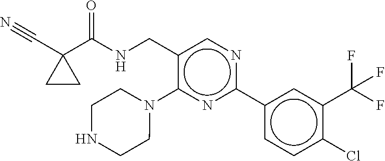 | LCMS (ESI): m/z calcd for C21H20ClF3N6O + H: 465.2, found: 465.2. 1H NMR (400 MHz, MeOD) δ 8.75 (d, J = 1.8 Hz, 1H), 8.59 (dd, J = 8.4, 1.8 Hz, 1H), 8.52 (s, 1H), 7.74 (d, J = 8.4 Hz, 1H), 4.50 (s, 2H), 3.82-3.67 (m, 4H), 3.53-3.37 (m, 4H), 1.64-1.49 (m, 4H). |
| 55 | 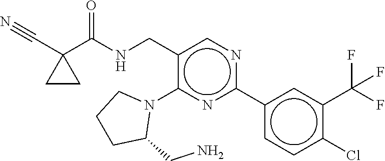 | LCMS (ESI): m/z calcd for C22H22ClF3N6O + H: 480.1, found: 480.2. 1H NMR (400 MHz, MeOD) δ 8.67 (s, 1H), 8.52 (d, J = 8.4 Hz, 1H), 8.37 (s, 1H), 7.75 (d, J = 8.4 Hz, 1H), 4.97-4.94 (m, 1H), 4.59 (d, J = 15.6 Hz, 1H), 4.46 (d, J = 15.6 Hz, 1H), 3.99-3.84 (m, 1H), 3.69- 3.66 (m, 1H), 3.38 (d, J = 5.2 Hz, 2H), 2.28-2.25 (m, 1H), 2.16-2.12 (m, 1H), 1.98-1.93 (m, 2H), 1.70-1.46 (m, 4H). |
| 56 | 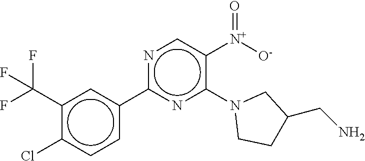 | LCMS (ESI): m/z calcd for C16H15ClF3N5O2 + H: 402.1, found: 402.0. 1H NMR (400 MHz, MeOD): δ 8.98 (s, 1H), 8.80 (d, J = 2.0 Hz, 1H), 8.63 (dd, J = 8.4, 1.6 Hz, 1H), 7.76 (d, J = 8.4 Hz, 1H), 3.73 (s, 2H), 3.48 (s, 2H), 3.18-3.06 (m, 2H), 2.65 (dt, J = 14.8, 7.6 Hz, 1H), 2.32 (td, J = 11.6, 5.6 Hz, 1H), 1.88 (dq, J = 12.4, 8.8 Hz, 1H). |
| 57 | 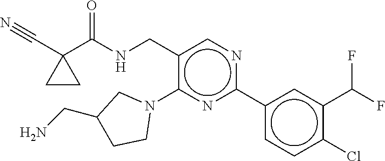 | LCMS (ESI): m/z calcd for C22H23ClF2N6O + H: 461, found: 461. 1H NMR (400 MHz, MeOD-d4) δ 8.53- 8.52 (m, 1 H), 8.73-8.34 (m, 1 H), 8.15 (s, 1 H), 7.75- 7.73 (m, 1 H), 7.29-7.01 (m, 1 H), 4.63 (s, 2 H), 4.27- 4.14 (m, 2 H), 4.04-3.97 (m, 1 H), 3.79-3.74 (m, 1 H), 3.20-3.08 (m, 2 H), 2.70-2.66 (m, 1 H), 2.37-2.33 (m, 1 H), 1.93-1.88 (m, 1 H), 1.62-1.57 (m, 4 H). |
| 58 | 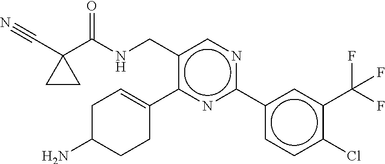 | See Ex. 11 |
| 59 | 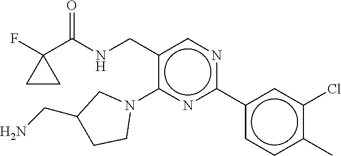 | LCMS (ESI): m/z calcd for C23H26ClF4N5O3 + H: 418.1, found: 418.2. 1H NMR (400 MHz, MeOD) δ 8.21 (d, J = 1.8 Hz, 1H), 8.10 (s, 1H), 8.04-7.89 (m, 1H), 7.54 (d, J = 8.1 Hz, 1H), 4.70 (s, 2H), 4.40-4.16 (m, 2H), 4.13-3.94 (m, 1H), 3.80 (dd, J = 12.1, 8.3 Hz, 1H), 3.15 (t, J = 8.0 Hz, 2H), 2.71 (s, 1H), 2.49 (s, 3H), 2.36 (d, J = 4.5 Hz, 1H), 2.02-1.79 (m, 1H), 1.48-1.21 (m, 4H). |
| 60 | 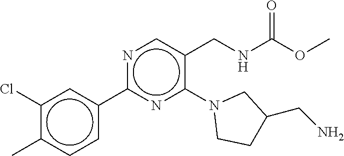 | LCMS (ESI): m/z calcd for C19H21F4N5O2 + H: 390.2, found: 390.2. 1H NMR (400 MHz, MeOD) δ 8.19 (dd, J = 8.6, 1.8 Hz, 1H), 8.11 (s, 1H), 8.02 (dd, J = 8.0, 1.9 Hz, 1H), 7.54 (d, J = 8.1 Hz, 1H), 4.52 (s, 2H), 4.29 (dd, J = 12.1, 7.4 Hz, 1H), 4.21 (ddd, J = 11.6, 8.1, 3.7 Hz, 1H), 4.10-3.99 (m, 1H), 3.78 (dd, J = 12.0, 8.3 Hz, 1H), 3.68 (s, 3H), 3.20-3.09 (m, 2H), 2.69 (dt, J = 15.1, 7.6 Hz, 1H), 2.47 (d, J = 12.5 Hz, 3H), 2.42-2.29 (m, 1H), 1.96-1.87 (m, 1H). |
| 61 | 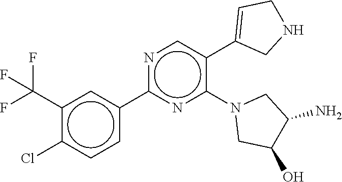 | LCMS (ESI): m/z calcd for C19H19ClF3N5O + H: 426.2, found: 426.0. 1H NMR (400 MHz, MeOD) δ 8.75 (d, J = 1.8 Hz, 1H), 8.58 (dd, J = 8.4, 1.8 Hz, 1H), 8.34 (s, 1H), 7.77 (d, J = 8.4 Hz, 1H), 6.03 (s, 1H), 4.52-4.32 (m, 5H), 4.20 (m, 1H), 4.06-3.97 (m, 1H), 3.90 (m, 1H), 3.82-3.75 (m, 1H), 3.60 (m, 1H). |
| 62 | 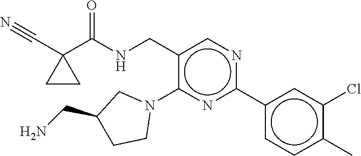 | LCMS (ESI): m/z calcd for C22H25ClN6O + H: 425.2, found: 425.0. 1H NMR (400 MHz, MeOD): δ 8.22 (s, 1H), 8.11 (s, 1H), 8.03 (d, J = 8.0 Hz, 1H), 7.53 (d, J = 7.6 Hz, 1H), 4.63 (s, 2H), 4.33-4.24 (m, 1H), 4.19 (s, 1H), 4.08-3.97 (m, 1H), 3.83-3.72 (m, 1H), 3.20-3.09 (m, 2H), 2.68 (d, J = 7.6 Hz, 1H), 2.48 (s, 3H), 2.36 (s, 1H), 1.99-1.86 (m, 1H), 1.61 (m, 4H). |
| 63 | 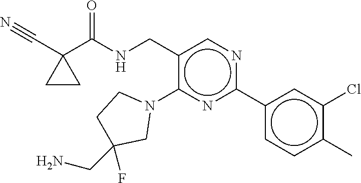 | LCMS (ESI): m/z calcd for C22H24ClFN6O + H: 443.2; found: 443.2. 1H NMR (400 MHz, MeOD) δ 8.22 (d, J = 1.2 Hz, 1H), 8.17 (s, 1H), 8.07 (t, J = 8.4 Hz, 1H), 7.54 (d, J = 8.0 Hz, 1H), 4.69-4.61 (m, 2H), 4.49-4.18 (m, 4H), 3.67-3.51 (m, 2H), 2.60-2.27 (m, 5H), 1.67- 1.58 (m, 4H). |
| 64 | 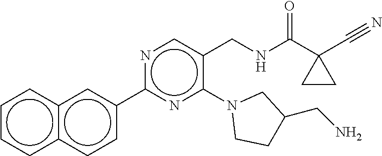 | LCMS (ESI): m/z calcd for C27H27F3N6O3 + H: 427.1, found: 427.1. 1H NMR (400 MHz, MeOD) δ 8.38 (s, 1H), 8.10 (s, 1H), 8.02-7.90 (m, 3H), 7.71 (d, J = 8.5 Hz, 1H), 7.63-7.51 (m, 2H), 4.44 (s, 2H), 3.97 (m, 1H), 3.85 (s, 1H), 3.65 (m, 1H), 3.40 (s, 1H), 3.10 (m, 2H), 2.67 (s, 1H), 2.31 (m, 1H), 1.88 (m, 1H), 1.36 (m, 4H). |
| 65 | 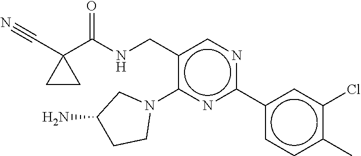 | LCMS (ESI): m/z calcd for C21H23ClN6O + H: 411.2, found: 411.1. 1H NMR (400 MHz, MeOD) δ 8.22 (d, J = 1.6 Hz, 1H), 8.17 (s, 1H), 8.04 (dd, J = 8.0, 1.6 Hz, 1H), 7.53 (d, J = 8.1 Hz, 1H), 4.64 (d, J = 2.3 Hz, 2H), 4.29 (d, J = 6.0 Hz, 1H), 4.24-4.06 (m, 4H), 2.65-2.51 (m, 1H), 2.48 (s, 3H), 2.38-2.19 (m, 1H), 1.62 (q, J = 3.5 Hz, 4H). |
| 66 | 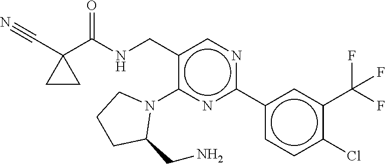 | LCMS (ESI): m/z calcd for C22H22ClF3N6O + H: 479.2; found: 479.0. 1H NMR (400 MHz, DMSO-d6) 8.65 (d, J = 1.60 Hz, 1H), 8.52-8.50 (m, 1H), 8.35 (s, 1H), 7.77 (d, J = 8.4 Hz, 1H), 4.97 (t, J = 4.8 Hz, 1H), 4.62-4.47 (m, 2H), 3.95 (t, J = 8.0 Hz, 1H), 3.74 (t, J = 2.8 Hz, 1H), 3.38 (t, J = 3.2 Hz, 2H), 2.30-2.24 (m, 1H), 2.16 (t, J = 3.2 Hz, 1H), 2.03-1.95 (m, 2H), 1.63-1.56 (m, 4H). |
| 67 | 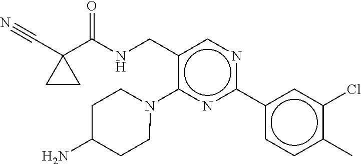 | LCMS (ESI): m/z calcd for C22H25ClN6O + H: 425.2; found: 425.3. 1H NMR (400 MHz, DMSO-d6) 8.29 (s, 1H), 8.24 (d, J = 2.0 Hz, 1H), 8.10-8.07 (m, 1H), 7.49 (d, J = 8.0 Hz, 1H), 4.45 (t, J = 13.6 Hz, 4H), 3.51 (s, 1H), 3.35 (s, 1H), 2.46 (s,3H), 2.22-2.18 (m, 2H), 1.82- 1.78 (m, 2H), 1.63-1.59 (m, 4H). |
| 68 | 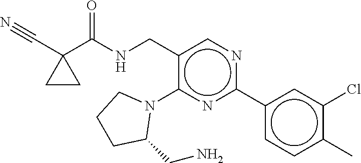 | LCMS (ESI): m/z calcd for C22H25ClN6O + H: 425.2; found: 425.3. 1H NMR (400 MHz, DMSO-d6) 8.24 (s, 1H), 8.19 (s, 1H), 8.05 (d, J = 8.0 Hz, 1H), 7.52 (d, J = 7.6 Hz, 1H), 5.04 (t, J = 4.4 Hz, 1H), 4.63-4.53 (m, 2H), 4.06-4.02 (m, 1H), 3.92-3.88 (m, 1H), 3.47-343 (m, 1H), 3.36 (s, 1H), 2.48 (s, 3H), 2.28-2.19 (m, 2H), 2.07- 2.00 (m, 2H), 1.60 m, 4H). |
| 69 | 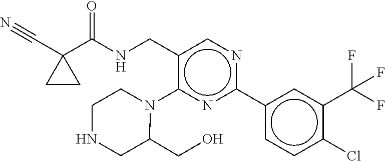 | LCMS (ESI): m/z calcd for C22H25ClN6O + H: 425.2; found: 425.3. 1H NMR (400 MHz, DMSO-d6) 8.24 (s, 1H), 8.19 (s, 1H), 8.05 (d, J = 8.0 Hz, 1H), 7.52 (d, J = 7.6 Hz, 1H), 5.04 (t, J = 4.4 Hz, 1H), 4.63-4.53 (m, 2H), 4.06-4.02 (m, 1H), 3.92-3.88 (m, 1H), 3.47-343 (m, 1H),3.36 (s, 1H), 2.48 (s,3H), 2.28-2.19 (m, 2H), 2.07- 2.00 (m, 2H), 1.60 (m, 4H). |
| 70 | 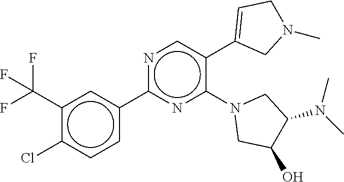 | LCMS (ESI): m/z calcd for C27H28ClF7N6O5 + H: 457.2, found 457.3. 1H NMR (400 MHz, MeOD) δ 8.77 (s, 1H), 8.62 (d, J = 8.7 Hz, 1H), 8.39 (s, 1H), 7.74 (d, J = 8.3 Hz, 1H), 5.97 (s, 1H), 4.72-4.61 (m, 2H), 4.16 (d, J = 8.2 Hz, 2H), 4.01-3.74 (m, 3H), 3.59-3.47 (m, 1H), 3.15 (s, 3H), 3.08 (s, 6H). |
| 71 | 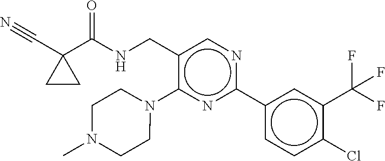 | LCMS (ESI): m/z calcd for C22H22ClF3N6O + H: 479.2, found 479.2. 1H NMR (400 MHz, MeOD) δ 8.83 (s, 1H), 8.76 (s, 1H), 8.60 (d, J = 8.2 Hz, 1H), 8.54 (s, 1H), 7.74 (d, J = 8.5 Hz, 1H), 4.50 (d, J = 4.6 Hz, 2H), 4.18 (s, 2H), 3.50 (d, J = 105.7 Hz, 6H), 2.99 (s, 3H), 1.60 (s, 4H). |
| 72 | 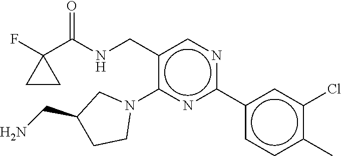 | LCMS (ESI): m/z calcd for C21H25ClFN5O + H: 417, found: 418. 1H NMR (400 MHz, MeOD-d4) δ 8.21- 8.20 (m, 1 H), 8.10 (s, 1 H), 8.03-8.01 (m, 1 H), 7.56- 7.54 (m, 1 H), 4.71-4.70 (m, 2 H), 4.34-4.20 (m, 2 H), 4.09-4.02 (m, 1 H), 3.83-3.78 (m, 1 H), 3.17-3.10 (m, 2 H), 2.72-2.68 (m, 1 H), 2.48 (s, 3 H), 2.38-2.35 (m, 1 H), 1.93-1.89 (m, 1 H), 1.41-1.27 (m, 4 H). |
| 73 | 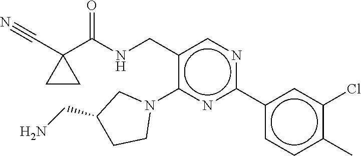 | LCMS (ESI): m/z calcd for C22H25ClN6O + H: 425.18; found: 425.2. 1H NMR (400 MHz, MeOD): δ 8.21 (d, J = 1.6 Hz, 1H), 8.09 (s, 1H), 8.02 (dd, J = 8.0, 1.6 Hz, 1H), 7.54 (d, J = 8.0 Hz, 1H), 4.64 (s, 2H), 4.30 (dd, J = 12.0, 7.6 Hz, 1H), 4.21 (ddd, J = 11.6, 8.0, 3.6 Hz, 1H), 4.05 (dd, J = 13.6, 5.8 Hz, 1H), 3.79 (dd, J = 12.0, 8.4 Hz, 1H), 3.23-3.08 (m, 2H), 2.77-2.63 (m, 1H), 2.49 (s, 3H), 2.36 (dd, J = 10.4, 5.8 Hz, 1H), 1.92 (dd, J = 12.4, 8.8 Hz, 1H), 1.67-1.56 (m, 4H). |
| 74 | 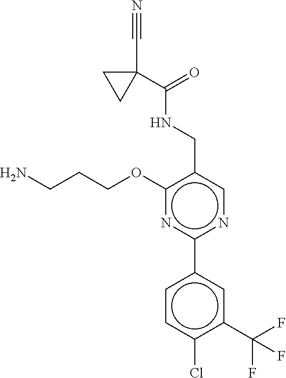 | LCMS (ESI): m/z calcd for C20H19ClF3N5O2 + H: 454.1, found: 454.2. 1H NMR (400 MHz, MeOD-d4) δ 8.72 (s, 1 H), 8.57-8.55 (m, 1 H), 8.51 (s, 1 H), 7.70 (d, J = 8.4, 1 H), 4.71-4.68 (m, 2 H), 4.45 (s, 2 H), 3.26- 3.23 (m, 2 H), 2.29-2.26 (m, 2 H), 1.65-1.57 (m, 4 H). |
| 75 | 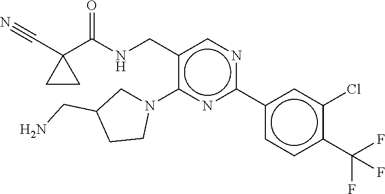 | LCMS (ESI): m/z calcd for C22H22ClF3N6O + H: 479.2 found: 479.3. 1H NMR (400 MHz, MeOD-d4) δ 8.46 (s, 1 H), 8.36-8.30 (m, 1 H), 8.21 (s, 1 H), 7.98-7.92 (m, 1 H), 4.62 (s, 2 H), 4.22-4.07 (m, 2 H), 3.98-3.94 (m, 1 H), 3.77-3.70 (m, 1 H), 3.19-3.09 (m, 2 H), 2.69-2.64 (m, 1 H), 2.34-2.33 (m, 1 H), 1.92-1.87 (m, 1 H), 1.64- 1.56 (m, 4 H). |
| 76 | 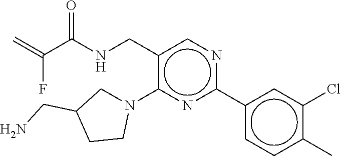 | LCMS (ESI): m/z calcd for C20H23ClFN5O + H: 404.2, found: 404.2. 1H NMR (400 MHz, MeOD): δ 8.21 (s, 1H), 8.11 (s, 1H), 8.02 (d, J = 8.0 Hz, 1H), 7.54 (d, J = 8.0 Hz, 1H), 5.64 (dd, J = 46.8, 3.4 Hz, 1H), 5.27 (dd, J = 15.2, 3.4 Hz, 1H), 4.69 (d, J = 3.8 Hz, 2H), 4.41-4.15 (m, 2H), 4.14-3.96 (m, 1H), 3.90-3.72 (m, 1H), 3.25- 3.08 (m, 2H), 2.83-2.61 (m, 1H), 2.49 (s, 3H), 2.44- 2.28 (m, 1H), 2.01-1.79 (m, 1H). |
| 77 | 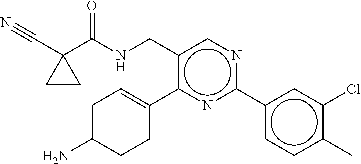 | LCMS (ESI): m/z calcd for C23H24ClN5O + H: 422.2, found: 422.2. 1H NMR (400 MHz, MeOD-d4) δ 8.77 (s, 2 H), 8.36 (d, 1 H), 8.26-8.18 (m, 1 H), 7.42 (d, 1 H), 5.98-5.91 (m, 1 H), 4.54 (s, 2 H), 3.66-3.53 (m, 1 H), 2.79-2.68 (m, 3 H), 2.47-2.21 (m, 5 H), 2.04-1.88 (m, 1 H), 1.65-1.52 (m, 4 H). |
| 78 | 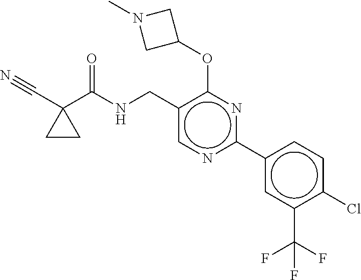 | LCMS (ESI): m/z calcd for C21H19ClF3N5O2 + H: 466.2; found: 466.2. 1H NMR (400 MHz, MeOD-d4) δ 8.72 (d, J = 2.0 Hz, 1H), 8.61 (s, 1H), 8.58-8.55 (m, 1H), 7.75 (d, J = 8.4 Hz, 1H), 5.82-5.70 (m, 1H), 4.95-4.91 (m, 2H), 4.63-4.61 (m, 1H), 4.50 (s, 2H) 4.37-3.32 (m, 1H), 3.09 (s, 3H), 1.64-1.57 (m, 4H). |
| 79 | 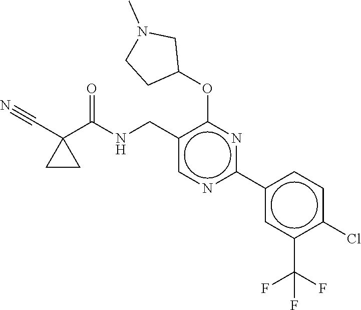 | LCMS (ESI): m/z calcd for C22H21ClF3N5O2 + H: 480.1; found: 480.1. 1H NMR (400 MHz, MeOD-d₄) δ 8.77 (d, J = 2.0 Hz, 1H), 8.63-8.58 (m, 2H), 7.76 (d, J = 8.4 Hz, 1H), 6.06-5.99 (m, 1H), 4.46 (s, 2H), 4.06-3.84 (m, 2H), 3.62-3.47 (m, 2H), 3.06 (s, 3H), 2.85-2.42 (m, 2H), 1.63-1.56 (m, 4H). |
| 80 | 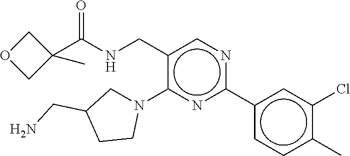 | LCMS (ESI): m/z calcd for C22H28ClN5O2 + H: 430.2, found: 430.2. 1H NMR (400 MHz, MeOD) δ 8.19 (t, J = 12.1 Hz, 1H), 8.11-7.96 (m, 2H), 7.55 (d, J = 8.1 Hz, 1H), 4.89 (d, J = 6.2 Hz, 3H), 4.63 (s, 2H), 4.43 (d, J = 6.1 Hz, 2H), 4.31 (m, 1H), 4.22 (m, 1H), 4.10-3.99 (m, 1H), 3.80 (m, 1H), 3.22-3.07 (m, 2H), 2.70 (m, 1H), 2.49 (s, 3H), 2.37 (m, 1H), 1.92 (m, 1H), 1.63 (s, 3H). |
| 81 | 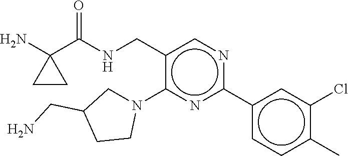 | LCMS (ESI): m/z calcd for C21H27ClN6O + H: 415.2, found: 415.2. 1H NMR (400 MHz, MeOD-d4) δ 8.21 (s, 1 H), 8.09 (s, 1 H), 8.04-8.01 (m, 1 H), 7.54 (d, J = 11.6, 1 H), 4.66-4.57 (m, 2 H), 4.30-4.16 (m, 2 H), 4.09-3.98 (m, 1 H), 3.80-3.75 (m, 1 H), 3.20-3.11 (m, 2H), 2.72- 2.69 (m, 1 H), 2.37 (s, 3H), 2.37-2.31 (m, 1H), 1.94- 1.89 (m, 1H), 1.63-1.53 (m, 2 H), 1.42-1.39 (m, 2 H) |
| 82 | 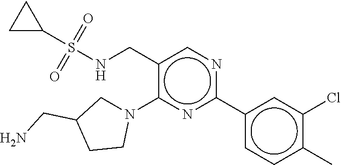 | LCMS (ESI): m/z calcd for C20H26ClN5O2S + H: 436.1; found: 436.2. 1H NMR (400 MHz, DMSO-d6) 8.22 (t, J = 2.0 Hz, 2H), 8.05-8.03 (m, 1H), 7.57 (d, J = 8.4 Hz, 1H), 4.54 (s, 2H), 4.40-4.33 (m, 2H), 4.13 (s, 1H), 3.86 (t, J = 8.0 Hz, 1H), 3.20-3.10 (m, 2H), 2.74- 2.68 (m, 2H), 2.50 (s,3H), 2.39-2.36 (m, 1H), 1.96-1.90 (m, 1H), 1.13-1.08 (m, 4H). |
| 83 | 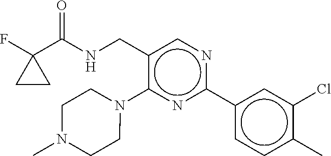 | LCMS (ESI): m/z calcd for C23H26ClF4N5O3 + H: 418.17, found 418.2. 1H NMR (400 MHz, MeOD) δ 8.46 (s, 1H), 8.30 (d, J = 1.7 Hz, 1H), 8.15 (dd, J = 8.0, 1.8 Hz, 1H), 7.44 (d, J = 8.1 Hz, 1H), 4.55 (d, J = 4.2 Hz, 2H), 4.21 (s, 1H), 3.48 (s, 5H), 2.99 (s, 3H), 2.37 (d, J = 64.6 Hz, 3H), 1.45-1.12 (m, 4H). |
| 84 | 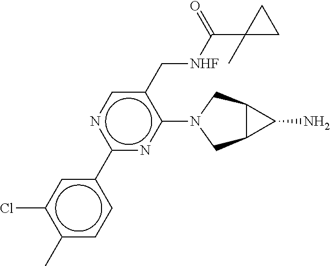 | LCMS (ESI): m/z calcd for C26H31ClFN5O3 + H: 416.1, found: 416.2. 1H NMR (400 MHz, MeOD) δ 8.22-8.12 (m, 2H), 8.03 (dd, J = 8.0, 1.9 Hz, 1H), 7.53 (d, J = 8.1 Hz, 1H), 4.65 (s, 2H), 4.40 (d, J = 12.3 Hz, 2H), 4.14 (dd, J = 10.0, 1.9 Hz, 2H), 2.57 (t, J = 2.4 Hz, 1H), 2.48 (s, 3H), 2.28 (s, 2H), 1.47-1.24 (m, 4H). |
| 85 | 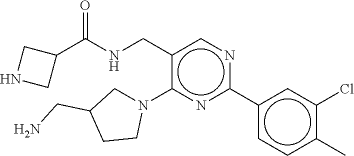 | LCMS (ESI): m/z calcd for C21H27ClN6O + H: 415.2, found: 415.3. 1H NMR (400 MHz, MeOD) δ 8.30-8.14 (m, 2H), 8.04 (dd, J = 8.0, 1.9 Hz, 1H), 7.52 (d, J = 8.1 Hz, 1H), 4.70-4.58 (m, 2H), 4.32-4.14 (m, 6H), 4.01 (dd, J = 17.8, 9.9 Hz, 1H), 3.85-3.69 (m, 2H), 3.23-3.10 (m, 2H), 2.78-2.63 (m, 1H), 2.48 (s, 3H), 2.35 (dd, J = 10.1, 6.1 Hz, 1H), 2.01-1.87 (m, 1H). |
| 86 | 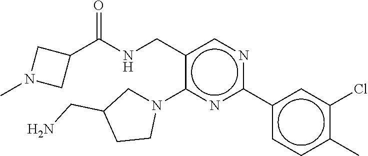 | LCMS (ESI): m/z calcd for C22H29ClN6O + H: 430.2, found: 430.3. 1H NMR (400 MHz, MeOD) δ 8.35 (s, 1H), 8.26 (s, 1H), 8.09 (d, J = 7.9 Hz, 1H), 7.50 (d, J = 8.0 Hz, 1H), 4.68-4.55 (m, 2H), 4.31-4.16 (m, 1H), 4.09 (s, 1H), 3.93 (dd, J = 17.3, 8.5 Hz, 1H), 3.78-3.65 (m, 1H), 3.24-3.07 (m, 3H), 3.04 (s, 11H), 2.75-2.62 (m, 1H), 2.47 (s, 3H), 2.37 (s, 1H), 2.00-1.86 (m, 1H). |
| 87 | 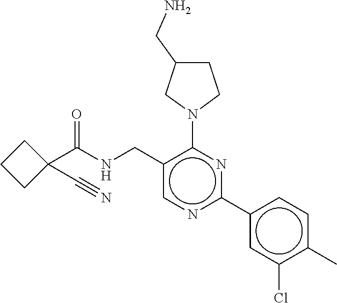 | LCMS (ESI): m/z calcd for C23H27ClN6O + H: 439.2; found: 439.2. 1H NMR (400 MHz, MeOD): δ 8.21 (d, J = 2.0 Hz, 1H), 8.12 (s, 1H), 8.03 (dd, J = 8.0, 2.0 Hz, 1H), 7.54 (d, J = 8.0 Hz, 1H), 4.63 (s, 2H), 4.29 (m, 1H), 4.24-4.16 (m, 1H), 4.08-3.98 (m, 1H), 3.78 (m, 1H), 3.22-3.08 (m, 2H), 2.78-2.67 (m, 3H), 2.59 (m, 2H), 2.49 (s, 3H), 2.36 (m, 1H), 2.24 (m, 1H), 2.11-2.00 (m, 1H), 1.92 (m, 1H). |
| 88 | 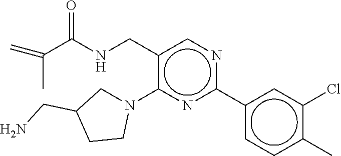 | LCMS (ESI): m/z calcd for C21H26ClN5O + H: 400.2, found: 400.2. 1H NMR (400 MHz, MeOD): δ 8.20 (d, J = 2.0 Hz, 1H), 8.06 (s, 1H), 8.02 (dd, J = 8.0, 2.0 Hz, 1H), 7.54 (d, J = 8.0 Hz, 1H), 5.80 (s, 1H), 5.51-5.46 (m, 1H), 4.65 (s, 2H), 4.31 (dd, J = 12.0, 7.2 Hz, 1H), 4.22 (td, J = 8.0, 4.0 Hz, 1H), 4.10-4.00 (m, 1H), 3.80 (dd, J = 12.0, 8.4 Hz, 1H), 3.16 (dd, J = 14.8, 6.8 Hz, 2H), 2.76-2.64 (m, 1H), 2.49 (s, 3H), 2.41-2.30 (m, 1H), 1.98 (d, J = 1.0 Hz, 3H), 1.92 (dd, J = 12.4, 8.8 Hz, 1H). |
| 89 | 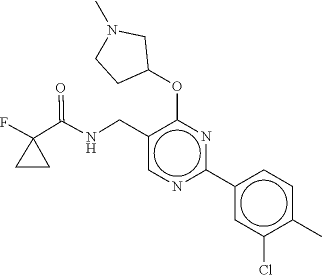 | LCMS (ESI): m/z calcd for C21H24ClFN4O2 + H: 419.2; found: 419.2. 1H NMR (400 MHz, MeOD-d4) δ 8.52 (s, 1H), 8.32 (d, J = 1.2 Hz, 1H), 8.20-8.18 (m, 1H), 7.40 (d, J = 8.0 Hz, 1H), 6.0 (s, 1H),4.52 (s, 2H), 4.30-3.82 (m, 2H), 3.76-3.39 (m, 2H), 3.13-2.43 (m, 5H), 1.39-1.27 (m, 4H). |
| 90 | 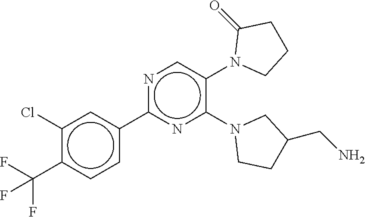 | See Ex. 12 |
| 91 | 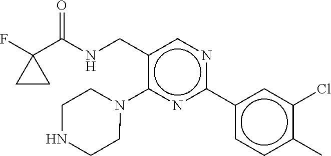 | LCMS (ESI): m/z calcd for C20H23ClFN5O + H: 404.2, found: 404.2. 1H NMR (400 MHz, MeOD) δ 9.02 (s, 1H), 8.45 (s, 1H), 8.29 (d, J = 1.7 Hz, 1H), 8.15 (dd, J = 8.0, 1.8 Hz, 1H), 8.15 (dd, J = 8.0, 1.8 Hz, 1H), 7.45 (d, J = 8.1 Hz, 1H), 4.55 (d, J = 4.6 Hz, 2H), 4.02-3.78 (m, 4H), 3.50-3.37 (m, 4H), 2.45 (s, 3H), 1.50-1.19 (m, 4H). |
| 92 | 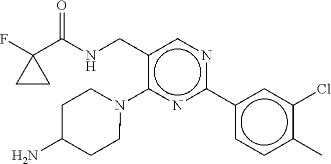 | LCMS (ESI): m/z calcd for C21H25ClFN5O + H: 418.2, found: 418.2. 1H NMR (400 MHz, MeOD) δ 8.30 (s, 1H), 8.24 (d, J = 1.8 Hz, 1H), 8.08 (dd, J = 8.0, 1.8 Hz, 1H), 7.48 (d, J = 8.9 Hz, 1H), 4.54 (s, 2H), 4.47 (d, J = 13.8 Hz, 2H), 3.51 (td, J = 11.4, 4.2 Hz, 1H), 3.38-3.31 (m, 2H), 2.46 (s, 3H), 2.20 (d, J = 14.4 Hz, 2H), 1.80 (qd, J = 12.4, 4.0 Hz, 2H), 1.43-1.25 (m, 4H). |
| 93 | 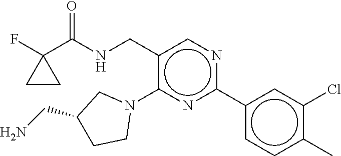 | LCMS (ESI): m/z calcd for C21H25ClFN5O + H: 418.2, found: 418.2. 1H NMR (400 MHz, MeOD) δ 8.22 (s, 1H), 8.11 (s, 1H), 8.03 (dd, J = 7.8, 1.5 Hz, 1H), 7.53 (d, J = 8.2 Hz, 1H), 4.69 (s, 2H), 4.34-4.13 (m, 2H), 4.11- 3.97 (m, 1H), 3.78 (t, J = 9.8 Hz, 1H), 3.22-3.06 (m, 2H), 2.73-2.62 (m, 1H), 2.36 (m, 1H), 1.99-1.82 (m, 1H), 1.42-1.22 (m, 4H). |
| 94 | 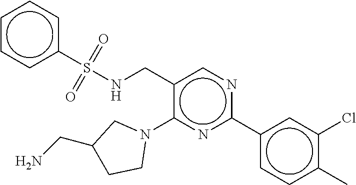 | LCMS (ESI): m/z calcd for C23H26ClN5O2S + H: 472.1, found: 472.2. 1H NMR (400 MHz, MeOD-d4) δ 8.14 (d, J = 1.6, 1 H), 8.04 (s, 1 H), 7.99-7.96 (m, 1 H), 7.85-7.83 (m, 2 H), 7.63-7.52 (m, 4H), 4.31-4.26 (m, 3 H), 4.21-4.15 (m, 1 H), 4.03-3.96 (m, 1 H), 3.77-3.72 (m, 1 H), 3.20-3.09 (m, 2H), 2.72-2.68 (m, 1 H), 2.48 (s, 3H), 2.36-2.32 (m, 1H), 1.92-1.87 (m, 1H). |
| 95 | 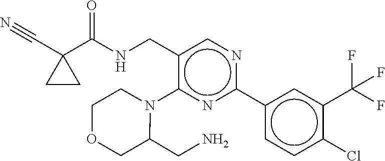 | LCMS (ESI): m/z calcd for C22H22ClF3N6O2 + H: 495.2, found: 495.2. 1H NMR (400 MHz, MeOD-d4) δ 8.73 (s, 1 H), 8.57-8.54 (m, 2 H), 7.74 (d, J = 6.8, 1 H), 4.53 (d, J = 15.2, 1 H), 4.28 (d, J = 15.6, 1 H), 4.09-4.06 (m, 1 H), 3.99-3.97 (m, 1 H), 3.89-3.81 (m, 2H), 3.74- 3.68 (m, 1 H), 3.56-3.53 (m, 1H), 3.35-3.31 (m, 1H), 3.29-3.27 (m, 2H), 1.60-1.48 (m, 4H). |
| 96 | 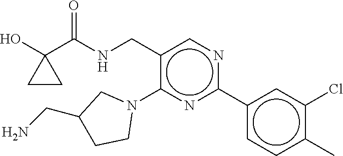 | LCMS (ESI): m/z calcd for C21H26ClN5O2 + H: 416.2, found: 416.3. 1H NMR (400 MHz, MeOD-d4) δ 8.20 (s, 1 H), 8.06-8.01 (m, 2 H), 7.56 (d, J = 8.4, 1 H), 4.67 (s, 2 H), 4.35-4.21 (m, 2 H), 4.09-4.02 (m, 1 H), 3.84-3.79 (m, 1 H), 3.21-3.10 (m, 2H), 2.75-2.67 (m, 1 H), 2.49 (s, 3H), 2.40-2.33 (m, 1H), 1.97-1.87 (m, 1H), 1.23-1.20 (m, 2H), 1.05-1.02 (m, 2H). |
| 97 | 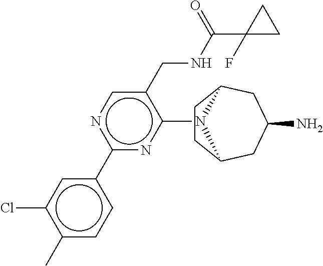 | LCMS (ESI): m/z calcd for C23H27ClFN5O + H: 444.2, found: 444.2. 1H NMR (400 MHz, MeOD) δ 8.27-8.15 (m, 2H), 8.11-7.98 (m, 1H), 7.54 (d, J = 8.1 Hz, 1H), 5.10 (s, 2H), 4.57 (s, 2H), 3.55-3.38 (m, 1H), 2.82-2.63 (m, 2H), 2.49 (s, 3H), 2.30 (dd, J = 9.3, 4.8 Hz, 2H), 2.06 (d, J = 8.2 Hz, 2H), 1.91 (dd, J = 14.2, 4.8 Hz, 2H), 1.47-1.23 (m, 4H). |
| 98 | 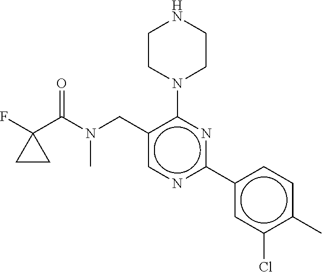 | LCMS (ESI): m/z calcd for C21H25ClFN5O + H: 418.2, found: 418.2. 1H NMR (400 MHz, MeOD) δ 8.35 (s, 1H), 7.55 (s, 1H), 7.38 (d, J = 10.4 Hz, 2H), 4.60 (s, 2H), 4.23-4.01 (m, 4H), 3.29 (d, J = 6.7 Hz, 4H), 2.93 (s, 3H), 2.43 (s, 3H), 1.22 (d, J = 19.0 Hz, 4H). |
| 99 | 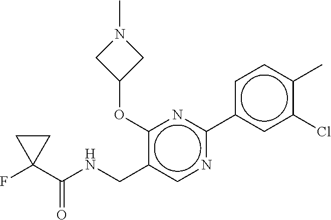 | LCMS (ESI): m/z calcd for C20H22ClFN4O2 + H: 405.2; found: 405.2. 1H NMR (400 MHz, MeOD-d4) δ 8.49 (s, 1H), 8.29 (d, J = 1.2 Hz, 1H), 8.17-8.14 (m, 1H), 7.40 (d, J = 8.0 Hz, 1H), 5.53 (d, J = 6.0 Hz, 1H), 4.49 (s, 2H), 4.13-4.09 (m, 2H), 3.58-3.54 (m, 2H), 2.58 (s, 3H), 2.43 (s, 3H), 1.37-1.31 (m, 4H). |
| 100 | 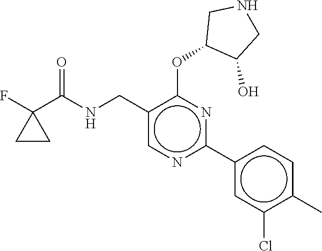 | Racemic; LCMS (ESI): m/z calcd for C20H22ClFN4O3 + H: 421.2; found: 421.2. 1H NMR (400 MHz, MeOD-d₄) δ 8.55 (s, 1H), 8.35 (d, J = 1.2 Hz, 1H), 8.23-8.21 (m, 1H), 7.41 (d, J = 8.0 Hz, 1H), 5.84-5.80 (m, 1H), 4.76-4.73 (m, 1H), 4.53 (d, J = 4.4 Hz, 2H), 3.93-3.88 (m, 1H), 3.60-3.51 (m, 2H), 3.46- 3.42 (m, 1H), 2.44 (s,3H), 1.36-1.31 (m, 4H). |
| 101 | 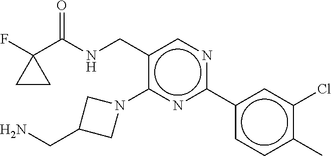 | LCMS (ESI): m/z calcd for C20H23ClFN5O + H: 404.1, found: 404.1. 1H NMR (400 MHz, MeOD) δ 8.19 (d, J = 1.8 Hz, 1H), 8.10-7.93 (m, 2H), 7.52 (d, J = 8.1 Hz, 1H), 4.78 (d, J = 9.6 Hz, 2H), 4.56-4.26 (m, 2H), 3.36 (d, J = 7.5 Hz, 2H), 3.31 (dt, J = 3.3, 1.6 Hz, 2H), 3.25- 3.11 (m, 1H), 2.48 (s, 3H), 1.50-1.17 (m, 4H). |
| 102 | 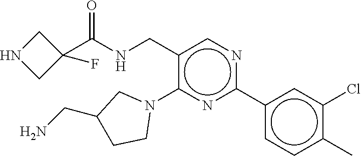 | LCMS (ESI): m/z calcd for C21H26ClFN6O + H: 433.2, found 433.3. 1H NMR (400 MHz, MeOD) δ 8.26-8.14 (m, 2H), 8.05 (dd, J = 8.0, 1.9 Hz, 1H), 7.51 (d, J = 8.2 Hz, 1H), 4.68 (s, 2H), 4.64-4.51 (m, 2H), 4.42 (dd, J = 18.8, 13.5 Hz, 2H), 4.25 (dd, J = 11.9, 7.4 Hz, 1H), 4.20-4.11 (m, 1H), 4.01 (dd, J = 17.5, 9.7 Hz, 1H), 3.75 (dd, J = 11.6, 8.3 Hz, 1H), 3.21-3.08 (m, 2H), 2.74-2.63 (m, 1H), 2.47 (s, 3H), 2.40-2.29 (m, 1H), 1.90 (dq, J = 17.3, 8.7 Hz, 1H). |
| 103 | 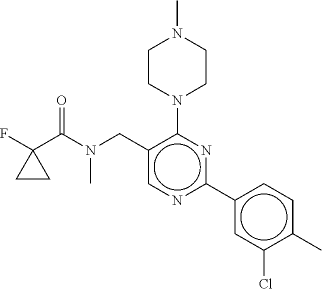 | LCMS (ESI): m/z calcd for C22H27ClFN5O + H: 432.2, found: 432.2. 1H NMR (400 MHz, MeOD) δ 8.50 (s, 1H), 8.34 (d, J = 1.7 Hz, 1H), 8.19 (dd, J = 8.0, 1.7 Hz, 1H), 7.41 (d, J = 8.0 Hz, 1H), 4.54 (d, J = 4.1 Hz, 2H), 3.94 (d, J = 4.6 Hz, 4H), 3.70-3.57 (m, 4H), 3.31 (d, J = 1.7 Hz, 6H), 2.44 (s, 3H), 1.45-1.21 (m, 4H). |
| 104 | 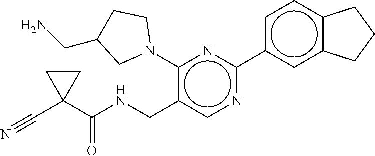 | LCMS (ESI): m/z calcd for C24H28N6O + H: 417.2, found: 417.2. 1H NMR (400 MHz, MeOD-d4) δ 8.09- 7.91 (m, 3 H), 7.46 (d, 1 H), 4.64 (s, 2 H), 4.38-4.18 (m, 2 H), 4.12-4.00 (m, 1 H), 3.86-3.75 (m, 1 H), 3.24-2.97 (m, 6 H), 2.78-2.64 (m, 1 H), 2.44-2.31 (m, 1 H), 2.24- 2.10 (m, 2 H), 2.00-1.85 (m, 1 H), 1.68-1.55 (m, 4 H). |
| 105 | 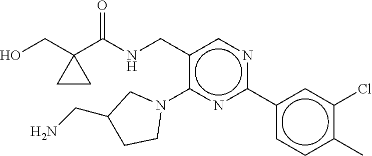 | LCMS (ESI): m/z calcd for C22H28ClN5O2 + H: 430.2, found: 430.3. 1H NMR (400 MHz, MeOD-d₄) δ 8.20 (d, 1 H), 8.10-7.97 (m, 2 H), 7.55 (d, 1 H), 4.65 (s, 2 H), 4.37-4.17 (m, 2 H), 4.11-3.99 (m, 1 H), 3.86-3.67 (m, 3 H), 3.23-3.08 (m, 2 H), 2.77-2.63 (m, 1 H), 2.49 (s, 3 H), 2.43-2.29 (m, 1 H), 1.99-1.84 (m, 1 H), 1.18-1.09 (m, 2 H), 0.84-0.74 (m, 2 H). |
| 106 | 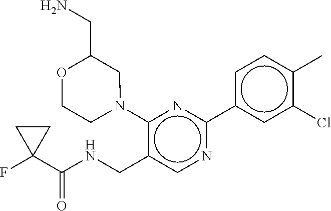 | LCMS (ESI): m/z calcd for C21H25ClFN5O2 + H: 434.2; found: 434.2. 1H NMR (400 MHz, DMSO-d6) 8.35 (s, 1H), 8.26 (d, J = 2.0 Hz, 1H), 8.12-8.10 (m, 1H), 7.47 (d, J = 8.0 Hz, 1H), 4.56 (d, J = 3.2 Hz, 2H), 4.43 (d, J = 94.8 Hz, 1H), 4.15-4.12 (m, 2H), 3.93 (s, 1H), 3.83 (d, J = 2.4 Hz, 1H), 3.38 (s, 1H), 3.27-3.23 (m, 2H), 3.14-3.07 (m, 2H), 2.46 (s, 1H), 1.46-1.30 (m, 4H). |
| 107 | 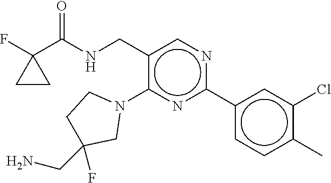 | LCMS (ESI): m/z calcd for C21H24ClF2N5O + H: 436.2; found: 436.3. 1H NMR (400 MHz, DMSO-d6) 8.24 (d, J = 1.60 Hz, 1H), 8.20 (s, 1H), 8.08-8.06 (m, 1H), 7.51 (d, J = 8.0 Hz, 1H), 4.69 (s, 2H), 4.43-4.14 (m, 4H), 3.65-3.48 (m, 2H), 3.13-2.47 (m, 5H), 1.39- 1.30 (m, 4H). |
| 108 | 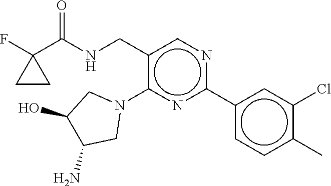 | LCMS (ESI): m/z calcd for C22H24ClF4N5O4 + H: 420.2, found: 420.1. 1H NMR (400 MHz, MeOD-d4) δ 8.21-8.19 (m, 2H), 8.05-8.02 (m, 1H), 7.54 (d, J = 8, 1H), 4.75-4.66 (m, 2H), 4.60-4.57 (m, 1H), 4.50-4.39 (m, 2H), 4.27-4.23 (m, 1H), 4.07-4.04 (m, 1 H), 3.88-3.85 (m, 1H), 2.48 (s, 3H), 1.41-1.31 (m, 4H). |
| 109 | 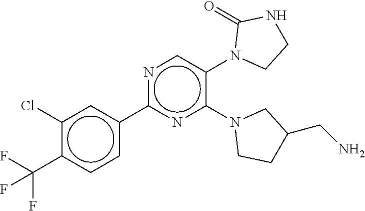 | See Ex. 13 |
| 110 | 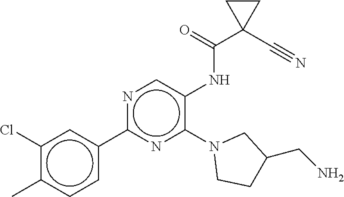 | LCMS calcd for C21H24ClN6O (M + H)+ m/z = 411.2; found: 411.0, 413.0. 1H NMR (400 MHz, CD3OD) δ 8.27 (d, J = 1.6 Hz, 1 H), 8.13 (dd, J = 8.0, 1.6 Hz, 1 H), 8.04 (s, 1 H), 7.36 (d, J = 8.0 Hz, 1 H), 3.96-4.03 (m, 1 H), 3.86-3.94 (m, 1 H), 3.68-3.77 (m, 1 H), 3.45- 3.52 (m, 1 H), 2.74-2.88 (m, 2 H), 2.28-2.45 (m, 4 H), 2.14-2.25 (m, 1 H), 1.61-1.82 (m, 5 H). |
| 111 | 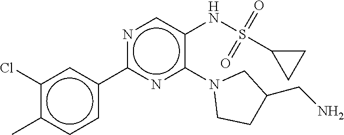 | LCMS calcd for C19H25ClN5O2S (M + H)+ m/z = 422.1; found: 422.0, 424.0. 1H NMR (400 MHz, CD3OD) δ 8.26 (d, J = 0.8 Hz, 2 H), 8.09 (d, J = 8.0 Hz, 1 H), 7.48 (d, J = 8.0 Hz, 1 H), 4.15-4.35 (m, 2 H), 3.95-4.06 (m, 1 H), 3.76 (dd, J = 12.0, 8.0 Hz, 1 H), 3.05-3.19 (m, 2 H), 2.57-2.79 (m, 2 H), 2.46 (s, 3 H), 2.26-2.37 (m, 1 H), 1.82-1.94 (m, 1 H), 1.01-1.17 (m, 4 H). |
| 112 | 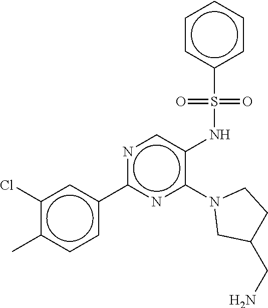 | LCMS calcd for C22H25ClN5O2S (M + H)+ m/z = 458.1; found: 458.0, 460.0. ¹H NMR (400 MHz, CD3OD) δ 8.15 (d, J = 1.6 Hz, 1 H), 7.99 (dd, J = 8.0, 1.6 Hz, 1 H), 7.73-7.80 (m, 2 H), 7.47-7.59 (m, 3 H), 7.46 (s, 1 H), 7.30 (d, J = 8.0 Hz, 1 H), 4.08 (dd, J = 11.6, 7.2 Hz, 1 H), 3.95-4.05 (m, 1 H), 3.65-3.84 (m, 2 H), 2.83-2.95 (m, 2 H), 2.39-2.48 (m, 1 H), 2.38 (s, 3 H), 2.09-2.19 (m, 1 H), 1.62-1.74 (m, 1 H). |
| 113 | 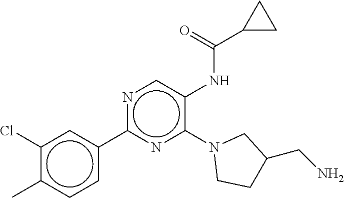 | LCMS calcd for C20H25ClN5O (M + H) + m/z = 386.2; found: 386, 388.0. ¹H NMR (400 MHz, CD3OD) δ 8.26 (d, J = 1.6 Hz, 1 H), 8.12 (dd, J = 8.0, 1.6 Hz, 1 H), 7.96 (s, 1 H), 7.35 (d, J = 8.0 Hz, 1 H), 3.93-3.97 (m, 1 H), 3.83-3.89 (m, 1 H), 3.65-3.74 (m, 1 H), 3.45 (dd, J = 11.2, 7.2 Hz, 1 H), 2.74-2.84 (m, 2 H), 2.41 (s, 3 H), 2.35-2.41 (m, 1 H), 2.14-2.20 (m, 1 H), 1.71-1.82 (m, 2 H), 0.80-0.99 (m, 4 H). |
| 114 | 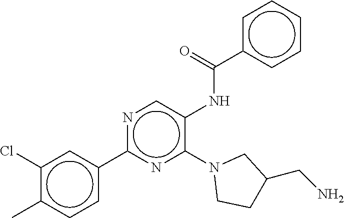 | LCMS calcd for C23H25ClN5O (M + H) + m/z = 422.2; found: 422.2, 424.2. 1H NMR (400 MHz, CD3OD) δ 8.31 (d, J = 1.6 Hz, 1 H), 8.16 (dd, J = 8.0, 1.6 Hz, 1 H), 8.09 (s, 1 H), 7.94-8.02 (m, 2 H), 7.59-7.66 (m, 1 H), 7.55 (t, J = 7.6 Hz, 2 H), 7.38 (d, J = 8.0 Hz, 1 H), 3.97 (dd, J = 11.2, 7.2 Hz, 1 H), 3.82-3.91 (m, 1 H), 3.66- 3.76 (m, 1 H), 3.47 (dd, J = 11.2, 7.6 Hz, 1 H), 2.65- 2.81 (m, 2 H), 2.43 (s, 3 H), 2.30-2.41 (m, 1 H), 2.07- 2.18 (m, 1 H), 1.66-1.77 (m, 1 H). |
| 115 | 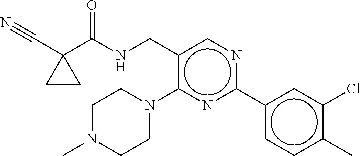 | LCMS (ESI): m/z calcd for C22H25ClN6O + H: 425.2, found 425.2. 1H NMR (400 MHz, MeOD) δ 8.48 (s, 1H), 8.32 (d, J = 1.6 Hz, 1H), 8.18 (dd, J = 8.0, 1.7 Hz, 1H), 7.41 (d, J = 8.0 Hz, 1H), 4.49 (s, 2H), 4.19 (s, 2H), 3.48 (d, J = 1.6 Hz, 6H), 2.99 (s, 3H), 2.44 (s, 3H), 1.61 (g, J = 3.5 Hz, 4H). |
| 116 | 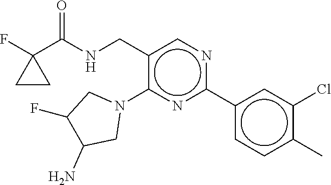 | LCMS (ESI): m/z calcd for C20H22ClF2N5O + H: 422.1, found: 422.2. 1H NMR (400 MHz, MeOD-d4) δ 8.24 (d, J = 8, 2 H), 8.06 (d, J = 8, 1 H), 7.52 (d, J = 8, 1 H), 5.57 (d, J = 49.2, 1 H), 4.74-4.65 (m, 2H), 4.59-4.25 (m, 5H), 2.48 (s, 3 H), 1.41-1.28 (m, 4 H). |
| 117 | 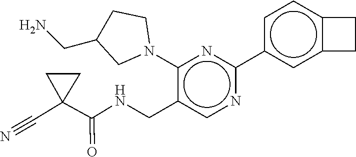 | LCMS (ESI): m/z calcd for C23H26N6O + H: 403.2, found: 403.2. 1H NMR (400 MHz, MeOD-d4) δ 8.85- 8.76 (m, 2 H), 8.67-8.59 (m, 1 H), 7.75 (d, 1 H), 5.97 (d, 1 H), 4.56 (s, 2 H), 3.65-3.53 (m, 1 H), 2.80-2.68 (m, 3 H), 2.43-2.22 (m, 2 H), 2.06-1.88 (m, 1 H), 1.62-1.54 (m, 4 H) |
| 118 | 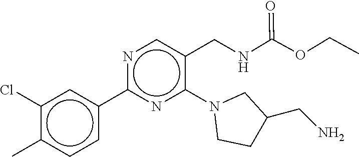 | LCMS (ESI): m/z calcd for C20H26ClN5O2 + H: 404.2, found: 404.1. 1H NMR (400 MHz, MeOD-d4) δ 8.85- 8.76 (m, 2 H), 8.67-8.59 (m, 1 H), 7.75 (d, 1 H), 5.97 (d, 1 H), 4.56 (s, 2 H), 3.65-3.53 (m, 1 H), 2.80-2.68 (m, 3 H), 2.43-2.22 (m, 2 H), 2.06-1.88 (m, 1 H), 1.62-1.54 (m, 4 H). |
| 119 | 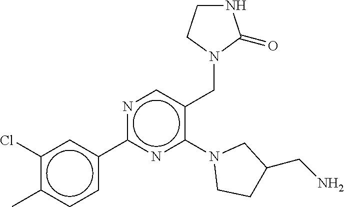 | LCMS (ESI): m/z calcd for C20H25ClN6O + H: 401.2, found: 401.2. 1H NMR (400 MHz, MeOD): δ 8.22 (d, J = 2.0 Hz, 1H), 8.10 (s, 1H), 8.04 (dd, J = 8.0, 2.0 Hz, 1H), 7.54 (d, J = 8.0 Hz, 1H), 4.54 (dd, J = 34.8, 15.8 Hz, 2H), 4.31 (dd, J = 12.0, 7.4 Hz, 1H), 4.23 (ddd, J = 12.0, 8.0, 3.8 Hz, 1H), 4.09-3.98 (m, 1H), 3.78 (dd, J = 12.2, 8.4 Hz, 1H), 3.48-3.38 (m, 4H), 3.14 (p, J = 5.6 Hz, 2H), 2.76-2.62 (m, 1H), 2.49 (s, 3H), 2.34 (dd, J = 11.0, 4.8 Hz, 1H), 1.96-1.83 (m, 1H) |
| 120 | 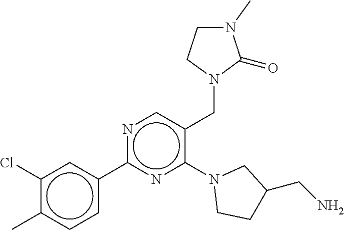 | LCMS (ESI): m/z calcd for C21H27ClN6O + H: 415.2, found: 415.2. 1H NMR (400 MHz, MeOD): δ 8.21 (d, J = 2.0 Hz, 1H), 8.09 (s, 1H), 8.04 (dd, J = 8.0, 2.0 Hz, 1H), 7.55 (d, J = 8.0 Hz, 1H), 4.55 (m, 2H), 4.33 (dd, J = 12.2, 7.4 Hz, 1H), 4.25 (ddd, J = 11.8, 8.0, 3.6 Hz, 1H), 4.05 (dd, J = 14.0, 5.8 Hz, 1H), 3.80 (dd, J = 12.2, 8.4 Hz, 1H), 3.47-3.37 (m, 2H), 3.37-3.32 (m, 2H), 3.14 (t, J = 6.6 Hz, 2H), 2.83 (s, 3H), 2.75-2.64 (m, 1H), 2.49 (s, 3H), 2.41-2.29 (m, 1H), 1.89 (dd, J = 12.4, 8.8 Hz, 1H) |
| 121 | 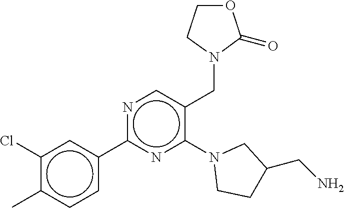 | See Ex. 14 |
| 122 | 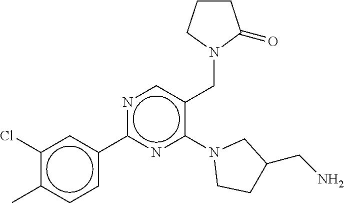 | LCMS (ESI): m/z calcd for C21H26ClN5O + H: 400.2, found: 400.2. 1H NMR (400 MHz, MeOD) δ 8.22 (d, J = 1.8 Hz, 1H), 8.05 (dd, J = 8.0, 1.9 Hz, 1H), 7.99 (s, 1H), 7.54 (d, J = 8.2 Hz, 1H), 4.72 (dd, J = 36.4, 15.9 Hz, 2H), 4.30-4.13 (m, 2H), 4.06-3.95 (m, 1H), 3.77 (dd, J = 12.0, 8.2 Hz, 1H), 3.44 (t, J = 7.2 Hz, 2H), 3.21-3.05 (m, 2H), 2.68 (dt, J = 15.8, 7.8 Hz, 1H), 2.48 (s, 3H), 2.34 (ddd, J = 17.0, 11.8, 5.4 Hz, 1H), 2.16- 2.06 (m, 2H), 1.95-1.82 (m, 1H) |
| 123 | 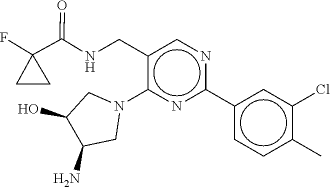 | LCMS (ESI): m/z calcd for C20H23ClFN5O2 + H: 420.1, found: 420.2. 1H NMR (400 MHz, MeOD-d4) δ 8.22-8.19 (m, 2 H), 8.05-8.03 (m, 1 H), 7.53 (m, 1 H), 4.69-4.56 (m, 3 H), 4.45-4.39(m, 1H), 4.29-4.23 (m, 1H), 4.16-4.04 (m, 2 H), 4.02-3.97 (m, 1 H), 2.48 (m, 3 H), 1.41-1.29 (m, 4 H) |
| 124 | 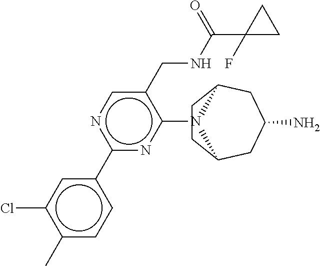 | LCMS (ESI): m/z calcd for C23H27ClFN5O + H: 444.2, found : 444.2. 1H NMR (400 MHz, MeOD) δ 8.93 (s, 1H), 8.25 (s, 1H), 8.21 (d, J = 1.8 Hz, 1H), 8.06 (dd, J = 8.0, 1.8 Hz, 1H), 7.51 (d, J = 8.0 Hz, 1H), 5.09 (s, 2H), 4.57 (d, J = 4.9 Hz, 2H), 3.82 (dt, J = 17.5, 5.8 Hz, 1H), 2.47 (s, 3H), 2.19 (dd, J = 12.3, 6.9 Hz, 4H), 2.05-1.89 (m, 4H), 1.46-1.23 (m, 4H) |
| 125 | 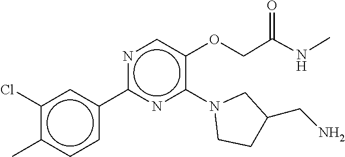 | See Ex. 15 |
| 126 | 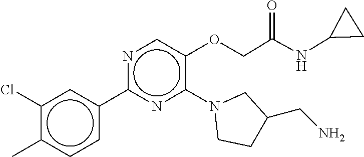 | LCMS calculated for C21H27ClN5O2 (M + H)+ m/z = 416.2; found: 416.2/418.1. 1H NMR (400 MHz, CD3OD) δ 8.19 (d, J = 1.6 Hz, 1 H), 8.04 (dd, J = 8.0, 1.6 Hz, 1 H), 7.76 (s, 1 H), 7.33 (d, J = 8.0 Hz, 1H), 4.52 (s, 2 H), 3.98-4.14 (m, 2 H), 3.76-3.89 (m, 1 H), 3.52-3.62 (m, 1 H), 2.80 (d, J = 7.2 Hz, 2 H), 2.71- 2.77 (m, 1 H), 2.40 (s, 3 H), 2.34-2.38 (m, 1 H), 2.11- 2.21 (m, 1 H), 1.66-1.78 (m, 1 H), 0.73-0.80 (m, 2 H), 0.53-0.58 (m, 2 H) |
| 127 | 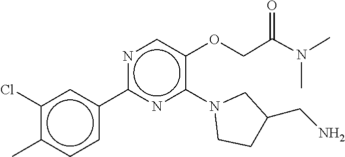 | LCMS calculated for C₂₀H₂₇ClN₅O₂ (M + H)⁺ m/z = 404.2; found: 404.2/406.2. 1H NMR (400 MHz, CD₃OD) δ 8.17 (d, J = 1.6 Hz, 1 H), 8.03 (dd, J = 8.0, 1.2 Hz, 1H), 7.78 (s, 1 H), 7.32 (d, J = 8.0 Hz, 1 H), 4.88 (s, 2 H), 4.03-4.14 (m, 2 H), 3.80-3.86 (m, 1 H), 3.58- 3.63 (m, 1 H), 3.07 (s, 3 H), 2.99 (s, 3 H), 2.81 (d, J = 7.2 Hz, 2 H), 2.36-2.43 (m, 1 H), 2.39 (s, 3 H), 2.12- 2.20 (m, 1 H), 1.67-1.76 (m, 1 H) |
| 128 | 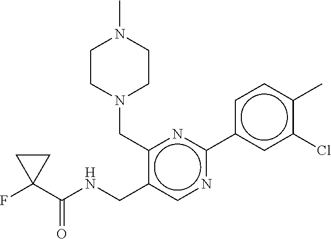 | See Ex. 16 |
| 129 | 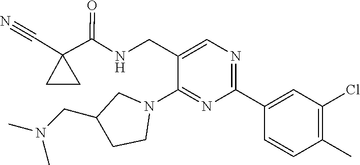 [SMILES: Cc1ccc(-c2ncc(CNC(=O)C3(C#N)CC3)c(N3CCC(CN(C)C)C3)n2)cc1Cl] | LCMS (ESI): m/z calcd for C₂₄H₂₉ClN₆O + H: 453.2, found: 453.3. ¹H NMR (400 MHz, MeOD) δ 8.22 (d, J = 1.8 Hz, 1H), 8.12 (s, 1H), 8.05 (dd, J = 8.0, 1.8 Hz, 1H), 7.53 (d, J = 8.1 Hz, 1H), 4.64 (s, 2H), 4.34 (dd, J = 11.7, 7.3 Hz, 1H), 4.20 (m, 1H), 4.01 (m, 1H), 3.79 (dd, J = 11.7, 8.7 Hz, 1H), 3.41-3.34 (m, 2H), 2.99 (s, 6H), 2.89 (m, 1H), 2.48 (s, 3H), 2.37 (m, 1H), 1.93 (m, 1H), 1.66-1.53 (m, 4H). |
| 130 | 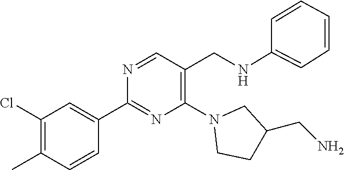 [SMILES: Cc1ccc(-c2ncc(CNc3ccccc3)c(N3CCC(CN)C3)n2)cc1Cl] | LCMS (ESI): m/z calcd for C₂₃H₂₆ClN₅ + H: 408.2, found: 408.2. ¹H NMR (400 MHz, MeOD) δ 8.20 (d, J = 1.9 Hz, 1H), 8.09 (s, 1H), 8.01 (dd, J = 8.0, 1.9 Hz, 1H), 7.55 (d, J = 8.1 Hz, 1H), 7.26-7.04 (m, 2H), 6.83- 6.59 (m, 3H), 4.48 (m, 2H), 4.35 (m, 2H), 4.20-4.04 (m, 1H), 3.84 (m, 1H), 3.12 (m, 2H), 2.69 (m, 1H), 2.49 (s, 3H), 2.33 (m, 1H), 1.90 (m, 1H). |
| 131 | 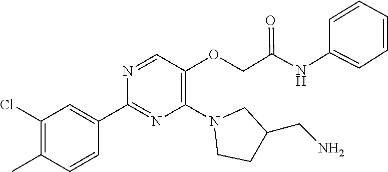 [SMILES: Cc1ccc(-c2ncc(OCC(=O)Nc3ccccc3)c(N3CCC(CN)C3)n2)cc1Cl] | LCMS calcd. for C₂₂H₂₇F₃N₅O (M + H)⁺ m/z = 451.97; found: 452.2. ¹H NMR (400 MHz, MeOD) δ 8.16 (d, J = 1.6 Hz, 1H), 7.98 (dd, J = 8.0, 1.6 Hz, 1H), 7.90 (s, 1H), 7.59 (d, J = 7.6 Hz, 2H), 7.51 (d, J = 8.0 Hz, 1H), 7.34 (t, J = 8.0 Hz, 2H), 7.14 (t, J = 7.6 Hz, 1H), 4.92 (s, 2H), 4.25-4.50 (m, 2H), 4.10 (s, 1H), 3.86 (s, 1H), 3.07- 3.19 (m, 2H), 2.65-2.69 (m, 1H), 2.47 (s, 3H), 2.31- 2.34 (m, 1H), 1.86-1.96 (m, 1H), 1.26-1.36 (m, 1H). |
| 132 | 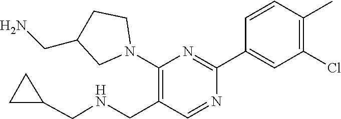 [SMILES: Cc1ccc(-c2ncc(CNCC3CC3)c(N3CCC(CN)C3)n2)cc1Cl] | LCMS (ESI): m/z calcd for C₂₁H₂₈ClN₅ + H+: 386.20, found: 386.3. ¹H NMR (400 MHz, MeOD-d₄) δ 8.42 (s, 1 H), 8.25 (d, J = 1.6, 1 H), 8.10-8.07 (m, 1H), 7.52 (d, J = 8, 1 H), 4.51 (m, 2 H), 4.26-4.21 (m, 1H), 4.14-4.08 (m, 1H), 4.02-3.94 (m, 1H), 3.77-3.72 (m, 1 H), 3.22- 3.12 (m, 2H), 3.10-3.08 (m, 2H), 2.76-2.68 (m, 1H), 2.48 (s, 3H), 2.2.42-2.35 (m, 1H), 1.98-1.88 (m, 1H), 1.20-1.13 (m, 1H), 0.76-0.71 (m, 2H), 0.47-0.43 (m, 2H). |
| 133 | 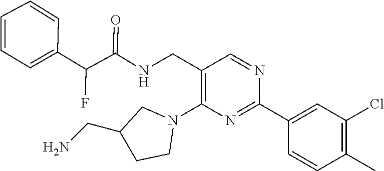 [SMILES: Cc1ccc(-c2ncc(CNC(=O)C(F)c3ccccc3)c(N3CCC(CN)C3)n2)cc1Cl] | LCMS (ESI): m/z calcd for C₂₅H₂₇ClFN₅O + H: 468.2, found: 468.2. ¹H NMR (400 MHz, MeOD) δ 8.21 (d, J = 1.6 Hz, 1H), 8.12-8.00 (m, 2H), 7.57-7.40 (m, 6H), 5.89 (dd, J = 47.2, 1.2Hz, 1H), 4.75-4.58 (m, 2H), 4.28- 4.08 (m, 2H), 4.02-3.88 (m, 1H), 3.77-3.61 (m, 1H), 3.17-2.96 (m, 2H), 2.67-2.53 (m, 1H), 2.48 (s, 3H), 2.33-2.23 (m, 1H), 1.89-1.74 (m, 1H). |
| 134 | 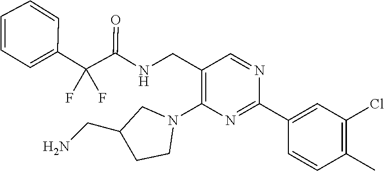 [SMILES: Cc1ccc(-c2ncc(CNC(=O)C(F)(F)c3ccccc3)c(N3CCC(CN)C3)n2)cc1Cl] | LCMS (ESI): m/z calcd for C₂₅H₂₆ClF₂N₅O + H: 486.2, found: 486.2. ¹H NMR (400 MHz, MeOD) δ 8.21 (d, J = 1.6 Hz, 1H), 8.10-7.99 (m, 2H), 7.68-7.46 (m, 6H), 4.73-4.58 (m, 2H), 4.25-4.02 (m, 2H), 3.98-3.85 (m, 1H), 3.73-3.61 (m, 1H), 3.15-2.97 (m, 2H), 2.68-2.54 (m, 1H), 2.48 (s, 3H), 2.33-2.20 (m, 1H), 1.89-1.73 (m, 1H). |
| 135 | 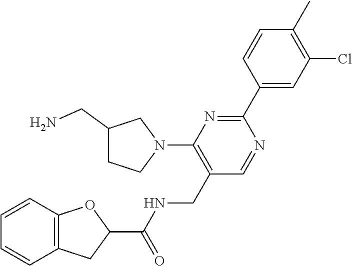 [SMILES: Cc1ccc(-c2ncc(CNC(=O)C3Cc4ccccc4O3)c(N3CCC(CN)C3)n2)cc1Cl] | LCMS (ESI): m/z calcd for C₂₆H₂₈ClN₅O₂ + H: 478.2, found: 478.2. ¹H NMR (400 MHz, MeOD) δ 8.19 (d, J = 2.0 Hz, 1H), 8.05-7.96 (m, 2H), 7.58-7.49 (m, 1H), 7.26-7.11 (m, 2H), 6.97-6.85 (m, 2H), 5.28-5.18 (m, 1H), 4.70-4.58 (m, 2H), 4.29-4.08 (m, 2H), 4.03-3.90 (m, 1H), 3.78-3.55 (m, 2H), 3.42-3.32 (m, 1H), 3.20- 3.00 (m, 2H), 2.69-2.56 (m, 1H), 2.48 (s, 3H), 2.35-2.22 (m, 1H), 1.91-1.76 (m, 1H). |
| 136 | 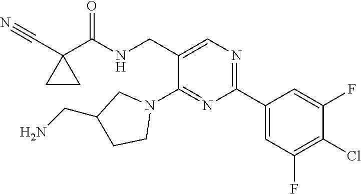 [SMILES: N#CC1(C(=O)NCc2cnc(-c3cc(F)c(Cl)c(F)c3)nc2N2CCC(CN)C2)CC1] | LCMS (ESI): m/z calcd for C₂₁H₂₁ClF₂N₆O + H: 447.14, found: 447.2. ¹H NMR (400 MHz, MeOD) δ 8.20 (s, 1H), 8.03 (d, J = 8.1 Hz, 2H), 4.62 (m, 2H), 4.14 (m, 1H), 4.04 (m, 1H), 3.92 (m, 1H), 3.67 (m, 1H), 3.14 (m, 2H), 2.74-2.50 (m, 1H), 2.43-2.22 (m, 1H), 1.98- 1.75 (m, 1H), 1.66-1.47 (m, 4H). |
| 137 | 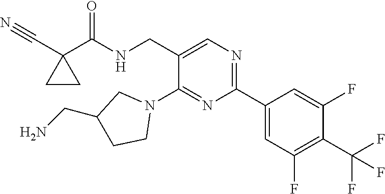 [SMILES: N#CC1(C(=O)NCc2cnc(-c3cc(F)c(C(F)(F)F)c(F)c3)nc2N2CCC(CN)C2)CC1] | LCMS (ESI): m/z calcd for C₂₂H₂₁F₅N₆O + H: 480.17, found: 481.2. ¹H NMR (400 MHz, MeOD) δ 8.24 (s, 1H), 8.07 (d, J = 11.1 Hz, 2H), 4.69-4.53 (m, 2H), 4.20-3.98 (m, 2H), 3.92 (m, 1H), 3.67 (m, 1H), 3.23- 3.04 (m, 2H), 2.65 (m, 1H), 2.32 (m, 1H), 1.98-1.79 (m, 1H), 1.67-1.53 (m, 4H). |
| 138 | 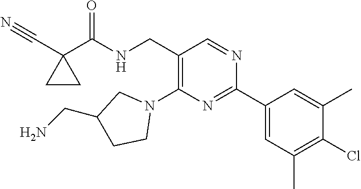 [SMILES: Cc1cc(-c2ncc(CNC(=O)C3(C#N)CC3)c(N3CCC(CN)C3)n2)cc(C)c1Cl] | LCMS (ESI): m/z calcd for C₂₃H₂₇ClN₆O + H: 439.19, found: 439.3. ¹H NMR (400 MHz, MeOD) δ 8.09 (s, 1H), 7.95 (s, 2H), 4.64 (s, 2H), 4.44-4.14 (m, 2H), 4.05 (m, 1H), 3.92-3.70 (m, 1H), 3.14 (m, 2H), 2.71 (s, 1H), 2.49 (s, 6H), 2.37 (m, 1H), 1.94 (m, 1H), 1.60 (m, 4H). |
| 139 | 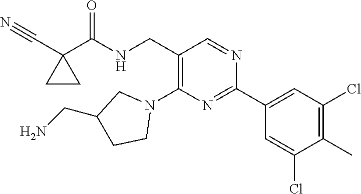 [SMILES: Cc1c(Cl)cc(-c2ncc(CNC(=O)C3(C#N)CC3)c(N3CCC(CN)C3)n2)cc1Cl] | LCMS (ESI): m/z calcd for C₂₂H₂₄Cl₂N₆O + H+: 459.14; found: 459.2. ¹H NMR (400 MHz, MeOD): δ 8.19 (s, 2H), 8.13 (s, 1H), 4.62 (s, 2H), 4.24 (m, 1H), 4.15 (m, 1H), 4.01 (m, 1H), 3.76 (m, 1H), 3.21-3.08 (m, 2H), 2.74-2.64 (m, 1H), 2.57 (s, 3H), 2.35 (m, 1H), 1.91 (m, 1H), 1.62 (m, 2H), 1.60 (m, 2H). |
| 140 | 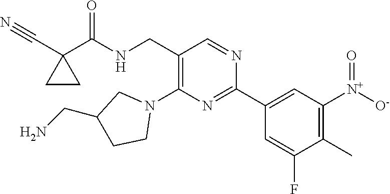 [SMILES: Cc1c(F)cc(-c2ncc(CNC(=O)C3(C#N)CC3)c(N3CCC(CN)C3)n2)cc1[N+](=O)[O-]] | LCMS (ESI): m/z calcd for C₂₂H₂₄FN₇O₃ + H+: 454.2, found: 454.3. ¹H NMR (400 MHz, MeOD-d₄) δ 8.64 (s, 1 H), 8.31-8.28 (m, 1 H), 8.21 (d, J = 3.6, 1 H), 4.61 (s, 2 H), 4.18-4.08 (m, 1H), 4.08-4.05 (m, 1H), 3.98-3.91 (m, 1H), 3.72-3.67 (m, 1H), 3.17-3.08 (m, 2H), 2.69- 2.64 (m, 1H), 2.51 (s, 3H), 2.36-2.31 (m, 1H), 1.94-1.86 (m, 1 H), 1.62-1.58 (m, 4H). |
| 141 | 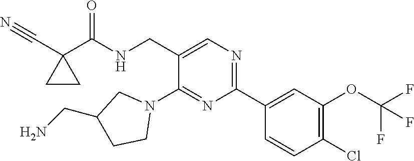 [SMILES: N#CC1(C(=O)NCc2cnc(-c3ccc(Cl)c(OC(F)(F)F)c3)nc2N2CCC(CN)C2)CC1] | LCMS (ESI): m/z calcd for C₂₄H₂₄ClF₆N₆O₄+: 495.15, found: 495.2. ¹H NMR (400 MHz, MeOD) δ 8.30 (s, 1H), 8.26 (dd, J = 8.5, 2.0 Hz, 1H), 8.18 (s, 1H), 7.76 (d, J = 8.5 Hz, 1H), 4.66-4.56 (m, 2H), 4.19 (m, 1H), 4.10 (s, 1H), 3.96 (m, 1H), 3.72 (m, 1H), 3.14 (m, 2H), 2.66 (m, 1H), 2.33 (m, 1H), 1.90 (m, 1H), 1.66-1.54 (m, 4H). |
| 142 | 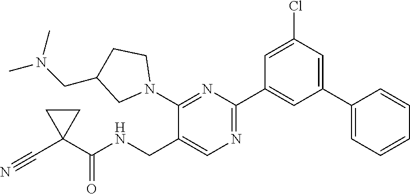 [SMILES: CN(C)CC1CCN(c2nc(-c3cc(Cl)cc(-c4ccccc4)c3)ncc2CNC(=O)C2(C#N)CC2)C1] | LCMS (ESI): m/z calcd for C29H31ClN6O + H: 515.23, found: 515.2. ¹H NMR (400 MHz, MeOD) δ 8.37 (s, 1H), 8.20 (d, J = 7.1 Hz, 2H), 7.90 (s, 1H), 7.71 (d, J = 7.3 Hz, 2H), 7.51 (t, J = 7.4 Hz, 2H), 7.45 (d, J = 7.1 Hz, 1H), 4.64 (s, 2H), 4.31 (m, 1H), 4.19 (s, 1H), 4.01 (m, 1H), 3.85-3.66 (m, 1H), 3.37 (m, 2H), 2.99 (s, 6H), 2.93-2.82 (m, 1H), 2.37 (s, 1H), 1.99-1.83 (m, 1H), 1.61 (m, 4H). |
| 143 | 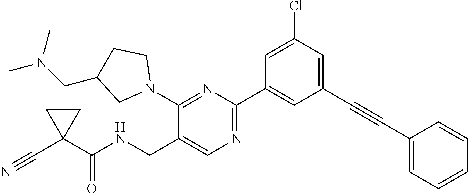 [SMILES: CN(C)CC1CCN(c2nc(-c3cc(Cl)cc(C#Cc4ccccc4)c3)ncc2CNC(=O)C2(C#N)CC2)C1] | LCMS (ESI): m/z calcd for C₃₁H₃₁ClN₆O + H: 539.22, found: 539.3. ¹H NMR (400 MHz, MeOD): δ 8.28 (t, J = 1.4 Hz, 1H), 8.22 (t, J = 1.8 Hz, 1H), 8.19 (s, 1H), 7.77 (s, 1H), 7.57 (dd, J = 6.8, 3.0 Hz, 2H), 7.45-7.40 (m, 3H), 4.62 (s, 2H), 4.27 (m, 1H), 4.14 (m, 1H), 3.99 (m, 1H), 3.78-3.70 (m, 1H), 3.37 (m, 2H), 2.99 (s, 6H), 2.87 (s, 1H), 2.37 (m, 1H), 1.92 (m, 1H), 1.62 (m, 2H), 1.60 (m, 2H). |
| 144 | 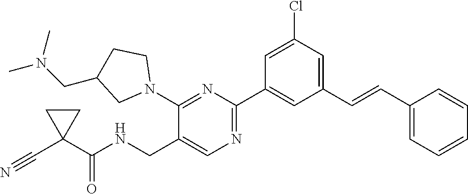 [SMILES: CN(C)CC1CCN(c2nc(-c3cc(Cl)cc(/C=C/c4ccccc4)c3)ncc2CNC(=O)C2(C#N)CC2)C1] | LCMS (ESI): m/z calcd for C₃₁H₃₃ClN₆O + H: 541.24; found: 541.3. ¹H NMR (400 MHz, MeOD): δ 8.29 (s, 1H), 8.19 (s, 1H), 8.09 (t, J = 1.6 Hz, 1H), 7.86 (s, 1H), 7.61 (d, J = 7.2 Hz, 2H), 7.38 (dd, J = 15.0, 7.0 Hz, 3H), 7.31 (t, J = 7.2 Hz, 1H), 7.25 (d, J = 16.4 Hz, 1H), 4.64 (s, 2H), 4.32 (m, 1H), 4.18 (m, 1H), 4.07-3.97 (m, 1H), 3.81-3.72 (m, 1H), 3.37 (m, 2H), 2.99 (s, 6H), 2.89 (m, 1H), 2.38 (s, 1H), 1.98-1.85 (m, 1H), 1.65- 1.61 (m, 2H), 1.60 (m, 2H). |
| 145 | 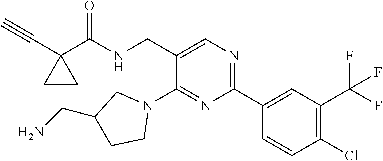 [SMILES: C#CC1(C(=O)NCc2cnc(-c3ccc(Cl)c(C(F)(F)F)c3)nc2N2CCC(CN)C2)CC1] | LCMS (ESI): m/z calcd for C₂₃H₂₃ClF₃N₅O + H+: 478.15, found: 478.1. ¹H NMR (400 MHz, MeOD): δ 8.62 (d, J = 1.6 Hz, 1H), 8.46 (dd, J = 8.4, 2.0 Hz, 1H), 8.13 (s, 1H), 7.82 (d, J = 8.4 Hz, 1H), 4.65 (d, J = 2.4 Hz, 2H), 4.22 (m, 1H), 4.19-4.08 (m, 1H), 3.99 (m, 1H), 3.74 (m, 1H), 3.14 (m, 2H), 2.79 (s, 1H), 2.74- 2.59 (m, 1H), 2.42-2.27 (m, 1H), 1.90 (m, 1H), 1.44 (m, 2H), 1.25 (m, 2H). |
| 146 | 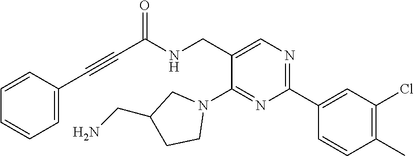 | LCMS (ESI): m/z calcd for C₂₁H₂₁ClF₂N₆O + H: 447.1, found: 447.2. ¹H NMR (400 MHz, MeOD) δ 8.23 (s, 1H), 8.18 (s, 1H), 8.06 (d, J = 8.1 Hz, 1H), 7.61-7.54 (m, 2H), 7.50 (dd, J = 13.3, 7.1 Hz, 2H), 7.42 (t, J = 7.4 Hz, 2H), 4.67 (s, 2H), 4.22 (m, 2H), 4.03 (s, 1H), 3.76 (s, 1H), 3.24-3.04 (m, 2H), 2.69 (s, 1H), 2.48 (s, 3H), 2.36 (s, 1H), 1.90 (m, 1H). |
| 147 | 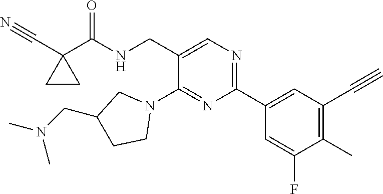 [SMILES: C#Cc1cc(-c2ncc(CNC(=O)C3(C#N)CC3)c(N3CCC(CN(C)C)C3)n2)cc(F)c1C] | See Example 17 |
| 148 | 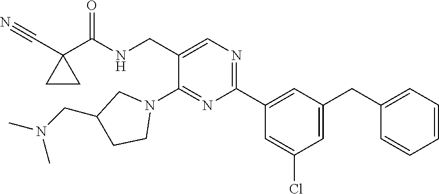 [SMILES: CN(C)CC1CCN(c2nc(-c3cc(Cl)cc(Cc4ccccc4)c3)ncc2CNC(=O)C2(C#N)CC2)C1] | LCMS (ESI): m/z calcd for C₃₀H₃₃ClN₆O + H: 529.25, found: 529.3. ¹H NMR (400 MHz, MeOD) δ 8.12 (s, 1H), 8.05 (s, 1H), 7.96 (s, 1H), 7.49 (s, 1H), 7.30 (m, 2H), 7.24 (m, 3H), 4.61 (s, 2H), 4.29 (m, 1H), 4.14 (m, 1H), 4.09 (s, 2H), 4.01-3.90 (m, 1H), 3.74 (m, 1H), 3.36 (m, 2H), 2.98 (s, 6H), 2.93-2.78 (m, 1H), 2.36 (m, 1H), 1.89 (m, 1H), 1.68-1.49 (m, 4H) |
| 149 | 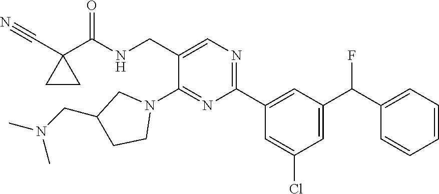 [SMILES: CN(C)CC1CCN(c2nc(-c3cc(Cl)cc(C(F)c4ccccc4)c3)ncc2CNC(=O)C2(C#N)CC2)C1] | LCMS (ESI): m/z calcd for C₃₀H₃₂ClFN₆O + H: 547.2, found: 547.1 ¹H NMR (400 MHz, MeOD) δ 8.23-8.11 (m, 3H), 7.61 (s, 1H), 7.47-7.32 (m, 5H), 6.64 (d, J = 46.8 Hz, 1H), 4.61 (s, 2H), 4.32-4.07 (m, 2H), 4.00- 3.87 (m, 1H), 3.77-3.67 (m, 1H), 3.38-3.32 (m, 2H), 2.98 (s, 6H), 2.95-2.77 (m, 1H), 2.41-2.31 (m, 1H), 1.96-1.82 (m, 1H), 1.66-1.54 (m, 4H). |
| 150 | 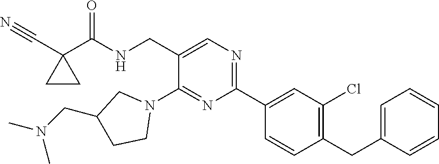 [SMILES: CN(C)CC1CCN(c2nc(-c3ccc(Cc4ccccc4)c(Cl)c3)ncc2CNC(=O)C2(C#N)CC2)C1] | LCMS (ESI): m/z calcd for C₃₀H₃₃ClN₆O + H: 529.24, found: 529.4. ¹H NMR (400 MHz, MeOD-d₄) δ 8.25 (s, 1 H), 8.09-8.06 (m, 2 H), 7.50 (d, J = 8 Hz, 1 H), 7.32- 7.21 (m, 5H), 4.67(s, 2 H), 4.47-4.39 (m, 1H), 4.28- 4.22 (m, 3H), 4.11-4.04 (m, 1H), 3.88-3.83 (m, 1H), 3.39 (m, 2 H), 2.98 (s, 6H), 2.93-2.89 (m, 1H), 2.42- 2.39 (m, 1H), 1.96-1.92 (m, 1H), 1.63-1.61(m, 4H) |

### Example 17: SHG Screening of Compounds

Compounds suspected to carry intrinsic SHG activity were tested in the presence of an unlabeled KRAS^{G12D}-FMe.RAF1^{RBDCRD} complex to monitor binding. PS bilayer was prepared from PS-enriched small unilamellar vesicles (SUVs) purchased from Biodesy, Inc. The concentrated SUVs were diluted in TBS buffer containing 5 mM CaCl₂ and incubated on a glass-bottom 384-well Biodesy Delta™ assay plate to allow bilayer formation. Equimolar amounts of GppNHp-loaded KRAS^{G12D}-FMe and RAF1^{RBDCRD} were mixed and incubated together on PS bilayer overnight at 4° C. The following day, untethered protein was removed by washing with assay buffer (20 mM Hepes, pH 7.4, 150 mM MgCl₂, 1 mM TCEP, 5 μM GppNHp). The SHG signal produced by bilayer-tethered complex was ready using the Biodesy Delta™ platform where each well was pulsed with an 800 nm laser and the resulting second harmonic signal intensity at 400 nm was collected. Compounds were diluted into the same assay buffer and applied to the complex at a final concentration range spanning 0.027 μM-40 μM and the SHG intensity of each well was monitored at 5 minute intervals over a 60 minute time period.

### Example 18: pERK Inhibition in SW-480 Cell Line

SW-480 cells were cultured in Leibovitz's L15 (Gibco Cat. No. 11415064), supplemented with 10% fetal bovine serum (Gibco, Cat. No. 1993708), and 1% penicillin and streptomycin. Cells were grown at 37° C., under atmospheric conditions without additional CO₂. Cells were seeded into a 96-well tissue-culture treated plate (Greiner, Cat. No. 655180) at a density of 30,000 cell per well, and allowed to adhere for 16 hours before the media was removed, and cells were treated with compounds diluted into culture media at the indicated concentrations. After 1 hour of incubation in the presence of compound, cells were harvested to measure pERK levels using the lysis buffer supplied with the Homogenous Time Resolved Fluorescence pERK kit (HTRF, Cisbio Advance ERK phospho-T202/Y204, Cat. No. 64AERPEG). Lysates were transferred to a 384-well low-volume white plate (PerkinElmer, Cat. No. 6008280) and then treated with the detection reagents supplied by the HTRF kit, according to the “2-plate” protocol supplied by the manufacturer, and incubated for 4-hours at ambient temperature in darkness. Assay plates were read at two fluorescent wavelengths (665 nm and 620 nm) using an Envision plate reader (Perkin Elmer) and data was processed and fitted to a 4-parameter logistic model using Graphpad Prism 8.0 to calculate IC₅₀ values.

### Example 19: pERK Inhibition in MiaPaca-2 Cell Line

MiaPaCa-2 cells (ATCC CRL-1420) were cultured in high glucose DMEM (Gibco 11965092) containing 10% fetal bovine serum (Gibco 1993708) and 1× Penicillin/Streptomycin, at 37° C. in a humid atmosphere of 5% CO2 in the air. Cells were plated in tissue-culture treated 96-well plates at a density of 10,000 cells/well and allowed to attach for 16 hours. The medium was then removed, and diluted compounds were added in a final concentration of 0.5% DMSO. After 1 hour of incubation, cells were lysed by the lysis buffer supplied with the Phospho-ERK (Thr202/Tyr204) cellular HTRF kit (Cisbio 64ERKPEH) and transferred to a 384-well low-volume white plate (PerkinElmer 6008280). The cell lysates were treated with the detection reagents supplied with the HTRF kit for 4 hours at ambient temperature in darkness. Two fluorescence intensities at the wavelengths (665 and 620 nm) were measured by the Envision plate reader (Perkin Elmer), and the data were processed and fitted to a 4-parameter logistic model for IC₅₀ calculation (GraphPad Prism 9).

SHG EC₅₀ (μM) SHG assay and pERK assay IC₅₀ (μM) for selected compounds are provided in Table 2.

TABLE 2
Compound potency in SHG assay and pERK assay
in SW-480 and MiaPaca-2 cell lines.

| | SHG | SW-480 | MiaPaca-2 |
|---|---|---|---|
| Cmpd | EC₅₀ | pERK | pERK |
| No. | μM^{a} | IC₅₀ μM^{a} | IC₅₀ μM^{a} |
| 1 | ++ | | + |
| 2 | +++ | | ++ |
| 3 | | | ++ |
| 4 | ++ | | |
| 5 | ++ | ++ | ++ |
| 6 | ++ | | |
| 7 | ++ | | ++ |
| 8 | | | ++ |
| 9 | ++ | | + |
| 10 | ++ | | ++ |
| 11 | | | ++ |
| 12 | ++ | | ++ |
| 13 | | | ++ |
| 14 | ++ | | + |
| 15 | | | ++ |
| 16 | | | ++ |
| 17 | ++ | | ++ |
| 18 | | | |
| 19 | ++ | | ++ |
| 20 | | | |
| 21 | | | + |
| 22 | | | ++ |
| 23 | ++ | | ++ |
| 24 | | | + |
| 25 | ++ | | + |
| 26 | | | |
| 27 | | | |
| 28 | + | | ++ |
| 29 | ++ | | ++ |
| 30 | + | | ++ |
| 31 | ++++ | | ++ |
| 32 | +++ | | ++ |
| 33 | ++ | | ++ |
| 34 | +++ | | ++ |
| 35 | + | | ++ |
| 36 | ++ | | ++ |
| 37 | + | | ++ |
| 38 | + | | ++ |
| 39 | ++ | | ++ |
| 40 | +++ | | ++ |
| 41 | ++ | ++ | +++ |
| 42 | ++ | | ++ |
| 43 | + | | + |
| 44 | ++ | | ++ |
| 45 | ++ | | ++ |
| 46 | ++ | | ++ |
| 47 | ++ | | ++ |
| 48 | ++ | | ++ |
| 49 | ++ | | ++ |
| 50 | ++ | | ++ |
| 51 | ++ | | ++ |
| 52 | + | ++ | ++ |
| 53 | | | |
| 54 | ++ | | ++ |
| 55 | + | | ++ |
| 56 | + | | + |
| 57 | + | ++ | ++ |
| 58 | ++ | | ++ |
| 59 | ++ | ++ | ++ |
| 60 | ++ | | ++ |
| 61 | ++ | | ++ |
| 62 | ++ | ++ | +++ |
| 63 | ++ | | +++ |
| 64 | | | + |
| 65 | ++ | | +++ |
| 66 | ++ | | ++ |
| 67 | ++ | ++ | +++ |
| 68 | + | | ++ |
| 69 | ++ | | ++ |
| 70 | ++ | | + |
| 71 | ++ | | ++ |
| 72 | | ++ | ++ |
| 73 | ++ | +++ | +++ |
| 74 | ++ | | ++ |
| 75 | ++ | | ++ |
| 76 | ++ | | ++ |
| 77 | ++ | | ++ |
| 78 | + | | |
| 79 | + | | |
| 80 | + | | ++ |
| 81 | + | | ++ |
| 82 | ++ | | ++ |
| 83 | + | + | ++ |
| 84 | ++ | ++ | ++ |
| 85 | ++ | | |
| 86 | ++ | | |
| 87 | ++ | ++ | ++ |
| 88 | ++ | | ++ |
| 89 | + | | ++ |
| 90 | ++ | ++ | ++ |
| 91 | ++ | ++ | ++ |
| 92 | ++ | | ++ |
| 93 | ++ | | ++ |
| 94 | ++ | | ++ |
| 95 | | | |
| 96 | ++ | | ++ |
| 97 | | | |
| 98 | ++ | | ++ |
| 99 | | | |
| 100 | | | + |
| 101 | + | | ++ |
| 102 | ++ | | ++ |
| 103 | | | |
| 104 | + | ++ | +++ |
| 105 | + | ++ | +++ |
| 106 | | | |
| 107 | | | |
| 108 | | | |
| 109 | ++ | | ++ |
| 110 | + | | ++ |
| 111 | | | ++ |
| 112 | ++ | ++ | ++ |
| 113 | + | | ++ |
| 114 | | | ++ |
| 115 | | | ++ |
| 116 | | | |
| 117 | | | ++ |
| 118 | + | | ++ |
| 119 | ++ | | ++ |
| 120 | + | | ++ |
| 121 | ++ | | ++ |
| 122 | ++ | | ++ |
| 123 | | | |
| 124 | ++ | | ++ |
| 125 | | | |
| 126 | ++ | | ++ |
| 127 | | | ++ |
| 128 | | | + |
| 129 | ++ | | |
| 130 | ++ | | |
| 131 | ++ | | |
| 132 | ++ | | |
| 133 | ++ | | |
| 134 | ++ | | |
| 135 | ++ | | |
| 136 | | | |
| 137 | ++ | | |
| 138 | | | |
| 139 | ++ | | |
| 140 | ++ | | |
| 141 | ++ | | |
| 142 | ++ | | |
| 143 | ++ | | |
| 144 | ++ | | |
| 145 | ++ | | |
| 146 | ++ | | |
| 147 | + | | |
| 148 | ++ | | |
| 149 | ++ | | |
| 150 | ++ | | |

^{a}10 μM =< IC₅₀ or EC₅₀ <= 50 μM, +; 1 μM =< IC₅₀ or EC₅₀ < 10 μM, ++; 0.1 μM =< IC₅₀ or EC₅₀ < 1μM, +++; IC₅₀ or EC₅₀ < 0.1 μM, ++++

We claim:

1-65. (canceled)

66. A compound represented by the structure of Formula (I): 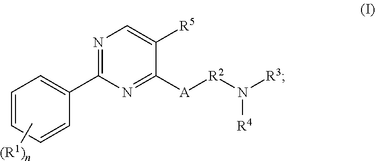
or a pharmaceutically acceptable salt thereof, wherein:
R¹ is independently selected at each occurrence from:
halogen, —OR⁶, —SR⁶, —N(R⁶)₂, —C(O)R⁶, —C(O)OR⁶, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, and —CN;
C₁₋₆ alkyl, C₂₋₆ alkenyl, and C₂₋₆ alkynyl, each of which is optionally substituted with one or more substituents independently selected from:
halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN; and
C₃₋₆ carbocycle and 3- to 6-membered heterocycle each of which is optionally substituted with one or more substituents independently selected from halogen, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and CN;
C₃₋₆ carbocycle optionally substituted with one or more substituents independently selected from halogen, —OR⁶, —SR⁶, —N(R⁶)₂, —C(O)R⁶, —C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, —CN;
3 to 6-membered heterocycle optionally substituted with one or more substituents independently selected from halogen, C₁₋₆ alkyl, —OR⁶, —SR⁶, —N(R⁶)₂, —C(O)R⁶, C(O)OR⁶, —OC(O)R⁶, —OC(O)N(R⁶)₂, —C(O)N(R⁶)₂, —N(R⁶)C(O)R⁶, —N(R⁶)C(O)OR⁶, —N(R⁶)C(O)N(R⁶)₂, —N(R⁶)₂S(O)₂(R⁶), —S(O)R⁶, —S(O)₂R⁶, —S(O)₂N(R⁶)₂, —NO₂, ═O, and —CN; and
two R¹ on adjacent carbon atoms can combine to form a C₄₋₆ carbocycle or 4- to 6-membered heterocycle together with the carbon atoms to which they are bound;
A is selected from a bond, —O—, —N(R¹¹)—, —S—, —S(═O)— and —S(═O)₂—, and a 4- to 9-membered heterocyclene optionally substituted with one or more R¹²;
R² is selected from C₁₋₄ alkylene, C₂₋₄ alkenylene, C₂₋₄ alkynylene, and C₃-C₇ carbocyclene and 4- to 9-membered heterocyclene, any of which are optionally substituted with one or more substituents independently selected from halogen, —OR⁷, —SR⁷, —N(R⁷)₂, —C(O)R⁷, C(O)OR⁷, —OC(O)R⁷, —OC(O)N(R⁷)₂, —C(O)N(R⁷)₂, —N(R⁷)C(O)R⁷, —N(R⁷)C(O)OR⁷, —N(R⁷)C(O)N(R⁷)₂, —N(R⁷)₂S(O)₂(R⁷), —S(O)R⁷, —S(O)₂R⁷, —S(O)₂N(R⁷)₂, —NO₂, ═O, and CN;
R³ and R⁴ are independently selected from hydrogen, C₁₋₃ alkyl, 3- to 7-membered heterocycle, and C₃-C₇ carbocycle, wherein C₁₋₃ alkyl, 3- to 7-membered heterocycle, and C₃-C₇ carbocycle, any of which are optionally substituted with one or more substituents independently selected from halogen, R⁸, —OR⁸, —SR⁸, —N(R⁸)₂, —C(O)R⁸, —C(O)OR⁸, —OC(O)R⁸, —OC(O)N(R⁸)₂, —C(O)N(R⁸)₂, —N(R⁸)C(O)R⁸, —N(R⁸)C(O)OR⁸, —N(R⁸)C(O)N(R⁸)₂, —N(R⁸)₂S(O)₂(R⁸), —S(O)R⁸, —S(O)₂R⁸, —S(O)₂N(R′)₂, —NO₂, and —CN; or
R³ and R⁴ can come together to form a 4- to 6-membered heterocycle optionally substituted with one or more substituents independently selected from:
halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, and —CN; and
C₁₋₆ alkyl optionally substituted with one or more substituents independently selected from halogen, —OR⁹, —SR⁹, —N(R⁹)₂, —C(O)R⁹, —C(O)OR⁹, —OC(O)R⁹, —OC(O)N(R⁹)₂, —C(O)N(R⁹)₂, —N(R⁹)C(O)R⁹, —N(R⁹)C(O)OR⁹, —N(R⁹)C(O)N(R⁹)₂, —N(R⁹)₂S(O)₂(R⁹), —S(O)R⁹, —S(O)₂R⁹, —S(O)₂N(R⁹)₂, —NO₂, ═O, and —CN; or
R² and R⁴ can come together to form a 4- to 6-membered heterocycle optionally substituted with one or more substituents independently selected from:
halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN; and
C₁₋₆ alkyl optionally substituted with one or more substituents independently selected from halogen, —OR¹⁵, —SR¹⁵, —N(R¹⁵)₂, —C(O)R¹⁵, —C(O)OR¹⁵, —OC(O)R¹⁵, —OC(O)N(R¹⁵)₂, —C(O)N(R¹⁵)₂, —N(R¹⁵)C(O)R¹⁵, —N(R¹⁵)C(O)OR¹⁵, —N(R¹⁵)C(O)N(R¹⁵)₂, —N(R¹⁵)₂S(O)₂(R¹⁵), —S(O)R¹⁵, —S(O)₂R¹⁵, —S(O)₂N(R¹⁵)₂, —NO₂, ═O, and —CN;
R⁵ is selected from:
—N(R¹⁰)₂, —NO₂, —C(O)R¹⁰, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), and —S(O)₂N(R¹⁰)₂;
C₁ alkyl substituted with one or more substituents independently selected from halogen, R^{C}, R^{D}, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN;
C₂₋₆ alkyl, C₂₋₆ alkenyl, and C₂₋₆ alkynyl, each of which is optionally substituted with one or more substituents independently selected from halogen, R^{C}, R^{D}, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and
3- to 6-membered saturated heterocycle, 3- to 6-membered unsaturated heterocycle, C₃-C₆ saturated carbocycle, C₃-C₆ unsaturated carbocycle, —O—(C₁-C₆ alkyl), —O-(3- to 6-membered heterocycle), and —O—(C₃-C₆ carbocycle), each of which is optionally substituted with one or more substituents independently selected from:
halogen, —OR¹⁰, —SR¹⁰, —N(R^{A})(R^{B}), —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN; and
C₁₋₆ alkyl, C₂₋₅ alkenyl, C₂₋₅ alkynyl, each of which is optionally substituted with one or more substituents independently selected from halogen, —OR¹⁰, —SR¹⁰, —N(R¹⁰)₂, —C(O)R¹⁰, —C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰, —S(O)₂R¹⁰, —S(O)₂N(R¹⁰)₂, —NO₂, ═O, and —CN;
R^{A} and R^{B} are each independently selected from:
hydrogen, —OR¹⁸, —C(O)R¹⁸, —C(O)OR¹⁸, —C(O)N(R¹⁸)₂, —S(O)R¹⁸, —S(O)₂R¹⁸, and —S(O)₂N(R¹⁸)₂; and
C₁₋₆ alkyl, C₂₋₆ alkenyl, C₂₋₆ alkynyl, C₃₋₆ carbocycle, and 3- to 6-membered heterocycle, any of which is optionally substituted with one or more substituents independently selected from:
halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, and —CN; and
C₃₋₆ carbocycle and 3- to 6-membered heterocycle each of which is optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —OC(O)N(R¹⁸)₂, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, and CN; or
R^{A} and R^{B} can come together to form a 4- to 6-membered heterocycle optionally substituted with one or more substituents independently selected from:
halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN; and
C₁₋₆ alkyl optionally substituted with one or more substituents independently selected halogen, —OR¹⁹, —SR¹⁹, —N(R¹⁹)₂, —C(O)R¹⁹, —C(O)OR¹⁹, —OC(O)R¹⁹, —OC(O)N(R¹⁹)₂, —C(O)N(R¹⁹)₂, —N(R¹⁹)C(O)R¹⁹, —N(R¹⁹)C(O)OR¹⁹, —N(R¹⁹)C(O)N(R¹⁹)₂, —N(R¹⁹)₂S(O)₂(R¹⁹), —S(O)R¹⁹, —S(O)₂R¹⁹, —S(O)₂N(R¹⁹)₂, —NO₂, ═O, and —CN;
R^{C} and R^{D} are each independently selected from C₁₋₆ alkyl, C₃₋₆ carbocycle, and 3- to 6-membered heterocycle, wherein C₁₋₆ alkyl, C₃₋₆ carbocycle, and 3- to 6-membered heterocycle are each optionally substituted with one or more substituents independently selected from halogen, —OR¹⁶, —SR¹⁶, —N(R¹⁶)₂, —C(O)R¹⁶, —C(O)OR¹⁶, —OC(O)R¹⁶, —OC(O)N(R¹⁶)₂, —C(O)N(R¹⁶)₂, —N(R¹⁶)C(O)R¹⁶, —N(R¹⁶)C(O)OR¹⁶, —N(R¹⁶)C(O)N(R¹⁶)₂, —N(R¹⁶)₂S(O)₂(R¹⁶), —S(O)R¹⁶, —S(O)₂R¹⁶, —S(O)₂N(R¹⁶)₂, —NO₂, ═O, and —CN; or
R^{C} and R^{D} can come together to form a 4- to 6-membered heterocycle optionally substituted with one or more substituents independently selected from:
halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN; and
C₁₋₆ alkyl optionally substituted with one or more substituents independently selected halogen, —OR¹⁷, —SR¹⁷, —N(R¹⁷)₂, —C(O)R¹⁷, —C(O)OR¹⁷, —OC(O)R¹⁷, —OC(O)N(R¹⁷)₂, —C(O)N(R¹⁷)₂, —N(R¹⁷)C(O)R¹⁷, —N(R¹⁷)C(O)OR¹⁷, —N(R¹⁷)C(O)N(R¹⁷)₂, —N(R¹⁷)₂S(O)₂(R¹⁷), —S(O)R¹⁷, —S(O)₂R¹⁷, —S(O)₂N(R¹⁷)₂, —NO₂, ═O, and —CN;
R¹⁸ is independently selected from:
hydrogen;
C₁₋₆ alkyl, C₂₋₆ alkenyl, C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
C₃₋₆ carbocycle and 3- to 6-membered heterocycle each of which are optionally substituted with one or more substituents independently selected from:
halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN; and
C₁₋₆ alkyl, C₂₋₆ alkenyl, and C₂₋₆ alkynyl each optionally substituted with one or more substituents independently selected from halogen, R²², —OR²², —SR²², —N(R²²)₂, —C(O)R²², —C(O)OR²², —OC(O)R²², —OC(O)N(R²²)₂, —C(O)N(R²²)₂, —N(R²²)C(O)R²², —N(R²²)C(O)OR²², —N(R²²)C(O)N(R²²)₂, —N(R²²)₂S(O)₂(R²²), —S(O)R²², —S(O)₂R²², —S(O)₂N(R²²)₂, —NO₂, ═O, and —CN;
each of R⁶, R⁷, R⁸, R⁹, R¹⁰, R¹¹, R¹², R¹⁵, R¹⁶, R¹⁷, R¹⁹, and R²² are independently selected at each occurrence from hydrogen, halogen, —OH, —NO₂, —CN, C₁₋₆ alkyl, C₁₋₆ haloalkyl, and C₃₋₆ carbocycle, 3- to 6-membered heterocycle, wherein the C₃₋₆ carbocycle and 3- to 6-membered heterocycle is optionally substituted with one or more substituents independently selected from halogen, —OH, —NO₂, —CN, C₁₋₆ alkyl, C₁₋₆ alkoxy, and C₁₋₆ haloalkyl; and
n is selected from 1, 2, and 3.

67. The compound or salt of claim 66, wherein R⁵ is selected from C₁ alkyl substituted with —N(R^{A})(R^{B}), and Formula (I) is represented by Formula (I-a):

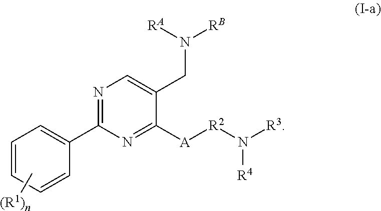

68. The compound or salt of claim 67, wherein R^{A} is selected from hydrogen and C₁₋₆ alkyl optionally substituted with one or more substituents independently selected halogen, —OR¹⁸, —N(R¹⁸)₂, —C(O)R¹⁸, —NO₂, ═O, and —CN; and
R^{B} is selected from: —C(O)R¹⁸, —C(O)OR¹⁸, —C(O)N(R¹⁸)₂, —S(O)₂R¹⁸; and C₁₋₆ alkyl optionally substituted with one or more substituents independently selected from halogen, —OR¹⁸, —SR¹⁸, —N(R¹⁸)₂, —C(O)R¹⁸, —C(O)OR¹⁸, —OC(O)R¹⁸, —C(O)N(R¹⁸)₂, —N(R¹⁸)C(O)R¹⁸, —N(R¹⁸)C(O)OR¹⁸, —N(R¹⁸)C(O)N(R¹⁸)₂, —N(R¹⁸)₂S(O)₂(R¹⁸), —S(O)R¹⁸, —S(O)₂R¹⁸, —S(O)₂N(R¹⁸)₂, —NO₂, ═O, and —CN.

69. The compound or salt of claim 68, wherein R^{B} is —C(O)R¹⁸ and R¹⁸ is selected from C₂₋₄ alkenyl, C₃₋₆ carbocycle and 3- to 6-membered heterocycle, each of which is optionally substituted with one or more substituents independently selected from: halogen, —OR²², —N(R²²)₂, —C(O)R²², —C(O)N(R²²)₂, —CN; and C₁₋₆ alkyl each of which are optionally substituted with one or more substituents independently selected from halogen, —OR²² and —N(R²²)₂.

70. The compound or salt of claim 69, wherein 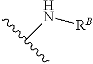
is selected from:

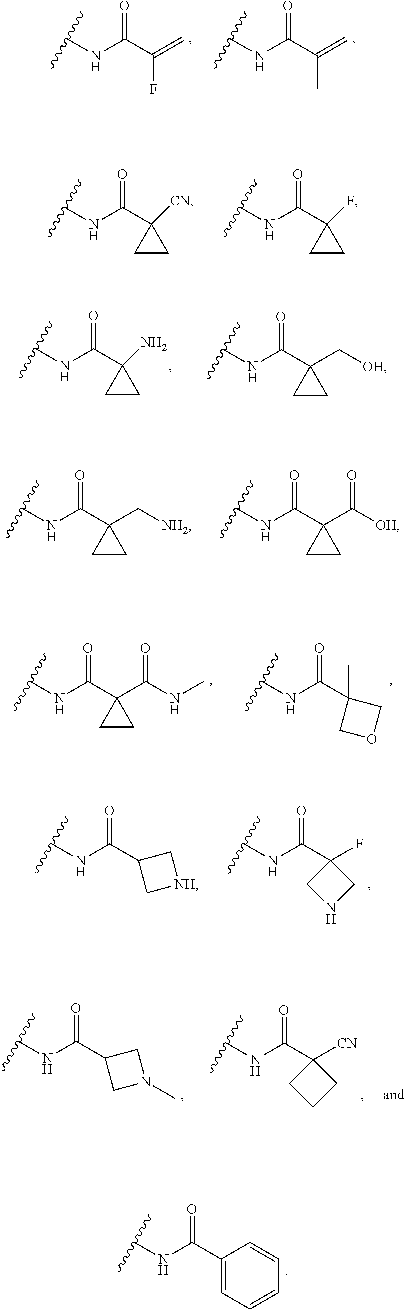

71. The compound or salt of claim 68, wherein R^{B} is —C(O)R¹⁸ and R¹⁸ is selected from C₂₋₄ alkenyl, C₃₋₆ carbocycle and 3- to 6-membered heterocycle, each of which is optionally substituted with one or more substituents independently selected from: halogen, —OR²², —N(R²²)₂, —C(O)R²², —C(O)N(R²²)₂, —CN; and C₁₋₆ alkyl, C₂₋₆ alkenyl, and C₂₋₆ alkynyl, each of which are optionally substituted with one or more substituents independently selected from halogen, —OR²² and —N(R²²)₂.

72. The compound or salt of claim 71, wherein 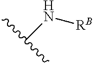
is selected from:

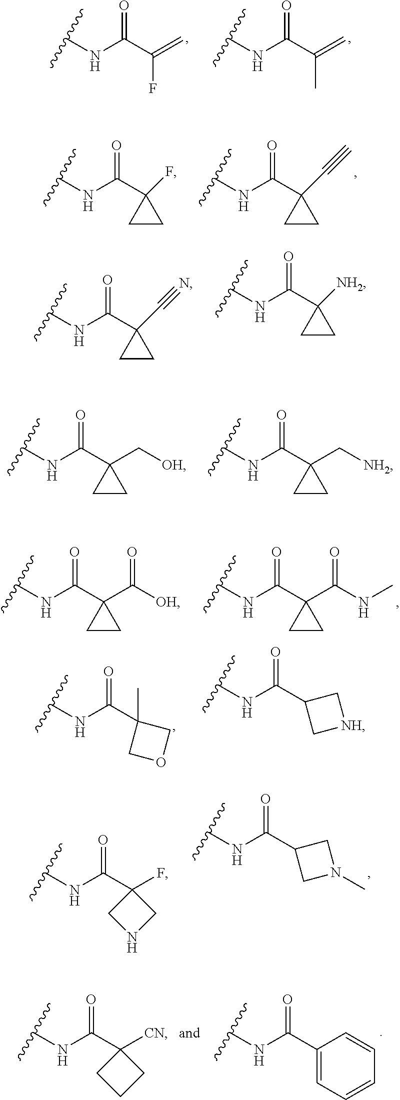

73. The compound or salt of claim 68, wherein 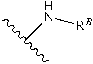
is selected from:

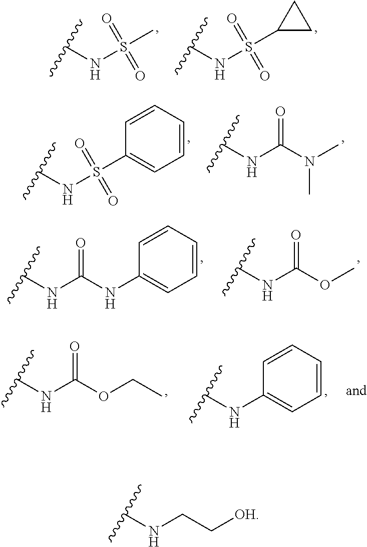

74. The compound or salt of claim 68, wherein 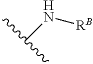
is selected from:

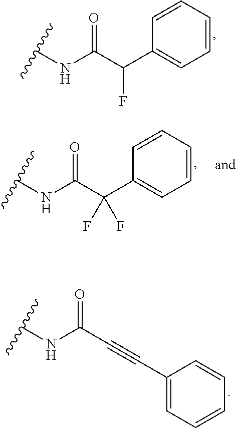 [SMILES: *NC(=O)C(F)c1ccccc1 ; *NC(=O)C(F)(F)c1ccccc1 ; *NC(=O)C#Cc1ccccc1]

75. The compound or salt of claim 67, wherein 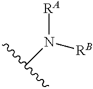
is selected from:

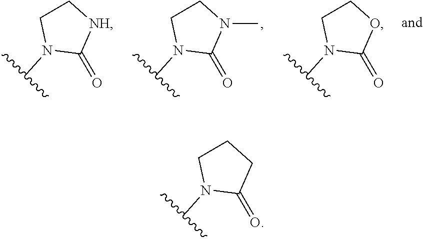

76. The compound or salt of claim 67, wherein 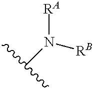
is selected from:

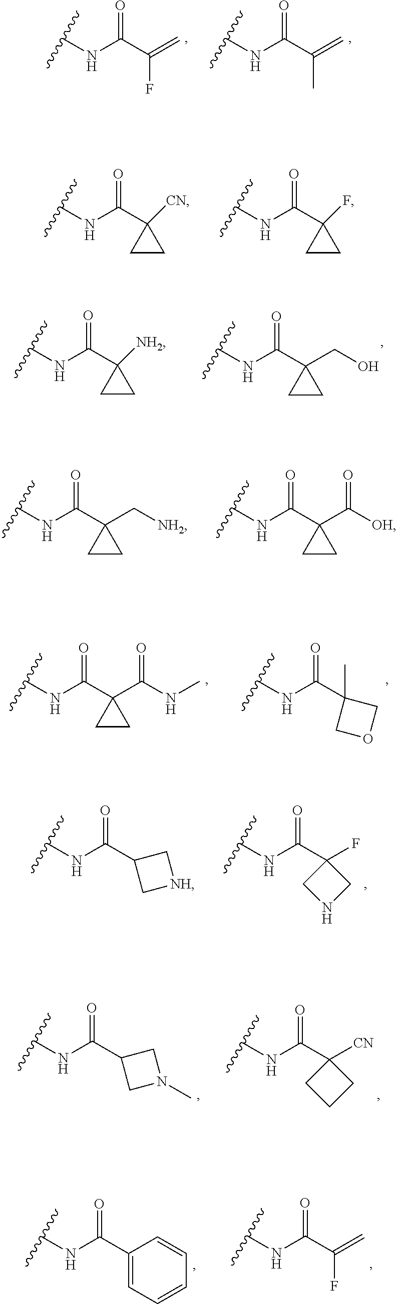

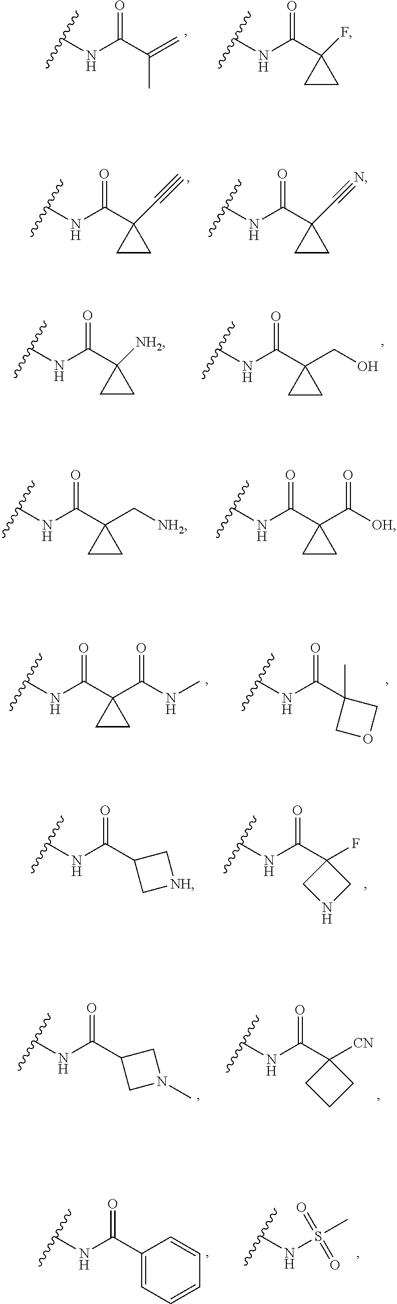

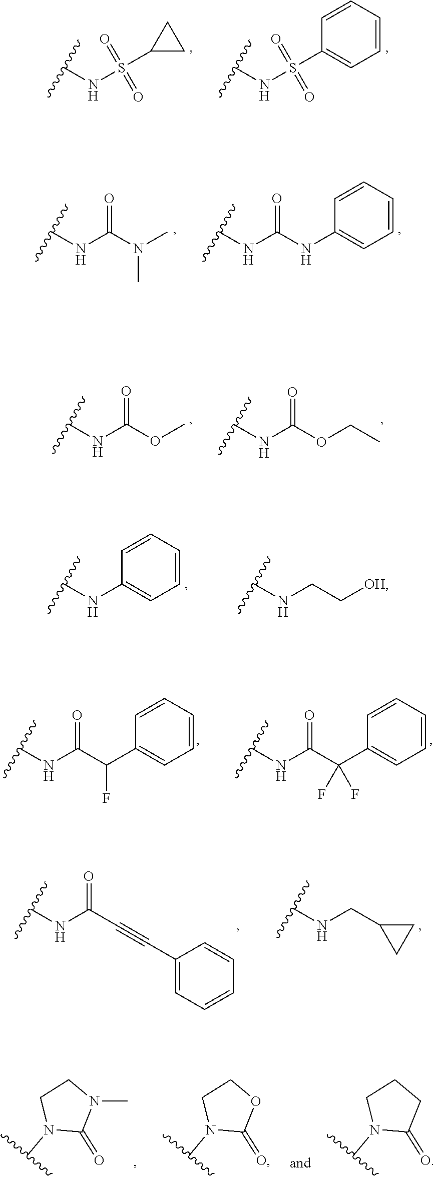

77. The compound or salt of claim 67, wherein R¹ is independently selected from halogen, —C(O)N(R⁶)₂, —CN, C₁₋₆ alkyl, and C₁₋₆ haloalkyl.

78. The compound or salt of claim 67, wherein R¹ is selected from chloro, fluoro, methyl, CHF₂, CF₃, —CN, and

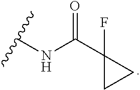 [SMILES: *NC(=O)C1(F)CC1]

79. The compound or salt of claim 67, wherein 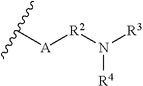
is selected from:

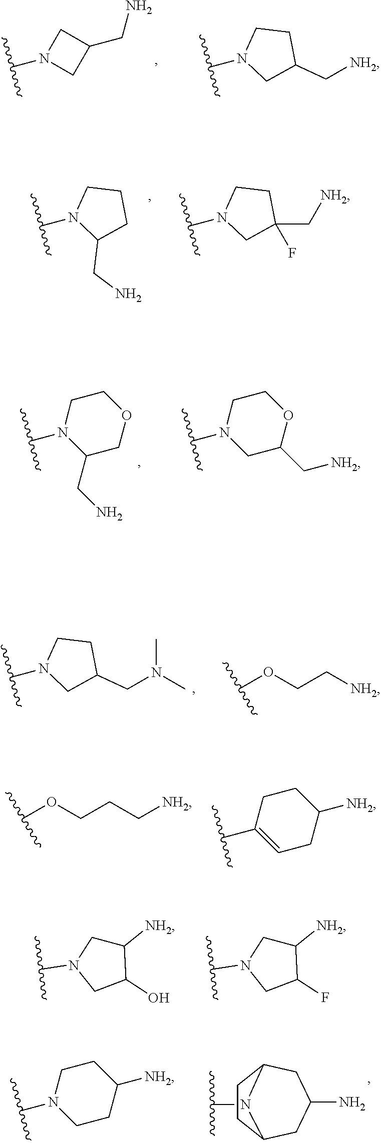

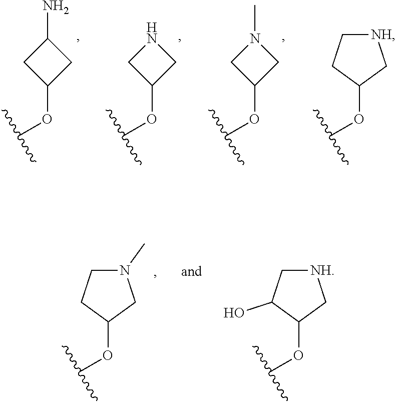

80. The compound or salt of claim 67, wherein 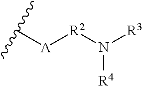
is selected from:

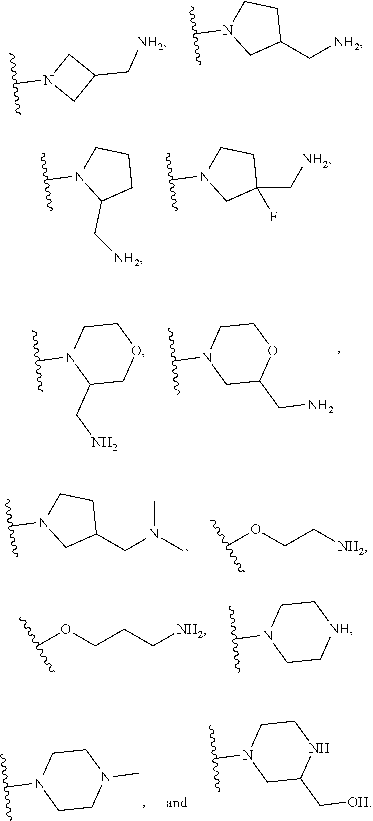

81. The compound or salt of claim 66, wherein R⁵ is selected from: C₁ alkyl substituted with one or more substituents selected from halogen, R^{C}, R^{D}, R¹⁰, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and C₂₋₆ alkyl, C₂₋₆ alkenyl, and C₂₋₆ alkynyl, each of which is optionally substituted with one or more substituents independently selected from halogen, R^{C}, R^{D}, R¹⁰, —OR¹⁰, —SR¹⁰, —N(R^{A})(R^{B}), —C(O)R¹⁰, C(O)OR¹⁰, —OC(O)R¹⁰, —OC(O)N(R¹⁰)₂, —C(O)N(R¹⁰)₂, —N(R¹⁰)C(O)R¹⁰, —N(R¹⁰)C(O)OR¹⁰, —N(R¹⁰)C(O)N(R¹⁰)₂, —N(R¹⁰)₂S(O)₂(R¹⁰), —S(O)R¹⁰—, —S(O)₂R¹⁰, —S(O)₂N(R¹⁰)₂, —NO₂, and CN; and 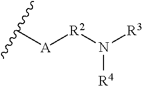
is selected from:

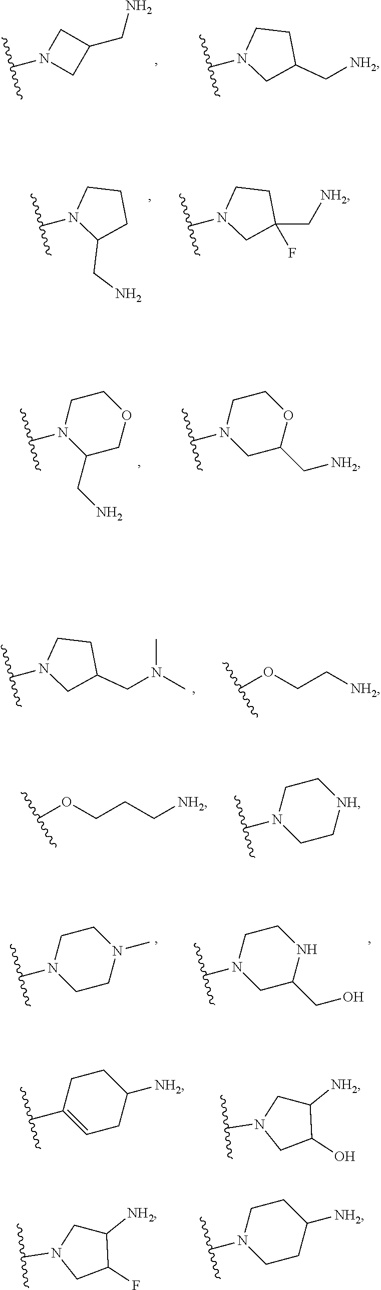

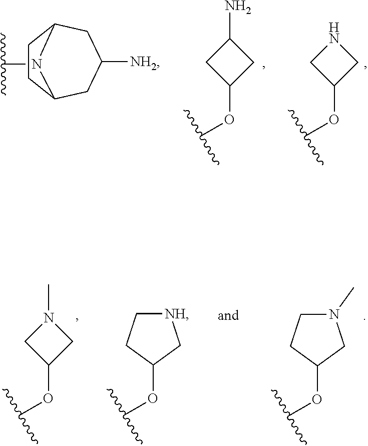

82. The compound or salt of claim 66, wherein the compound is selected from: 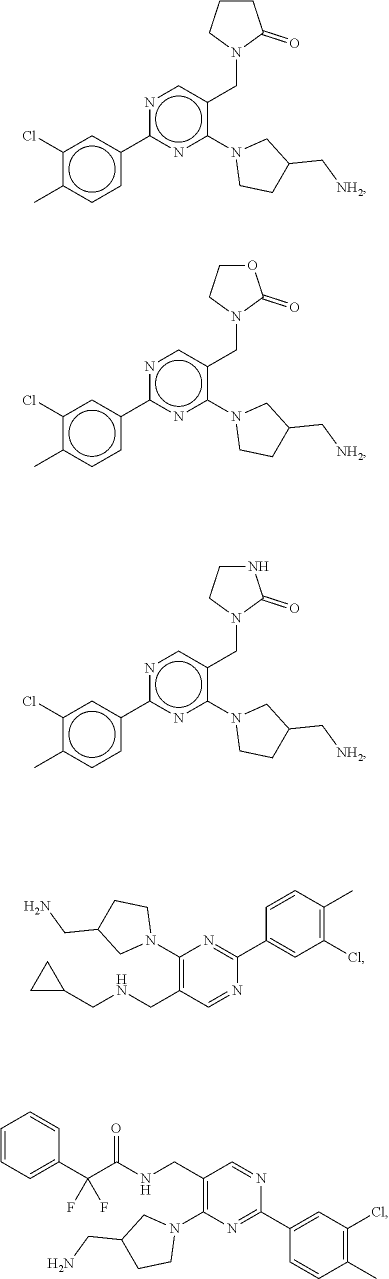 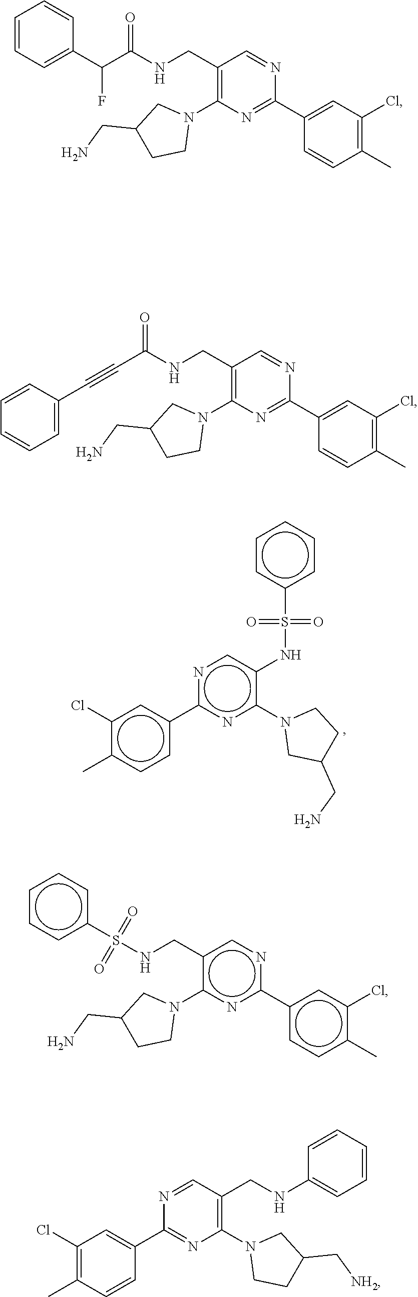 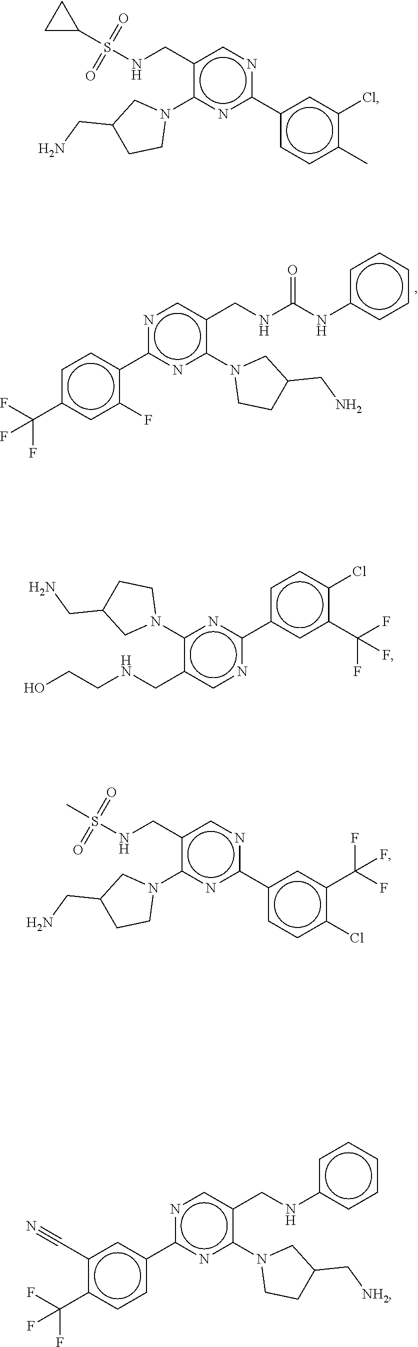 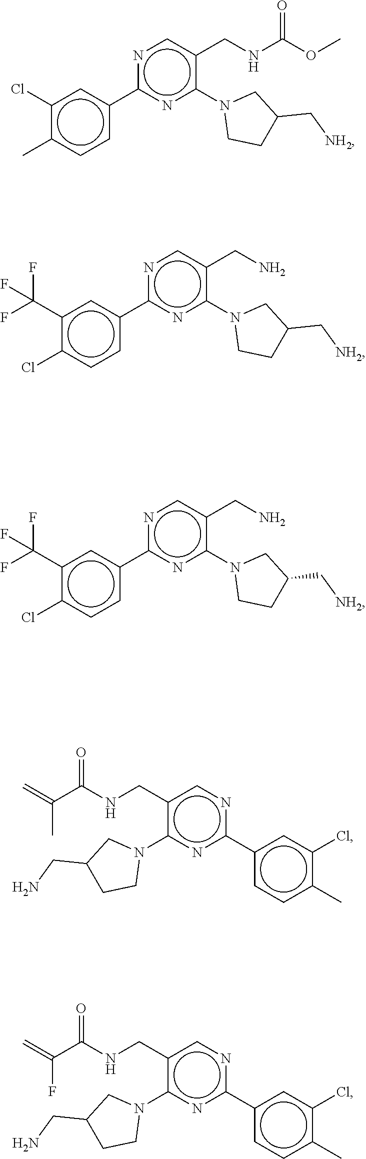 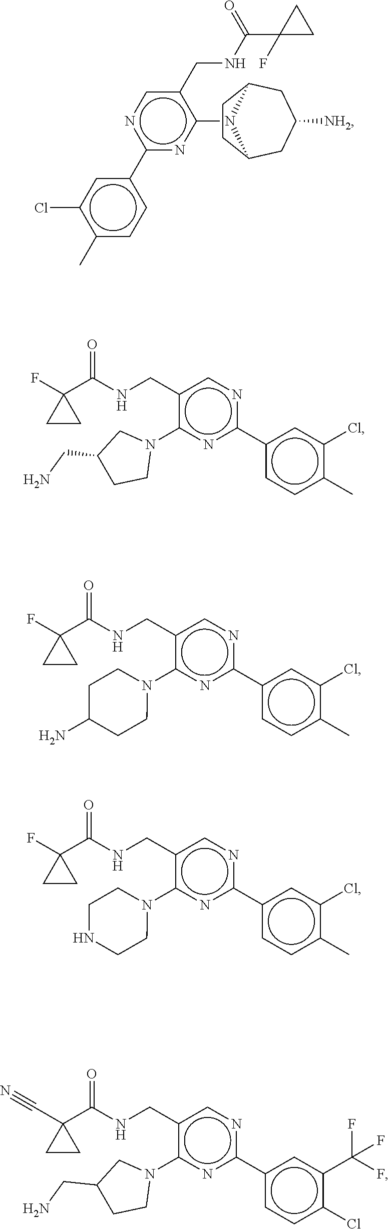 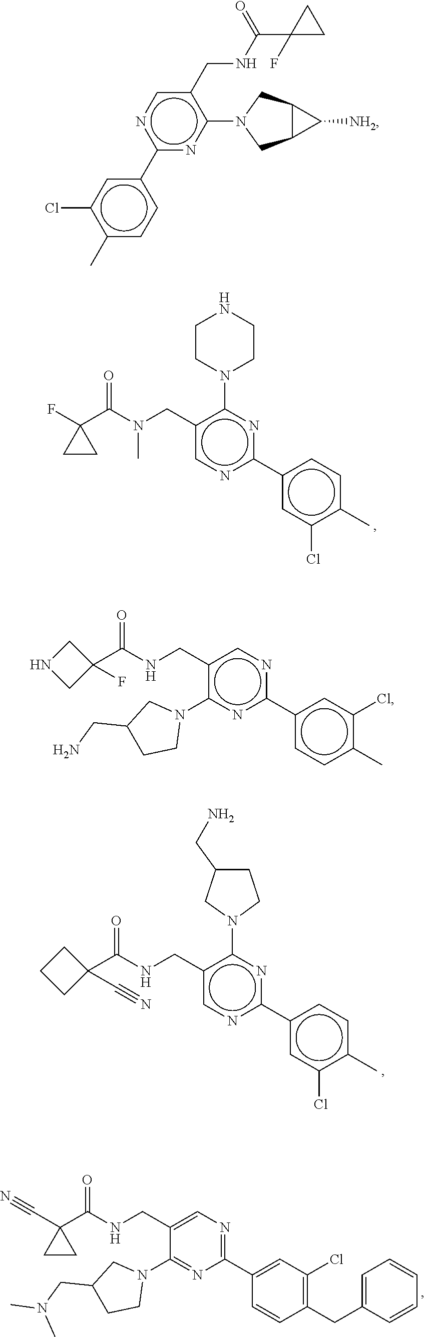 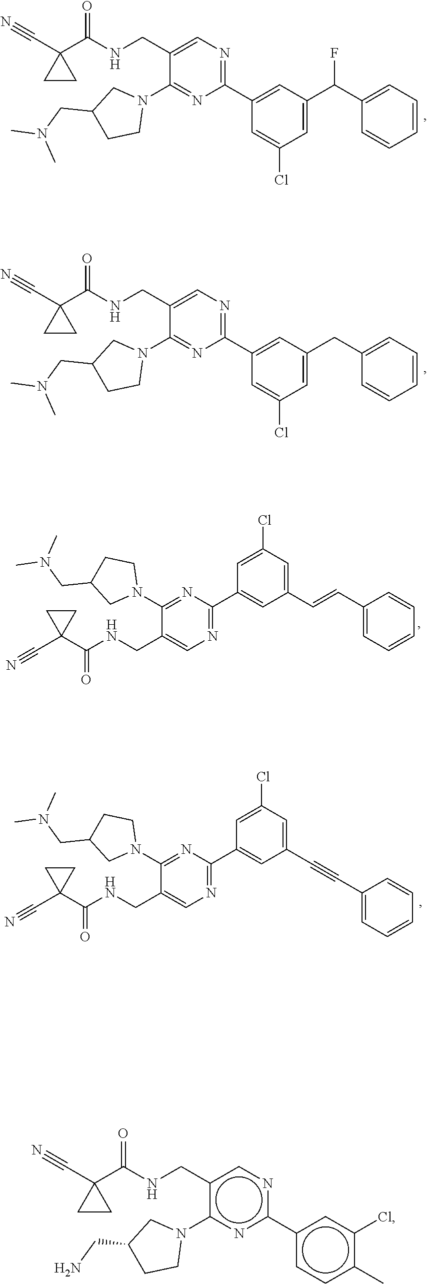 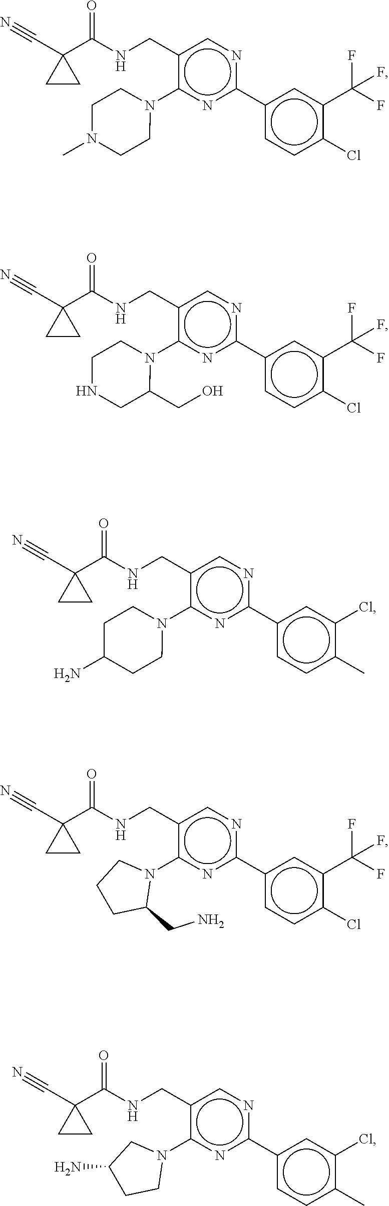 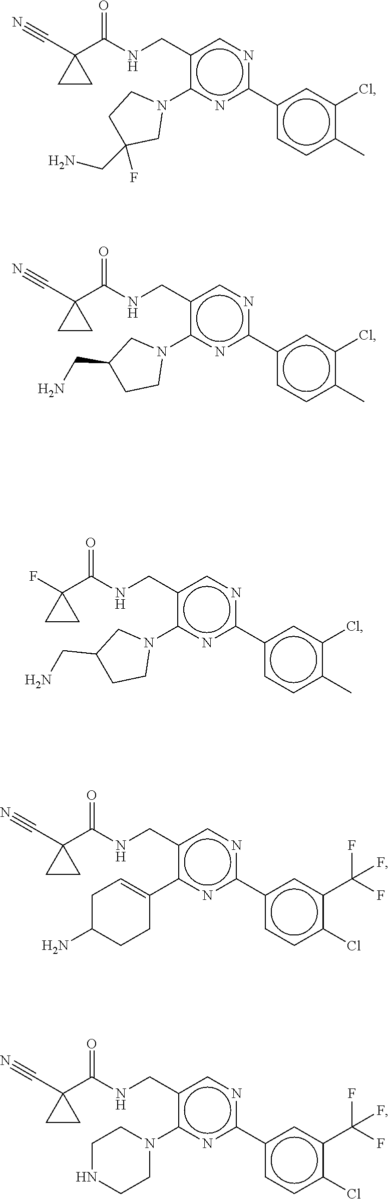 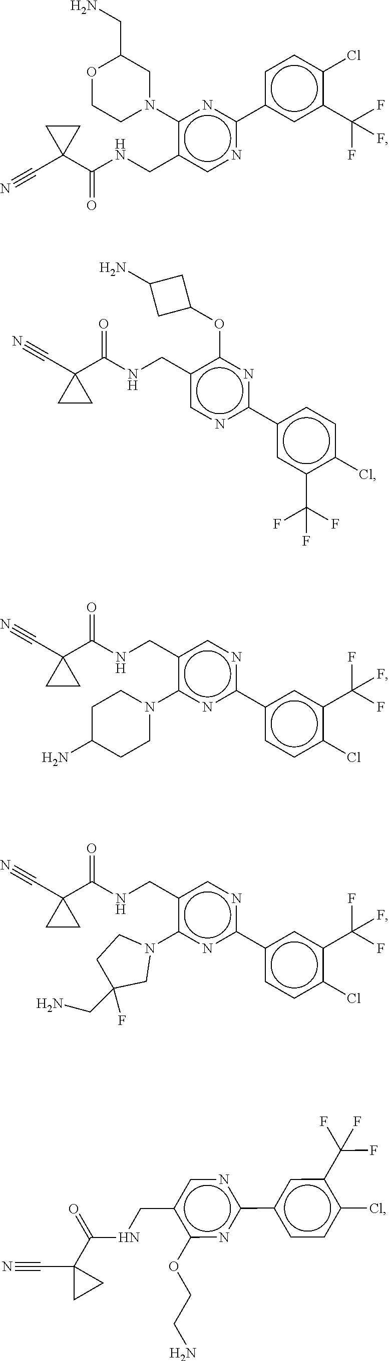 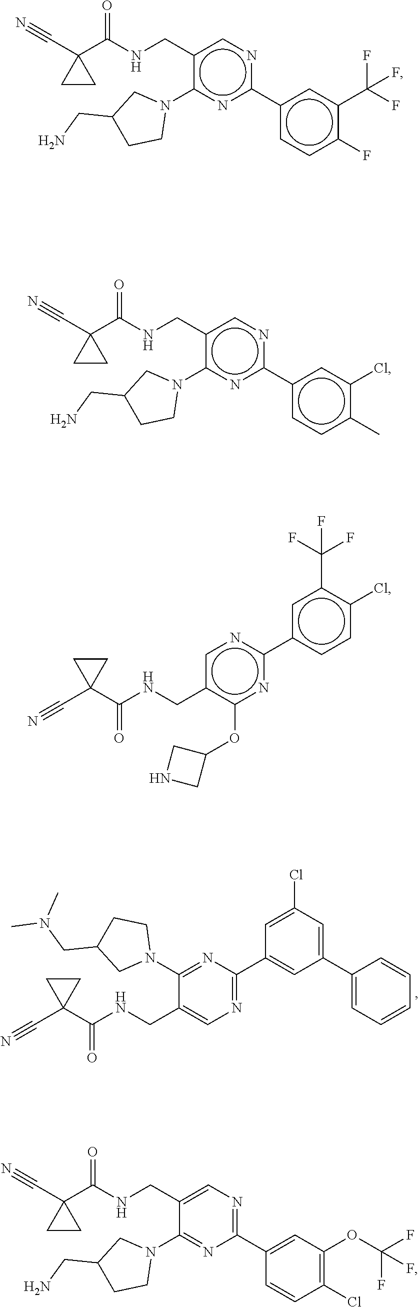 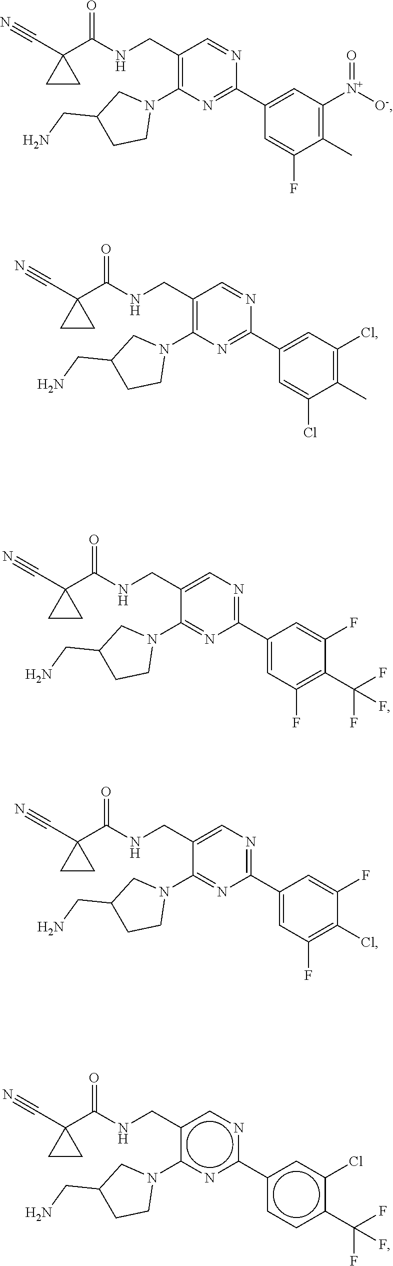 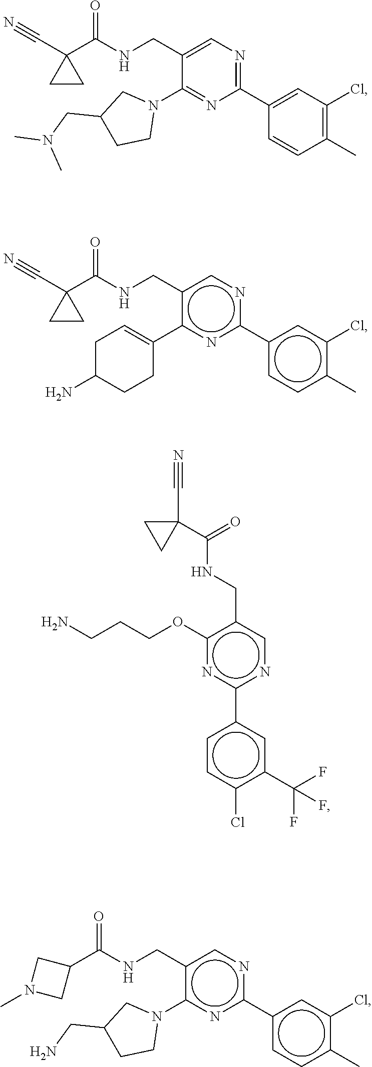 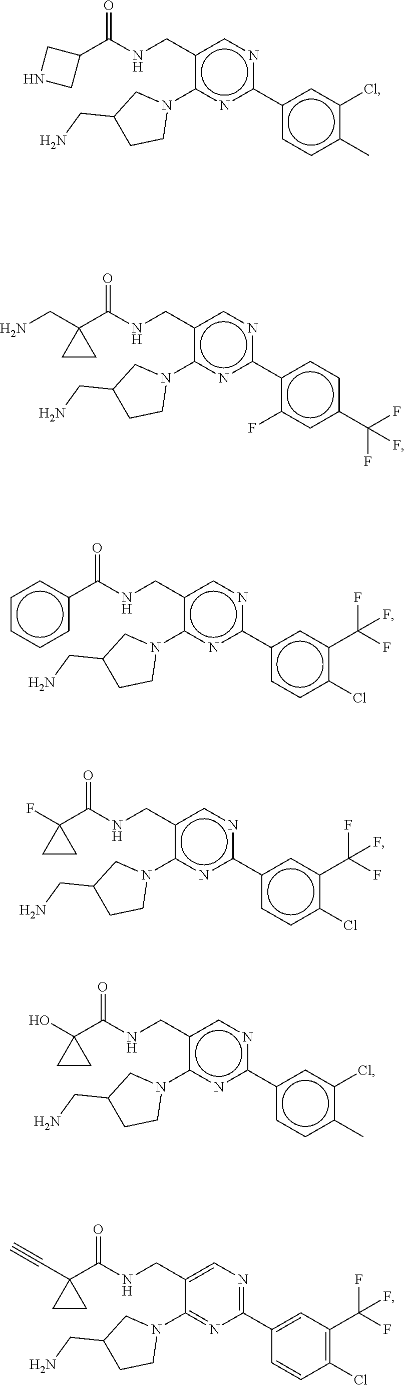
or a pharmaceutically acceptable salt of any one thereof.

83. The compound or salt of claim 66, wherein the compound is selected from: 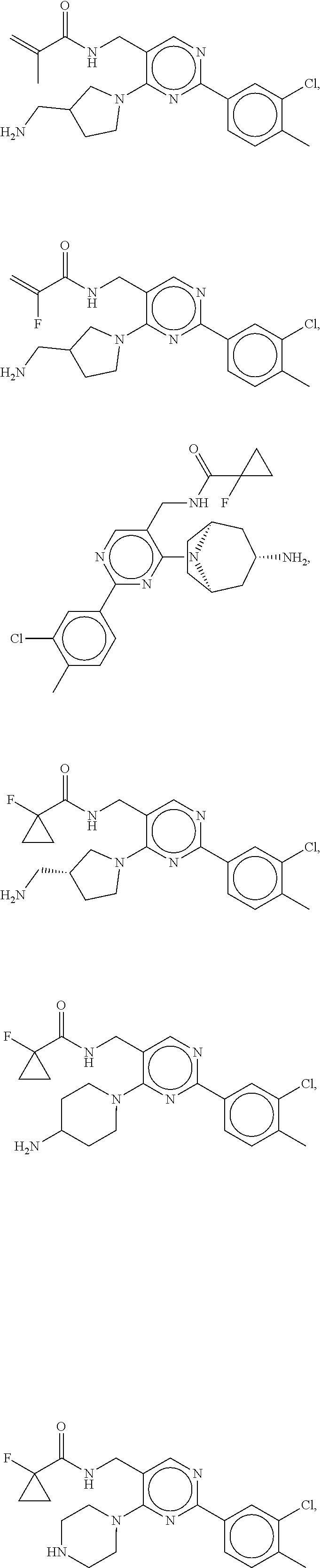 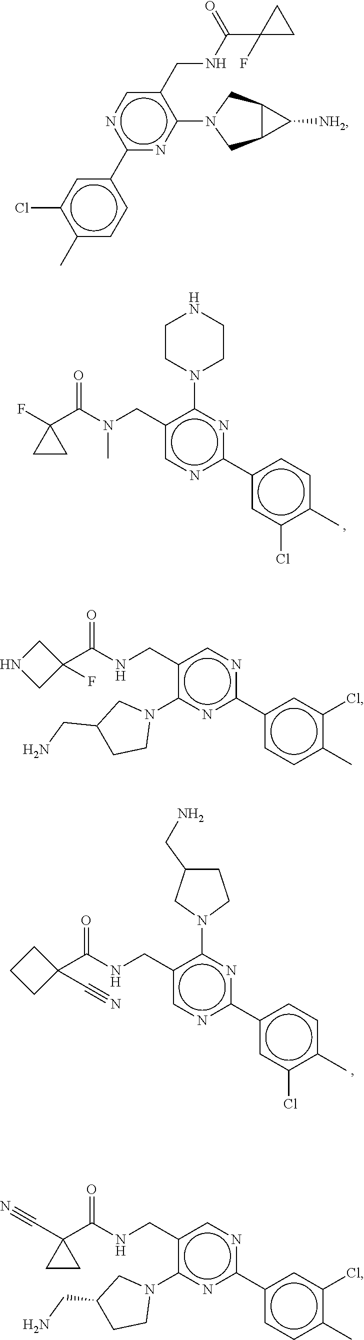 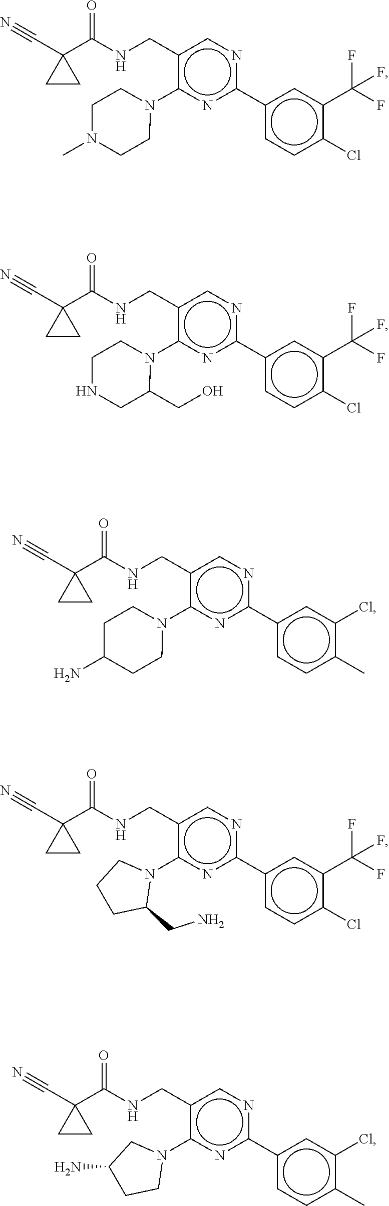 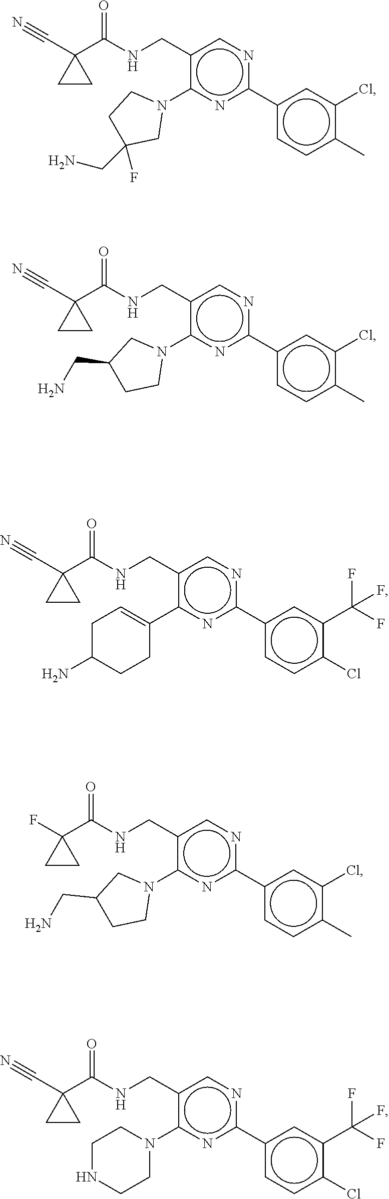 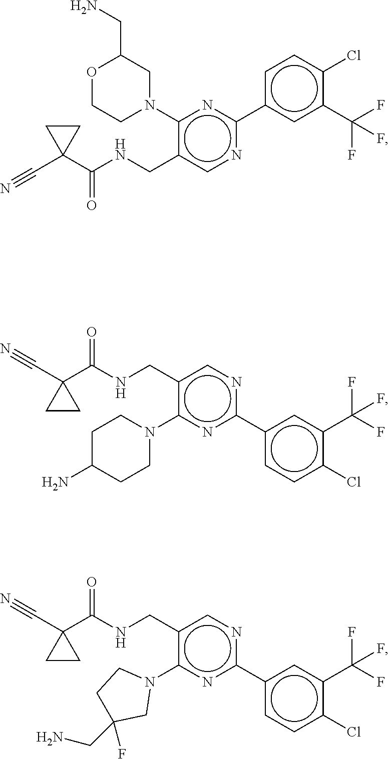 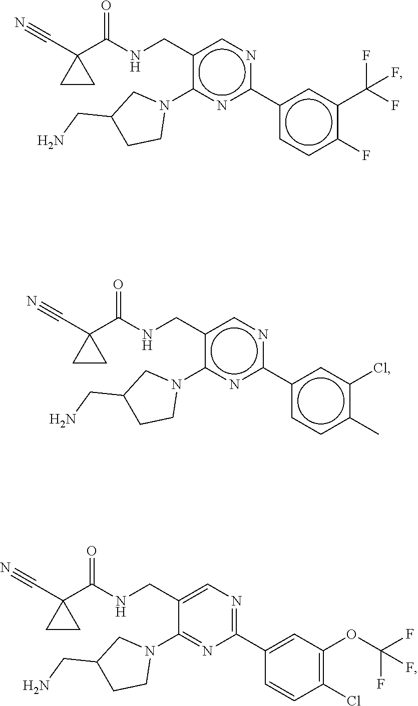 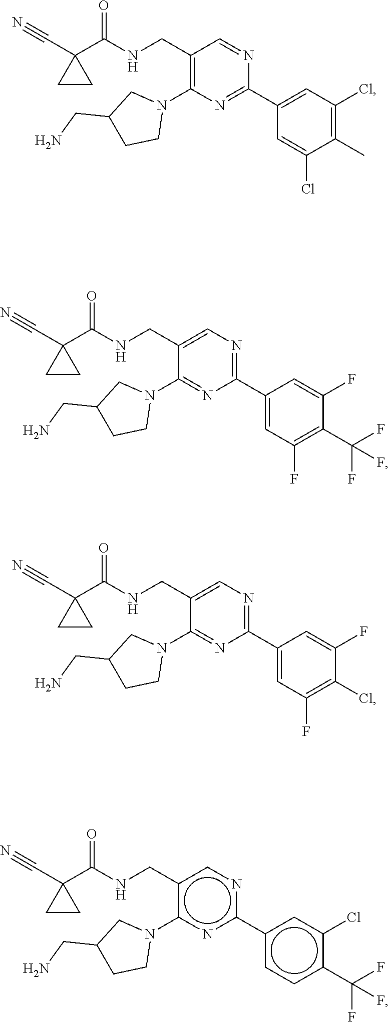 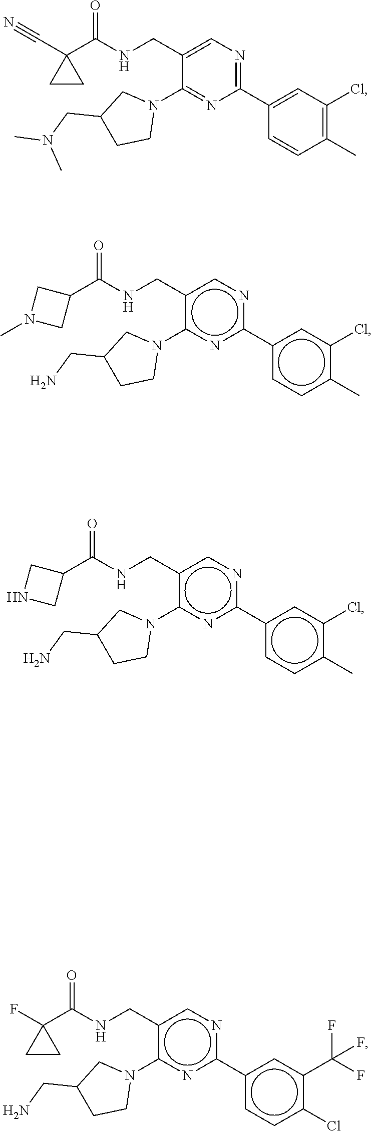
or a pharmaceutically acceptable salt of any one thereof.

84. A pharmaceutical composition comprising the compound or salt of claim 66 and a pharmaceutically acceptable excipient.

85. A method of treating cancer comprising administering to a subject in need thereof an effective amount of the pharmaceutical composition of claim 84.
